(19) United States
(12) Patent Application Publication
(10) Pub. No.: US 2026/0327621 A1
(43) Pub. Date: Oct. 22, 2026

(54) MODULATING STROMAL AND IMMUNE CELLS IN ATRIAL DISEASE

(71) Applicants: Arnab RUDRA, Brookline, MA (US); Daniel Griffith ANDERSON, Framingham, MA (US); The General Hospital Corporation, Boston, MA (US)

(72) Inventors: Maarten Hulsmans, Somerville, MA (US); Matthias Nahrendorf, Swampscott, MA (US); Kamila N. Nahrendorf, Swampscott, MA (US); Maximilian J. Schloss, Brookline, MA (US); I-Hsiu Lee, Brookline, MA (US); Noor Momin, Cambridge, MA (US); Daniel Griffith Anderson, Framingham, MA (US); Arnab Rudra, Brookline, MA (US)

(21) Appl. No.: 19/483,158

(22) PCT Filed: May 10, 2024
(86) PCT No.: PCT/US2024/028950

§ 371 Date: Nov. 10, 2025

Related U.S. Application Data
(60) Provisional application No. 63/501,286, filed on May 10, 2023.
(60) Provisional application No. 63/525,135, filed on Jul. 5, 2023.

(51) Int. Cl. C12N 15/113; A61K 47/68; A61P 9/06; C07K 16/28

(52) U.S. Cl. CPC C12N 15/1136; A61K 47/6807; A61K 47/6849; A61P 9/06; C07K 16/2803; C12N 2310/14; C12N 2310/3125; C12N 2310/314; C12N 2310/315; C12N 2310/321; C12N 2310/322; C12N 2310/3231; C12N 2310/3233; C12N 2310/332; C12N 2310/3513

(57) ABSTRACT

Provided are compositions comprising inhibitory nucleic acids targeting SPP1, optionally linked to an antibody that targets macrophages such as TREM2, and methods of use thereof to treat atrial disease, e.g., AFib.

### CLAIM OF PRIORITY

This application claims the benefit of U.S. Provisional Patent Application Ser. Nos. 63/501,286, filed May 10, 2023; and 63/525,135, filed Jul. 5, 2023. The entire contents of the foregoing applications are hereby incorporated by reference.

### FEDERALLY SPONSORED RESEARCH OR DEVELOPMENT

This invention was made with Government support under Grant Nos. HL 155097 and HL139598 awarded by the National Institutes of Health. The Government has certain rights in the invention.

### SEQUENCE LISTING

This application contains a Sequence Listing that has been submitted electronically as an XML file named “29539-0668WO1 SL ST26.XML.” The XML file, created on May 10, 2024, is 174,778 bytes in size. The material in the XML file is hereby incorporated by reference in its entirety.

### TECHNICAL FIELD

Provided are compositions comprising inhibitory nucleic acids targeting SPP1, while the inhibitory nucleic acids are optionally linked to an antibody that targets macrophages that express TREM2, and methods of use thereof to treat atrial disease, e.g., atrial fibrillation (AFib).

### BACKGROUND

In healthy atria, excitations arising in the sinus node coordinate atrial contractions that help fill the ventricles in diastole. Cardiomyocytes comprise 30% of atrial cells (see, e.g., M. Litvinukova et al., Cells of the adult human heart. Nature 588, 466-472 (2020)). The remaining 70% of atrial cells are fibroblasts, endothelial cells, and resident immune cells. Together, these provide cardiomyocytes with housekeeping functions including matrix scaffold, blood supply, waste removal, and immunosurveillance (see, e.g., J. A. Nicolás-Ávila, A. Hidalgo, I. Ballesteros, Specialized functions of resident macrophages in brain and heart; and J Leukoc Biol 104, 743-756 (2018); and E. Forte, M. B. Furtado, N. Rosenthal, The interstitium in cardiac repair: role of the immune-stromal cell interplay. Nat Rev Cardiol 15, 601-616 (2018)). Atrial fibrillation (AFib) is the most common arrhythmia (see, e.g., E. J. Benjamin et al., Heart Disease and Stroke Statistics-2019 Update: A Report From the American Heart Association. Circulation 139, e56-e528 (2019)). During AFib, the lack of coordinated atrial excitation and contraction slows blood flow into the ventricles. This sluggishness can diminish cardiac performance and give rise to atrial blood clots that cause thromboembolic stroke. AFib and the risk of its lethal complications are worsened by atrial fibrosis.

### SUMMARY

Provided herein are compositions comprising inhibitory nucleic acids (e.g., siRNA agents) targeting SPP1, optionally linked to an antibody that targets macrophages especially those expressing TREM2, and methods of use thereof to treat atrial disease, e.g., AFib, as described herein.

Also provided herein are siRNA sequences that can be carried by antibodies and/or nanoparticle compositions targeting a unique subset of cardiac macrophages involved in atrial remodeling and associated conduction disorders. Also provided herein is the use of antibody and/or nanoparticle composition carrying siRNAs targeting a specific subset of macrophages for the treatment of atrial remodeling and associated conduction disorders. Cell and gene targets have been identified in human samples and have been validated using appropriate mouse models. Unique siRNA sequences with or without cross-reactivity between human and mouse gene target have been developed and validated in vitro and in vivo.

Accordingly, in one aspect, provided herein are compositions comprising a double-stranded RNA (dsRNA) agent targeting human secreted phosphoprotein 1 (SPP1), wherein the dsRNA agent comprises a sense strand and an antisense strand forming a double stranded region, wherein the antisense strand comprises a nucleotide sequence comprising at least 15 contiguous nucleotides, with 0, 1, 2, or 3 mismatches, from one of the antisense sequences listed in Table 3 or 4 and wherein the sense strand comprises a nucleotide sequence comprising at least 15 contiguous nucleotides, with 0, 1, 2, or 3 mismatches, from a sense sequence listed in Table 2 or Table 3 that corresponds to the antisense sequence.

In some embodiments, the dsRNA agent targets a sequence selected from any one of SEQ ID NOs: 1-24.

In some embodiments, the sense strand comprises a nucleotide sequence selected from any one of SEQ ID NOs: 25-48, 73-96 and 121-144.

In some embodiments, the antisense strand comprises a nucleotide sequence selected from any one of SEQ ID NOs: 49-72, 97-120, 145-168.

In some embodiments, the dsRNA agent comprises at least one modified nucleotide.

In some embodiments, no more than five of the sense strand nucleotides and not more than five of the nucleotides of the antisense strand are unmodified nucleotides.

In some embodiment, all of the nucleotides of the sense strand and all of the nucleotides of the antisense strand comprise a modification.

In some embodiments, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide; and combinations thereof.

In some embodiments, the sense strand comprises a nucleotide sequence selected from any one of SEQ ID NOs: 25-48.

In some embodiments, the antisense strand comprises a nucleotide sequence selected from any one of SEQ ID NOs: 49-72.

In some embodiments, the sense strand comprises a nucleotide sequence of SEQ ID NO: 74 and antisense strand comprises a nucleotide sequence of SEQ ID NO: 98.

In some embodiments, the sense strand comprises a nucleotide sequence of SEQ ID NO: 122 and antisense strand comprises a nucleotide sequence of SEQ ID NO: 146.

In some embodiments, the sense strand comprises a nucleotide sequence of SEQ ID NO: 26 and antisense strand comprises a nucleotide sequence of SEQ ID NO: 50.

In some embodiments, the sense strand comprises a nucleotide sequence of SEQ ID NO: 75 and antisense strand comprises a nucleotide sequence of SEQ ID NO: 99.

In some embodiments, the sense strand comprises a nucleotide sequence of SEQ ID NO: 123 and antisense strand comprises a nucleotide sequence of SEQ ID NO: 147.

In some embodiments, the sense strand comprises a nucleotide sequence of SEQ ID NO: 27 and antisense strand comprises a nucleotide sequence of SEQ ID NO: 51.

In some embodiments, at least one strand comprises a 3′ overhang of at least 2 nucleotides.

In some embodiments, the double stranded region is 15-30 nucleotide pairs in length.

In some embodiments, the double stranded region is 17-23 nucleotide pairs in length.

In some embodiments, each strand is about 19-30 nucleotides in length.

In one aspect, provided herein are pharmaceutical compositions comprising any one of the compositions described herein and a pharmaceutically acceptable carrier.

In some embodiments, the dsRNA agent is conjugated with an antibody that binds to TREM2.

In some embodiments, the inhibitory nucleic acid molecule is conjugated to the antibody via a linker.

In one aspect, provided herein are methods for treating or reducing the risk of progression of development or recurrence of an atrial disease, comprising administering any one of the compositions described herein, or any one of the pharmaceutical compositions described herein.

In some embodiments, the atrial disease is an osteopontin-associated disease.

In some embodiments, the atrial disease is atrial fibrillation (AFib).

In some embodiments, the composition or pharmaceutical composition is administered via intravenous injection.

In some embodiments, the subject is a human subject.

Also provided herein are compositions or pharmaceutical composition described herein, for use in a method for treating or reducing the risk of progression of development or recurrence of an atrial disease.

In some embodiments, the atrial disease is an osteopontin-associated disease.

In some embodiments, the atrial disease is atrial fibrillation (AFib).

In some embodiments, the composition or pharmaceutical composition is administered via intravenous injection.

In some embodiments, the subject is a human subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1L show the single-cell atlas of human atrial disease. (A) scRNA-seq was performed in five controls and seven AFib patients. (B) Uniform manifold approximation and projection (UMAP) delineates six major cardiac cell types. (C) Distribution of cell populations. Color code corresponds to (B). (D) Gene ontology biological process (GOBP) gene sets (48, 49) up- or downregulated in AFib from GSEA of MP/DC cluster. Circle size denotes number of enriched genes. (E) Gene expression in MP/DC cluster represented by z-scores of the log-transformed normalized counts. Rows, samples grouped by disease state; columns, genes in the three gene modules significantly associated with disease state; gene coexpression modules and exemplary genes are labeled. (F) Select MSigDB C2 gene sets (50) overrepresented in module 1 described in (E). (G) Genes in MP/DC cluster ranked according to log₂-fold changes between seven AFib and five control patients. (H) Subclustering of 10,555 MP/DCs from five controls and seven AFib patients. (I) Difference in abundance of cells belonging to MP/DC subclusters between seven AFib and five control patients. Pie size indicates log₂-fold changes; **P<0.01. (J) Fraction of SPPI⁺ macrophages in control and AFib patients. Bar graph shows mean±SEM, n=5-6 per group, two-tailed Student's t test. (K) Gene expression levels (represented by log-transformed normalized counts) across macrophage clusters; Kruskal-Wallis (P<2.2×10⁻¹⁶) followed by two-tailed Wilcoxon rank sum test; ****adjusted P=2×10⁻¹⁶. (L) SPP1 expression in MP/DCs from control and AFib patients.

FIGS. 2A-2P show the single-cell atlas of mouse atria. (A) HOMER mice are exposed to mitral valve regurgitation (MR), high-fat diet (HFD), and angiotensin 2 (Ang2). (B) Doppler of MR. (C) Left, MRI of left atrium (LA), scale bars represent 3 mm; right, LA end-systolic volume, n=6-10 per group from two independent experiments, two-tailed Mann-Whitney test. (D) AFib inducibility and burden. n=8-12 per group from two independent experiments; left, two-sided Fisher's exact test; right, two-tailed Mann-Whitney test. Other parameters are shown in FIG. 48. (E) scRNA-seq was performed in four sham and four HOMER mice. (F) Left, UMAP; right, cell population distributions. (G) Log₂-fold changes of cell abundance in mouse and human atria. (H) Flow cytometry of sham and HOMER atria. n=21 per group from five independent experiments, two-tailed Student's t test. (I) Scatterplot showing C5 GOBP GSEA species concordance. GSEA is performed on lists of genes ranked according to differential expression in the disease state. −log₁₀ (FDR) is plotted for mouse (x-axis) and human (y-axis). −log₁₀ (FDR) are negative if gene set is downregulated. High and low gene set density are denoted. (J) Genes in MP/DC cluster ranked according to log₂-fold changes between four HOMER and four sham mice. y-axis was broken at −5 for displaying top downregulated genes. (K) Subclustering of MP/DCs from four sham and four HOMER mice. (L) Atrial Spp1⁺ macrophages by scRNA-seq. n=4 per group, two-tailed Student's t test. (M) Gene expression levels (represented by log-transformed normalized counts) across macrophage clusters. (N) Spp1 expression in MP/DCs from sham and HOMER mice. (O) Spp1⁺ subpopulations in sham and HOMER mice. n=4 per group, two-tailed Student's t test. (P) Flow cytometry of atrial macrophages in sham and HOMER mice. n=6-7 per group from two independent experiments, two-tailed Student's t test. All bar graph data are mean±SEM with individual values for data distribution.

FIGS. 3A-3E show that CCR2-dependent monocyte recruitment contributes to left atrial macrophage expansion and atrial disease. (A) Flow cytometric quantification of recruited (YFP⁺Td⁻) and locally sourced (YFP⁺Td⁺) left atrial macrophages in Cx3cr1^{CreER/+}Ai9^{fl/+} sham and HOMER mice. n=8-12 per group from two independent experiments, two-tailed Student's t test. (B) Relative Spp1 expression levels by qPCR in recruited (YFP⁺Td⁻) and locally sourced (YFP⁺Td⁺) left atrial macrophages in Cx3cr1^{CreER/+}Ai9^{fl/+} HOMER mice. n=8 per group from two independent experiments, two-tailed Wilcoxon matched-pairs signed rank test. (C) Flow cytometric quantification of myeloid cell populations in left atrial tissues from C57BL/6 and Ccr2^{-/-} HOMER mice. Macrophages: 21 C57BL/6 and 19 Ccr2^{-/-} HOMER mice from five independent experiments; monocytes and neutrophils: 12 C57BL/6 and 10 Ccr2^{-/-} HOMER mice from three independent experiments; two-tailed Student's t test. (D) Flow cytometric quantification of TREM2+CD9⁺ left atrial macrophages in C57BL/6 and Ccr2^{-/-} HOMER mice. n=7-9 per group from two independent experiments, two-tailed Student's t test. (E) AFib inducibility and burden in C57BL/6 and Ccr2^{-/-} HOMER mice by EP study. n=8-9 per group from two independent experiments; left, two-sided Fisher's exact test; right, two-tailed Mann-Whitney test. Other EP parameters are shown in FIG. 49. All bar graph data are mean±SEM with individual values for data distribution.

FIGS. 4A-4E show that macrophage-derived SPP1 promotes atrial disease. (A) EP study of AFib inducibility and burden in C57BL/6 BMT HOMER and Spp1^{-/-} BMT HOMER mice. n=13 per group from three independent experiments; left, two-sided Fisher's exact test; right, two-tailed Mann-Whitney test. Other EP parameters are shown in FIG. 50. (B) Heatmap of SPP1-receptor interactions between SPP1⁺ macrophages (mac (e) and mac (g)) and other non-cardiomyocytes (non-CM) in four HOMER mice. SPP1 and cognate receptors are shown on the x-axis. Cell populations that express Spp1 and the receptor are shown on the y-axis and are color-coded according to the major cardiac cell types identified in FIG. 2F. Scale bar denotes average Spp1 and receptor expression levels in their respective interacting subpopulations (represented by mean normalized counts) if the enrichment of an interacting SPP1-receptor pair in the given interacting subpopulations was statistically significant (P<0.05). Non-significant interactions were assigned a value of 0. (C) Representative immunofluorescent staining of macrophages (CD68), collagen deposition (COL1), and nuclei (DAPI) and histological staining of fibrosis (Masson's trichrome staining) in left atrial tissue from C57BL/6 BMT HOMER and Spp1^{-/-} BMT HOMER mice. Scale bars represent 50 μm. (D) Quantification of CD68⁺, COL1⁺, and fibrotic area in left atrial tissue from C57BL/6 BMT HOMER and Spp1^{-/-} BMT HOMER mice. Bar graphs show percentage of positive staining per field of view (FOV); n=5 per group from two independent experiments, two-tailed Student's t test. (E) Scatterplot showing concordance between C5 GOBP GSEA results in human and mouse fibroblasts in AFib/HOMER (as in FIG. 2I). Inset shows detailed GSEA results for gene set “GOBP collagen fibril organization” in human and mouse fibroblast clusters. All bar graph data are mean±SEM with individual values for data distribution.

FIGS. 5A-5D show dual dose analysis of siRNA candidates. Spp1-silencing siRNA candidate sequences and their modifications are detailed in FIG. 5B. The dual-dose silencing potency of each Spp1-targeting siRNA in Hepa1-6 cells is listed on the right side of the table. Briefly, Hepa1-6 cells were seeded at a density of 20,000 cells per well in a 96-well tissue culture plate and transfected with a mixture of lipofectamine 2000 with siRNA candidates at either a 20 or 2 nM concentration for 24 hours at 37 C and 5% CO₂ in a humidified incubator. Cells were lysed and mRNA encoding Gapdh and Spp1 were quantified using a branched DNA assay to monitor the on-target Spp1 mRNA silencing relative to the Gapdh mRNA levels. The specific potency of each siRNA candidate in silencing Spp1 relative to Gapdh was quantified (left graphs and enumerated in the table). The normalized expression of Gapdh remained static indicating none of the candidates tested have off-target silencing of Gapdh (right graphs and enumerated on the table). (FIG. 5A is an enlarged version of FIGS. 5C-5D). In FIG. 5B, predicted siRNA sequences directed against mouse Spp1 (mmSpp1). Gray boxes denote siRNA candidates with predicted cross-reactivity to human SPP1. In FIGS. 5C-5D, the effect of each candidate siRNA in Hepa1-6 cells at 20 nM (b) and 2 nM (c) concentrations on target mmSpp1 expression normalized to mmGapdh expression (top) and absolute mmGapdh expression normalized to mock-transfected cells (bottom). n=4 to 8 biological replicates per candidate.

FIGS. 6A-6C show the dose response of siRNA candidates. The most potent Spp1-silencing siRNA candidates from the initial dual dose study were candidates 110, 111, 487, 814, 943, and 1050. Again, Hepa1-6 celles were seeded at a density of 20,000 cells per well in a 96-well tissue culture plate and transfected with a mixture of lipofectamine 2000 with siRNA candidates at several concentration for 24 hours at 37 C and 5% CO₂ in a humidified incubator. Cells were lysed and mRNA encoding Gapdh and Spp1 were quantified using a branched DNA assay to monitor the on-target Spp1 mRNA silencing relative to the Gapdh mRNA levels. The specific potency of each siRNA candidate in silencing Spp1 relative to Gapdh was quantified (left graphs). The normalized expression of Gapdh remained static indicating none of the candidates tested have off-target silencing of Gapdh (right graphs). In FIGS. 6A-6B, the effect of siRNA candidates 110, 111, 487, 814, 943, and 1050 in Hepa1-5 cells at different doses on target mmSpp1 expression normalized to mmGapdh expression (a) and absolute mmGapdh expression normalized to mock-transfected cells (b). n=4 biological replicates per dose per candidate. In FIG. 6C, the silencing potency (IC50) and maximum inhibition (max. inhib.) of top siRNA candidates generated from a sigmoidal dose-response curve fit.

FIGS. 7A-7D show the TREM2 expression on cells in the heart. (A), Experimental outline of C57BL/6 mice infused with saline or Ang2 via minipumps for 28 days. (B), Representative flow cytometry gating strategies to identify cell subsets in heart tissue from saline- and Ang2-treated mice. (C), Enumeration of MHCII^{hi} and MHCII^{lo} macrophages in hearts from saline- and Ang2-treated mice by flow cytometry. n=8 to 12 animals per group from two independent experiments, two-tailed unpaired t-test. (D), Flow cytometry quantification of TREM2 on lymphocytes, neutrophils, MHCII^{lo} macrophages, MHCII^{hi} macrophages, endothelial cells, and fibroblasts from hearts of saline- and Ang2-treated mice.

FIGS. 8A-8B show that ARC silencing half-life is 6 days. (A), Experimental outline of C57BL/6 mice infused with Ang2 via minipumps for 28 days followed by treatment with vehicle or anti-TREM2-siSpp1 at 1.5 mg siRNA/kg body weight total dose equally divided between days 6 and 9. Hearts were excised either on day 12 or day 15. (B), Spp1 expression in TREM2^{hi} (left) and TREM2^{lo} (right) cardiac macrophages isolated on day 12 or 15 from anti-TREM2-siSpp1-treated hypertensive mice normalized to vehicle-treated hypertensive mice.

FIGS. 9A-9B show the annotating mouse left atrial nuclei. (A), Clustering of 48,647 nuclei from left atrial tissues of three vehicle- and three anti-Trem2-Spp1-treated HOMER mice. (B), Dot plot annotating nuclei clusters by signature gene expression. z-score of average gene expression is denoted; circle size indicates percentage of cells expressing the gene.

FIGS. 10A-10C show the results of isolating and annotating human left atrial non-cardiomyocytes. (A) Representative plots of the flow cytometry gating strategy for isolating single, viable non-cardiomyocytes (non-CM) from human left atrial appendages. VDR, vybrant dyecycle ruby. (B) Clustering of 41,609 non-CM from five controls and seven AFib left atrial appendages. (C) Dot plot annotating non-CM clusters by signature gene expression. z-score of average gene expression is denoted; circle size indicates percentage of cells expressing the gene. Mural cells collectively refer to smooth muscle cells and pericytes of the vasculature.

FIGS. 11A-11E show the detailed description of human scRNA-seq data. (A) UMAP plots of individual patients delineating six major cardiac cell types. (B) Fraction of cells belonging to the different cell types for each individual patient. (C) Proportion of cells originating from each patient for all six cardiac cell types. (D) Average number of differentially expressed genes (DEGs, adjusted P-value <0.01 based on Bonferroni correction) for all six cell types by pairwise comparison of the different control samples. The bar on the right indicates the average number of DEGs by pairwise comparison of the fibroblast and MP/DC cluster for all control samples. Each dot represents the number of DEGs between a pair of samples. The differential gene expression analysis was performed with Seurat's FindMarkers function. (E) Same analysis as in (D) but for AFib patients. All bar graph data are mean±SEM with individual values for data distribution.

FIGS. 12A-12C show the transcriptional changes in non-cardiomyocyte clusters in human AFib. (A) Heatmaps of the 10 most up- and the 10 most downregulated genes in each cell type, comparing AFib patients with controls. Only genes with unadjusted P-values <0.05 were included; each column represents a patient; color denotes z-scores of the log-transformed normalized counts for each individual patient. Panel further shows all genes in volcano plots for the same cells in all patients, highlighting the top five upregulated (downregulated) genes in AFib with the highest log₂-fold change and P-value <0.05. (B) Number of DEGs in AFib patients versus control cases, showing genes with unadjusted P-values <0.05 (upper panel) and genes with a log₂-fold change >2 (lower panel) for each respective cell population. (C) Significant gene ontology biological process (GOBP) gene sets up- or downregulated in AFib patients from GSEA of lymphocyte, fibroblast, endothelial and mural cell clusters (a similar analysis for MP/DCs is shown in FIG. 1D). Circle size denotes number of genes in the core enrichment set that belong to a given GOBP gene set.

FIG. 13 shows the ischemia and vascular remodeling in diseased left atrial tissue. GSEA results of endothelia (left) and mural cells (right) in five controls versus seven AFib patients. NES, normalized enrichment score.

FIGS. 14A-14C show the resolution selection maximizing transcriptional changes between macrophage subsets. (A) UMAP plots at indicated resolutions. (B) Average number of significant DEGs (with adjusted P-values <0.01) between pairs of macrophage subclusters for resolutions ranging from 0.2 to 0.7. Bar graph data are mean±SEM with individual values for data distribution.
(C) Dot plot identifying the MP/DC clusters by signature gene expression. Color denotes z-score of average gene expression; circle size indicates percentage of cells expressing the gene.

FIG. 15 shows the transcriptional signatures of the human macrophage clusters. Heatmap depicting the 10 most upregulated genes in each of the five macrophage clusters. These DEGs were obtained with Seurat's FindAllMarkers function comparing a subcluster against all other macrophages. The rows show the 10 genes with the highest fold changes for each cluster; each column represents a cell.

FIGS. 16A-16B show that SPP1 expression in human non-cardiomyocytes. (A) Violin plots of indicated gene expression levels (represented by log-transformed normalized counts) across monocyte, macrophage and DC clusters. (B) Violin plots of indicated gene expression levels (represented by log-transformed normalized counts) across all major non-CM clusters.

FIGS. 17A-17F show the SPP1-producing and CCR2⁺ macrophages in human atrial disease. (A) In situ staining of SPP1 and CD68 mRNA on human left atrial tissue from controls, and “MR only”, “AFib only” and “AFib with MR” patients. Dashed area in representative images denotes a collagen-rich area; higher magnification of the squared area is shown on the right; arrowheads indicate SPP1⁺CD68⁺ macrophages; scale bars represent 50 μm. (B) Bar graph shows SPP1⁺CD68⁺ macrophages as absolute number per mm²; n=8-10 per group, ANOVA followed by Tukey's multiple comparisons test, all P-values are compared with controls unless indicated differently. (C) Representative immunofluorescent staining of SPP1, collagen 1A1 (COL1A1), an extracellular matrix protein produced by fibroblasts, and DAPI on left atrial tissue from controls and AFib patients. Scale bars represent 25 μm. (D) Bar graph shows percentage of SPP1-positive staining per COL1A1-positive area in left atrial tissue from controls and AFib patients; n=10 per group, two-tailed Student's t test. (E) Representative immunofluorescent staining of CD68, CCR2, and DAPI on left atrial tissue from 41 controls, and 26 “MR only”, 34 “AFib only” and 48 “AFib with MR” patients. Higher magnification of the squared area is shown on the right; arrowheads indicate recruited CCR2⁺CD68⁺ macrophages; scale bars represent 50 μm. (F) Bar graphs show total CD68⁺ and recruited CCR2⁺CD68⁺ macrophages as absolute number per mm²; n=26-48 per group, Kruskal-Wallis test followed by Dunn's multiple comparisons test, all P-values are compared with controls unless indicated differently. All bar graph data are mean±SEM with individual values for data distribution.

FIG. 18 shows the blood pressure measurements in HOMER mice. Systolic and diastolic blood pressure in sham and HOMER mice. Data are mean±SEM with individual values for data distribution;
n=11 per group from two independent experiments, two-tailed Student's t test.

FIGS. 19A-19B show the ECG recordings obtained during invasive EP studies. Representative surface ECG and right atrial (RA) and right ventricular (RV) intracardiac electrograms (EGMs) during an invasive EP study in (A) a sham control mouse showing sinus rhythm and (B) a HOMER mouse showing AFib after rapid intracardiac pacing. Scale bars represent 100 ms.

FIGS. 20A-20B show the individual contribution of HOMER components to atrial disease. (A) EP study of AFib inducibility and burden in sham mice and mice exposed to MR, HFD or Ang2 infusion. n=6-12 per group from two independent experiments; left, two-sided Fisher's exact test; right, Kruskal-Wallis test followed by Dunn's multiple comparisons test; all P-values are compared with sham mice. (B) Cardiac MRI was used to quantify atrial size in sham mice and mice exposed to single components. n=6-9 per group from two independent experiments, ANOVA followed by Dunnett's multiple comparisons test, all P-values are compared with sham mice. All bar graph data are mean±SEM with individual values for data distribution.

FIGS. 21A-21E show the characterization of HOMER mouse model. (A) Cardiac MRI evaluation of left ventricular (LV) function, volumes and mass in sham and HOMER mice. n=6-10 per group from two independent experiments, two-tailed Student's t test. (B) Flow cytometric quantification of leukocytes, monocytes, neutrophils and lymphocytes in blood from sham and HOMER mice. n=16-20 per group from three independent experiments, two-tailed Student's t test. (C) Pie charts indicating the percentage of Ly6C^{lo} and Ly6C^{hi} monocytes in blood from sham and HOMER mice. Data are mean; n=16-17 per group from three independent experiments, two-tailed Mann-Whitney test, bold and italicized percentages denote P<0.01 compared with sham. (D) Representative immunofluorescent staining of macrophages (CD68), collagen deposition (COL1), and nuclei (DAPI) and histological staining of fibrosis (Masson's trichrome staining) in left atrial tissue from sham and HOMER mice. Scale bars represent 50 μm. (E) Quantification of CD68⁺, COL1⁺ and fibrotic area in left atrial tissue from sham and HOMER mice. Bar graphs show percentage of positive staining per field of view (FOV); n=6 per group from two independent experiments, two-tailed Student's t test. All bar graph data are mean±SEM with individual values for data distribution.

FIGS. 22A-22C show the contribution of single HOMER components to macrophage expansion and atrial fibrosis. (A) Representative immunofluorescent staining of macrophages (CD68) and nuclei (DAPI) and histological staining of fibrosis (Masson's trichrome staining) in left atrial tissue from sham mice and mice exposed to single conditions. Scale bars represent 50 μm. (B, C) Quantification of CD68⁺ (B) and fibrotic (C) area in left atria from sham mice and mice exposed to single conditions. Bar graphs show percentage of positive staining per FOV; n=6 per group from two independent experiments, ANOVA followed by Dunnett's multiple comparisons test, all P-values are compared with sham mice. All bar graph data are mean±SEM with individual values for data distribution.

FIGS. 23A-23C show the isolating and annotating mouse left atrial non-cardiomyocytes. (A) Representative plots of the flow cytometry gating strategy for isolating single, viable non-CM from mouse left atrial tissues. (B) Clustering of 40,112 non-CM from left atrial tissues of four sham and four HOMER mice. (C) Dot plot annotating non-CM clusters by signature gene expression. z-score of average gene expression is denoted; circle size indicates percentage of cells expressing the gene.

FIGS. 24A-24E show the quantifying left atrial non-cardiomyocytes in HOMER mice. (A) Flow cytometry gating strategy to quantify cardiac macrophages in left atrial tissue. (B) Flow cytometry gating strategy to quantify cardiac endothelial cells (ECs), fibroblasts and mural cells. RMCs, resident mesenchymal cells. (C) Flow cytometry to quantify left atrial Ly6C^{hi} monocytes, neutrophils, endothelial cells, fibroblasts and mural cells in sham and HOMER mice. Ly6C^{hi} monocytes and neutrophils: 15 sham and 12 HOMER mice from three independent experiments; endothelial cells, fibroblasts and mural cells: 6 sham and 8 HOMER mice from two independent experiments; two-tailed Student's t test. (D) Pie charts indicating the percentage of MHCII^{lo} CCR2⁻, MHCII^{hi} CCR2⁻, MHCII^{hi} CCR2⁺ macrophages and recruited monocytes in left atrial tissue from sham and HOMER mice. Data are mean; n=13-15 per group from three independent experiments, two-tailed Student's t test, bold and italicized percentages denote P<0.001 compared with sham. (E) Absolute macrophage subset numbers per left atrial tissue from sham and HOMER mice. n=13-15 per group from three independent experiments, two-tailed Student's t test. All bar graph data are mean±SEM with individual values for data distribution.

FIGS. 25A-25D show Spp1 expression in non-cardiomyocytes of the mouse atria. (A) Violin plots of indicated gene expression levels (represented by log-transformed normalized counts) across MP/DC subclusters. (B) Violin plots of indicated gene expression levels (represented by log-transformed normalized counts) across all major non-CM clusters. (C) Circular plot showing the difference in abundance of cells belonging to MP/DC subclusters between four HOMER and four sham mice. Pie size indicates log₂-fold changes. (D) Flow cytometry gating strategy to quantify cardiac CD9⁺ macrophages in left atrial tissue. Gates were set using a CD9 isotype control antibody.

FIGS. 26A-26B show the correlation of atrial myeloid cell phenotypes between human patients and mice. (A) Heatmap comparing human and mouse MP/DC subclusters using their top 100 DEGs compared to all other MP/DC subclusters. Only genes expressed in both species were included. The significance of the gene overlap between human and mouse clusters was obtained by two-sided Fisher's exact test with multiple testing corrected by the Benjamini-Hochberg method. The −log₁₀(FDR) is denoted. (B) Same as in (A), but here only considering macrophage subsets.

FIGS. 27A-27G show the individual contribution of HOMER components to atrial expression of inflammatory and fibrotic genes. (A-G) Relative expression levels of inflammatory and fibrosis-related genes by qPCR in left atria from sham mice, mice exposed to single components and HOMER mice. Data are mean±SEM with individual values for data distribution; n=6-8 per group from two independent experiments, ANOVA followed by Dunnett's multiple comparisons test, all P-values are compared with sham mice.

FIGS. 28A-28C show the cardiac function and left atrial cellular frequencies in mice exposed to mitral regurgitation and high-fat diet only. (A) LA volume and LV function, volumes and mass in HOMER mice and mice exposed to MR and HFD only. n=5-10 per group from two independent experiments, two-tailed Student's t test. (B) Flow cytometry to quantify left atrial macrophages, Ly6C^{hi} monocytes, neutrophils, endothelial cells, fibroblasts and mural cells in HOMER mice and mice exposed to MR and HFD only. n=6 per group from two independent experiments, two-tailed Student's t test. (C) Relative Spp1 expression levels by qPCR in left atrial macrophages in HOMER mice and mice exposed to MR and HFD only. n=6 per group from two independent experiments, two-tailed Student's t test. All bar graph data are mean=SEM with individual values for data distribution.

FIGS. 29A-29F show the transcriptional comparison of left atria and ventricles from sham and HOMER mice. (A, B) Left, UMAP plots of cell clusters in left atria (LA, A) and left ventricles (LV, B) from four sham and four HOMER mice; right, cell population distributions in LA (A) and LV (B) from these mice. (C) HOMER-induced cell frequency changes comparing atria and ventricles. (D) Number of significant DEGs (with FDR<0.05) in MP/DC, fibroblast and endothelial subsets by pseudobulk differential analyses comparing the LA with LV. (E) Venn diagrams of these genes illustrating limited overlap of DEGs between atria and ventricles.
(F) PCA plots for MP/DC, fibroblast and endothelial subsets of LA and LV samples from four sham and four HOMER mice.

FIGS. 30A-30E show the sources of left atrial macrophage expansion in HOMER mice. (A) Fate-mapping in Cx3cr1^{CreER/+}Ai9^{fl/+} mice and quantification of tdTomato (Td) signal in YFP+Ly6C^{hi} monocytes three and 42 days after final tamoxifen injection. n=20 per group from two independent experiments, two-tailed Wilcoxon matched-pairs signed rank test. (B) Flow cytometric quantification of recruited (YFP⁺Td⁻) macrophages in LV and LA from Cx3cr1^{CreER/+}Ai9^{fl/+} sham mice. n=8 per group from two independent experiments, two-tailed paired t test. (C) GSEA (gene set “leukocyte proliferation”) of left atrial MP/DCs in four sham and four HOMER mice. (D) Representative immunofluorescent staining for the macrophage marker CD68, proliferation marker Ki67, and DAPI in left atria from sham and HOMER mice. Arrowheads indicate Ki67⁺CD68⁺ macrophages; scale bars represent 50 μm. (E) Bar graph shows the number of Ki67⁺CD68⁺ macrophages per FOV in left atria from sham and HOMER mice; n=6 per group from two independent experiments, two-tailed Student's t test. All bar graph data are mean±SEM with individual values for data distribution.

FIGS. 31A-31E show the blood leukocytes, left atrial non-cardiomyocytes, blood pressure and cardiac function in Ccr2^{-/-} HOMER mice. (A) Flow cytometric quantification of leukocytes, monocytes, neutrophils and lymphocytes in blood from C57BL/6 and Ccr2^{-/-} HOMER mice. n=9-16 per group from three independent experiments, two-tailed Student's t test. (B) Pie charts indicating the percentage of Ly6C^{lo} and Ly6C^{hi} monocytes in blood from C57BL/6 and Ccr2^{-/-} HOMER mice. Data are mean; n=10-16 per group from three independent experiments, two-tailed Student's t test, bold and italicized percentages denote P<0.0001 compared with C57BL/6 HOMER mice. (C) Flow cytometric quantification of left atrial endothelial cells, fibroblasts and mural cells in C57BL/6 and Ccr2^{-/-} HOMER mice. n=8-9 per group from two independent experiments, two-tailed Student's t test. (D) Systolic and diastolic blood pressure in C57BL/6 and Ccr2^{-/-} HOMER mice. n=10-11 per group from two independent experiments, two-tailed Student's t test. (E) LA volume and LV function, volumes and mass in C57BL/6 and Ccr2^{-/-} HOMER mice by cardiac MRI. n=7-10 per group from two independent experiments; LA end-systolic volume: two-tailed Mann-Whitney test; other parameters: two-tailed Student's t test. All bar graph data are mean±SEM with individual values for data distribution.

FIGS. 32A-32D show that CCR2 inhibition reduces atrial fibrosis. (A) Experimental outline. (B) Repre-sentative histological staining of fibrosis (Masson's trichrome staining) in left atrial tissue from PBS-treated HOMER mice and HOMER mice that were treated with a CCR2 inhibitor for 4 weeks. Scale bars represent 50 μm. (C) Quantification of fibrotic area in left atrial tissue from PBS-treated HOMER mice and HOMER mice that were treated with a CCR2 inhibitor for 4 weeks. Bar graph shows percentage of positive staining per FOV; n=6 per group from two independent experiments, two-tailed Student's t test. (D) Invasive EP studies in PBS-treated HOMER mice and HOMER mice that were treated with a CCR2 inhibitor for 4 weeks. n=12 per group from two independent experiments; AFib inducibility: two-sided Fisher's exact test; burden: two-tailed Mann-Whitney test. All bar graph data are mean±SEM with individual values for data distribution.

FIGS. 33A-33F show the Spp1 deletion in HOMER mice. (A, B) Percentage of SPP1 normalized counts across all major non-CM clusters in human (A) and mouse (B) left atrial tissues. (C) Systolic and diastolic blood pressure in C57BL/6 BMT HOMER and Spp1^{-/-} BMT HOMER mice. n=6-8 per group from two independent experiments, two-tailed Student's t test. (D) Flow cytometric quantification of leukocytes, monocytes, neutrophils and lymphocytes in blood from C57BL/6 BMT HOMER and Spp1^{-/-} BMT HOMER mice. n=10-11 per group from two independent experiments, two-tailed Student's t test. (E) Pie charts indicating the percentage of Ly6C^{lo} and Ly6C^{hi} monocytes in blood from C57BL/6 BMT HOMER and Spp1^{-/-} BMT HOMER mice. Data are mean; n=10-11 per group from two independent experiments, two-tailed Student's t test, bold and italicized percentages denote P<0.05 compared with C57BL/6 BMT HOMER mice. (F) LA volume and LV function, volumes and mass in C57BL/6 BMT HOMER and Spp1^{-/-} BMT HOMER mice by cardiac MRI. n=7-9 per group from two independent experiments, two-tailed Student's t test. All bar graph data are mean±SEM with individual values for data distribution.

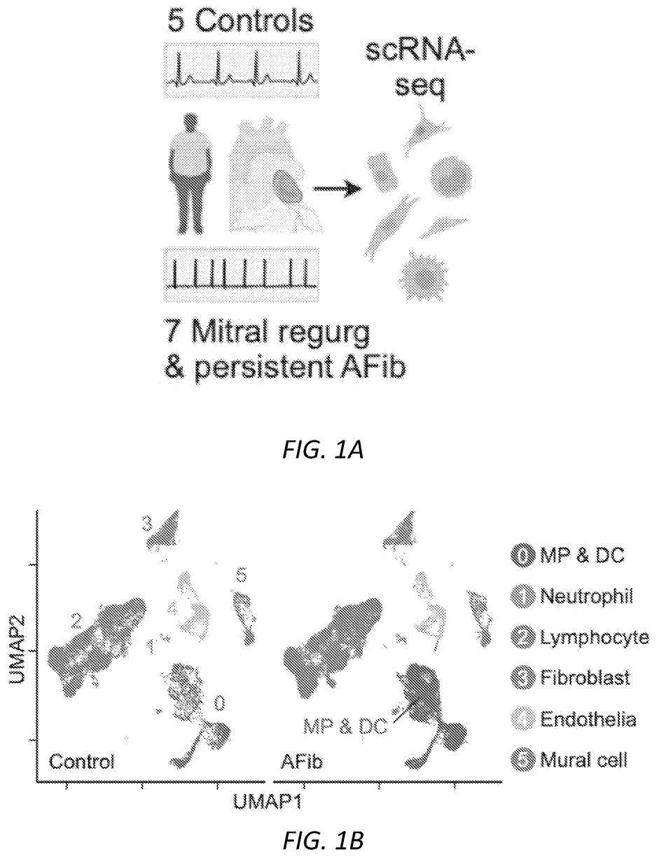

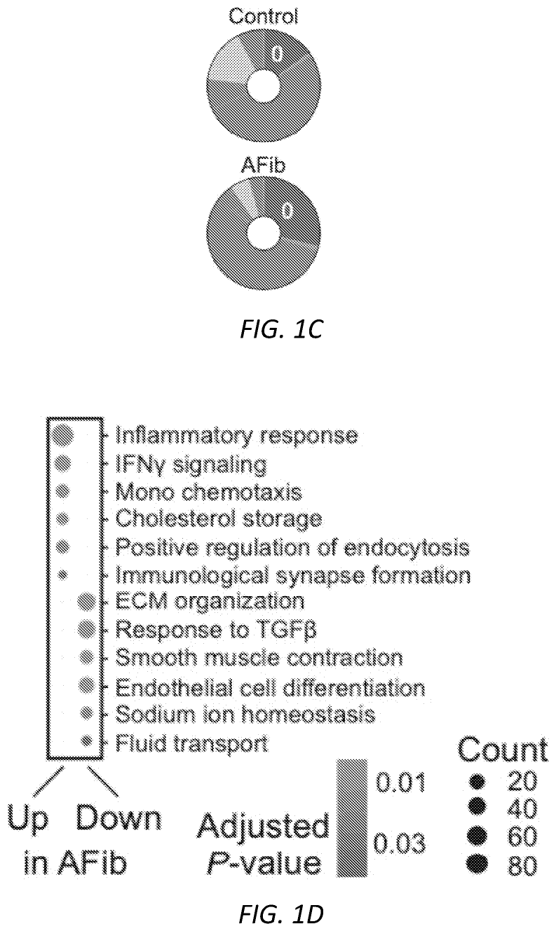

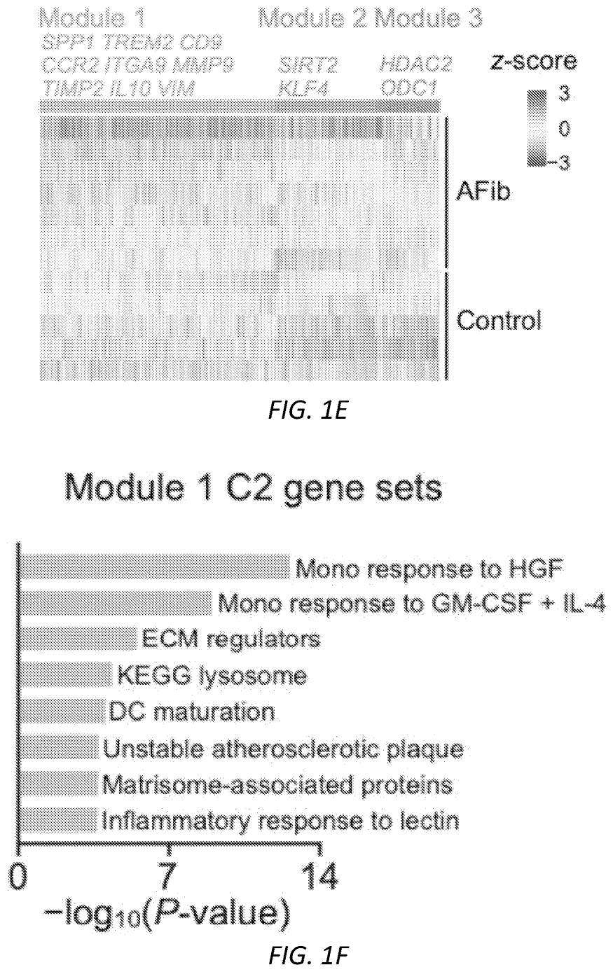

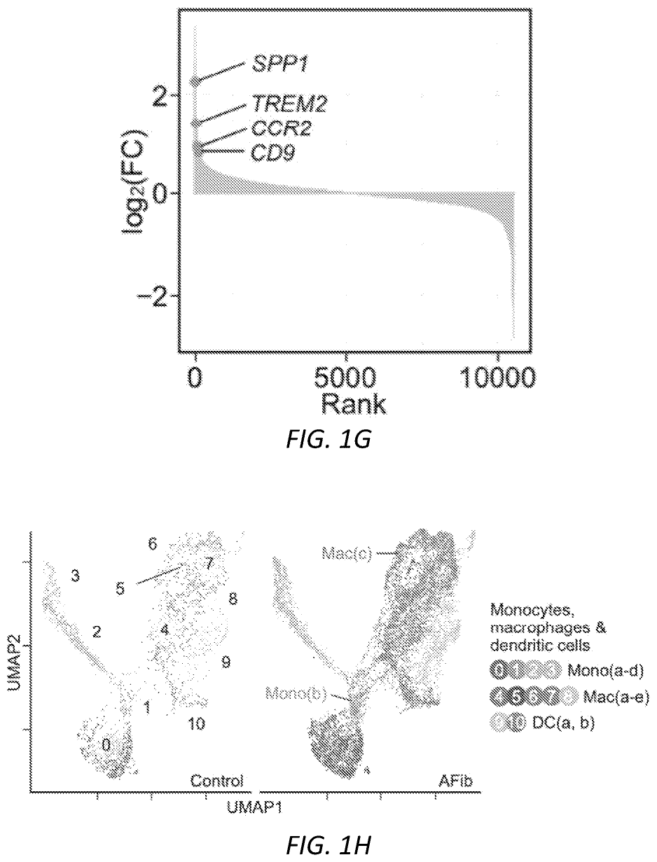

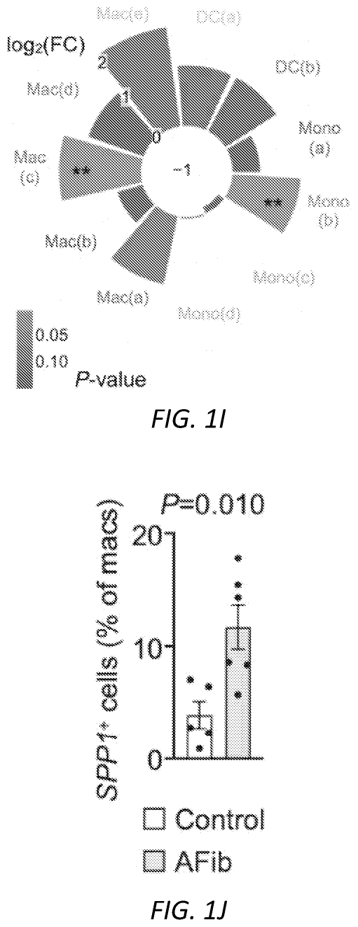

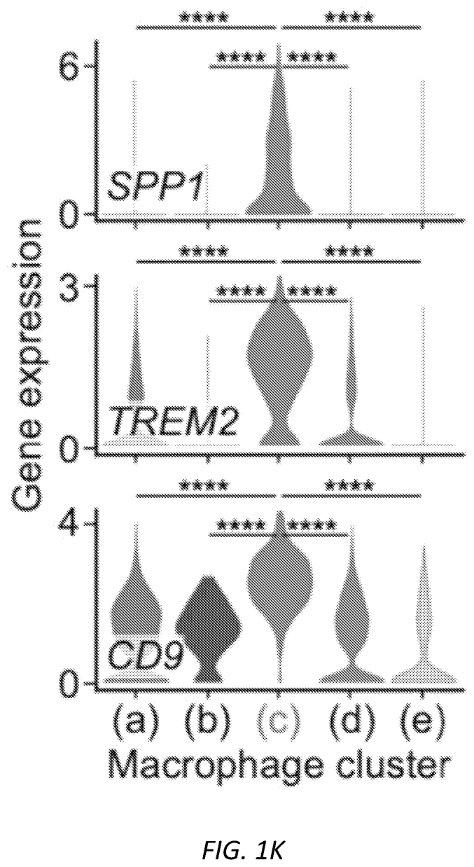

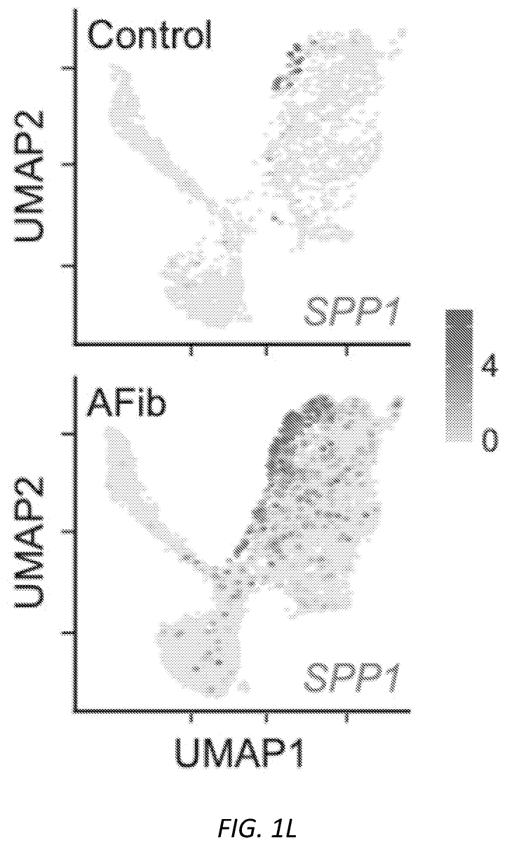

FIG. 34 shows the SPP1 interactions in human atrial disease. Heatmap of SPP1-receptor interactions between SPP1⁺ macrophages (mac (c)) and other non-CM in seven AFib patients. SPP1 and cognate receptors are shown on the x-axis. Cell populations that express SPP1 and the receptor are shown on the y-axis and are coded according to the major cardiac cell types identified in FIG. 1B. Scale bar denotes average SPP1 and receptor expression levels in their respective interacting subpopulations (represented by mean normalized counts) if the enrichment of an interacting SPP1-receptor pair in the given interacting subpopulations was statistically significant (P<0.05). Non-significant interactions were assigned a value of 0.

FIGS. 35A-35K show the Spp1 deletion effects in HOMER atria. (A) Flow cytometric quantification of left atrial Ly6C^{hi} monocytes in C57BL/6 BMT HOMER and Spp1^{-/-} BMT HOMER mice. n=7-13 per group from two independent experiments, two-tailed Student's t test. (B) Immuno-fluorescent quantification of TUNEL⁺ macrophages in left atrial tissue obtained from C57BL/6 BMT HOMER and Spp1^{-/-} BMT HOMER mice. Bar graph shows the number of TUNEL⁺ macrophages per FOV; n=4-5 per group from two independent experiments, two-tailed Student's t test. (C) Representative immunofluorescent staining for the macrophage marker CD68, proliferation marker Ki67, and DAPI in left atria from C57BL/6 BMT HOMER and Spp1^{-/-} BMT HOMER mice. Arrowheads indicate Ki67⁺CD68⁺ macrophages. (D) Bar graph shows the number of Ki67⁺CD68⁺ macrophages per FOV in left atria from C57BL/6 BMT HOMER and Spp1^{-/-} BMT HOMER mice; n=4-5 per group from two independent experiments, two-tailed Student's t test. (E) Violin plots of indicated gene expression levels (represented by log-transformed normalized counts) in left atrial fibroblasts from four sham and four HOMER mice; P-values determined by two-sample Kolmogorov-Smirnov test. (F) Relative collagen expression levels by qPCR in FACS-purified left atrial fibroblasts from sham and HOMER mice. n=4-6 per group, two-tailed Student's t test.
(G) Representative immunofluorescent staining for α SMA and DAPI in left atria from sham and HOMER mice. (H) Bar graph shows percentage of αSMA⁺ staining per FOV in left atria from sham and HOMER mice; n=5 per group from two independent experiments, two-tailed Student's t test. (I) Representative immunofluorescent staining for αSMA and DAPI in left atria from C57BL/6 BMT HOMER and Spp1^{-/-} BMT HOMER mice. (J) Bar graph shows percentage of αSMA⁺ staining per FOV in left atria from C57BL/6 BMT HOMER and Spp1^{-/-} BMT HOMER mice; n=4-5 per group from two independent experiments, two-tailed Student's t test. (K) Relative collagen expression levels by qPCR in FACS-purified left atrial fibroblasts from C57BL/6 BMT HOMER and Spp1^{-/-} BMT HOMER mice. n=4-8 per group, two-tailed Mann-Whitney test. All bar graph data are mean±SEM with individual values for data distribution. All scale bars represent 50 μm.

FIGS. 36A-36F show SPP1 pathways in HOMER mice. (A, B) Spp1 expression levels (represented by log-transformed normalized counts) across the macrophage clusters (A) and major cell types (B) in left atrial tissue from two C57BL/6 BMT HOMER and two Spp1^{-/-} BMT HOMER mice. y-axis was limited to 2.5 for data visualization. (C) UMAP plot of left atrial fibroblasts in two C57BL/6 BMT HOMER and two Spp1^{-/-} BMT HOMER mice. Cluster 10 was omitted from further analyses shown in (D) and (E) due to its small size. (D) Heatmap depicting expression of collagen genes (represented by z-scores of the log-transformed normalized counts) in each of the 10 fibroblast clusters. The rows show the collagen genes; each column represents a cell. (E) Heatmap and hierarchical clustering of the fibroblast clusters according to the collagen gene expression levels (represented by z-scores of the average normalized counts). (F) GSEA results of fibroblast clusters 5 and 7 in two C57BL/6 BMT HOMER versus two Spp1^{-/-} BMT HOMER mice.

FIG. 37 shows macrophage functions in atrial remodeling. In human and mouse atria, recruited macrophages upregulate SPP1 expression, which augments inflammation and fibrosis. Through these functions, macrophages contribute to the electrophysiological substrate of atrial fibrillation.

FIGS. 38A-38M show that ARC silences Spp1 in TREM2⁺ macrophages. (A), Uniform manifold approximation and projection (UMAP) of deposited human scRNA-seq data derived from atrial appendages of five controls and seven patients with AFib highlighting co-expression of SPP1 and TREM2. The major cell clusters identified were mononuclear phagocytes (M), neutrophils (N), lymphocytes (L), fibroblasts (F), endothelial cells (E), and mural cells (V). (B), UMAP of deposited murine scRNA-seq data derived from left atria of four sham and four HOMER AFib mice highlighting co-expression of Spp1 and Trem2. (C), Schematic of a TREM2 antibody bearing an inert IgG2c Fc with LALAPG mutations (anti-TREM2) fused to siRNAs directed against Spp1 (siSpp1). (D), Experimental outline to assess the cellular and organ biodistribution of fluorescently labeled isotype control antibody, anti-TREM2, and anti-TREM2-siSpp1 injected intravenously. (E), Representative fluorescence images of heart, liver lobe, spleen, and kidney. Scale bars, 5 mm. (F), Quantification of mean organ fluorescence in heart's left atria (LA) and left ventricle (LV), liver lobe, spleen, and kidney normalized to vehicle-treated control. (G), Median fluorescence intensity histogram of internalized isotype control antibody, anti-TREM2, and anti-TREM2-siSpp1 in lymphocytes (lympho), MHCII^{lo} macrophages (MHCII^{lo} Mφ), MHCII^{hi} macrophages (MHCII^{hi} Mφ), endothelial cells (EC), and fibroblasts from the heart. (H), Percentage of total cardiac MHCII^{lo} and MHCII^{hi} macrophages with internalized fluorescence. (I), Experimental outline to differentiate bone-marrow-derived macrophages (BMDM) from wild-type (WT) C57BL/6 mice and Trem2^{-/-} mice. (J,K), Quantification of Spp1 mRNA (j) and protein (k) expression in WT (left) and Trem2^{-/-} (right) BMDM normalized to vehicle-treated control. (L), Experimental outline of C57BL/6 mice undergoing chronic hypertension for 28 days followed by treatment with vehicle or anti-TREM2-siSpp1 on days 6 and 9. (M), Expression of Spp1 mRNA in TREM2^{hi} (left) and TREM2ₗₒ (right) cardiac macrophage subsets isolated on day 12 normalized to vehicle-treated cells.

FIGS. 39A-39J show that ARC therapy suppresses AFib by reducing atrial fibrosis. (A), Experimental outline to treat HOMER mice with established atrial pathology arising from hypertension (angiotensin 2-Ang2), obesity (high-fat diet-HFD) and mitral valve regurgitation (MR) with weekly intravenous injections of vehicle or anti-TREM2-siSpp1 for one month. (B), Representative ECG recordings from a cardiac electrophysiological (EP) study of vehicle- and anti-TREM2-siSpp1-treated HOMER mice at week 25. Scale bar, 200 ms. (C), EP study of AFib inducibility and burden in vehicle- and anti-TREM2-siSpp1-treated HOMER mice. n=11 to 12 per group from two independent experiments. (Left) Two-sided Fisher's exact test. (Right) Two-tailed Mann-Whitney test. (D), Representative images (left) and quantification (right) of fibrosis (Masson's trichrome staining) in left atrial tissue from vehicle- and anti-TREM2-siSpp1-treated HOMER mice. Scale bars, 50 μm. n=8 to 9 per group from two independent experiments, two-tailed Student's t-test. (E), Representative cardiac MRI images (left) and left atrial (LA) end-systolic volumes (right) of vehicle- and anti-TREM2-siSpp1-treated HOMER mice. Scale bars, 10 mm. n=6 to 10 per group from two independent experiments, two-tailed Mann-Whitney test. (F), UMAP delineates 9 major cardiac cell types in three vehicle- and three anti-Trem2-siSpp1-treated HOMER mice. Cell identities were inferred from known marker gene expression. (G), UMAP plot of left atrial fibroblasts in three vehicle- and three anti-Trem2-siSpp1-treated HOMER mice. (H), Heatmap and hierarchical clustering of the 6 fibroblast clusters according to the gene expression levels of fibrosis-related genes⁴² (represented by z-scores of the average normalized counts). (I), Fraction of pro-fibrotic fibroblasts (cluster 4) in three vehicle- and three anti-Trem2-siSpp1-treated HOMER mice. Two-tailed Student's t-test. (J), Expression levels of collagen genes in pro-fibrotic fibroblasts (cluster 4) of three vehicle- and three anti-Trem2-siSpp1-treated HOMER mice. Kolmogorov-Smirnov test.

FIGS. 40A-40D show the binding and internalization of anti-TREM2. (A), Antibody binding of AF647-conjugated anti-TREM2 in RAW264.7 macrophages by flow cytometry. Expi293 cells lacking TREM2 expression served as negative control cells, while commercially available TREM2-APC antibody served as a positive control antibody. (B), RAW264.7 macrophages exposed to AF488-conjugated anti-TREM2 for 0, 0.5, 1, 3, and 5 hours with or without exposure to the anti-AF488 fluorescence quenching antibody. (C), MFI quantification attributed to cell surface-binding antibody, cell internalized antibody, and total antibody signal. (D), Linear fit of the internalization rate using measurements of the internalized fraction of antibody versus the surface integral over time.

FIGS. 41A-41D show the antibody and siRNA conjugation. (A) Method for functionalizing the anti-TREM2 antibody with MeTz-Peg4-NHS. (B), Method for functionalizing the 3′ end of the siRNA sense strand with a TCO-Peg4-NHS moiety. (C), Click chemistry reaction scheme for conjugating the MeTz groups on the antibody with TCO-tagged siRNAs to form antibody-siRNA conjugates (ARC). (D). Size exclusion chromatography purification of ARC based on 280 nm absorbance.

FIG. 42 shows patient demographic and clinical information.

FIG. 43 shows the distribution of cell populations in human LA by scRNA-seq.

FIG. 44 shows patient demographics and clinical information (histology cohort).

FIG. 45 shows the general characteristics of sham and HOMER mice.

FIG. 46 shows the distribution of cell populations in mouse LA by scRNA-seq.

FIG. 47 shows the distribution of cell populations in mouse LA and LV by scRNA-seq.

FIG. 48 shows the electrophysiological parameters of sham and HOMER mice by in vivo EP study.

FIG. 49 shows the electrophysiological parameters of C57BL/6 and Ccr2−/− HOMER mice by in vivo EP study.

FIG. 50 shows the electrophysiological parameters of C57BL/6 BMT and Spp1−/− BMT HOMER mice by in vivo EP study.

FIG. 51 shows information on flow cytometry antibodies.

FIG. 52 shows information on histology antibodies.

### DETAILED DESCRIPTION

Inflammation may contribute to AFib via electrical remodeling, during which altered ion handling perturbs the action potential. Fibrosis, another consequence of inflammation, induces heterogeneous atrial depolarization which provides an opportunity for electrical conduction re-entry and subsequent AFib.

Atrial fibrillation (AFib) is the most common irregular heart rhythm, affecting 10% of adults over 65 years old (see, e.g., Kornej, J., Börschel, C. S., Benjamin, E. J. & Schnabel, R. B. Epidemiology of Atrial Fibrillation in the 21st Century: Novel Methods and New Insights. Circ Res 127, 4-20 (2020)). If untreated, debilitating complications like stroke and heart failure can arise (see, e.g., Michaud, G. F. & Stevenson, W. G. Atrial Fibrillation. N Engl J Med 384, 353-361 (2021)). Unfortunately, effective AFib treatments remain elusive, in part, due to its multifaceted etiology.

To better dissect the relationship between macrophages, fibrosis, and AFib, we developed a mouse model that recapitulates human AFib by combining common clinical risk factors: hypertension, obesity, and mitral valve regurgitation (referred to as HOMER). We identified osteopontin, encoded by SPP1 in humans and Spp1 in mice, as a top-upregulated gene in recruited atrial macrophages in both AFib patients and HOMER mice (see, e.g., Hulsmans, M. et al. Recruited macrophages elicit atrial fibrillation. Science 381, 231-239 (2023)). Osteopontin is a conserved pleiotropic matricellular protein that binds several integrins and CD44 family receptors. Osteopontin drives fibroblasts to promote tissue fibrosis and has been correlated with the progression of several chronic fibrotic diseases (see, e.g., Lund, S. A., Giachelli, C. M. & Scatena, M. The role of osteopontin in inflammatory processes. J Cell Commun Signal 3, 311-322 (2009)). Fibrosis in the heart can cause atrial tissue to become heterogeneous, which hinders homogeneous conduction and acts as a structural substrate to promote AFib (see, e.g., Harada, M. & Nattel, S. Implications of Inflammation and Fibrosis in Atrial Fibrillation Pathophysiology. Card Electrophysiol Clin 13, 25-35 (2021)). Most notably, transgenic deletion of osteopontin in monocyte-derived macrophages of HOMER mice reduced atrial fibrosis and inducible AFib (see, e.g., Hulsmans, M. et al. Recruited macrophages elicit atrial fibrillation. Science 381, 231-239 (2023)).

These results motivate SPP1/Spp1 silencing in macrophages as a potential immunotherapy for AFib; however, engineering such treatment remains challenging. Although small interfering RNA (siRNA) has the potential to reversibly silence any gene and several are under clinical development, their delivery to specific cells, especially outside the liver, is a major obstacle (see, e.g., Ahn, I., Kang, C. S. & Han, J. Where should siRNAs go: applicable organs for siRNA drugs. Exp Mol Med 55, 1283-1292 (2023)). Antibodies, known for their strength and specificity in binding antigens and their established use as drug conjugates, are promising delivery vehicles for siRNA delivery (see, e.g., Dugal-Tessier, J., Thirumalairajan, S. & Jain, N. Antibody-Oligonucleotide Conjugates: A Twist to Antibody-Drug Conjugates. J Clin Med 10, 838 (2021)).

Thus provided herein are therapeutic strategies that target recruitment of inflammatory macrophages (CCR2) or macrophage-derived signals (SPP1).

Provided herein are compositions and pharmaceutical compositions comprising inhibitory nucleic acids (e.g., siRNA agents) targeting SPP1.

Accordingly, the disclosure is related to inhibitory nucleic acid molecules that are targeted to a selected target RNA, e.g., antisense or double-stranded siRNA that target SPP1. Exemplary human sequences of SPP1 are known in the art and include RefSeq Gene ID NG_030362.1 Range 5065-12762 (genomic sequence), and the sequences in the following table:

TABLE 1
Examples of Human Sequences of SPP1

| Nucleic Acid | Protein | name |
|---|---|---|
| NM_000582.3 | NP_000573.1 | osteopontin isoform OPN-b precursor |
| NM_001040058.2 | NP_001035147.1 | osteopontin isoform OPN-a precursor |
| NM_001040060.2 | NP_001035149.1 | osteopontin isoform OPN-c precursor |
| NM_001251829.2 | NP_001238758.1 | osteopontin isoform 4 precursor |
| NM_001251830.2 | NP_001238759.1 | osteopontin isoform 5 |

Also provided herein are methods for treating or reducing the risk of progression of development or recurrence of an atrial disease, comprising administering any of the compositions described herein, or any of the pharmaceutical compositions described herein.

The term “about” when referring to a number or a numerical range means that the number or numerical range referred #10%.

The terms “or more’ and “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20-nucleotide nucleic acid molecule” means that 17, 18, 19, or 20 nucleotides have the indicated property. When “at least” is present before a series of numbers or a range, it is understood that “at least” can modify each of the numbers in the series or range.

As used herein, “or less” and “no more than” are understood as including the value adjacent to the phrase and logical lower values or integers, as logical from context, to zero. For example, a duplex with mismatches to a target site of “no more than 2 nucleotides” has a 2, 1, or 0 mismatches. When “no more than” is present before a series of numbers or a range, it is understood that “no more than” can modify each of the numbers in the series or range.

As used herein, “less than” is understood as not including the value adjacent to the phrase and including logical lower values or integers, as logical from context, to zero. For example, a duplex with mismatches to a target site of “less than 3 nucleotides” has 2, 1, or 0 mismatches. When “less than” is present before a series of numbers or a range, it is understood that “less than” can modify each of the numbers in the series or range.

As used herein, “more than” is understood as not including the value adjacent to the phrase and including logical higher values or integers, as logical from context, to infinity. For example, a duplex with mismatches to a target site of “more than 3 nucleotides” has 4, 5, 6, or more mismatches. When “more than” is present before a series of numbers or a range, it is understood that “more than” can modify each of the numbers in the series or range.

As used herein, “up to” as in “up to 10” is understood as up to and including 10, i.e., 0, 1, 2, 3, 4, 5, 6, 7, 8, 9, or 10. Ranges provided herein are understood to include all individual integer values and all subranges within the ranges.

siRNA Molecules

RNAi is a process whereby double-stranded RNA (dsRNA, also referred to herein as dsRNA agents, for double-stranded small interfering RNAs,) induces the sequence-specific degradation of homologous mRNA in animals and plant cells (Hutvagner and Zamore, Curr. Opin. Genet. Dev.: 12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99 (9): 6047-6052 (2002); McManus et al., RNA 8:842-850 (2002); Sui et al., Proc. Natl. Acad. Sci. USA 99 (6): 5515-5520 (2002)).

As used herein, the term “iRNA,” “RNAi”, “iRNA agent,” or “RNAi agent” or “RNAi molecule” refers to an agent that contains RNA as that term is defined herein, and which mediates the targeted cleavage of an RNA transcript, e.g., via an RNA-induced silencing complex (RISC) pathway. In some embodiments, an iRNA as described herein effects inhibition of SPP1 expression, e.g., in a cell or mammal. Inhibition of SPP1 expression may be assessed based on a reduction in the level of SPP1 mRNA or a reduction in the level of the osteopontin protein. In some embodiments, an iRNA agent is a dsRNA agent. In some embodiments, an iRNA agent is an antisense RNA agent.

The term “antisense strand” or “guide strand” refers to the strand of an iRNA, e.g., a dsRNA, which includes a region that is substantially complementary to a target sequence. An “antisense” nucleic acid can include a nucleotide sequence that is complementary to a “sense” nucleic acid encoding a protein, e.g., complementary to the coding strand of a double-stranded cDNA molecule or complementary to a SPP1 mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

Target gene expression can be inhibited by targeting nucleotide sequences complementary to a regulatory region (e.g., promoters and/or enhancers) to form triple helical structures that prevent transcription of the SPP1 gene in target cells. See generally, Helene, C. Anticancer Drug Des. 6:569-84 (1991); Helene, C. Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. In some embodiments, the region of complementarity comprises 0, 1, or 2 mismatches.

The term “sense strand” or “passenger strand” as used herein, refers to the strand of an iRNA that includes a region that is substantially complementary to a region of the antisense strand as that term is defined herein.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

As used herein, the term “overhang” refers to unpaired nucleotide(s) or nucleotide analog(s) at a given terminal end of one or both strands of the iRNA (e.g., dsRNA) agent. The overhang of the iRNA (e.g., dsRNA) agent can be at the 3′ and/or the 5′ end of the sense and/or antisense strand. In some embodiments, an overhang is at the 3′ end of the sense and/or antisense strand. The overhang can have 1, 2, 3 or more nucleotides. In some embodiments, the overhang is 1, 2, or 3 nucleotides long.

The terms “complementary,” “fully complementary” and “substantially complementary” herein may be used with respect to the base matching between two oligonucleotides or polynucleotides, such as the sense strand and the antisense strand of a dsRNA, or between the antisense strand of an iRNA agent and a target sequence, as will be understood from the context of their use. As used herein, a polynucleotide that is “substantially complementary to at least part of” a messenger RNA (mRNA) refers to a polynucleotide that is substantially complementary to a contiguous portion of the mRNA of interest (e.g., an mRNA encoding a SPP1 protein). For example, a polynucleotide is complementary to at least a part of a SPP1 mRNA if the sequence is substantially complementary to a non-interrupted portion of an mRNA encoding SPP1. The term “complementarity” refers to the capacity for pairing between nucleobases of a first nucleic acid and a second nucleic acid.

The term “double-stranded RNA,” “dsRNA,” or “siRNA” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA, e.g., through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 17-23 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. In some embodiments, the two strands are connected covalently by means other than a hairpin loop, and the connecting structure is a linker. In some embodiments, the iRNA agent may be a “single-stranded siRNA” that is introduced into a cell or organism to inhibit a target mRNA. In some embodiments, single-stranded RNAi agents can bind to the RISC endonuclease Argonaute 2, which then cleaves the target mRNA. The single-stranded siRNAs are generally 15-30 nucleotides and are optionally chemically modified. The design and testing of single-stranded siRNAs are described in U.S. Pat. No. 8,101,348 and in Lima et al., (2012) Cell 150:883-894, the entire contents of each of which are hereby incorporated by reference.

Any of the antisense nucleotide sequences described herein (e.g., sequences provided in Tables 2 or 3) may be used as a single-stranded siRNA as described herein and optionally as chemically modified, e.g., as described herein, e.g., by the methods described in Lima et al., (2012) Cell 150:883-894. In some embodiments, an RNA interference agent includes a single stranded RNA that interacts with a target RNA sequence to direct the cleavage of the target RNA.

Without wishing to be bound by theory, long double stranded RNA introduced into cells is broken down into siRNA by a Type III endonuclease known as Dicer (Sharp et al., Genes Dev.2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev.15:188).

The term “linker” or “linking group” means an organic moiety that connects two parts of a compound, e.g., covalently attaches two parts of a compound.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties or analogs thereof (e.g., phosphorothioate). However, the terms “ribonucleoside” and “ribonucleotide” can be considered to be equivalent as used herein. The RNA can be modified in the nucleobase structure, in the ribose structure, or in the ribose-phosphate backbone structure, e.g., as described herein below. However, the molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, an acyclic nucleoside, a glycol nucleotide (e.g., glycol nucleic acid, GNA), a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, or in combination, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule.

For clarity, it is understood that the term “iRNA” does not encompass a naturally occurring double stranded DNA molecule or a 100% deoxynucleoside-containing DNA molecule. In some aspects, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. In certain embodiments, the RNA molecule comprises a percentage of deoxyribonucleosides of at least 5, 10, 15, 20, 25, 30, 35, 40, 45, 50, 55, 60, 65, 70, 75, 80, 85, 90, 95% or higher (but not 100%) deoxyribonucleosides, e.g., in one or both strands.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, or at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA. In some embodiments, the antisense strand of a dsRNA has a 1-10 nucleotide overhang at the 3′ end and/or the 5′ end. In some embodiments, the sense strand of a dsRNA has a 1-10 nucleotide overhang at the 3′ end and/or the 5′ end. In some embodiments, one or more of the nucleotides in the overhang is replaced with a nucleoside thiophosphate.

In some embodiments, the siRNA agent (e.g., dsRNA agent) inhibits the expression of SPP1 in a cell or mammal. In some embodiments, the siRNA agent (e.g., dsRNA agent) includes double-stranded ribonucleic acid (dsRNA) molecules for inhibiting the expression of SPP1 in a cell or in a subject (e.g., in a mammal, e.g., in a human), where the siRNA agent (e.g., dsRNA agent) includes an antisense strand having a region of complementarity which is complementary to at least a part of an mRNA formed in the expression of SPP1, and where the region of complementarity is 30 nucleotides or less in length, generally 19-24 nucleotides in length, and where the dsRNA, upon contact with a cell expressing SPP1, inhibits the expression of SPP1, e.g., by about or at least 10%, 15%, 20%, 25%, 30%, 35%, 40%, 45%, 50%, 55%, 60%, 65%, 70%, 75%, 80%, 85%, 90%, 95%, 96%, 97%, 98%, or 99%.

The modulation (e.g., inhibition) of expression of SPP1 can be assayed by, for example, a PCR or branched DNA (bDNA)-based method, or by a protein-based method, such as by Western blot. Expression of SPP1 in cell culture, such as in COS cells, ARPE-19 cells, hTERT RPE-1 cells, HeLa cells, primary hepatocytes, HepG2 cells, primary cultured cells or in a biological sample from a subject can be assayed by measuring SPP1 mRNA levels, such as by bDNA or TaqMan assay, or by measuring protein levels, such as by immunofluorescence analysis, using, for example, Western Blotting or flow cytometric techniques. A dsRNA typically includes two RNA strands that are sufficiently complementary to hybridize to form a duplex structure under conditions in which the dsRNA will be used. One strand of a dsRNA (the antisense strand) typically includes a region of complementarity that is substantially complementary, and generally fully complementary, to a target sequence, derived from the sequence of an mRNA formed during the expression of SPP1. The other strand (the sense strand) typically includes a region that is complementary to the antisense strand, such that the two strands hybridize and form a duplex structure when combined under suitable conditions.

The terms “silence,” “inhibit expression of,” “down-regulate expression of,” “suppress expression of,” and the like, in so far as they refer to SPP1, herein refer to the at least partial suppression of the expression of SPP1, as assessed, e.g., based on SPP1 mRNA expression, SPP1 protein expression, or another parameter functionally linked to SPP1 expression. For example, inhibition of SPP1 expression may be manifested by a reduction of the amount of SPP1 mRNA which may be isolated from or detected in a first cell or group of cells in which SPP1 is transcribed and which has or have been treated such that the expression of SPP1 is inhibited, as compared to a control. The control may be a second cell or group of cells substantially identical to the first cell or group of cells, except that the second cell or group of cells have not been so treated (control cells). The degree of inhibition is usually expressed as a percentage of a control level, e.g.,

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to SPP1 expression, e.g., the amount of protein encoded by a SPP1 gene. The reduction of a parameter functionally linked to SPP1 expression may similarly be expressed as a percentage of a control level. In principle, SPP1 silencing may be determined in any cell expressing SPP1, either constitutively or by genomic engineering, and by any appropriate assay. For example, in certain instances, expression of SPP1 is suppressed by at least about 10%, 15%, 20%, 25%, 30%, 35%, 40%, 45%, 50%, 55%, 60%, 65%, 70%, 75%, 80%, 85%, 90%, 95%, 98%, 99%, or more by administration of an iRNA as described herein.

The nucleic acid molecules or constructs can include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand.

In some embodiments, the duplex structure is between 15 and 30 base pairs in length, between 15 and 25 base pairs in length, between 15 and 20 base pairs in length, between 20 and 30 base pairs in length, between 20 and 25 base pairs in length, or between 25 and 30 base pairs in length. In some embodiments, the duplex structure is between 18 and 25 base pairs in length. In some embodiments, the duplex structure is between 19 and 24 base pairs in length. In some embodiments, the duplex structure is between 19 and 21 base pairs in length.

In some embodiments, the dsRNA is between 15 and 30 nucleotides in length, between 15 and 25 nucleotides in length, between 15 and 20 nucleotides in length, between 20 and 30 nucleotides in length, between 20 and 25 nucleotides in length, or between 25 and 30 nucleotides in length. In some embodiments, the dsRNA is between 15 and 20 nucleotides in length. In some embodiments, the dsRNA is between 25 and 30 nucleotides in length.

As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, e.g., 15-30 nucleotides in length.

A person skilled in the art will also recognize that the duplex region is a primary functional portion of a dsRNA, e.g., a duplex region of 9 to 36, e.g., 15-30 base pairs. Thus, in some embodiments, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in some embodiments, then, an miRNA is a dsRNA. In some embodiments, a dsRNA is not a naturally occurring miRNA. In some embodiments, an dsRNA agent useful to target SPP1 expression is not generated in the target cell by cleavage of a larger dsRNA.

A dsRNA as described herein may further include one or more single-stranded nucleotide overhangs. In some embodiments, the sense strand of the dsRNA agent described herein includes about 0, 1, 2, 3, 4, 5 or more overhangs at the 3′ or 5′ end. In some embodiments, the antisense strand of the dsRNA agent described herein includes about 0, 1, 2, 3, 4, 5 or more overhangs at the 3′ or 5′ end. In some embodiments, the sense strand of the dsRNA agent described herein includes about 2 overhangs at the 3′ or 5′ end. In some embodiments, the antisense strand of the dsRNA agent described herein includes about 2 overhangs at the 3′ or 5′ end.

In some embodiments, the SPP1 is a human SPP1 gene. In some embodiments, the dsRNA comprises a sense strand that comprises or consists of a sense sequence selected from the sense sequences provided in Table 3 or Table 4 and an antisense strand that comprises or consists of an antisense sequence selected from the antisense sequences provided in Table 3 or Table 4.

In some embodiments, a dsRNA will include at least sense and antisense nucleotide sequences, whereby the sense strand is selected from the sequences provided in Table 3 or Table 4 and the corresponding antisense strand is selected from the sequences provided in Table 3 or Table 4. In these aspects, one of the two sequences is complementary to the other of the two sequences, with one of the sequences being substantially complementary to a sequence of an mRNA generated by the expression of SPP1. As such, a dsRNA will include two oligonucleotides, where one oligonucleotide is described as the sense strand, and the second oligonucleotide is described as the corresponding antisense strand. As described elsewhere herein and as known in the art, the complementary sequences of a dsRNA can also be contained as self-complementary regions of a single nucleic acid molecule, as opposed to being on separate oligonucleotides.

The skilled person in the art is well aware that dsRNAs having a duplex structure of between 20 and 23, but specifically 21base pairs have been hailed as particularly effective in inducing RNA interference (Elbashir et al., EMBO 2001, 20:6877-6888). However, others have found that shorter or longer RNA duplex structures can be effective as well. In the embodiments described above, by virtue of the nature of the oligonucleotide sequences provided in Table 3 or Table 4, dsRNAs described herein can include at least one strand of a length of minimally 19 nucleotides. It can be reasonably expected that shorter duplexes having one of the sequences of Table 3 or Table 4 minus only a few nucleotides on one or both ends will be similarly effective as compared to the dsRNAs described above. In some embodiments, the dsRNA has a partial sequence of at least 15, 16, 17, 18, 19, 20, or more contiguous nucleotides from one of the sequences of Table 3 or 4.

In some embodiments, the dsRNA has an antisense sequence that comprises at least 15, 16, 17, 18, or 19 contiguous nucleotides of an antisense sequence provided in Table 2 or Table 3 and a sense sequence that comprises at least 15, 16, 17, 18, or 19 contiguous nucleotides of a corresponding sense sequence provided in Table 3 or Table 4. In some embodiments, the dsRNA comprises an antisense sequence that comprises at least 15, 16, 17, 18, 19, 20, 21, 22, or 23 contiguous nucleotides of an antisense sequence provided in Tables 3 and 4 and a sense sequence that comprises at least 15, 16, 17, 18, 19, 20, or 21 contiguous nucleotides of a corresponding sense sequence provided in Table 3 or Table 4. In some such embodiments, the dsRNA, although it comprises only a portion of the sequences provided in Table 3 or Table 4 is equally effective in inhibiting a level of SPP1 expression as is a dsRNA that comprises the full-length sequences provided in Table 3 or 4.

The nucleic acid compositions can include both siRNA and modified siRNA derivatives, e.g., siRNAs modified to alter a property such as the pharmacokinetics of the composition, for example, to increase half-life in the body, as well as engineered RNAi precursors. The dsRNA molecules can be chemically synthesized, or can transcribed be in vitro from a DNA template, or in vivo from, e.g., shRNA. The dsRNA molecules can be designed using any method known in the art; a number of algorithms are known, and are commercially available. Gene walk methods can be used to optimize the inhibitory activity of the siRNA.

siRNAs can be delivered into cells by methods known in the art, e.g., cationic liposome transfection and electroporation. siRNA duplexes can be expressed within cells from engineered RNAi precursors, e.g., recombinant DNA constructs using mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra).

“Contacting,” as used herein, includes directly contacting a cell, as well as indirectly contacting a cell. For example, a cell within a subject may be contacted when a composition comprising an iRNA agent (e.g., dsRNA agent) is administered (e.g., intravenously, intrathecally, intracranially, intracerebrally, or intraventricularly) to the subject.

“Introducing into a cell,” when referring to an iRNA agent (e.g., dsRNA agent), means facilitating or effecting uptake or absorption into the cell. Absorption or uptake of an iRNA agent (e.g., dsRNA agent) can occur through unaided diffusive or active cellular processes, or by auxiliary agents or devices. The meaning of this term is not limited to cells in vitro; an iRNA agent (e.g., dsRNA agent) may also be “introduced into a cell,” wherein the cell is part of a living organism. In such an instance, introduction into the cell will include the delivery to the organism. For example, for in vivo delivery, an iRNA agent (e.g., dsRNA agent) can be injected into a tissue site or administered systemically. In vitro introduction into a cell includes methods known in the art such as electroporation and lipofection. Further approaches are described herein below or known in the art.

In some embodiments, the siRNA agent (e.g., dsRNA agent) targets a sequence selected from any one of SEQ ID NOs: 1-24.

In some embodiments, the sense strand of the siRNA agent (e.g., dsRNA agent) comprises a nucleotide sequence selected from any one of SEQ ID NOs: 25-48, 73-96 and 121-144.

In some embodiments, the antisense strand of the siRNA agent (e.g., dsRNA agent) comprises a nucleotide sequence selected from any one of SEQ ID NOs: 49-72, 97-120, 145-168.

In some embodiments, the sense strand of the siRNA agent (e.g., dsRNA agent) comprises a nucleotide sequence selected from any one of SEQ ID NOs: 25-48. In some embodiments, the antisense strand of the siRNA agent (e.g., dsRNA agent) comprises a nucleotide sequence selected from any one of SEQ ID NOs: 49-72.

In some embodiments, the sense strand of the siRNA agent (e.g., dsRNA agent) comprises a nucleotide sequence of SEQ ID NO: 74 and antisense strand comprises a nucleotide sequence of SEQ ID NO: 98.

In some embodiments, the sense strand of the siRNA agent (e.g., dsRNA agent) comprises a nucleotide sequence of SEQ ID NO: 122 and antisense strand comprises a nucleotide sequence of SEQ ID NO: 146.

In some embodiments, the sense strand of the siRNA agent (e.g., dsRNA agent) comprises a nucleotide sequence of SEQ ID NO: 26 and antisense strand comprises a nucleotide sequence of SEQ ID NO: 50.

In some embodiments, the sense strand of the siRNA agent (e.g., dsRNA agent) comprises a nucleotide sequence of SEQ ID NO: 75 and antisense strand comprises a nucleotide sequence of SEQ ID NO: 99.

In some embodiments, the sense strand of the siRNA agent (e.g., dsRNA agent) comprises a nucleotide sequence of SEQ ID NO: 123 and antisense strand comprises a nucleotide sequence of SEQ ID NO: 147.

In some embodiments, the sense strand of the siRNA agent (e.g., dsRNA agent) comprises a nucleotide sequence of SEQ ID NO: 27 and antisense strand comprises a nucleotide sequence of SEQ ID NO: 51.

### Modified Inhibitory Nucleic Acid Molecules

The inhibitory nucleic acid molecules (e.g., siRNA agents) described herein can include one or more nucleotide modification. In some embodiments, no more than five of the sense strand nucleotides and no more than five of the nucleotides of the antisense strand are unmodified nucleotides. In some embodiments, all of the nucleotides of the sense strand and all of the nucleotides of the antisense strand comprise a modification.

In some embodiments, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide; and combinations thereof. In some embodiments, no more than five of the sense strand nucleotides and not more than five of the nucleotides of the antisense strand include modifications other than 2′-O-methyl modified nucleotide, a 2′-fluoro modified nucleotide, a 2′-deoxy-modified nucleotide, unlocked nucleic acids (UNA) or glycerol nucleic acid (GNA).

Compositions comprising the inhibitory nucleic acid molecules (e.g., siRNA agents), alone or conjugated to an antibody are provided herein.

The inhibitory nucleic acid molecules can be modified such that they specifically bind to receptors or antigens expressed on a selected cell surface, e.g., by linking the inhibitory nucleic acid nucleic acid molecules to peptides or antibodies that bind to macrophage surface receptors or antigens, e.g., a TREM2 antibody.

Triggering receptor expressed on myeloid cells 2 (TREM2) is a membrane protein that forms a receptor signaling complex with the TYRO protein tyrosine kinase binding protein. This protein functions in immune response and is involved in chronic inflammation by triggering the production of constitutive inflammatory cytokines. Defects in the TREM2 are a cause of polycystic lipomembranous osteodysplasia with sclerosing leukoencephalopathy (PLOSL). Alternative splicing of the TREM2 gene results in multiple transcript variants encoding different isoforms. Example accession numbers of the human TREM2 protein are NCBI Genbank Accession No. AAH32362.1 NP_061838.1 or NP_001258750.1. A RefSeq for the human TREM2 gene is NCBI Accession No. NM 018965.4.

TREM2 antibodies are known in the art, and include the TREM2 antibody described in Wang et al., J Exp Med (2020) 217 (9): e20200785, as well as those commercially available, e.g., from Abbexa Ltd; Abcam; Abnova Corporation; Affinity Biosciences; Alomone Labs, Ltd.; antibodies-online; AntibodySystem; Aves Labs, Inc.; Aviva Systems Biology; Bioassay Technology Laboratory; Biomatik; Biorbyt; Bioss Inc.; BosterBio; Cell Signaling Technology; Creative Biolabs; Creative Diagnostics; DIMA Biotechnology Ltd.; Enzo Life Sciences, Inc.; EpiGentek; G Biosciences; GeneTex; HUABIO; IBL-America; Leading Biology; LifeSpan BioSciences; MilliporeSigma; MyBioSource.com; Novus Biologicals; OriGene Technologies; ProSci, Inc; Proteintech Group Inc; R&D Systems; RayBiotech; Santa Cruz Biotechnology, Inc.; Sino Biological, Inc.; St John's Laboratory; Thermo Fisher Scientific; and United States Biological. Methods for conjugating inhibitory nucleic acids to antibodies are known in the art an include covalent and non-covalent methods as described in Cao et al., Medicine in Drug Discovery, Volume 15, September 2022, 100128, and references cited therein, all of which are incorporated by reference herein.

### Pharmaceutical Compositions

In one aspect, the disclosure provides pharmaceutical compositions containing an inhibitory nucleic acid molecule (e.g., siRNA agent), as described herein, and a pharmaceutically acceptable carrier. The pharmaceutical composition containing the inhibitory nucleic acid molecule (e.g., siRNA agent) is useful for treating a disease or disorder related to the expression or activity of SPP1) (e.g., atrial diseases such as AFib).

Such pharmaceutical compositions are formulated based on the mode of delivery. In some embodiments, compositions can be formulated for localized delivery (e.g., into a heart tissue). In another example, compositions can be formulated for systemic administration via parenteral delivery, e.g., by intravenous (IV) delivery, intramuscular (IM), or subcutaneous delivery (subQ). In some embodiments, the pharmaceutical composition described herein is administered intravenously.

As used herein, a “pharmaceutical composition” comprises a pharmacologically effective amount of a therapeutic agent (e.g., an iRNA) and a pharmaceutically acceptable carrier.

As used herein, “pharmacologically effective amount,” “therapeutically effective amount” or simply “effective amount” refers to that amount of an agent (e.g., iRNA) effective to produce the intended pharmacological, therapeutic or preventive result. For example, in a method of treating a disorder related to SPP1 expression (e.g., atrial diseases such as AFib), an effective amount includes an amount effective to reduce one or more symptoms associated with the disorder (e.g., an amount effective to (a) inhibit pain or (b) inhibit or reduces the expression or activity of SPP1) or an amount effective to reduce the risk of developing conditions associated with the disorder. For example, if a given clinical treatment is considered effective when there is at least a 10% reduction in a measurable parameter associated with a disease or disorder, a therapeutically effective amount of a drug for the treatment of that disease or disorder is the amount necessary to obtain at least a 10% reduction in that parameter. For example, a therapeutically effective amount of an iRNA targeting SPP1 can reduce a level of SPP1 mRNA or a level of SPP1 protein by any measurable amount, e.g., by at least 10%, 15%, 20%, 25%, 30%, 35%, 40%, 45%, 50%, 55%, 60%, 65%, 70%, 75%, 80%, 85%, 90%, or 95%.

The term “pharmaceutically acceptable carrier” refers to a carrier for administration of a therapeutic agent. Such carriers include, but are not limited to, saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. Agents included in drug formulations are described further herein below.

As used herein, a “subject” to be treated according to the methods described herein, includes a human or non-human animal, e.g., a mammal. The mammal may be, for example, a rodent (e.g., a rat or mouse) or a primate (e.g., a monkey). In some embodiments, the subject is a human. A “subject in need thereof” includes a subject having, suspected of having, or at risk of developing a disorder related to SPP1 expression, e.g., atrial diseases such as AFib. In some embodiments, the subject has, or is suspected of having, a disorder related to SPP1 expression or overexpression. In some embodiments, the subject is at risk of developing a disorder related to SPP1 expression or overexpression.

As used herein, “target sequence” refers to a contiguous portion of the nucleotide sequence of an mRNA molecule formed during the transcription of a gene, e.g., SPP1, including mRNA that is a product of RNA processing of a primary transcription product. The target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the phrases “therapeutically effective amount” and “prophylactically effective amount” and the like refer to an amount that provides a therapeutic benefit in the treatment, prevention, or management of any disorder or pathological process related to SPP1 expression (e.g., atrial diseases such as AFib). The specific amount that is therapeutically effective may vary depending on factors known in the art, such as, for example, the type of disorder or pathological process, the patient's history and age, the stage of the disorder or pathological process, and the administration of other therapies.

In some embodiments, a composition provided herein is formulated for intravenous delivery. The pharmaceutical compositions featured herein are administered in a dosage sufficient to inhibit expression of SPP1. In general, a suitable dose of inhibitory nucleic acid molecule (e.g., siRNA agent) will be in the range of 0.01 to 200.0 milligrams per kilogram body weight of the recipient per day, generally in the range of 1 to 50 mg per kilogram body weight per day. For example, the dsRNA can be administered at 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose.

In some embodiments, a repeat-dose regimen may include administration of a therapeutic amount of an inhibitory nucleic acid molecule (e.g., siRNA agent) on a regular basis, such as monthly to once every six months. In certain embodiments, the inhibitory nucleic acid molecule (e.g., siRNA agent) is administered about once per quarter (i.e., about once every three months) to about twice per year. After an initial treatment regimen (e.g., loading dose), the treatments can be administered on a less frequent basis. In other embodiments, the pharmaceutical composition may be administered once daily, or the inhibitory nucleic acid molecule (e.g., siRNA agent) may be administered as two, three, or more sub-doses at appropriate intervals throughout the day or even using continuous infusion or delivery through a controlled release formulation. In that case, the inhibitory nucleic acid molecule (e.g., siRNA agent) contained in each sub-dose must be correspondingly smaller in order to achieve the total daily dosage. The dosage unit can also be compounded for delivery over several days, e.g., using a conventional sustained release formulation which provides sustained release of the inhibitory nucleic acid molecule (e.g., siRNA agent) over a several day period. Sustained release formulations are well known in the art and are particularly useful for delivery of agents at a particular site, such as can be used with the agents of the present disclosure. In this embodiment, the dosage unit contains a corresponding multiple of the daily dose. The effect of a single dose on osteopontin levels can be long lasting, such that subsequent doses are administered at not more than 3, 4, or 5-day intervals, or at not more than 1, 2, 3, 4, 12, 24, or 36-week intervals. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments.

Estimates of effective dosages and in vivo half-lives for the individual inhibitory nucleic acid molecules (e.g., siRNA agents) encompassed by the disclosure can be made using conventional methodologies or on the basis of in vivo testing using a suitable animal model. A suitable animal model, e.g., a mouse or a cynomolgus monkey, e.g., an animal containing a transgene expressing human SPP1, can be used to determine the therapeutically effective dose and/or an effective dosage regimen administration of SPP1 siRNA. In some embodiments, the inhibitory nucleic acid molecule (e.g., siRNA agent) described herein can be delivered in a manner to target a particular tissue, such as the CNS (e.g., optionally the brain or spine tissue, e.g., cortex, cerebellum, dorsal root ganglia, substantia nigra, cerebellar dentate nucleus, pallidum, striatum, brainstem, thalamus, subthalamic, red, and pontine nuclei, cranial nerve nuclei and the anterior horn; and Clarke's column of the spinal cord cervical spine, lumbar spine, or thoracic spine). The present disclosure also includes pharmaceutical compositions and formulations that include the inhibitory nucleic acid molecule (e.g., siRNA agent) featured herein. The pharmaceutical compositions of the present disclosure may be administered in a number of ways depending upon whether local or systemic treatment is desired and upon the area to be treated. Administration may be local (e.g., by intrathecal, intraventricular, intracerebroventricular, intracranial, epidural, or intraganglionic injection), topical (e.g., buccal and sublingual administration), oral, intravitreal, transdermal, airway (aerosol), nasal, rectal, or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal, or intraventricular administration.

In some embodiments, the administration is via a bolus injection. In some embodiments, the administration is via a depot injection. A depot injection may release the inhibitory nucleic acid molecule (e.g., siRNA agent) in a consistent way over a prolonged time period. Thus, a depot injection may reduce the frequency of dosing needed to obtain a desired effect, e.g., a desired inhibition of SPP1, or a therapeutic or prophylactic effect. In some embodiments, the administration is via a pump. The pump may be an external pump or a surgically implanted pump. In other embodiments, the pump is an infusion pump. An infusion pump may be used for intracranial, intravenous, or epidural infusions. In certain embodiments, the pump is a surgically implanted pump that delivers the inhibitory nucleic acid molecule (e.g., siRNA agent) to the CNS. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the inhibitory nucleic acid molecule (e.g., siRNA agent) featured in the disclosure are in admixture with a topical delivery agent such as lipids, liposomes, fatty acids, fatty acid esters, steroids, chelating agents and surfactants.

In some embodiments, the composition comprising the inhibitory nucleic acid molecule is conjugated with an antibody that binds to a macrophage surface receptor or antigen. In some embodiments, the antibody is an anti-TREM2 antibody. In some embodiments, the inhibitory nucleic acid molecule is conjugated to the anti-TREM2 antibody. In some embodiments, the inhibitory nucleic acid molecule is conjugated to the anti-TREM2 antibody via a linker.

Certain compositions of the present disclosure also incorporate carrier compounds in the formulation. As used herein, “carrier compound” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183).

In contrast to a carrier compound, a pharmaceutical carrier or excipient may comprise, e.g., a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

### Methods of Treatment

Provided herein are methods for treating or reducing the risk of progression of development or recurrence of an atrial disease, e.g., atrial fibrillation (AFib), by administering an inhibitory nucleic acid molecule targeting SPP1 described herein or a pharmaceutical composition described herein.

In the context of the present disclosure, the terms “treat,” “treatment,” and the like mean to prevent, delay, relieve or alleviate at least one symptom associated with a disorder related to SPP1 expression, or to slow or reverse the progression or anticipated progression of such a disorder. For example, the methods featured herein, when employed to treat pain, e.g., chronic pain or a pain-related disorder, may serve to reduce or prevent one or more symptoms of the pain, e.g., chronic pain, as described herein, or to reduce the risk or severity of associated conditions. Thus, unless the context clearly indicates otherwise, the terms “treat,” “treatment,” and the like are intended to encompass prophylaxis, e.g., prevention of disorders and/or symptoms of disorders related to SPP1 expression. Treatment can also mean prolonging survival as compared to expected survival in the absence of treatment. By “lower” in the context of a disease marker or symptom is meant any decrease, e.g., a statistically or clinically significant decrease in such level. The decrease can be, for example, at least 10%, at least 20%, at least 30%, at least 40%, at least 40%, at least 50%, at least 60%, at least 70%, at least 80%, or at least 90%. The decrease can be down to a level accepted as within the range of normal for an individual without such disorder.

The inhibitory nucleic acid molecules described herein can be administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, inhibitory nucleic acid molecules can be modified to target selected cells and then administered systemically. For systemic administration, inhibitory nucleic acid molecules can be modified such that they specifically bind to receptors or antigens expressed on a selected cell surface, e.g., by linking the inhibitory nucleic acid nucleic acid molecules to peptides or antibodies that bind to cell surface receptors or antigens, e.g., as described herein.

The inhibitory nucleic acid nucleic acid molecules can also be delivered using lipid-based nanoparticle (LNP) systems as known in the art. Optionally, the LNPs can also include antibodies that bind to macrophage surface receptors or antigens, e.g., a TREM2 antibody as described herein.

The inhibitory nucleic acid nucleic acid molecules can also be delivered to cells using vectors known in the art. To achieve sufficient intracellular concentrations of the inhibitory nucleic acid molecules, vector constructs in which the inhibitory nucleic acid nucleic acid molecule is placed under the control of a strong promoter can be used.

In some embodiments, the inhibitory nucleic acid nucleic acid molecule disclosed herein is administered in combination with a second therapy (e.g., one or more additional therapies) known to be effective in treating a disorder related to SPP1 expression (e.g., atrial diseases such as AFib) or a symptom of such a disorder. The dsRNA agent may be administered before, after, or concurrent with the second therapy. In some embodiments, the inhibitory nucleic acid nucleic acid molecule is administered before the second therapy. In some embodiments, inhibitory nucleic acid nucleic acid molecule is administered after the second therapy. In some embodiments, the inhibitory nucleic acid nucleic acid molecule is administered concurrent with the second therapy. The second therapy may be an additional therapeutic agent.

The inhibitory nucleic acid molecule and the additional therapeutic agent can be administered in combination in the same composition or the additional therapeutic agent can be administered as part of a separate composition.

In some embodiments, the administration is repeated, for example, on a regular basis, such as, daily, biweekly (i.e., every two weeks) for one month, two months, three months, four months, six months or longer. After an initial treatment regimen, the treatments can be administered on a less frequent basis. For example, after administration biweekly for three months, administration can be repeated once per month, for six months or a year or longer.

In some embodiments, the inhibitory nucleic acid molecule is administered in two or more doses. In some embodiments, the number or amount of subsequent doses is dependent on the achievement of a desired effect, e.g., to (a) reduce pain; (b) inhibit or reduce the expression or activity of osteopontin or the achievement of a therapeutic or prophylactic effect, e.g., reduction or prevention of one or more symptoms associated with the disorder. In some embodiments, the inhibitory nucleic acid molecule is administered according to a schedule. For example, the inhibitory nucleic acid molecule may be administered once per week, twice per week, three times per week, four times per week, or five times per week. In some embodiments, the schedule involves regularly spaced administrations, e.g., hourly, every four hours, every six hours, every eight hours, every twelve hours, daily, every 2 days, every 3 days, every 4 days, every 5 days, weekly, biweekly, or monthly. In some embodiments, the inhibitory nucleic acid molecule is administered at the frequency required to achieve a desired effect. In some embodiments, the schedule involves closely spaced administrations followed by a longer period of time during which the agent is not administered. For example, the schedule may involve an initial set of doses that are administered in a relatively short period of time (e.g., about every 6 hours, about every 12 hours, about every 24 hours, about every 48 hours, or about every 72 hours) followed by a longer time period (e.g., about 1 week, about 2 weeks, about 3 weeks, about 4 weeks, about 5 weeks, about 6 weeks, about 7 weeks, or about 8 weeks) during which the inhibitory nucleic acid molecule is not administered.

In some embodiments, the inhibitory nucleic acid molecule is initially administered hourly and is later administered at a longer interval (e.g., daily, weekly, biweekly, or monthly). In some embodiments, the inhibitory nucleic acid molecule is initially administered daily and is later administered at a longer interval (e.g., weekly, biweekly, or monthly). In certain embodiments, the longer interval increases over time or is determined based on the achievement of a desired effect. Before administration of a full dose of the inhibitory nucleic acid molecule, patients can be administered a smaller dose, such as a 5% infusion dose, and monitored for adverse effects, such as an allergic reaction, or for elevated lipid levels or blood pressure. In another example, the patient can be monitored for unwanted effects.

### EXAMPLES

The invention is further described in the following examples, which do not limit the scope of the invention described in the claims.

### Materials and Methods

The following materials and methods were used in Examples 1 and 2 below.

Human Samples for scRNA-Seq

Human left atrial appendages were obtained from well-characterized patients undergoing cardiac surgery at the Massachusetts General Hospital. The enrollment specifically involved patients with or without persistent AFib. Patients with AFib had pre-existing moderate to severe mitral valve regurgitation (MR). Control patients never had AFib. General inclusion criterion was age >18 years, with no specific exclusion criteria on the basis of sex, gender, race or ethnicity. Human tissue collection was conducted according to the Declaration of Helsinki and was approved by the Mass General Brigham Institutional Review Board (2019P002446). All patients gave written, informed consent before sample collection, as required. The excised atrial tissue samples were placed in a specimen container containing ice-cold PBS in the operating room and processed immediately for single-cell transcriptional profiling.

### Human Samples for Histology

Existing formalin-fixed paraffin-embedded (FFPE) blocks of human left atrial samples, collected during cardiac surgery or autopsy at different clinical sites (Massachusetts General Hospital, Brigham and Women's Hospital, University Hospital Wuerzburg, University Hospital Goettingen and John Radcliffe Hospital), were obtained for histology with approval from the local Research Ethics Committee at Mass General Brigham (2012P001135 and 2019P003227), University Hospital Wuerzburg (59/26-sc), University Hospital Goettingen (14/9/11) and John Radcliffe Hospital (07/Q1607/38 and 18/SW/0243). Patients were categorized in four groups based on their MR and/or AFib diagnoses. MR was defined significant if it was recorded as moderate or severe by echocardiography prior to sampling. Recorded electrocardiogramata of paroxysmal, persistent or permanent AFib prior to sampling was defined as AFib. Left and right atria were defined as dilated if diameter, area or volume were larger than reported upper reference values (51, 52). Detailed patient characteristics of the scRNA-seq and histology cohorts are shown in tables S1 and S3, respectively.

### Mice

Wildtype C57BL/6 (stock 000664), B6.129P2 (Cg)-Cx3cr1^{tm2.1 (cre/ERT2)Litt}/WganJ (Cx3cr1^{CreER}, stock 021160), B6.Cg-Gt(ROSA)26Sor^{tm9(CAG-tdTomato)Hze}/J (Ai9^{fl/fl}, stock 007909), B6.129S4-Ccr2^{tm1lfc}/J (Ccr2^{-/-}, stock 004999), B6.SJL-Ptprc^{a} Pepc^{b}/BoyJ (Cd45.1, stock 002014) and B6.129S6 (Cg)-Spp1^{tm1Blh}/J (Spp1^{-/-}, stock 004936) were purchased from the Jackson Laboratory. Genotyping for each strain was performed as described on the Jackson Laboratory website. All experiments were performed with 8-to-12-week-old male and female animals and using age- and sex-matched groups. Where appropriate, mice were randomly assigned to interventions. All mice were group-housed under standard conditions with free access to food and water. All animal experiments were approved by the Institutional Animal Care and Use Committee at the Massachusetts General Hospital (2014N000078).

### In Vivo Interventions

### HOMER Mouse Model

To induce atrial remodeling and a disease condition that combines all three clinical risk factors (hypertension, obesity and mitral valve regurgitation (HOMER)), mice first underwent surgery to generate MR, were then fed a high-fat diet and finally had hypertension induced. In detail, mice were anesthetized (2.5% isoflurane in 95% O₂), intubated, ventilated, and placed on a heating pad. A left lateral thoracotomy at the level of the second or third intercostal space was performed and the pericardial sack was opened to gain access to the heart. The mouse was then rotated to the right in order to expose the anterior and lateral aspects of the left atrium and ventricle. The left atrium was carefully elevated and a 8.0 suture was inserted into the inferior part of the left atrium just above the mitral annulus. The needle was guided through the mitral valve without visual control and exerted through the left ventricle just inferior of the mitral annulus and superior to the left circumflex artery. A 5-throw surgical knot was performed, thereby mechanically snaring the mitral valve to the left ventricular wall. The intercostal space and skin were surgically closed with a 5.0 suture. The animals were allowed to recover with oxygen on a heating pad until extubation. For sham surgery, mice underwent left thoracotomy as described above, and the pericardium was incised. In an additional cohort of sham-operated mice the same surgical steps were undertaken as described above (including needle insertion through the left atrium and mitral valve as well as exertion through the left ventricle) but without tying a surgical knot. Four weeks after surgery, either regular chow diet or high-fat diet (Research Diets, D12492) was initiated and continued for 17 weeks. Finally, 20 weeks after mitral valve surgery, animals were exposed to continuous infusion of either saline or angiotensin 2 (Sigma-Aldrich, 1 μg/kg/min) for 1 week via a subcutaneously implanted osmotic minipump (Alzet).

### EP Study

EP studies were performed under general anesthesia with isoflurane. A Millar EPR-800 octapolar catheter was inserted into the right jugular vein and positioned in the right atrium and ventricle. Sinus node function was determined by measuring the sinus node recovery time following 30 s of pacing at two cycle lengths (100 and 80 ms). The Wenckebach cycle length was measured with progressively faster atrial pacing rates. Atrial and atrioventricular node effective refractory periods were measured using programmed electrical stimulation with overdrive pacing trains at 100 ms followed by single extrastimuli. Provocative testing for arrhythmia induction was performed with double extrastimuli (S1-S2-S3) as well as burst pacing at gradually faster rates to a pacing cycle length of 10 ms. Two different drive train cycle lengths (S1) were used (120 ms and 100 ms), and each set of double extrastimuli was done twice. Burst pacing was done twice at a duration of 3 s and then 6 s, with cycle length starting at 50 ms and progressing to 10 ms in intervals of 5 ms. AFib was defined as a rapid atrial rhythm with an atrial rate faster than the ventricular rate and irregular ventricular response (R-R intervals). The duration was measured from the end of the pacing cycle to the first P wave. A mouse was deemed inducible if it had at least one episode of AFib lasting more than 500 ms. The total AFib burden for any given mouse was obtained by summing all episodes of AFib >250 ms during the course of the entire EP study.

### Ambulatory ECG Telemetry

Continuous ambulatory ECG telemetry was performed by implanting an ETA-F10 transmitter (Data Sciences International) during general anesthesia with isoflurane. The transmitter was implanted in the abdomen and the leads were tunneled subcutaneously to the upper right and lower left chest resulting in a lead II position. Telemetry data were recorded continuously via a receiver placed under the mouse cage. Data analysis was performed using LabChart Pro v8 (AD Instruments) software.

### Blood Pressure Measurements

Blood pressure in conscious mice was measured with a non-invasive tail-cuff system (Kent Scientific Corporation) per manufacturer's instructions.

### Bone Marrow Transplantation

For whole bone marrow transplants, recipient mice were lethally irradiated (single dose of 10 Gy) and received 4×10⁶ bone marrow cells from donor mice by intravenous injection. Mice were exposed to the HOMER protocol 8 weeks after transplantation.

### Tamoxifen Injection

Tamoxifen (Sigma-Aldrich) was administered as a solution in corn oil (Sigma-Aldrich) to Cx3cr1^{CreER/+}Ai9^{fl/+} mice by intraperitoneal injection. Animals received five doses (100 μl each) of 2 mg tamoxifen with 24 hours between doses.

### Synthesis of CCL2-Fc

A plasmid was generated to encode murine CCL2 mutant P8A (35) fused to the N terminus of a mouse CH2-CH3 IgG2c Fc domain bearing three point mutations (LALAPG) that ablate Fc/FcR-mediated effector functions. This plasmid was transfected into Expi293 cells to produce CCL2-Fc, a protein capable of inhibiting endogenous CCL2 from binding CCR2. The construct was then isolated by protein A sepharose affinity chromatography and dialyzed into PBS solution to a concentration of 1 mg/ml. SDS-PAGE confirmed that this construct was produced as a monomeric protein, and in vitro assays confirmed specific binding to CCR2.

### Cells

### Cell Collection

Mouse peripheral blood was collected by retro-orbital bleeding using heparinized capillary tubes, and red blood cells were lysed with 1× red blood cell lysis buffer (BioLegend). For organ harvest, mice were perfused through the left ventricle with 10 ml of ice-cold PBS, hearts were excised and left atrial tissues were microdissected using a dissection microscope. After harvest, tissues were minced into small pieces and subjected to enzymatic digestion with 450 U/ml of collagenase I, 125 U/ml of collagenase XI, 60 U/ml of DNase I, and 60 U/ml of hyaluronidase (all Sigma-Aldrich) for 40 min at 37° C. under agitation. Tissues were triturated and cells were filtered through a 40-μm nylon mesh, washed and centrifuged (340 g for 7 min) to obtain single-cell suspensions.

### Flow Cytometry

To detect leukocyte subsets in processed blood samples, cell suspensions were stained at 4° C. in FACS buffer (PBS supplemented with 0.5% bovine serum albumin) with anti-CD11b, -CD19, -CD90.2, -CD115, -Ly6C, and -Ly6G. For non-cardiomyocyte cell staining on processed heart samples, isolated cells were stained with anti-CCR2, -CD9, -CD11b, -CD31, -CD45, -CD64, -CD146, -MEFSK4, -MHCII, and/or-TREM2. Detailed antibody information is provided in FIG. 51. DAPI was used as a cell viability marker. Data were acquired on an LSRII (BD Biosciences) and analyzed with FlowJo software.

### Flow Cytometry Gating

Blood monocytes were identified as CD19⁻CD90.2⁻CD11b⁺CD115^{hi}Ly6C^{lo/hi}, neutrophils as CD19⁻CD90.2⁻CD11b⁺CD115^{lo/int}Ly6G⁺ and B and T cells as CD19⁺CD90.2⁻ and CD19⁻CD90.2⁺, respectively. Cardiac endothelial cells were identified as CD31⁺CD45⁻, fibroblasts as CD31⁻CD45⁻CD146⁻ MEFSK4⁺, mural cells as CD31⁻ CD45⁻ MEFSK4^{lo/int} CD146⁺, neutrophils as CD45⁺CD11b⁺CD64⁻CCR2⁻ and monocytes as CD45⁺CD11b⁺CD64⁻CCR2⁺. Cardiac macrophages were identified as CD45⁺CD11b⁺CD64⁺ and were further parsed by CCR2, MHCII, CD9, TREM2, YFP, and Td.

### Cell Sorting

Cardiac cell suspensions were stained to identify the indicated cell populations and cells were sorted on a FACSAria II cell sorter (BD Biosciences) directly into RLT Plus lysis buffer (Qiagen).

Human and Mouse scRNA-Seq

### Tissue Preparation

For human scRNA-seq, cardiac tissues were minced into small pieces and subjected to enzymatic digestion with 450 U/ml of collagenase I, 60 U/ml of DNase I, and 60 U/ml of hyaluronidase (all Sigma-Aldrich) for 1 hour at 37° C. under mild agitation. Digested tissues were triturated, filtered, washed and centrifuged (340 g for 7 min) to obtain single-cell suspensions. Red blood cells were lysed with ACK buffer (Lonza). For mouse scRNA-seq, cardiac tissues were processed as described above. Two male mice were pooled for each sequencing replicate. Cell suspensions were then stained with three viability dyes (DAPI, calcein and VDR, all ThermoFisher Scientific) as previously described (53, 54). Viable (DAPI), metabolically active (calcein⁺), nucleated (VDR⁺) single cells were FACS-purified from digested tissues on a FACSAria II cell sorter and subsequently loaded on a 10× Genomics Chromium Single Cell B or G chip as outlined by the 10× Genomics Chromium Single Cell 3′ Reagent kits v3 or v3.1 user guide. Libraries were prepared according to the manufacturer's protocol with appropriate modifications to PCR cycles based on the calculated cDNA concentration. Constructed libraries were validated and quantified with the Agilent High Sensitivity DNA chip and by real-time qPCR and subsequently sequenced on an Illumina NextSeq 500 in high-output mode or an Illumina NovaSeq 6000 system as recommended by 10× Genomics.

Pre-Processing scRNA-Seq Data

The sequencing reads for the human samples were aligned to the GRCh38 reference genome and processed with 10× Genomics Cell Ranger 6.0.2, which generated a gene-cell barcode count matrix for each sample. With the output from Cell Ranger, Seurat 4.0 (55, 56) was used for further analysis. Quality-control metrics were first applied to filter out low-quality cells and rarely expressed genes. Specifically, this process removed cells that expressed fewer than 250 genes or had fewer than 500 unique molecular identifiers (UMIs), as well as those with greater than 25% of total UMI count from mitochondrial genes. Genes expressed in fewer than 10 cells were also filtered out. Count normalization was performed with the R package sctransform (57), followed by an integration workflow (55) which aimed to identify shared cell states present across all samples under consideration. Principal component analysis was carried out on the integrated data, and the top 40 principal components were used to cluster the cells. The FindMarkers function in Seurat was used to identify markers for each cluster and the FindConservedMarkers function was used to identify markers conserved across the groups within each cluster. In both functions, a logistic regression model coupled with a likelihood ratio test was used to determine the differentially expressed genes in a given cluster compared to all other cells. The scRNA-seq data for the mouse samples were analyzed in the same way as detailed above except that the reference genome and versions of Cell Ranger and Seurat used were GRCm38 (mm10), Cell Ranger 3.0.2 or 7.0.1 and Seurat 3.1 or 4.1, respectively, and that slightly different filtering thresholds were applied. In particular, cells expressing fewer than 200 genes (as well as those with greater than 25% of total UMI count from mitochondrial genes) and genes expressed in fewer than three cells were filtered out.

### Differential Expression and Gene Set Enrichment Analyses

To detect differentially expressed genes between groups of cells with different disease conditions, pseudobulk differential expression analyses (58) were performed, taking into account biological replicates in our data sets. Specifically, for a given cell cluster, the cells and the UMI counts were aggregated to the sample level and the differential expression analysis was carried out with a quasi-likelihood F test based on the generalized linear model implementation of edgeR 3.34.1 (10, 59, 60). The output of the differential expression analysis was used to rank the genes by their fold change values. This rank list became the input for the gene set enrichment analysis (GSEA), which was performed with the GSEAPreranked tool from GSEA 4.2.0 (11, 61) and the collections of gene sets in the molecular signatures database MSigDB 7.4 or 7.5 (11, 62).

Weighted correlation network analysis and overrepresentation analysis

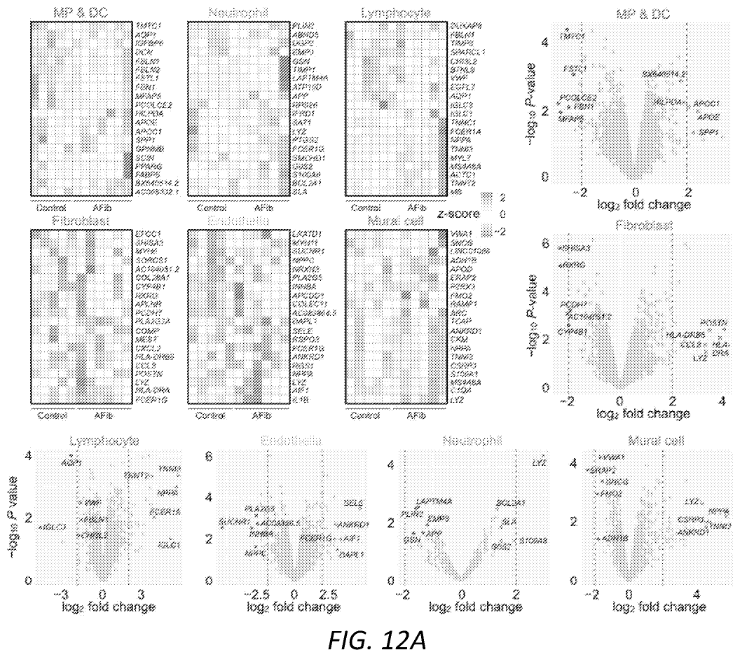

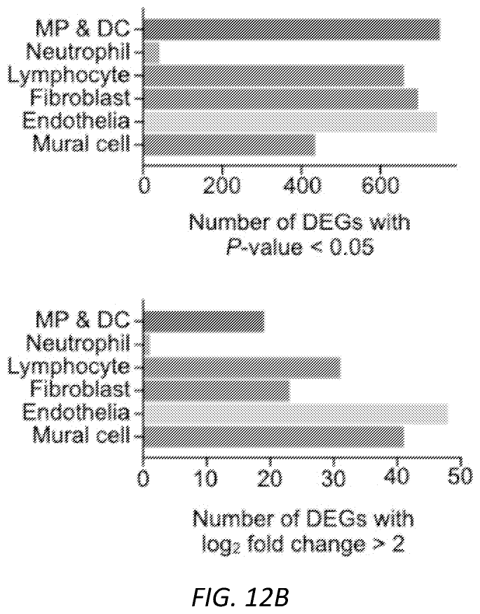

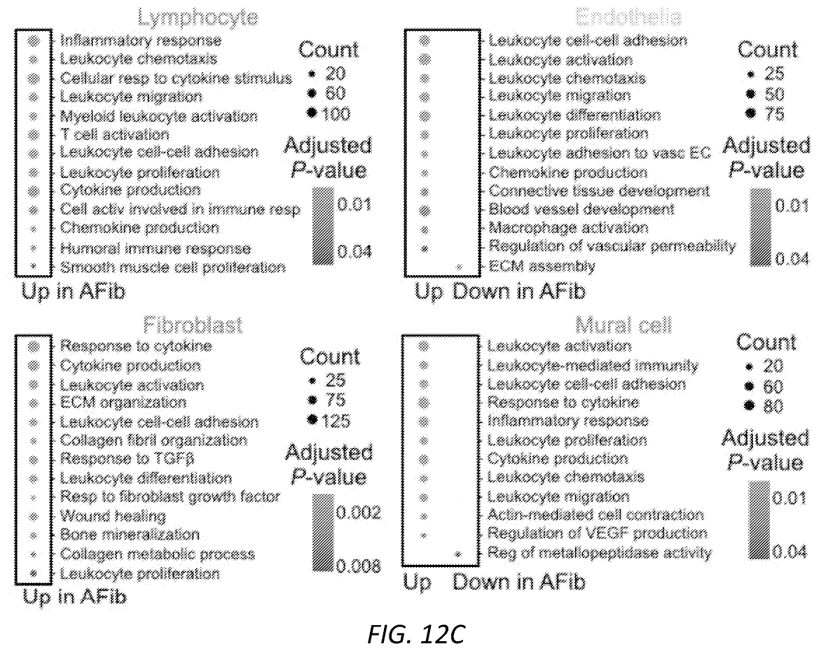

The R package WGCNA 1.70-3 (12, 63) was used to identify groups of genes (gene modules) with highly correlated expressions and significant up- or downregulation in the diseased condition. Overrepresentation analysis (64) from the R package clusterProfiler 4.1.4 (65) was used to identify the gene sets that were overrepresented in these gene modules. GSEA as implemented in the R package clusterProfiler 4.1.4 (65) was used in FIG. 1D and FIG. 12C.

Interactions Between Cells from Different Lineages

CellPhoneDB 2.1.4 (36, 66) was used to infer interlineage cell-cell interactions.

### Imaging and Histology

### Cardiac MRI

MRI was performed with a 4.7-Tesla horizontal bore Pharmascan system (Bruker) equipped with a custom-built mouse cardiac coil (Rapid Biomedical) to obtain cine images of the left atrial and ventricular short axes. ECG and respiratory gating (SA Instruments), a gradient echo FLASH-sequence and a dedicated mouse cardiac volume coil in bird cage design were used. Imaging parameters were as follows: echo time (TE), 2.7 to 5 ms; 16 frames per RR interval (TR 7.0-15 ms depending on heart rate); in-plane resolution, 150×150 μm; slice thickness, 1 mm; NEX 4; flip angle, 30 degrees. Anatomical and functional parameters were quantified using Horos software.

### Cardiac Ultrasound

Doppler ultrasound was used to assess successfully induced MR. Mice were imaged under general anesthesia using a GE Healthcare Vivid E90 system equipped with a 15 MHz linear probe (L8-18i-D) and the mitral Doppler inflow spectrum was recorded from the parasternal long axis view.

### Mouse Histology

To detect atrial fibrosis, hearts were embedded in OCT compound and frozen 7-μm sections were stained with Masson's trichrome. Briefly, sections were fixed with 10% formalin for 30 min at room temperature and incubated in Bouin's fixative solution (Electron Microscopy Sciences) overnight at room temperature, and the nuclei were stained with a mixture of Weigert's iron hematoxylin solution A and B (Electron Microscopy Sciences). The sections were then stained with Biebrich scarlet-acid fuchsin (StatLab), followed by phosphomolybdic acid/phosphotungstic acid solution (Sigma-Aldrich) and aniline blue stain (StatLab). TUNEL staining was carried out by using an In Situ Cell Death Detection kit (Roche) according to the manufacturer's protocol. For immunofluorescent staining, sections were stained with rabbit anti-mouse collagen I, rat anti-mouse CD68, rabbit anti-mouse Ki67, and/or FITC anti-mouse aSMA overnight at 4° C., followed by a biotinylated goat anti-rabbit IgG antibody and streptavidin DyLight 594, and/or an Alexa Fluor 488 goat anti-rat IgG antibody for 1 hour at room temperature. Detailed antibody information is provided in FIG. 52. The nuclei were stained with DAPI. All slides were scanned by a digital slide scanner NanoZoomer 2.0RS (Hamamatsu). To analyze cellular signals in mice, six fields of views (FOVs) from the left atrium per animal were exported at 40× using NanoZoomer NDP.view2 software. For each FOV, the percentage of positive signal was measured using ImageJ v2.9.0 (NIH) software. Ki67⁺ and TUNEL⁺ cells were manually counted per FOV.

### Human Histology

Left atrial FFPE samples were obtained from five different clinical sites (Massachusetts General Hospital, Brigham and Women's Hospital, University Hospital Wuerzburg, University Hospital Goettingen and John Radcliffe Hospital) and sectioned at 5 μm. The sections were then deparaffinized and rehydrated before immunofluorescent staining. Heat-induced antigen retrieval was performed using Retrievagen A (pH6.0, BD Biosciences) and the sections were permeabilized with 0.3% Triton X-100 in PBS for 10 min at room temperature. After the sections were blocked with 2% normal horse serum and 2% normal goat serum in PBS, primary antibodies, mouse anti-human CCR2 and rabbit anti-human CD68, were incubated at 4° C. overnight. For SPP1/COL1A1 detection, left atrial FFPE sections were treated with citrate unmasking solution (Cell Signaling) followed by staining with primary antibodies mouse anti-human SPP1 and rabbit anti-human COL1A1 at 4° C. overnight. The sections were then stained with a biotinylated horse anti-mouse IgG antibody and streptavidin DyLight 594, and an Alexa Fluor 488 goat anti-rabbit IgG antibody. Detailed antibody information is provided in FIG. 52. The nuclei were stained with DAPI. To remove lipofuscin and autofluorescence signal, tissues were incubated with TrueBlack Plus quencher (Biotium) for 10 min at room temperature. All the slides were scanned using the NanoZoomer 2.ORS scanner or imaged using a Zeiss LSM 800 Airyscan confocal microscope.

### CCR2⁺Macrophage Density Analysis

Atrial macrophages were enumerated in 3-5 FOVs per patient from 149 clinical cases using semiautomated image analysis (67). During a preprocessing stage, we reduced fluorescence background and increased the signal contrast to optimize cell segmentation. Image contrast was optimized using a contrast-limited adaptive histogram equalization algorithm and the images were spatially filtered and denoised. A watershed algorithm was used to count individual cells and a size threshold was employed to reject areas smaller than cells. After segmentation of macrophages in the Alexa Fluor 488 channel encoding for CD68, the resulting areas were considered in the DyLight 594 channel encoding for CCR2 to generate a reduced segmentation mask. The CCR2 signal within individual CD68 masks was then calculated and macrophages were marked positive for CCR2 if the value was above a threshold determined using the standard deviation of the CCR2 signal across the entire image. Segmentation results were inspected individually for quality control. Software (67) was written in Python 3.7.4 using OpenCV, NumPy and Scikit-learn packages.

### Human RNA In Situ Hybridization

In situ hybridization was performed on a Leica Biosystems BOND RX system using human FFPE samples and the RNAscope 2.5 LS Duplex assay with RNAscope 2.5 LS Green Accessory pack (both Advanced Cell Diagnostics) following the manufacturer's protocol with minor modifications. These modifications include an antigen retrieval step for 15 min at 88° C. and a protease incubation step for 15 min at 40° C. Each FFPE sample was first stained with the RNAscope 2.5 LS Duplex positive (Hs—PPIB-C1, Hs-POLR2A-C2) and negative (DapB-C1, DapB-C2) control probes to assess tissue quality. If the sample passed quality control metrics (i.e., sufficient positive control probe signal in the absence of negative control probe signal), the adjacent tissue section was stained with the RNAscope 2.5 LS Hs-CD68-C1 (Cat #560598) and Hs-SPP1-C2 (Cat #420108-C2) probes to detect CD68 and SPP1 signal, respectively. For quantification, three FOVs per sample were exported at 40× using NanoZoomer NDP.view2 software. For each FOV, SPP1⁺CD68⁺ macrophages were manually counted using ImageJ software. Cells with more than two spots for a given marker were considered positive for that marker.

### Molecular Biology

Real-Time qPCR

Total RNA from left atrial tissue was extracted using the RNeasy Plus Mini kit or from FACS-purified cells using the RNeasy Plus Micro kit (both Qiagen) according to the manufacturer's protocol. First-strand cDNA was synthesized using the High-Capacity RNA-to-cDNA kit (ThermoFisher Scientific) according to the manufacturer's instructions. TaqMan Fast Universal PCR Master Mix and TaqMan gene expression assays (both ThermoFisher Scientific) were used to quantify target genes. The relative changes were normalized to Gapdh mRNA using the 2-44Ct method.

### Statistics and Reproducibility

Results are shown as mean±SEM. Statistical analysis was performed using GraphPad Prism 9.5.1. Where appropriate, statistical outliers identified by the ROUT method (Q=1%) were excluded. Statistical tests included two-tailed Student's t tests, two-tailed paired t tests, ANOVA followed by Dunnett's or Tukey's multiple comparisons test, two-tailed Mann-Whitney tests, two-tailed Wilcoxon matched-pairs signed rank tests and Kruskal-Wallis tests followed by Dunn's multiple comparisons test (the latter three if Gaussian distribution was not assumed). For correlation analysis, the r or ρ values were computed using two-tailed Pearson correlation coefficients or two-tailed Spearman correlation coefficients, respectively. P-values of 0.05 or less were considered to denote significance. Animal group sizes were as low as possible and empirically chosen. No statistical methods were used to predetermine sample size. Animals were randomly assigned to treatment groups and analyzed in a blinded fashion where appropriate.

### Example 1. Recruited Macrophages Elicit Atrial Fibrillation

To examine how immune and stromal cells contribute to AFib, we performed single-cell RNA-sequencing (scRNA-seq) of freshly isolated normal and diseased human atrial tissue. In this study, we comprehensively assayed atria from patients and mice and documented large-scale shifts in the cellular frequencies and phenotypes during AFib. Macrophages expanded more than any other cell type in AFib. Experiments in HOMER knockout mice demonstrated that targeting macrophages strengthens resilience against AFib (FIG. 37).

### Macrophages Expand in Human Atrial Disease

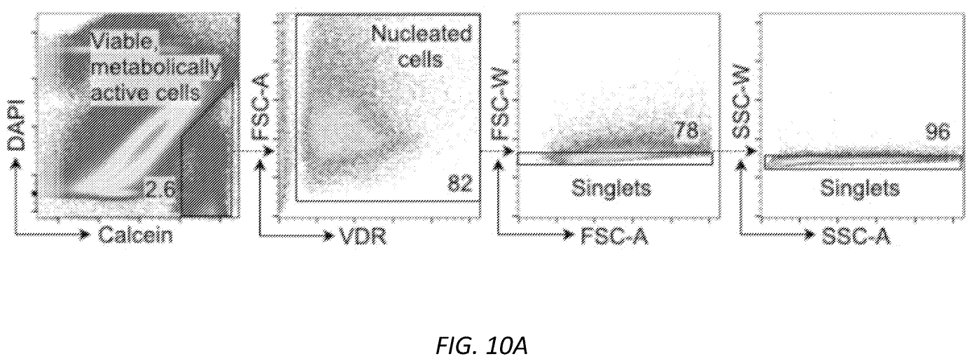

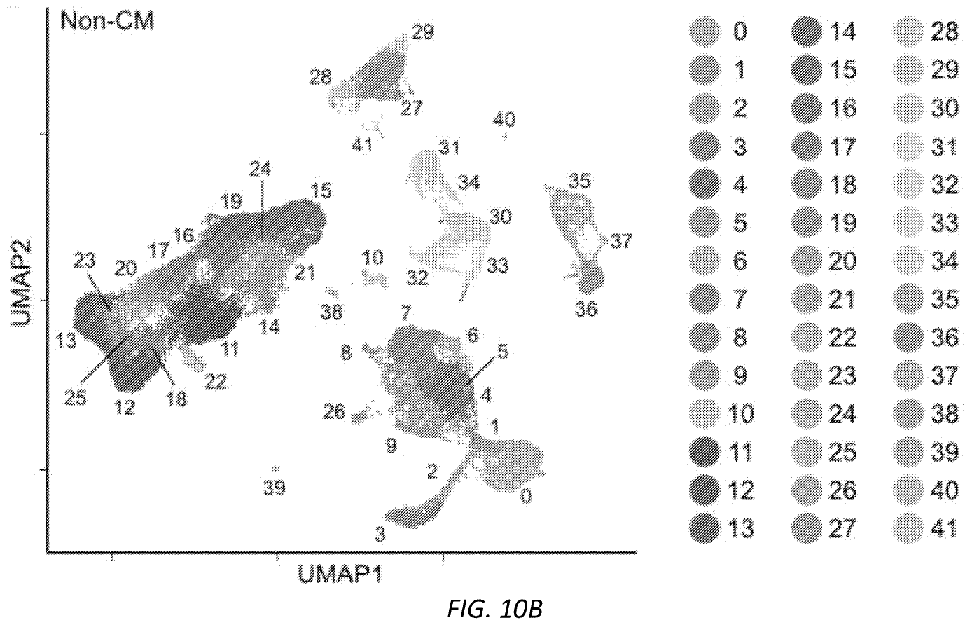

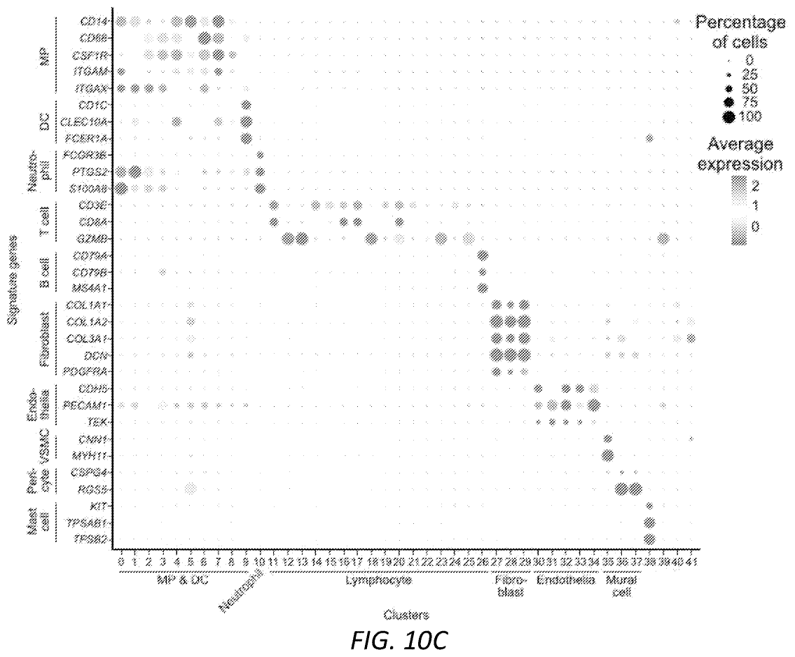

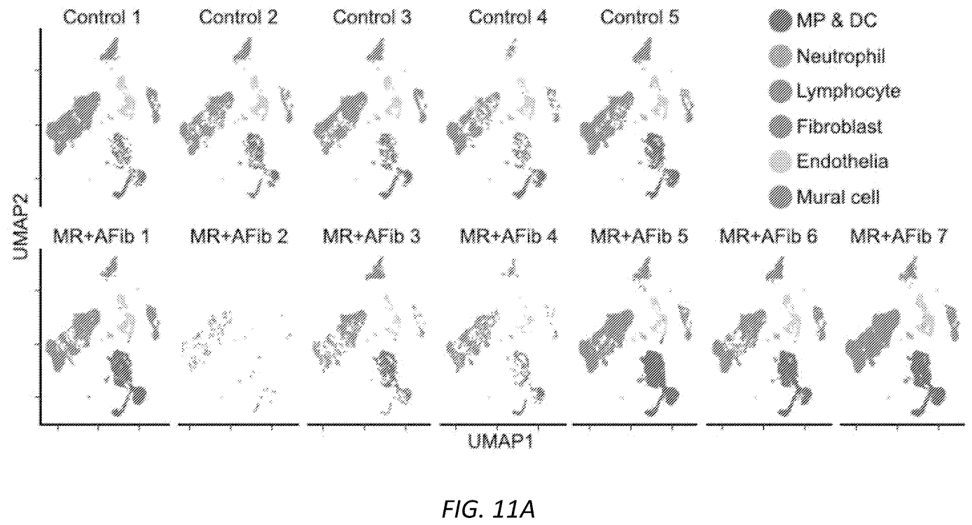

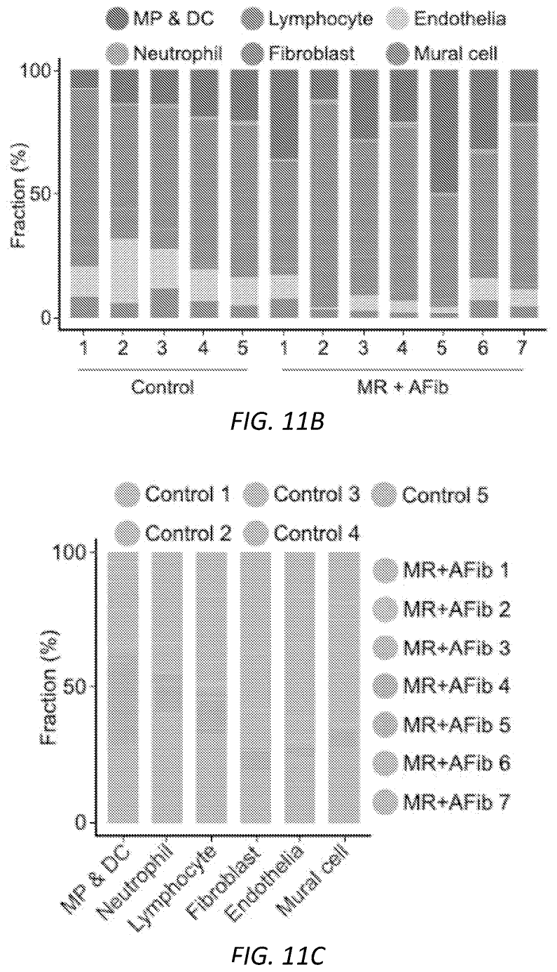

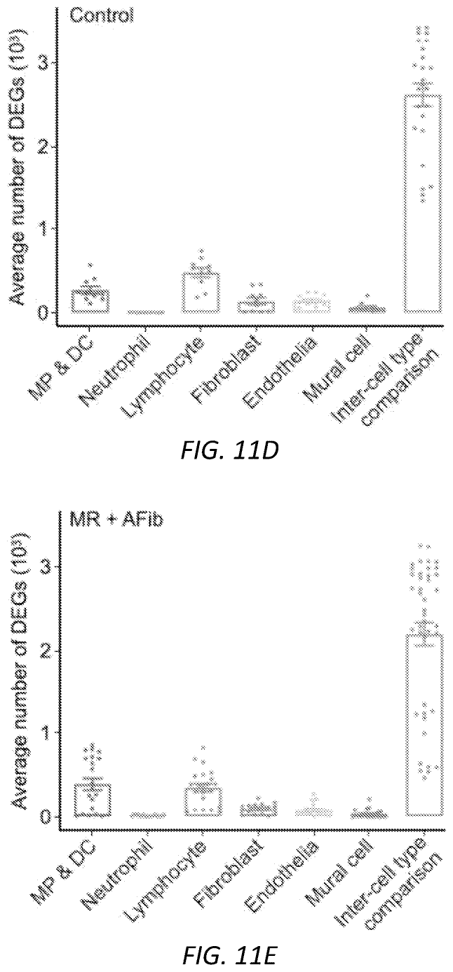

We collected left atrial tissue from five control subjects and seven patients with persistent AFib undergoing heart surgery (FIG. 42) and performed scRNA-seq on the 10× Genomics Chromium platform (FIG. 1A and FIG. 10A). A total of 41,609 viable, single cells from both AFib and control patients were partitioned into clusters (FIG. 10B), which we annotated using known lineage markers (FIG. 10C). We identified six major non-cardiomyocyte cell types which included, in order of decreasing frequency: lymphocytes, mononuclear phagocytes and dendritic cells (MP/DCs), endothelial cells, fibroblasts, mural cells, and neutrophils (FIGS. 1B and 1C; FIG. 11; and FIG. 43).

In diseased atria, the MP/DC cluster expanded twofold, whereas endothelia and mural cells decreased in frequency. The other cell classes remained stable (FIG. 1C and FIG. 43). Because the MP/DC cluster increased dramatically, we scrutinized the transcriptomes of these cells more closely. We identified genes that were differentially expressed in MP/DCs from AFib and control atria with a pseudobulk approach that aggregated cells from the same individual to determine differential gene expression between the two patient groups (10). We then performed gene set enrichment analysis (GSEA) (11) on the resulting list of differentially expressed genes to define the pathways and functional gene groups that were altered in MP/DCs from AFib patients. In this disease setting, MP/DCs upregulated gene sets related to the inflammatory response, IFNγ signaling, monocyte recruitment, and cholesterol metabolism (FIG. 1D). Thus, atrial MP/DCs undergo significant immunometabolic remodeling during their expansion in AFib patients.

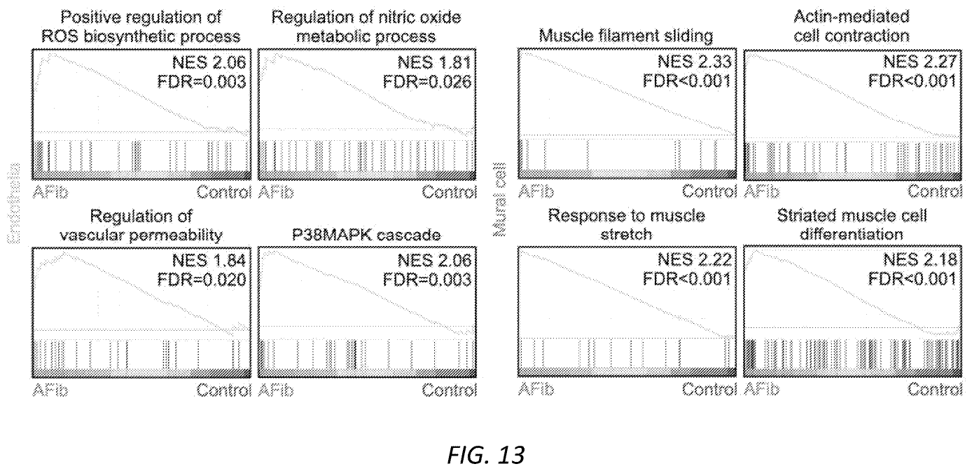

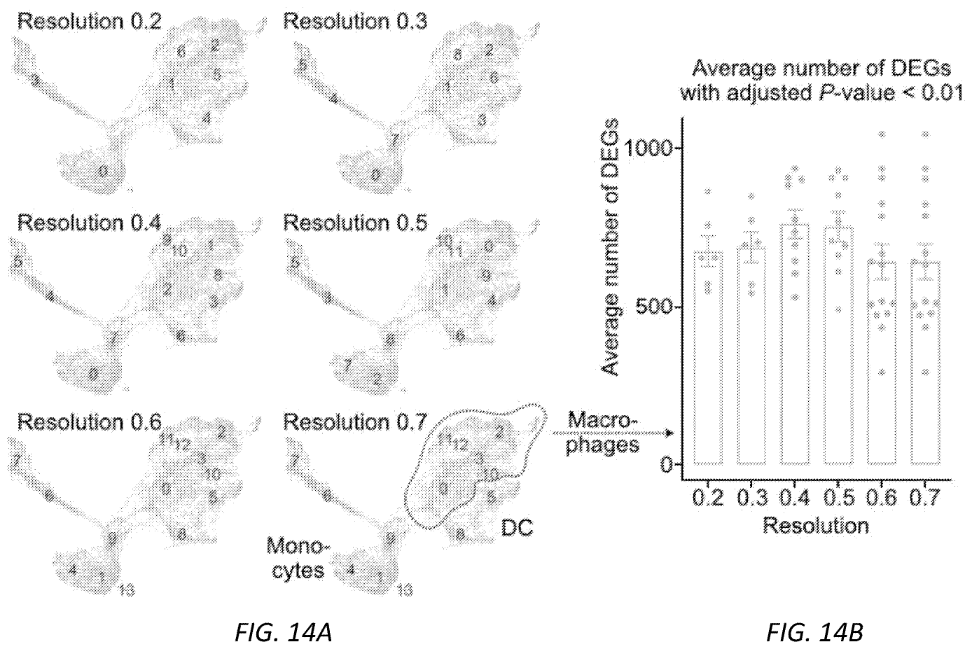

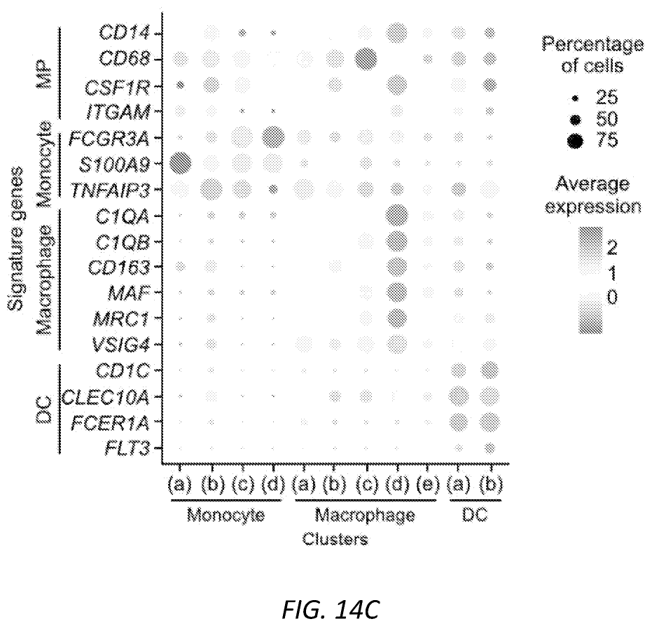

To gain a broader understanding of the transcriptional programs that are activated or repressed in MP/DCs, we used weighted correlation network analysis to study groups of correlated genes that were differentially expressed between patients and controls (12). We identified 24 modules of correlated genes across all participants, three of which were significantly correlated with disease state. Module 1 was upregulated in AFib and contained inflammatory and profibrotic genes such as CCR2, IL10, ITGA9, MMP9, SPP1, TIMP2, and VIM (13-15) (FIG. 1E). Overrepresentation analysis confirmed that inflammatory and extracellular matrix-related gene sets characterized module 1 (FIG. 1F). Module 1 genes SPP1, CCR2 and the cell surface markers TREM2 and CD9 were among the top upregulated genes in MP/DCs from AFib patients (FIG. 1G). SPP1 encodes the matricellular protein osteopontin, which participates in bone remodeling and fibrosis by promoting cell survival, adhesion, and migration as well as inflammatory cell activation (13). The chemokine receptor CCR2 mediates monocyte egress from the bone marrow (16) and is essential for monocyte recruitment (14). Gene modules 2 and 3, whose expression was downregulated in MP/DCs from AFib patients, included genes involved in protecting against cardiac disease (SIRT2 (17)), cardiac resident macrophage proliferation (KLF4 (18)), and epigenetic constraint of cytokine production (HDAC2 (19) and ODCI (20)) (FIG. 1E). These data imply that the atria of AFib patients have elevated numbers of myeloid cells that assume a more inflammatory phenotype, thereby implicating macrophages in the tissue remodeling that disrupts coordinated excitation of atrial cardiomyocytes. Other leukocytes and stromal cells also altered their gene expression (FIG. 12). Fibroblasts, for example, responded by upregulating inflammatory pathways and matrix production (FIG. 12C). In endothelial and mural cells, we observed upregulation of genes indicative of ischemia and vascular remodeling (FIG. 13).

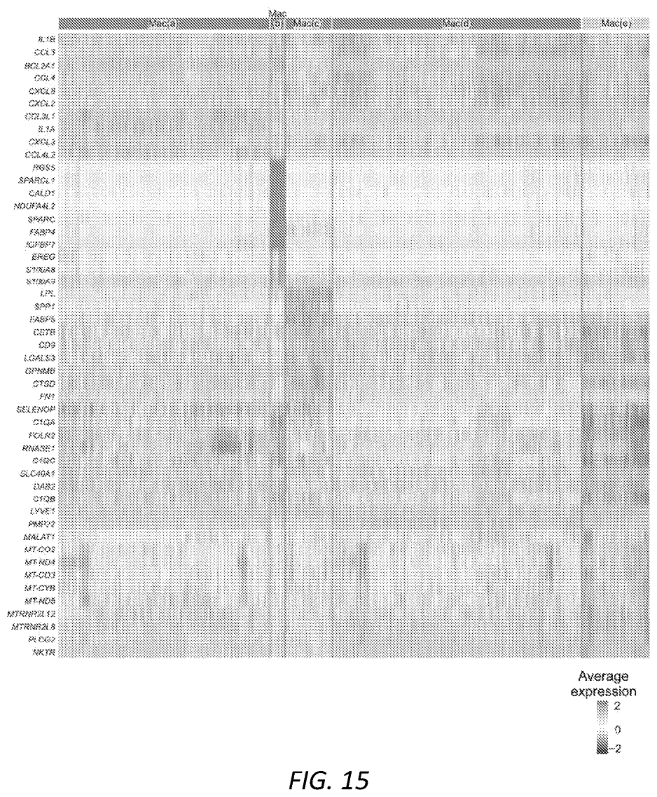

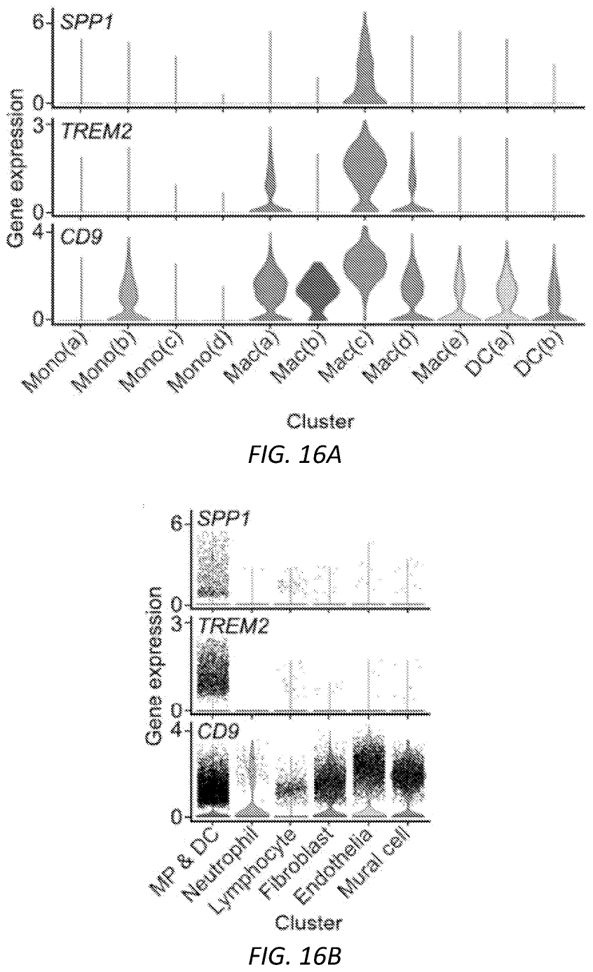

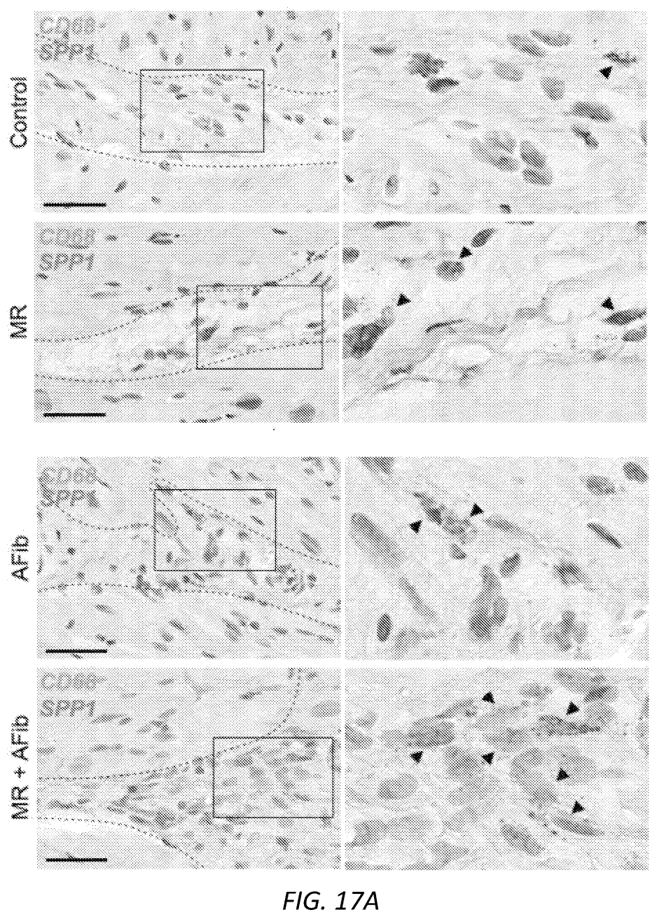

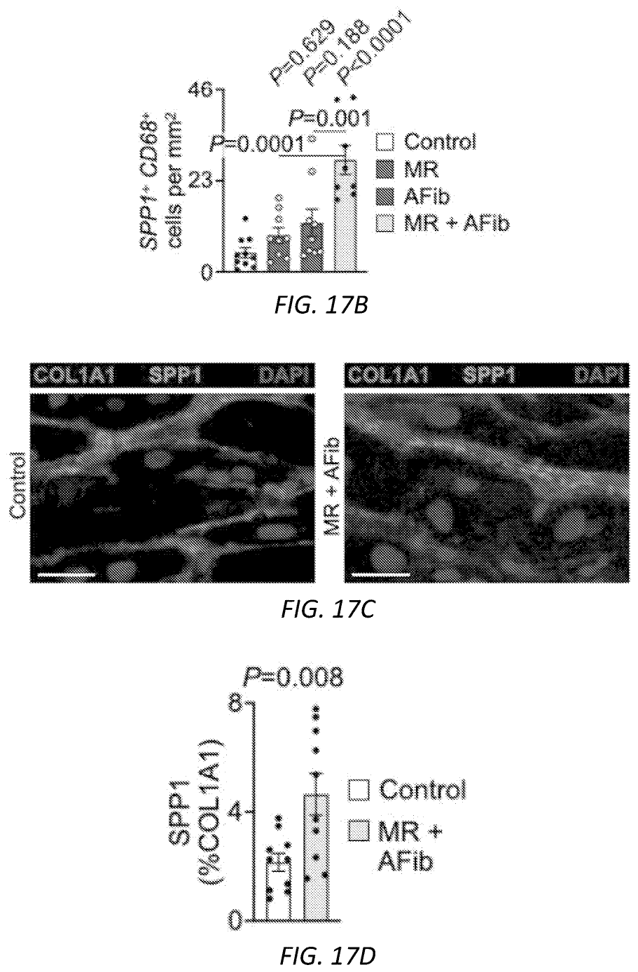

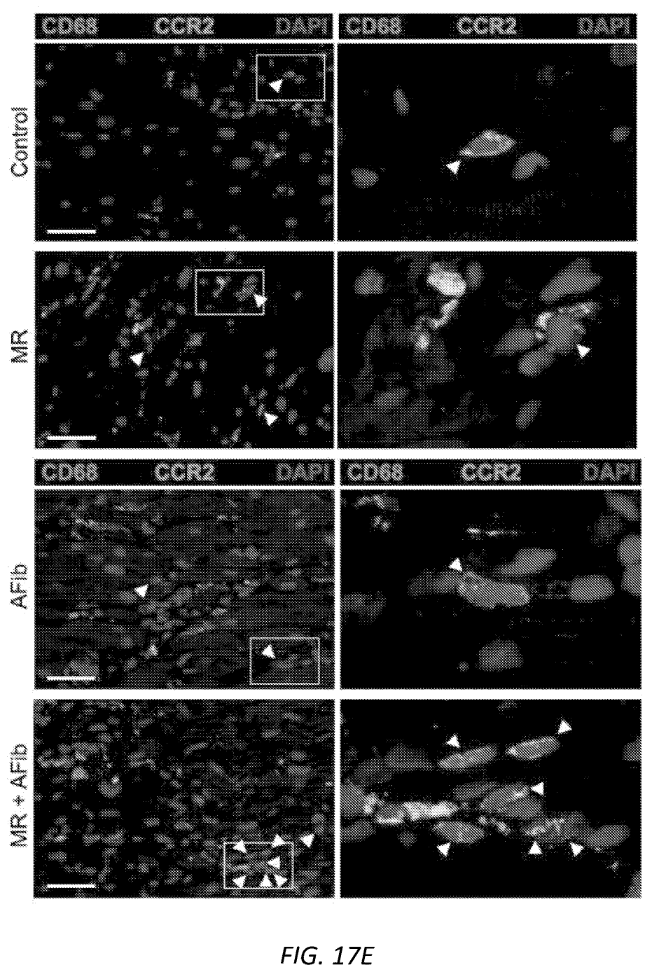

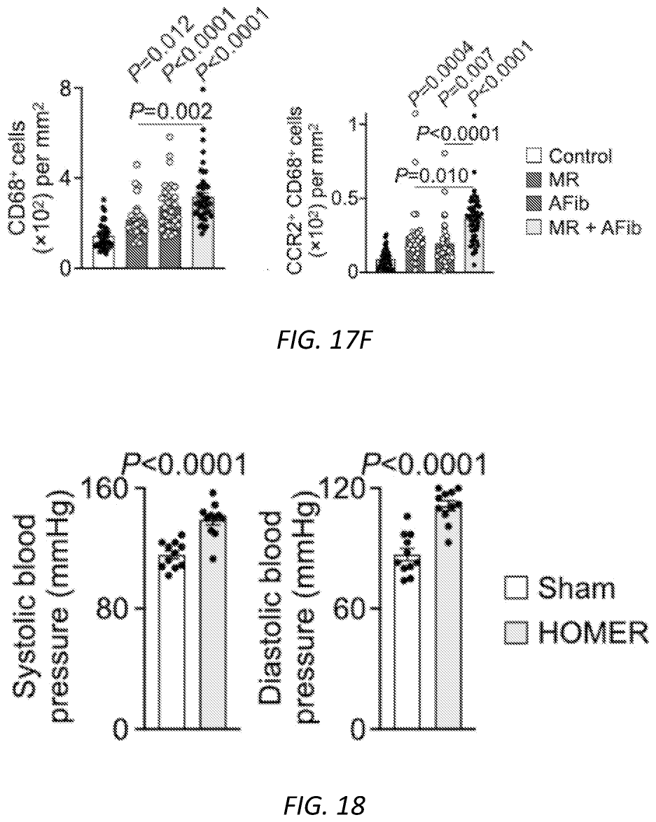

Depending on their origin and microenvironment, macrophages and their subsets take on phenotypes that enable a range of diverging—and even opposing—functions (3, 21). To identify cell types contributing to atrial remodeling, we therefore examined atrial myeloid cells in detail. We reclustered MP/DCs choosing a resolution that maximized the number of differentially expressed genes between subclusters (FIGS. 14A and 14B). Reclustering separated three monocyte subsets, an established number in human blood (21), and a fourth inflammatory monocyte cluster whose expression profile (FIG. 14C) and UMAP position adjacent to macrophages (FIG. 1H) suggested an intermediate phenotype during monocyte-to-macrophage differentiation. At this resolution, we could distinguish five macrophage clusters (FIG. 1H; FIG. 14C and FIG. 15) and two DC clusters (FIG. 1H and FIG. 14C). The inflammatory mono(b) cluster and the SPP1⁺ mac (c) cluster expanded 2.8- and 3.4-fold, respectively, in diseased atria (FIG. 1I). The frequency of SPP1⁺ atrial cells increased in patients with AFib when compared to non-AFib controls (FIG. 1J). The expanding mac (c) cluster was the dominant SPP1 source in atria of AFib patients (FIGS. 1K and 1L), as other immune and stromal cells did not express SPP1 at high levels (FIG. 16). TREM2 and CD9, which encode proteins that can be detected by cell surface staining (22), further distinguished the SPP1⁺ mac (c) cluster from other cells (FIG. 1K and FIG. 16). RNA in situ hybridization confirmed that macrophages express SPP1 in atria of AFib patients (FIGS. 17A and 17B). Once secreted by macrophages, SPP1 associates with extracellular matrix (13). Accordingly, immunofluorescence microscopy identified expanding colocalization of SPP1 protein with collagen in AFib patients (FIGS. 17C and 17D).

To explore atrial macrophage expansion in a larger cohort, we used immunofluorescence histology on left atrial appendages of 108 patients with AFib and/or mitral regurgitation and 41 controls in sinus rhythm (FIG. 44). Atrial macrophages and their monocyte-derived CCR2⁺ subset expanded most in AFib patients with mitral regurgitation (FIGS. 17E and 17F). In patients that had mitral regurgitation without AFib or AFib without mitral regurgitation macrophages expanded to a lesser degree (FIGS. 17E and 17F). In AFib patients without mitral regurgitation, AFib chronicity was less pronounced (FIG. 44) which may explain why we observed fewer atrial macrophages in this cohort.

### A Mouse Model of Atrial Disease

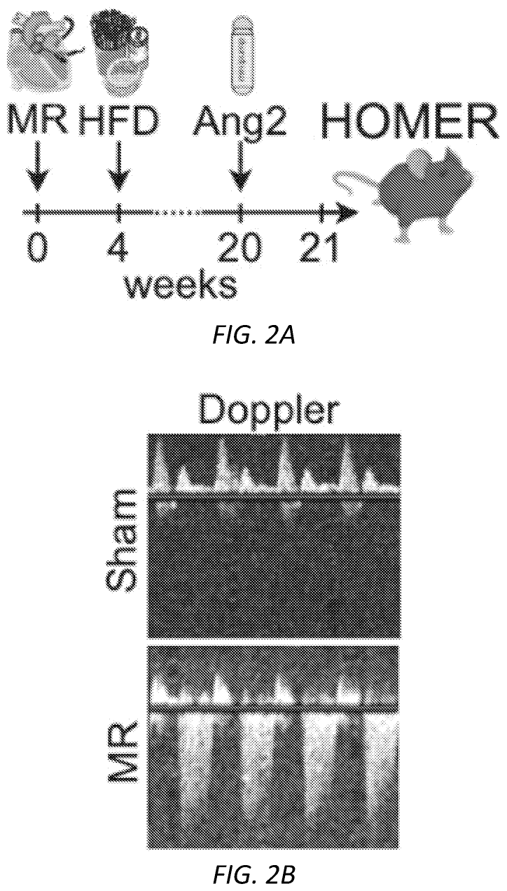

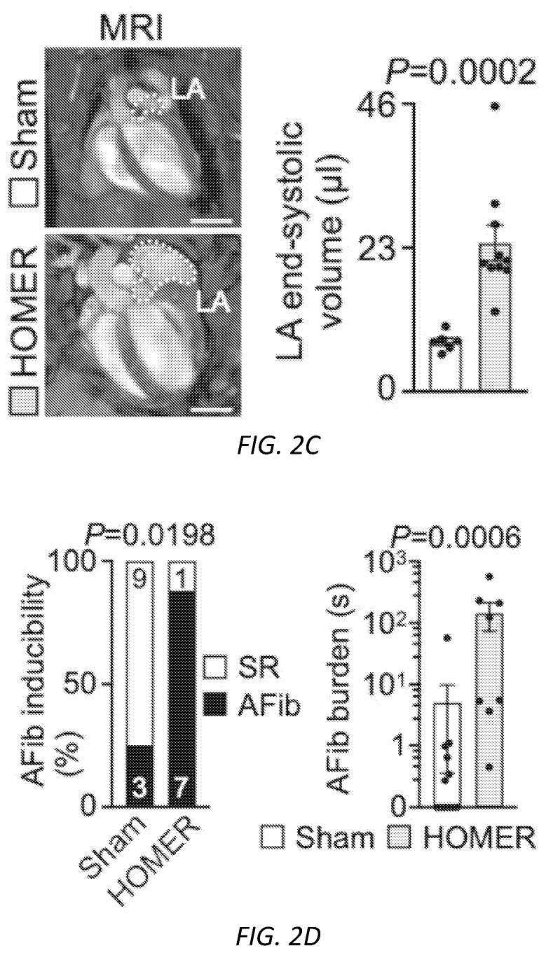

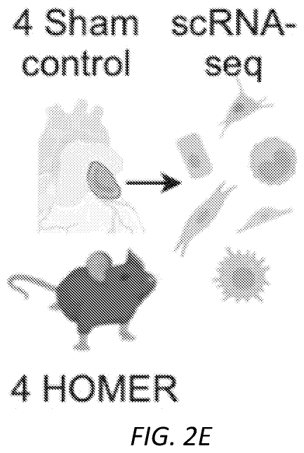

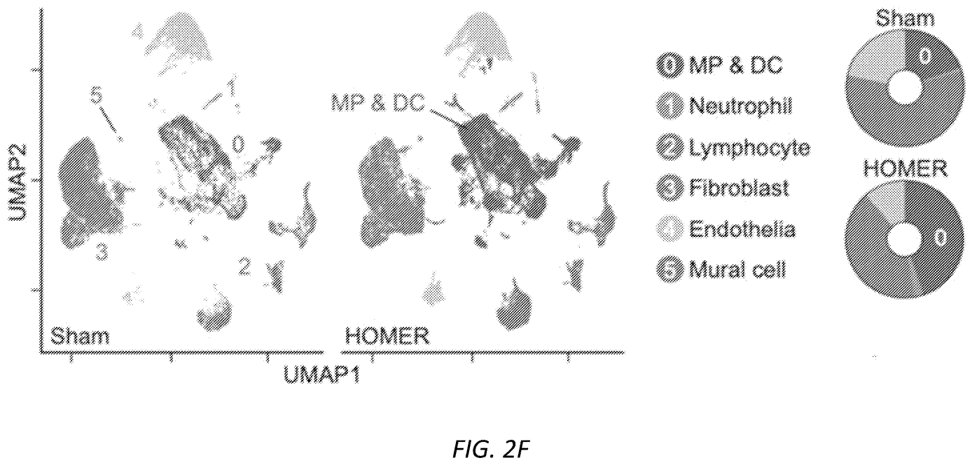

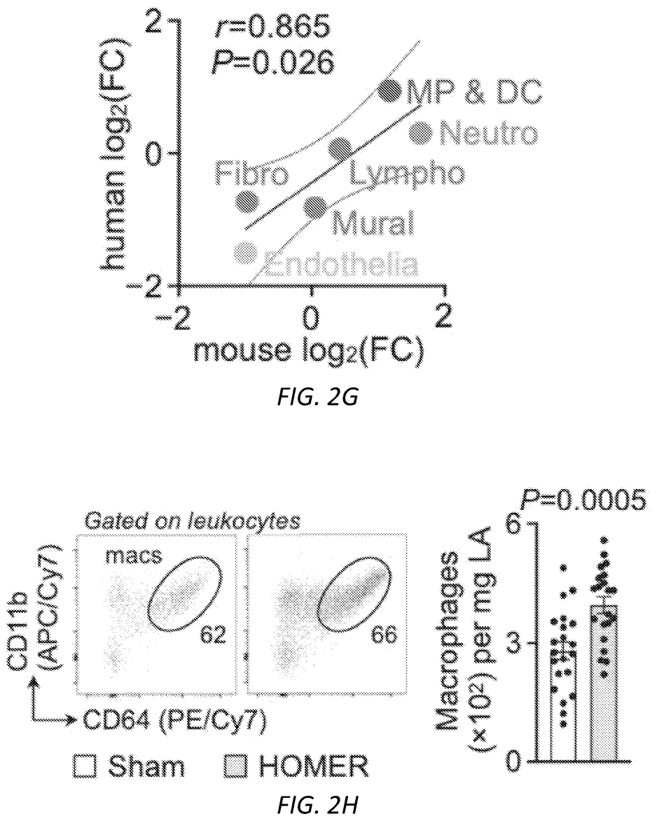

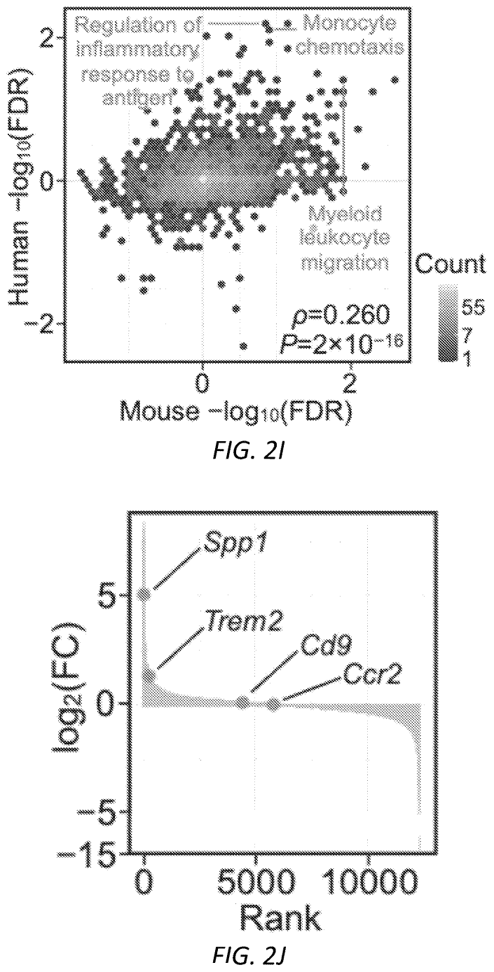

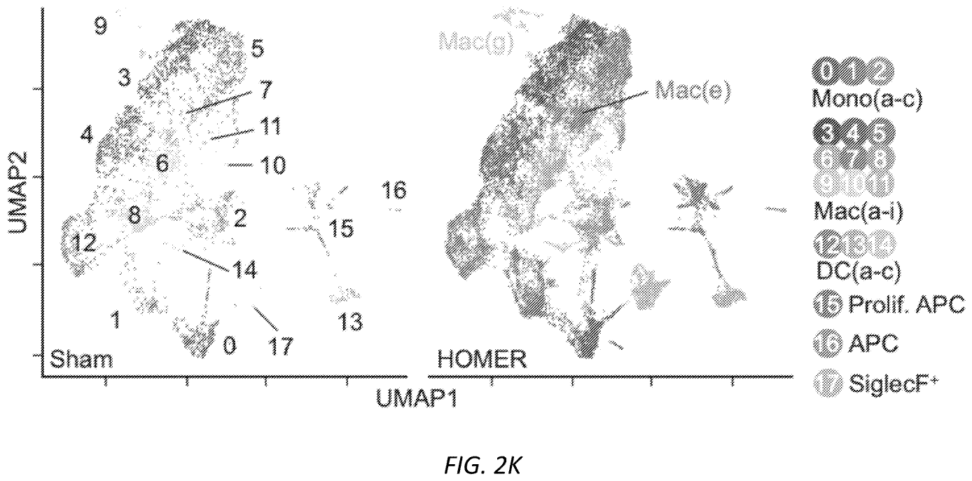

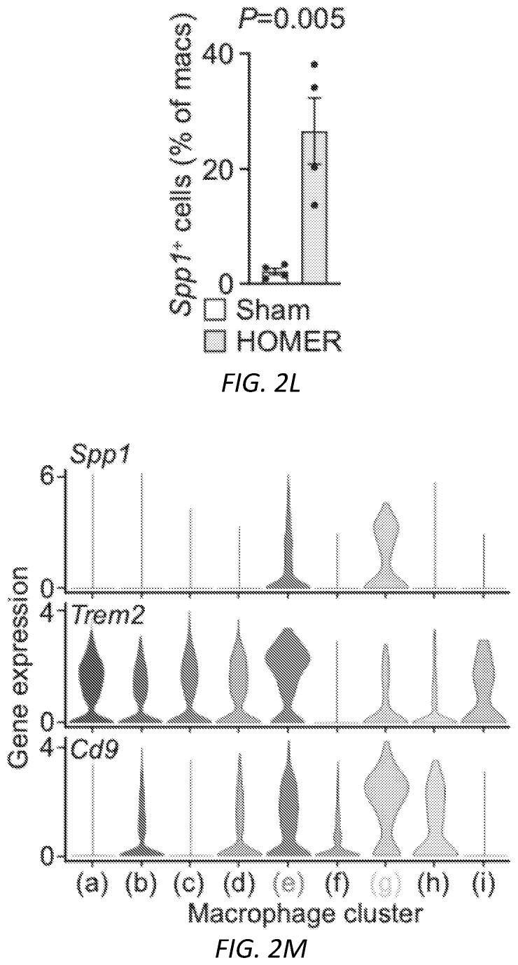

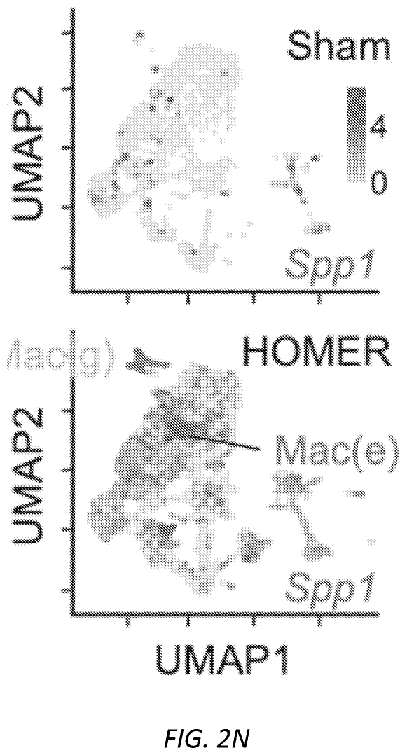

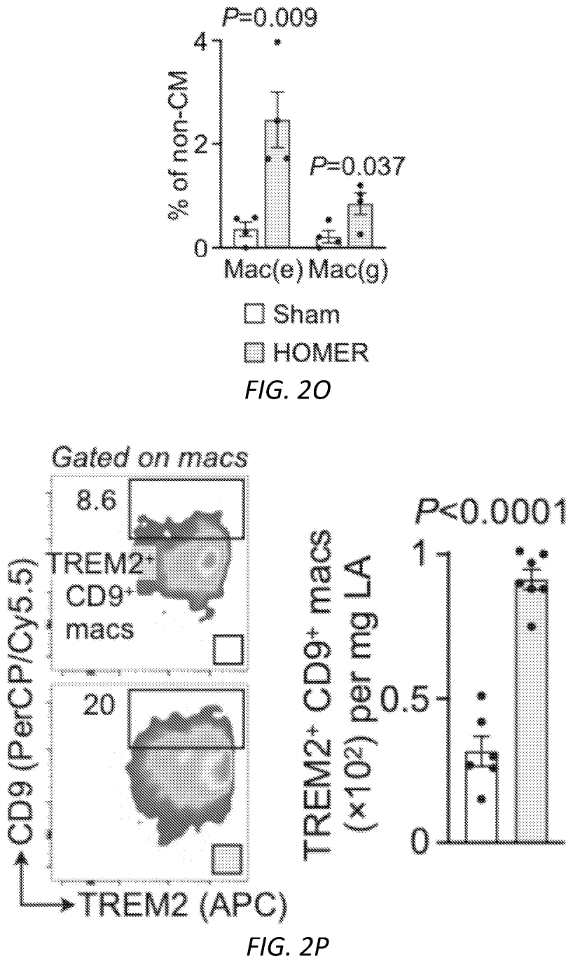

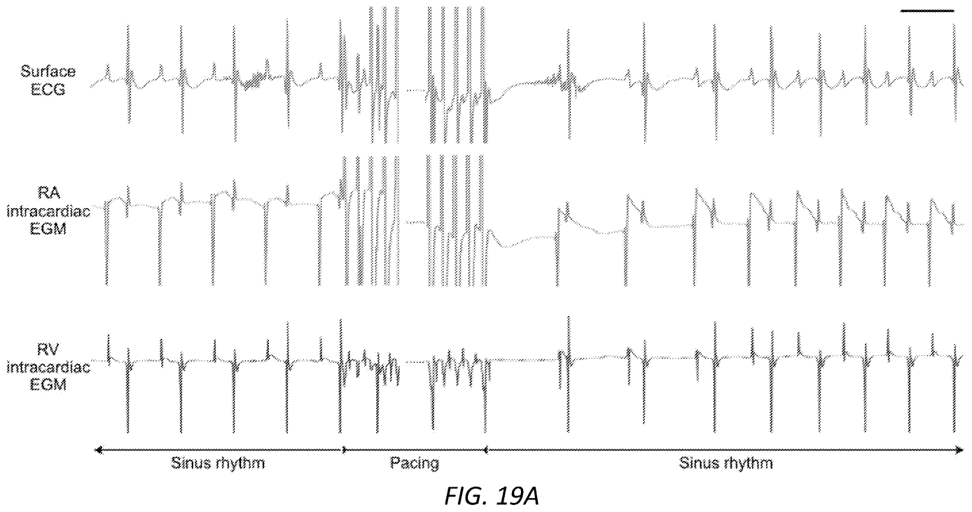

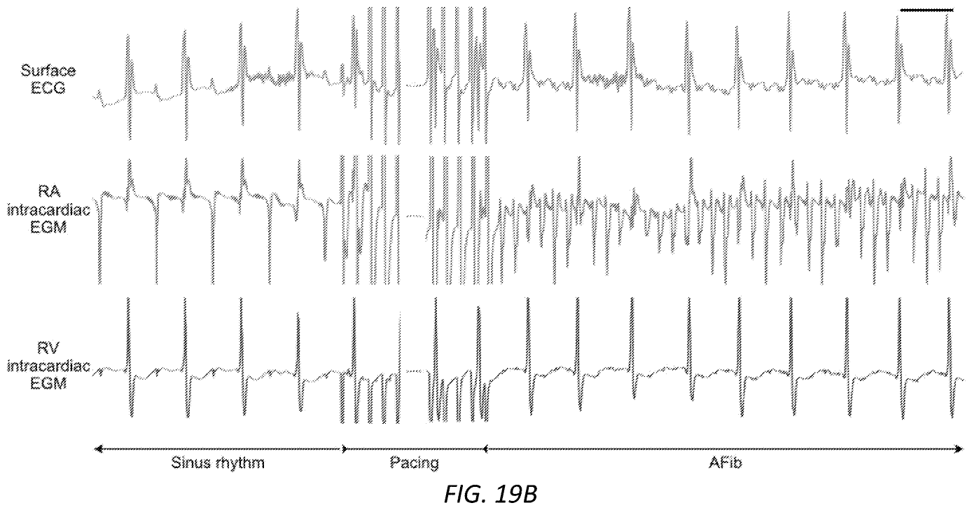

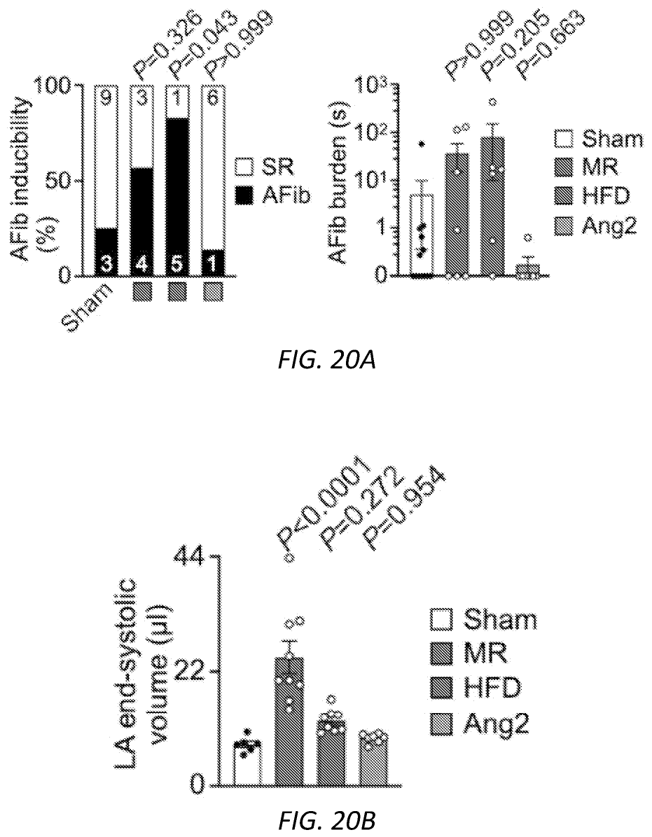

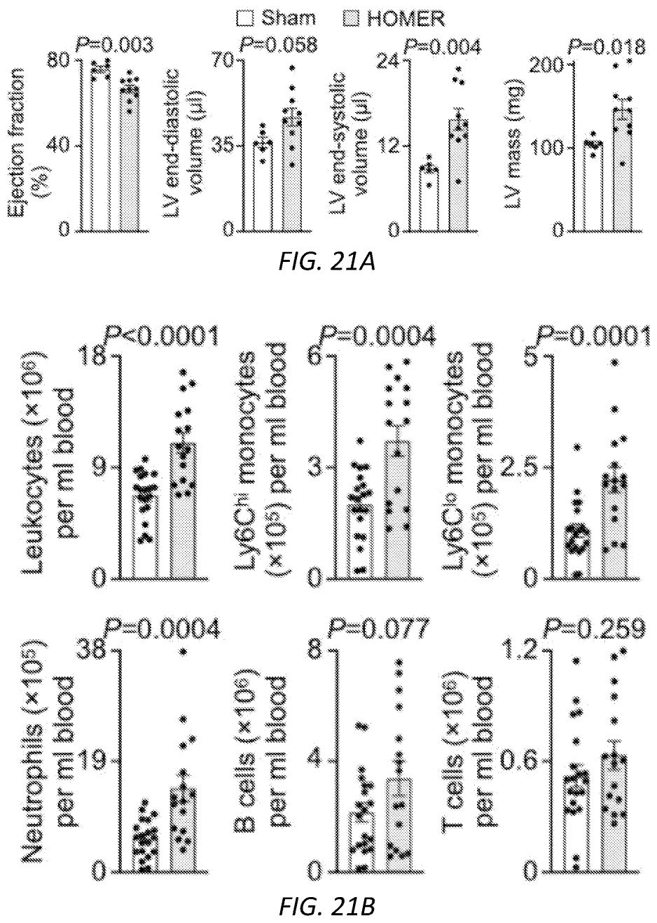

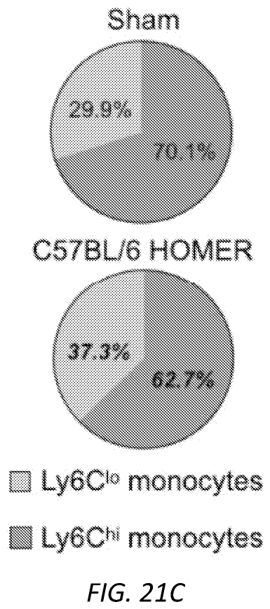

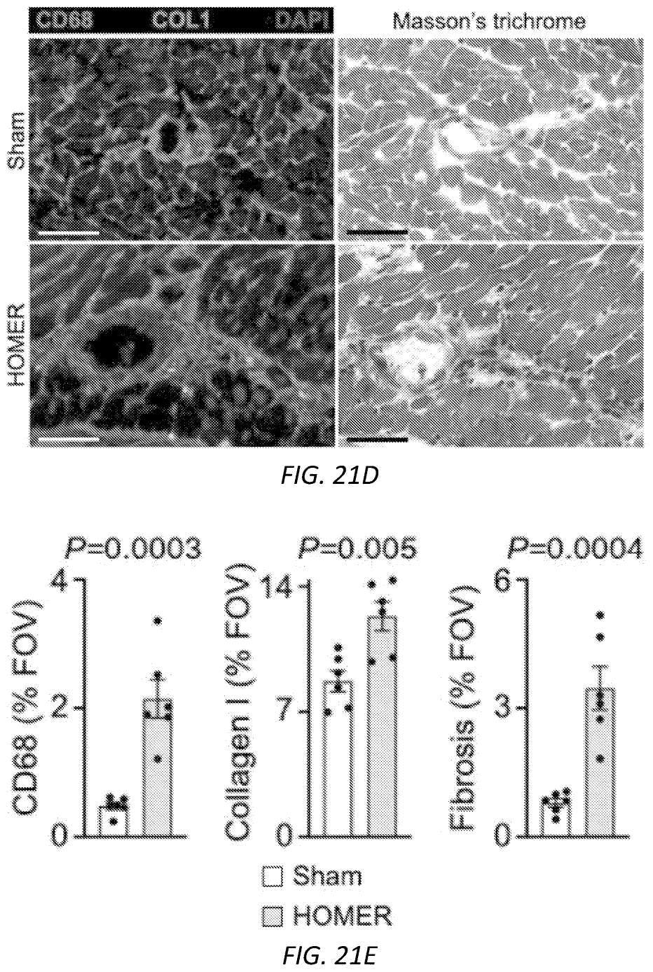

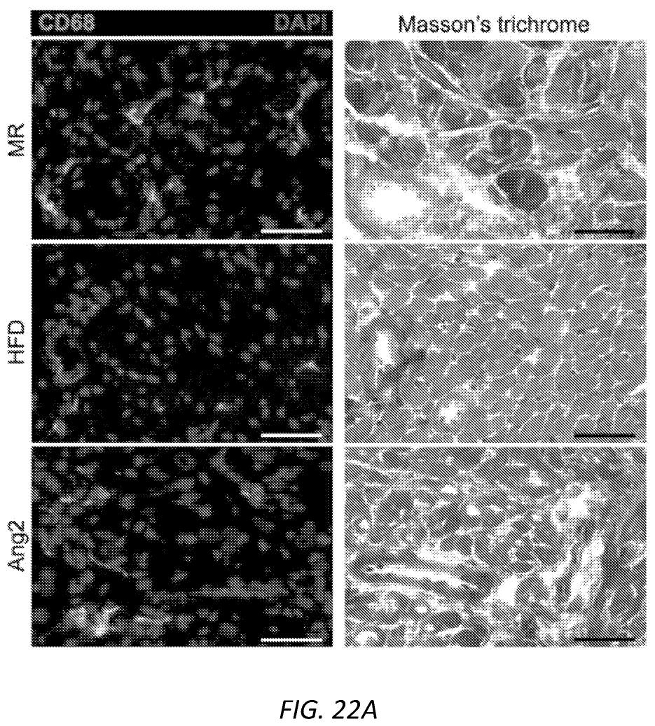

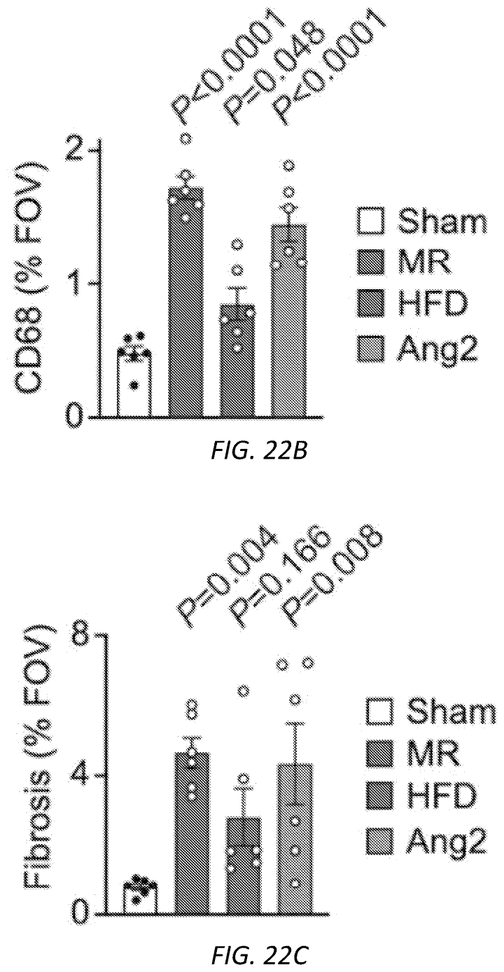

To dissect immune pathways involved in human AFib mechanistically, we employed a mouse model that combined hypertension, obesity and mitral valve regurgitation (HOMER) (FIG. 2A). Snaring the mitral valve to the ventricular wall produced mitral valve regurgitation (FIG. 2B). Mice consumed an obesogenic diet for 4 months, mirroring the risk obesity engenders for AFib (23). Hypertension, also a risk factor for AFib, was induced with angiotensin 2 (FIG. 18). The HOMER model elicited left atrial enlargement (FIG. 2C) commonly found in patients with AFib (24). HOMER mice developed increased AFib inducibility and burden compared to sham-operated normotensive control mice that consumed standard chow (hereafter referred to as controls) (FIG. 2D and FIG. 19). Among individual HOMER components, obesity elicited the highest AFib inducibility, whereas mitral valve regurgitation enlarged atria the most (FIG. 20). Spontaneous AFib episodes, which were monitored using ECG telemeters, were absent in HOMER mice. In HOMER hearts, left ventricles hypertrophied and dilated mildly and left ventricular systolic function was slightly reduced (FIG. 21A and FIG. 45). Blood leukocyte counts, including Ly6C^{hi} monocytes, rose in HOMER mice (FIGS. 21B and 21C). Histological analysis revealed more numerous macrophages, fibrosis, and collagen deposition in HOMER atria compared to controls (FIGS. 21D and 21E), results that align with the fibrosis observed in patients with AFib (25) and the effects of hypertension and metabolic stress on the ventricles (15, 26, 27). Hypertension and mitral valve regurgitation most clearly increased atrial macrophage numbers and fibrosis (FIG. 22).

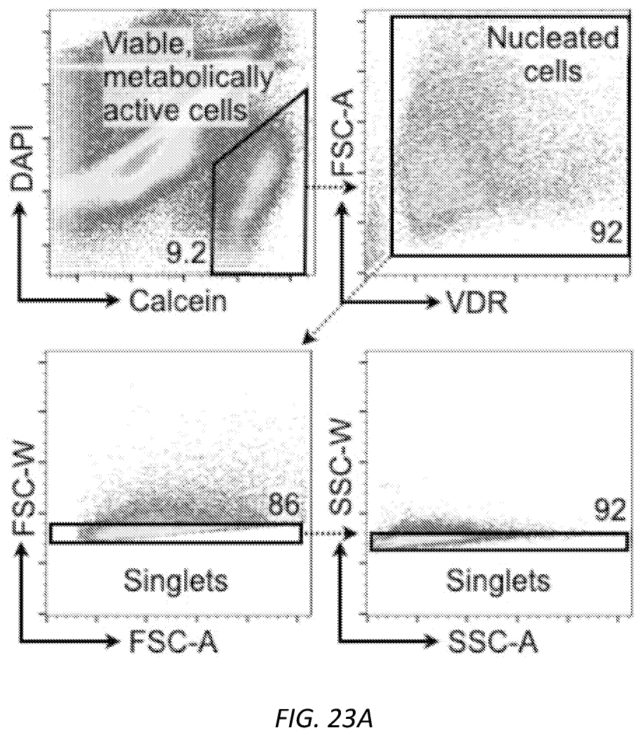

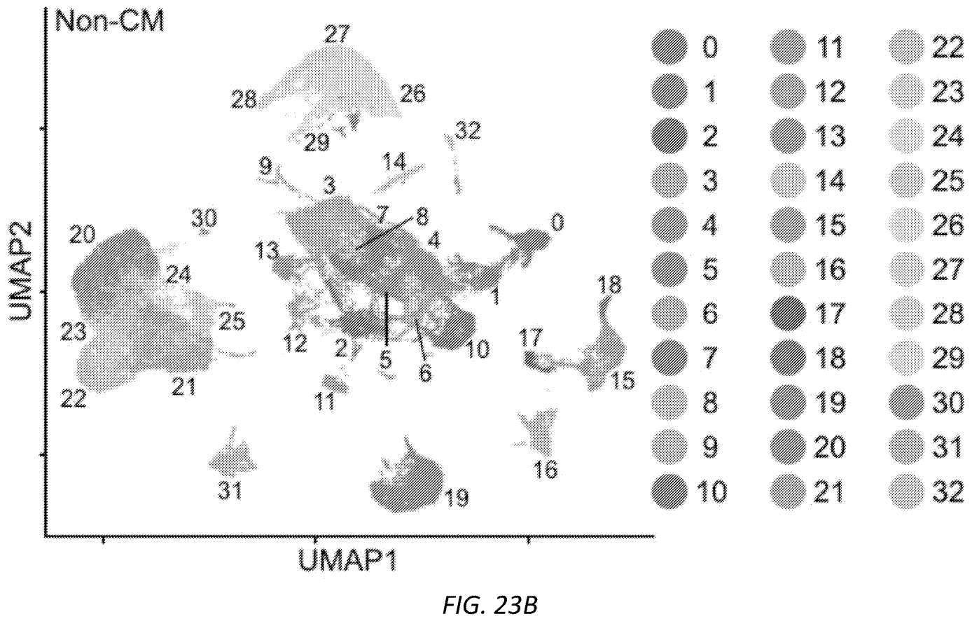

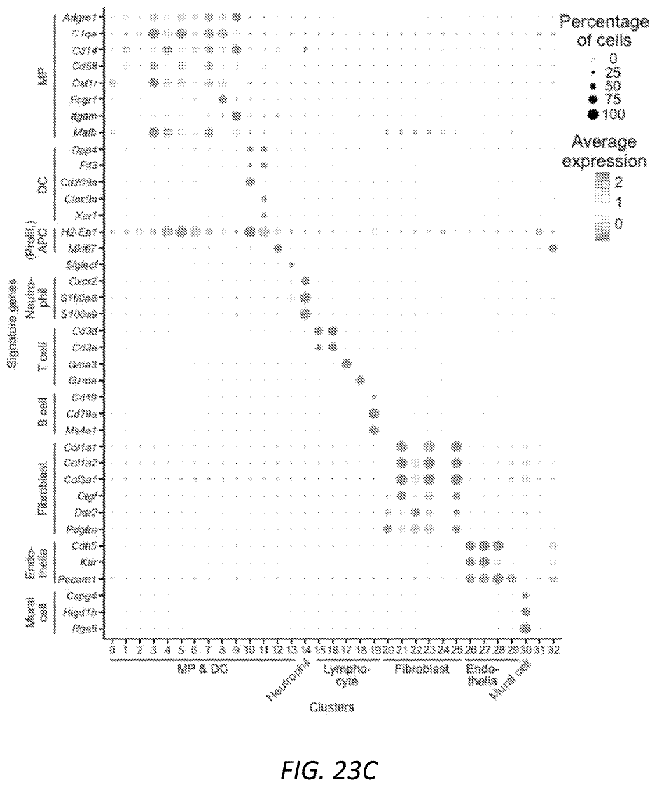

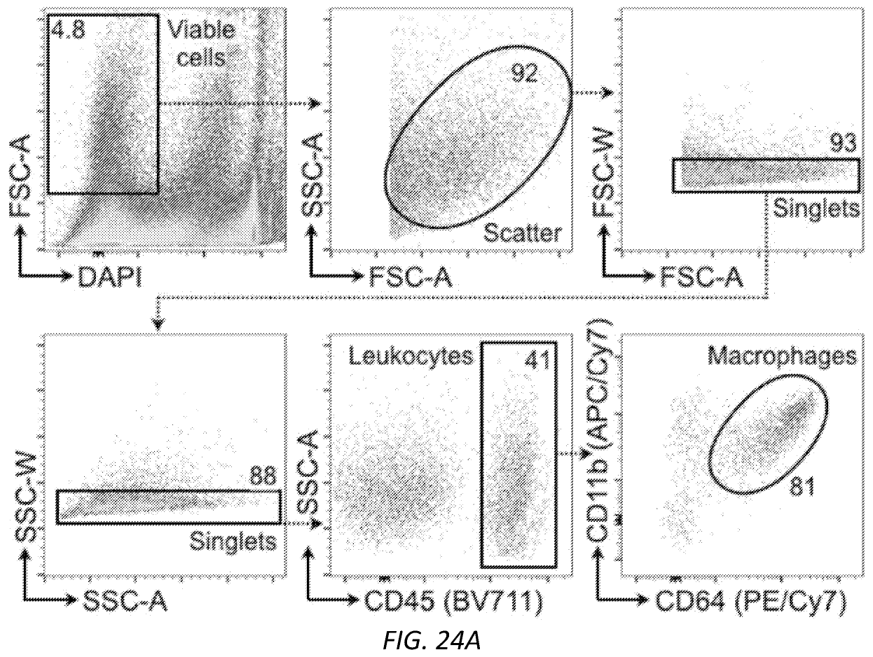

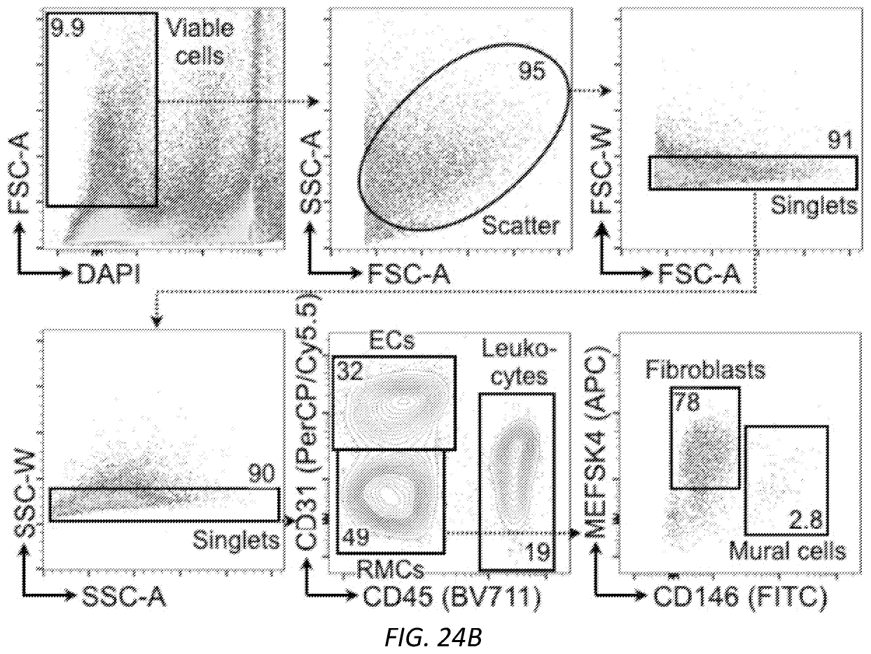

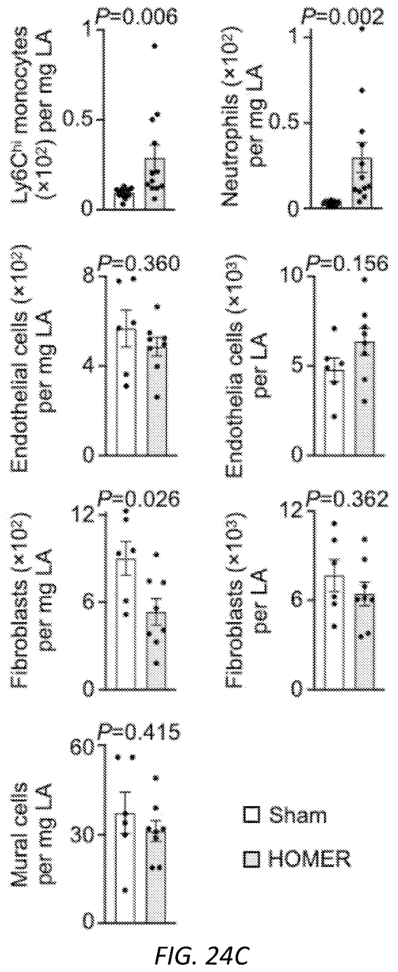

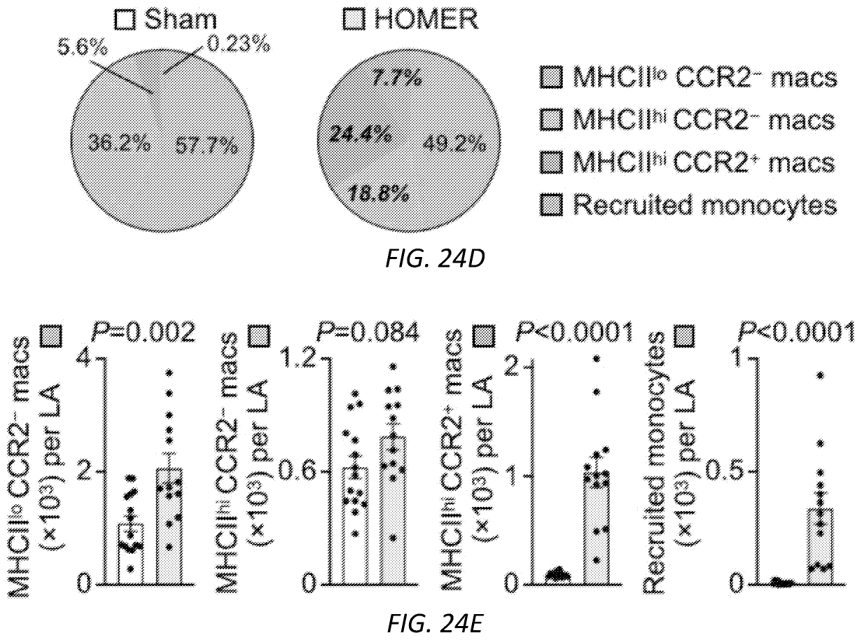

Replicating our human scRNA-seq pipeline using left atrial tissues dissected from four HOMER mice and four control animals (FIG. 2E and FIG. 23A), we successfully obtained 40,112 viable single cells, among which we identified the same cell types as in human atria (FIG. 2F; FIGS. 23B and 23C; and FIG. 46). The cellular composition changes in diseased atria correlated strongly between both species (Pearson's r=0.87) (FIG. 2G). We observed a twofold expansion of MP/DCs in HOMER mice similar to the human disease setting (FIG. 2F and FIG. 46). Flow cytometry confirmed the HOMER-induced changes in the abundance of macrophages (FIG. 2H and FIGS. 24A to 24C). Cardiac macrophage subsets can be distinguished by expression of CCR2 and MHCII (28, 29). In atria of HOMER mice, CCR2⁺ macrophages and monocytes rose (FIGS. 24D and 24E).

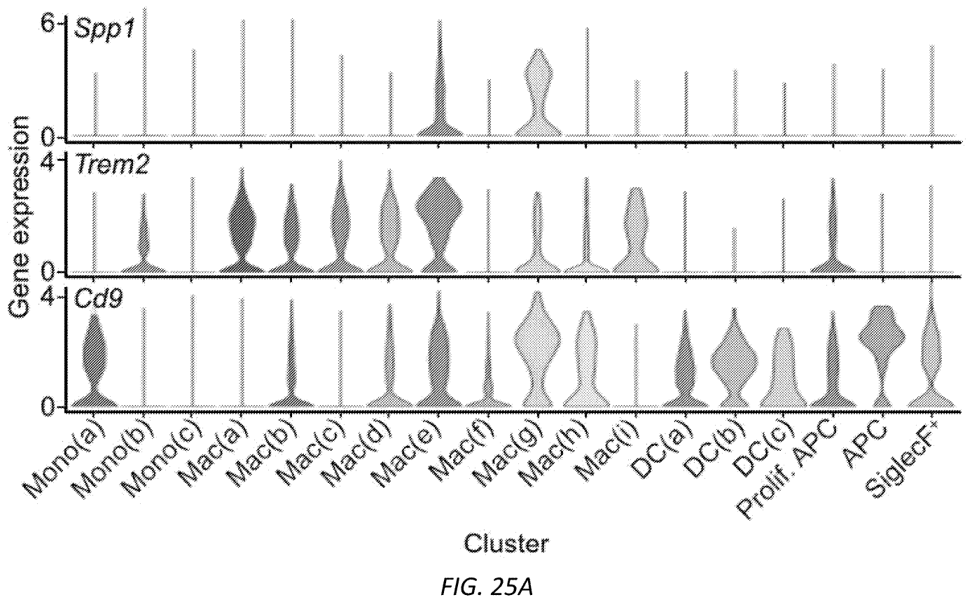

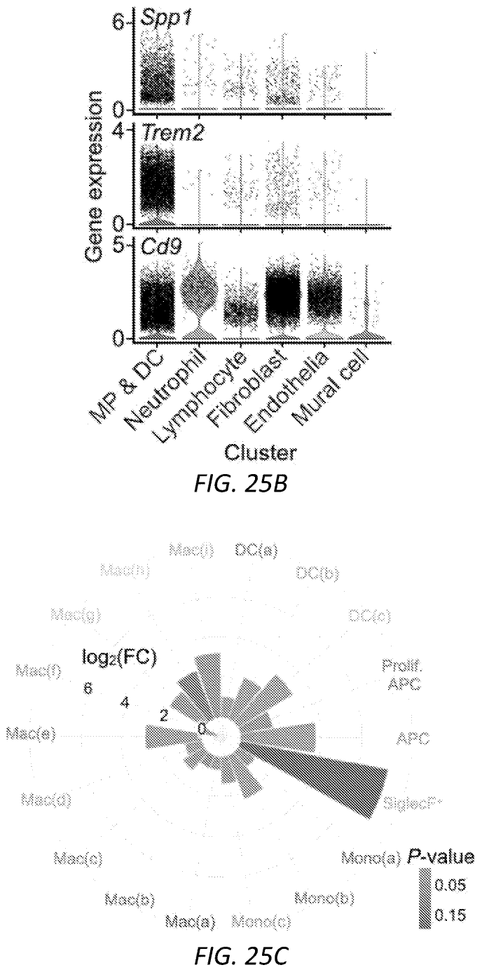

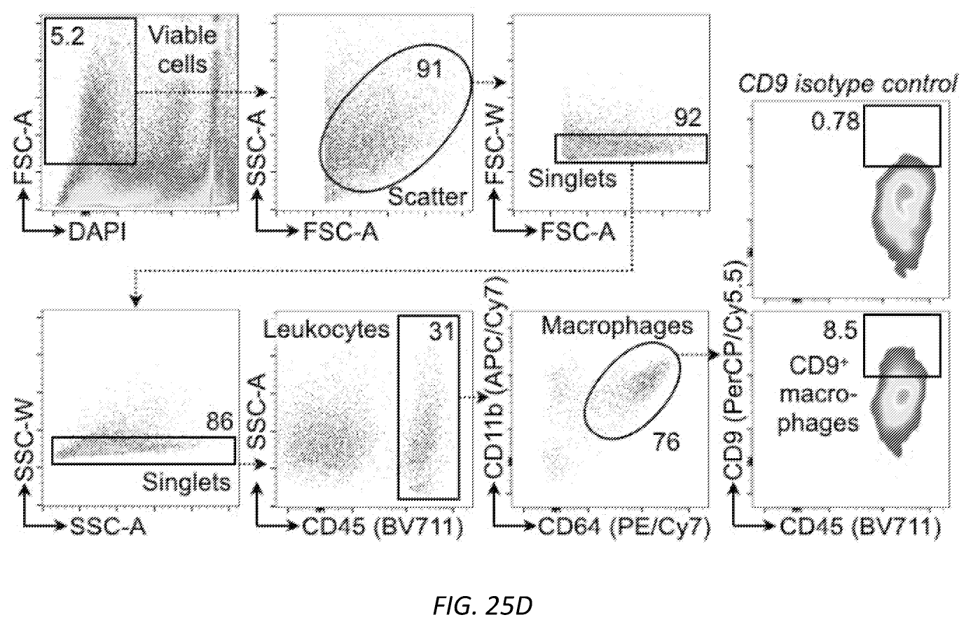

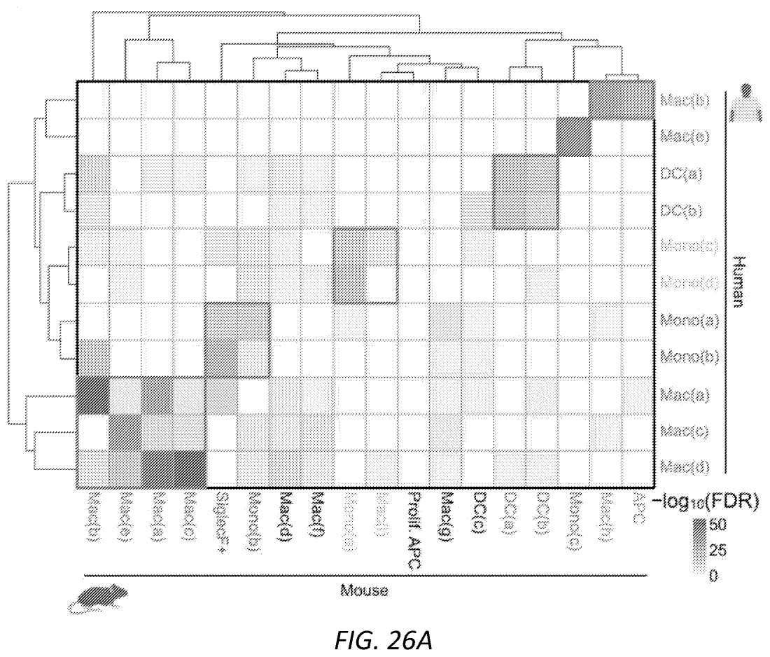

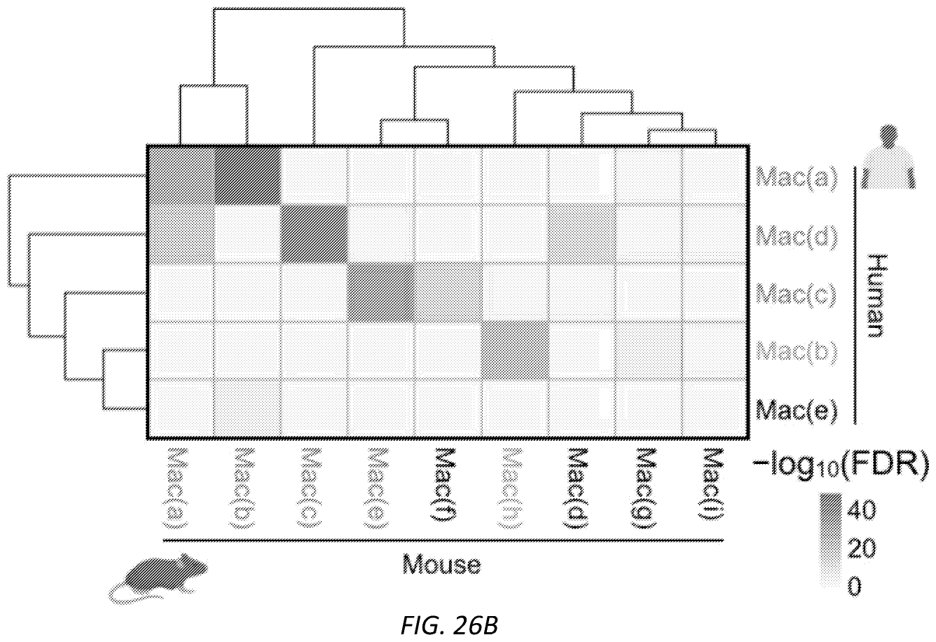

We next examined the gene expression changes that occurred in MP/DCs of HOMER mice in relation to our human data. As before, we first performed GSEA on differentially expressed genes from a pseudobulk analysis comparing MP/DCs isolated from atria of HOMER or control mice. We then plotted the significance and direction of change for all MSigDB C5 (Gene Ontology) gene sets against the corresponding values in humans and found significant concordance between the two GSEA data sets (Spearman's p=0.26, P<2×10⁻¹⁶) (FIG. 2I), indicating that the HOMER model recapitulated the pathway-level gene expression changes observed in patients. As in humans, Spp1 was among the top upregulated genes in MP/DCs of HOMER atria (FIG. 2J). We then reclustered MP/DCs to obtain a higher-resolution view of these cells, choosing a resolution that separated three monocyte clusters (a number previously described in mouse blood (30)), nine macrophage clusters, three DC clusters, two antigen-presenting cell clusters, and one SiglecF⁺ cell cluster (FIG. 2K). Spp1⁺ cell frequencies increased in HOMER atria (FIG. 2L), a rise driven by the macrophage subpopulations mac (e) and mac (g) and not by other immune or stromal cells (FIGS. 2M and 2N, and FIGS. 25A and 25B). In close concordance with our human findings, Spp1⁺ mac (e) and mac (g) clusters coexpressed Trem2 and Cd9 (s. 2M) and expanded in HOMER mice (FIGS. 2O and 2P, and FIGS. 25C and 25D). Comparing MP/DC clusters across the two species (using the top 100 genes that distinguished each subcluster), we noted that each human MP/DC cluster significantly matched at least one mouse cluster and that the human SPP1⁺ cluster mac (c) was similar to the mouse Spp1⁺ cluster mac (e) (FIG. 26). This strong interspecies correlation is likely caused by similar risk factors in patients and HOMER mice.

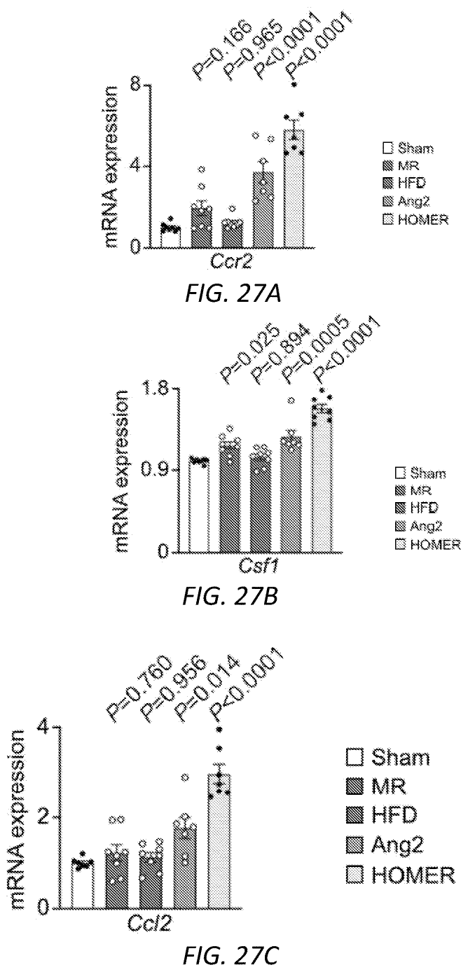

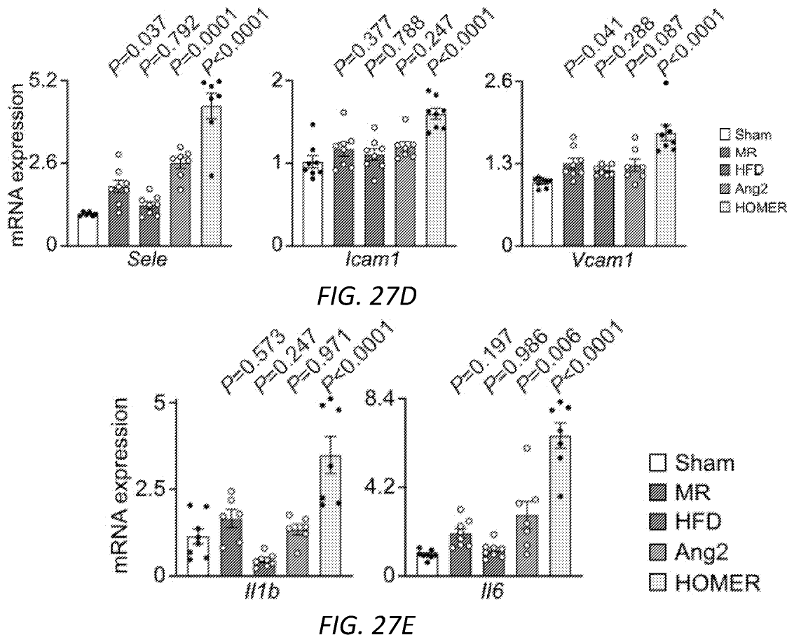

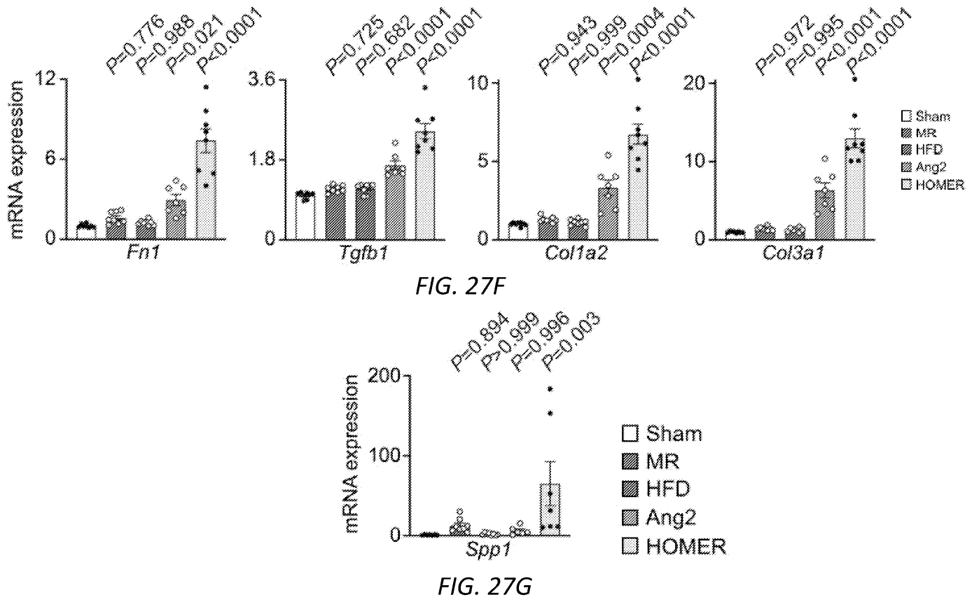

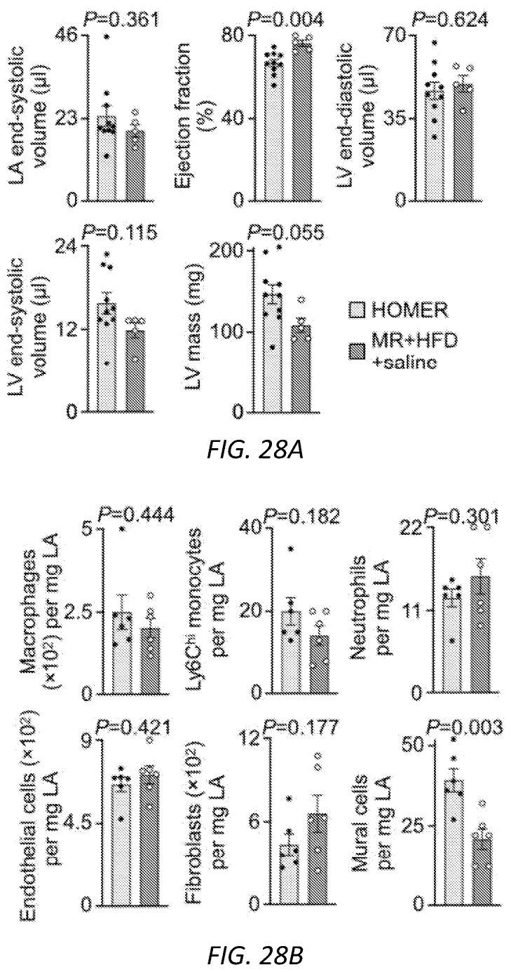

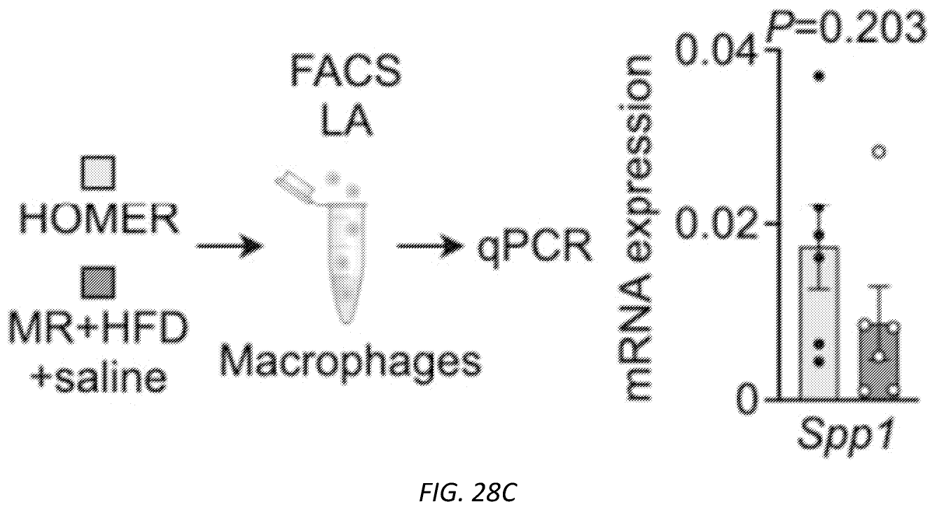

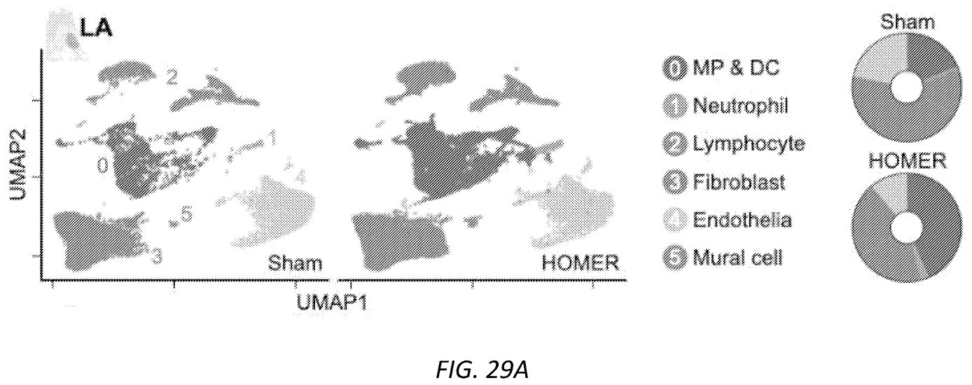

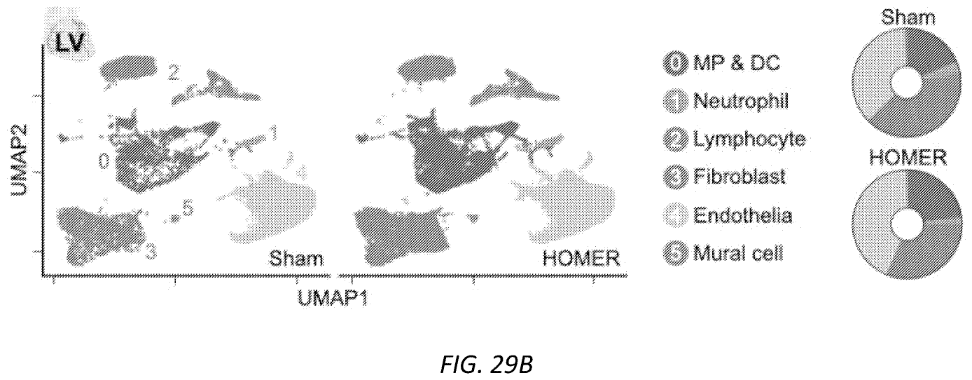

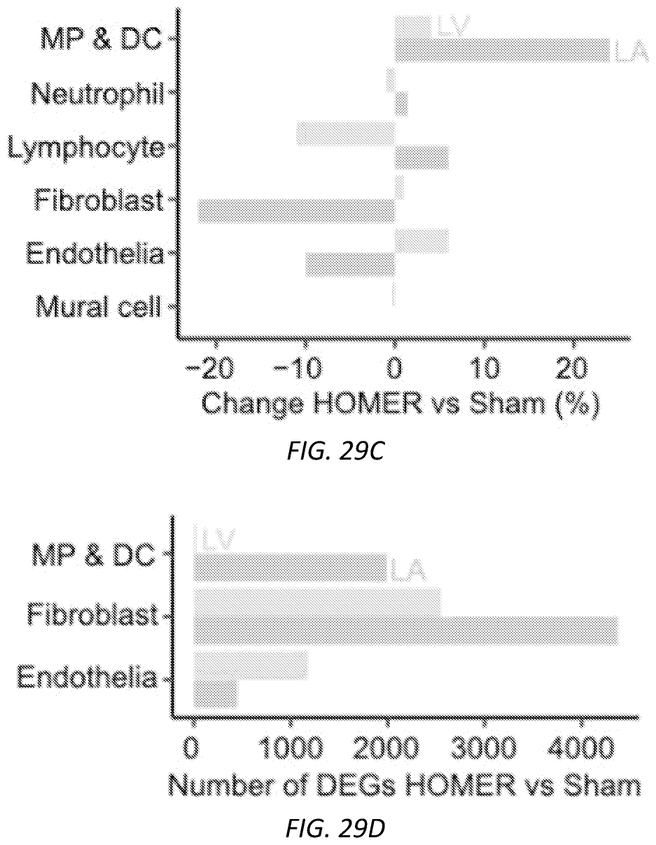

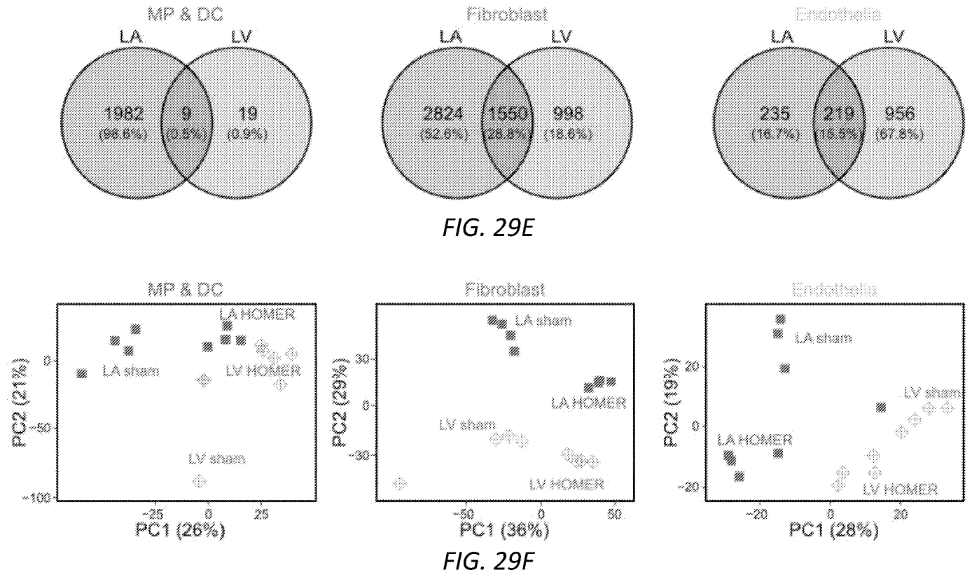

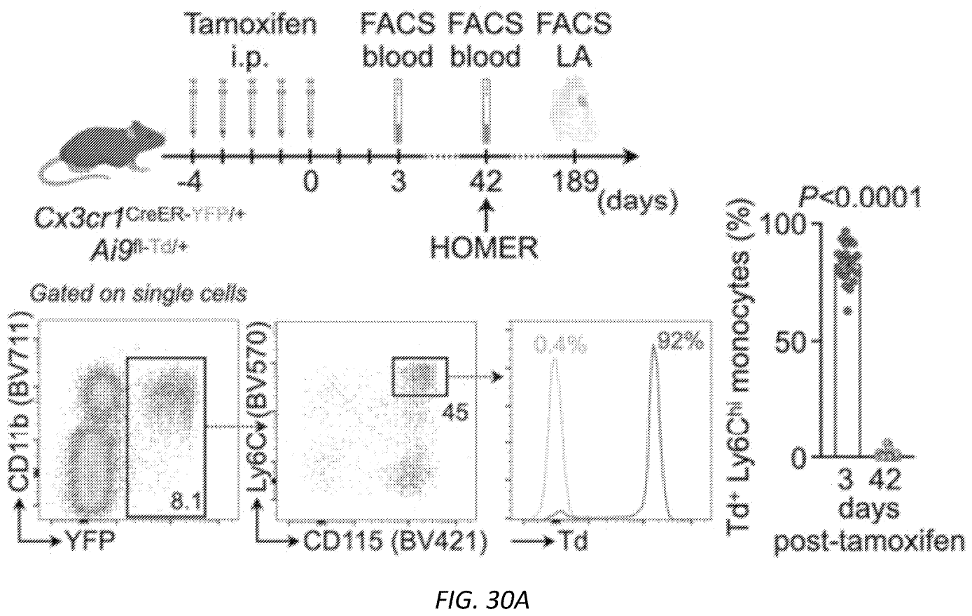

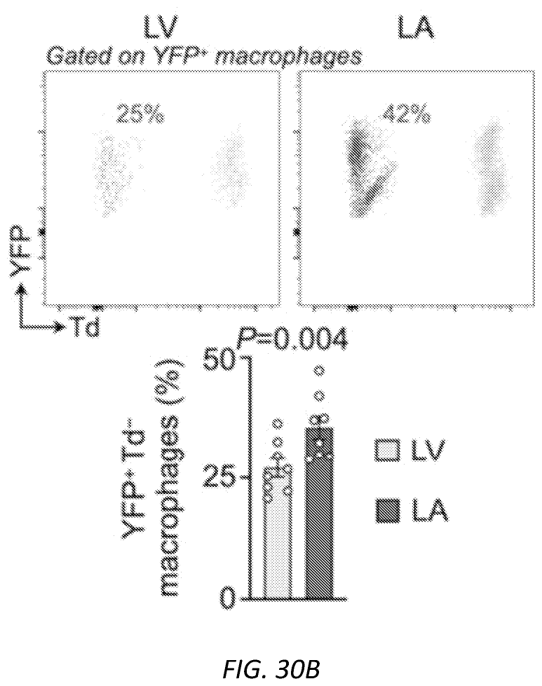

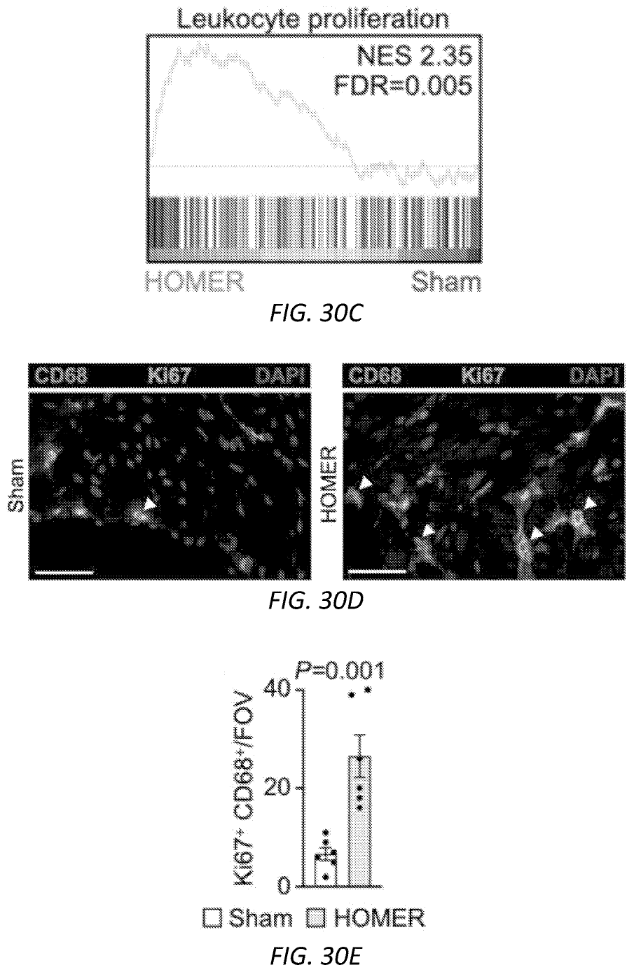

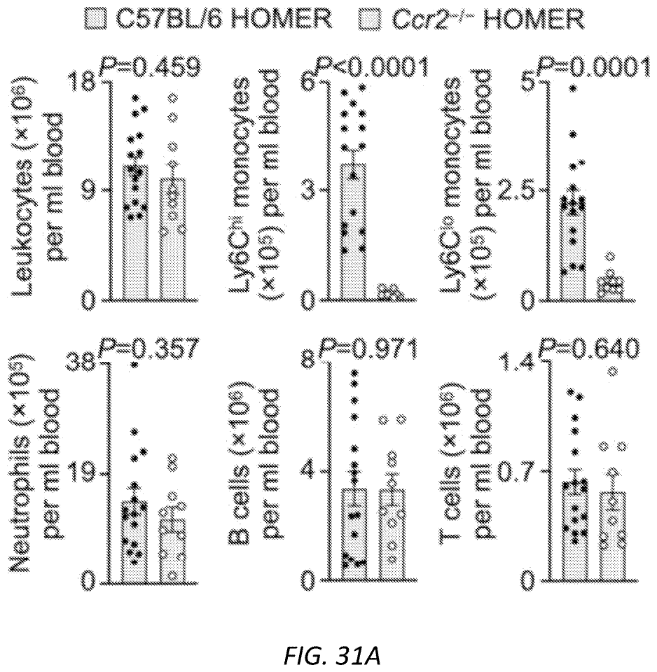

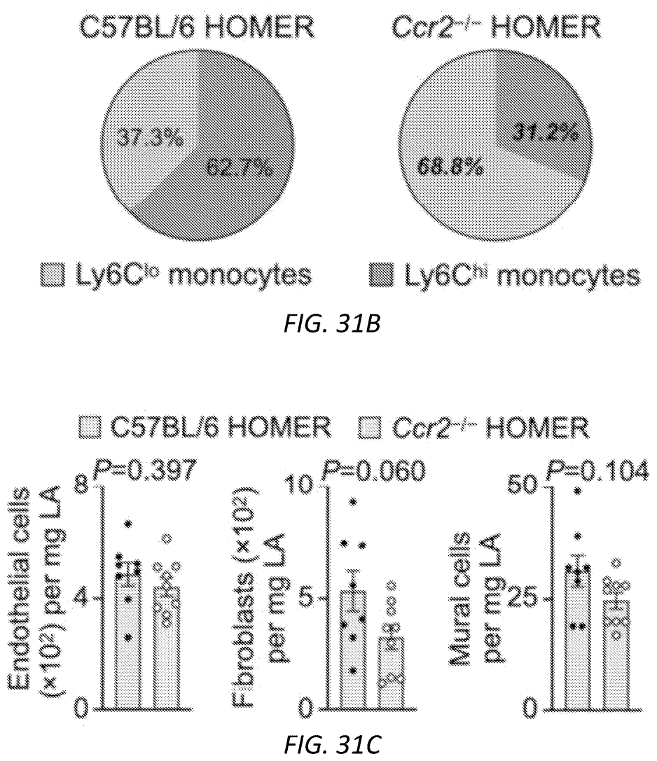

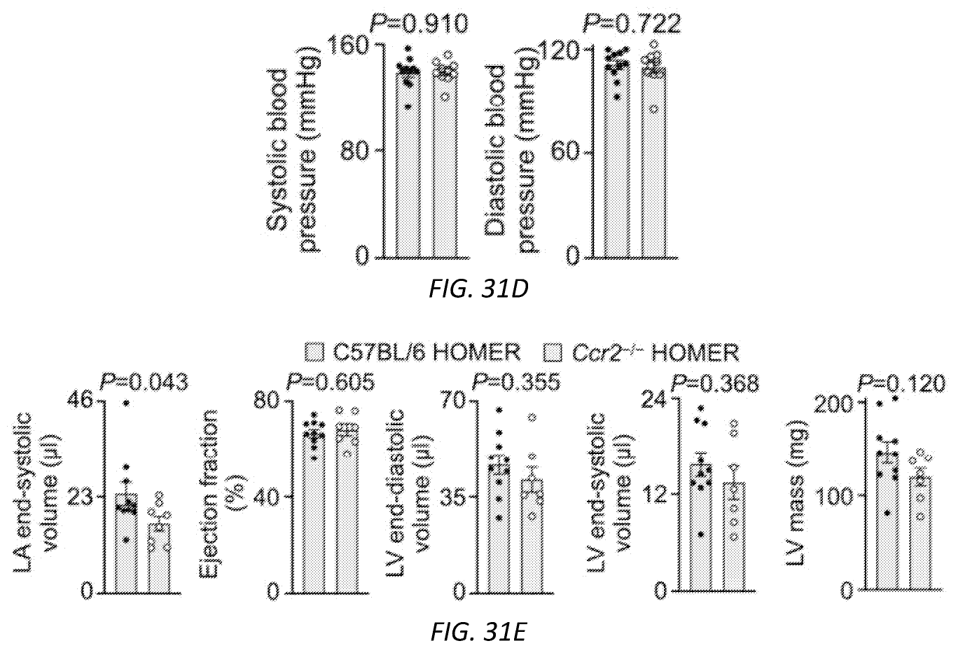

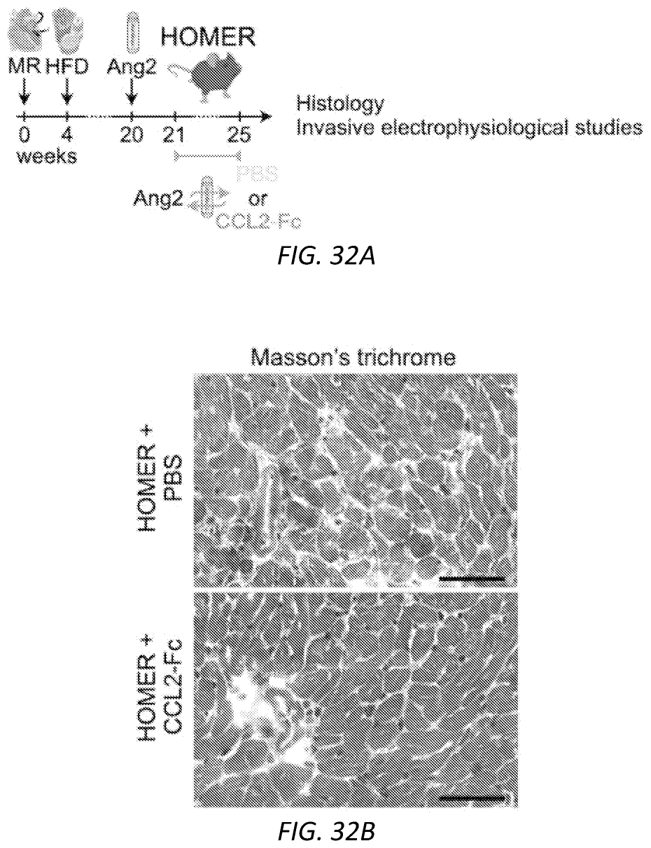

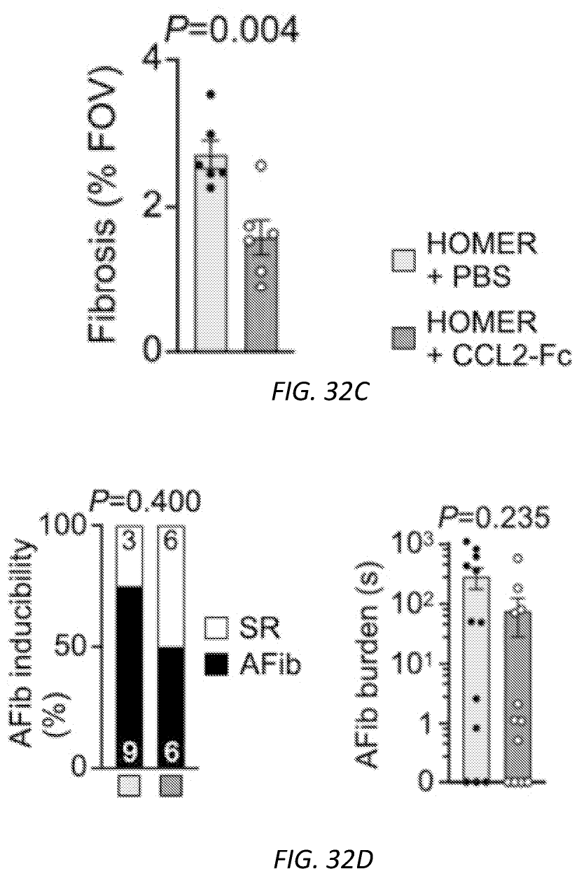

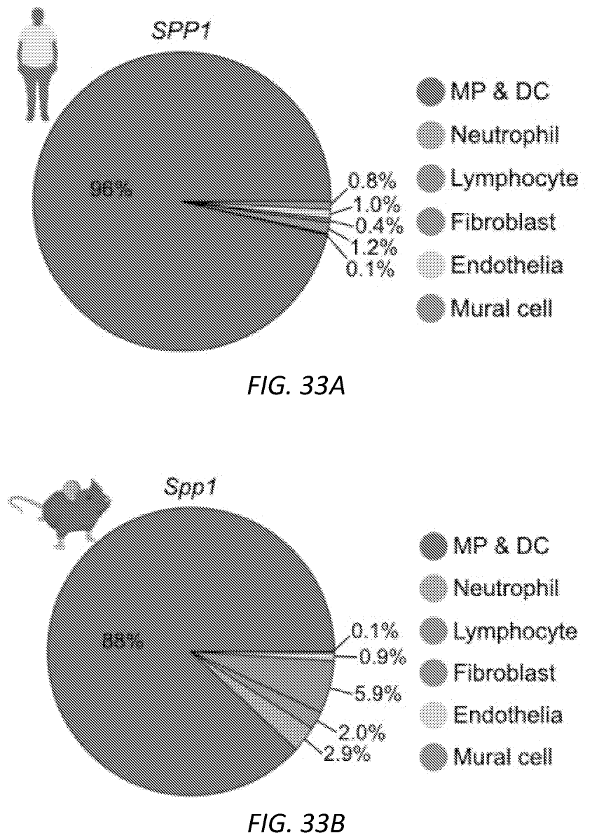

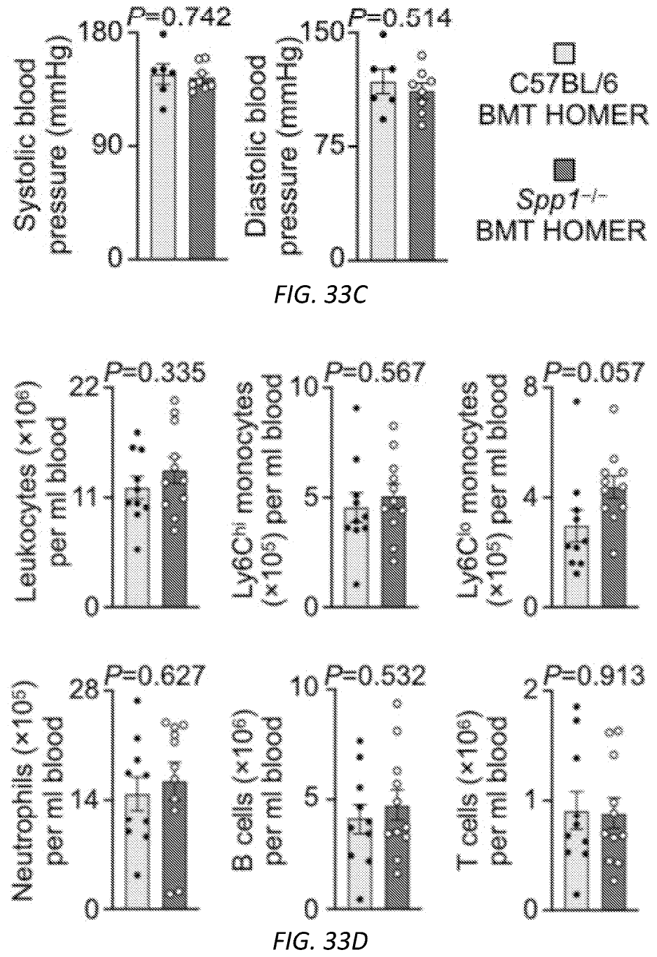

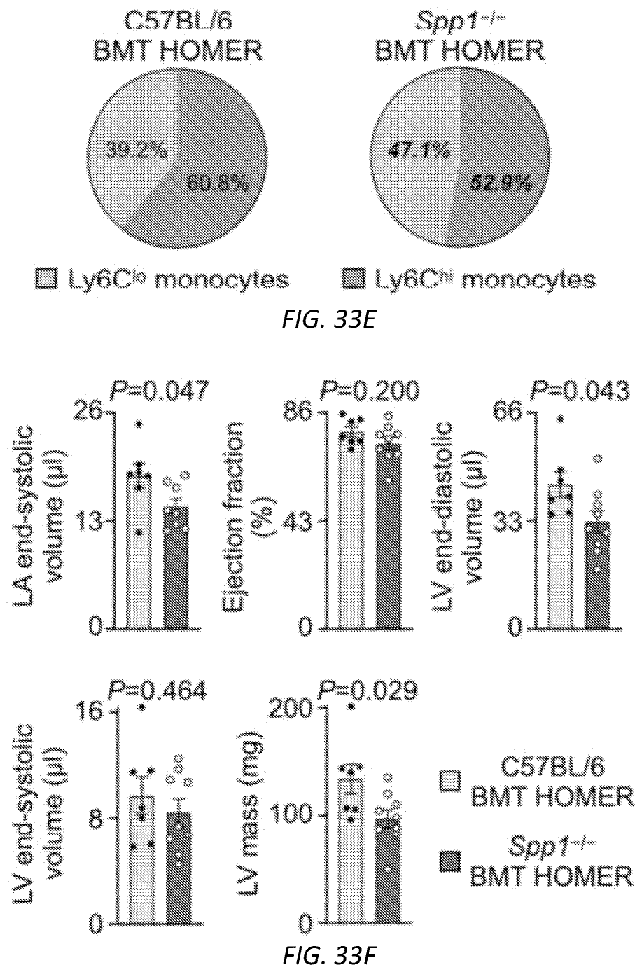

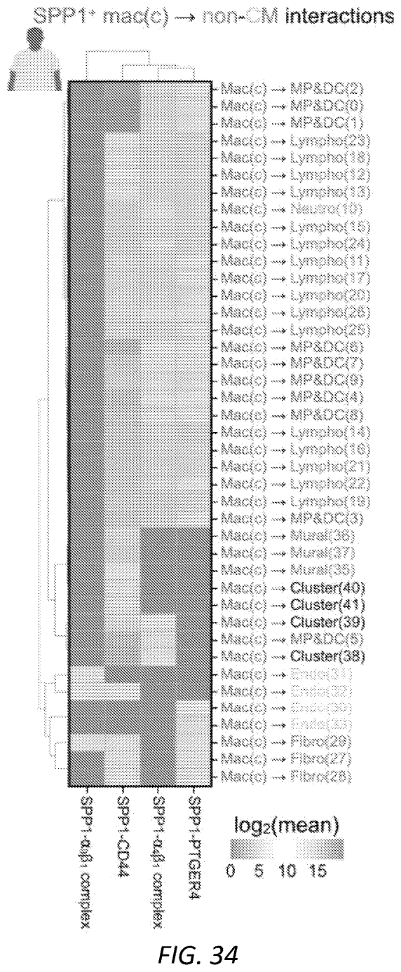

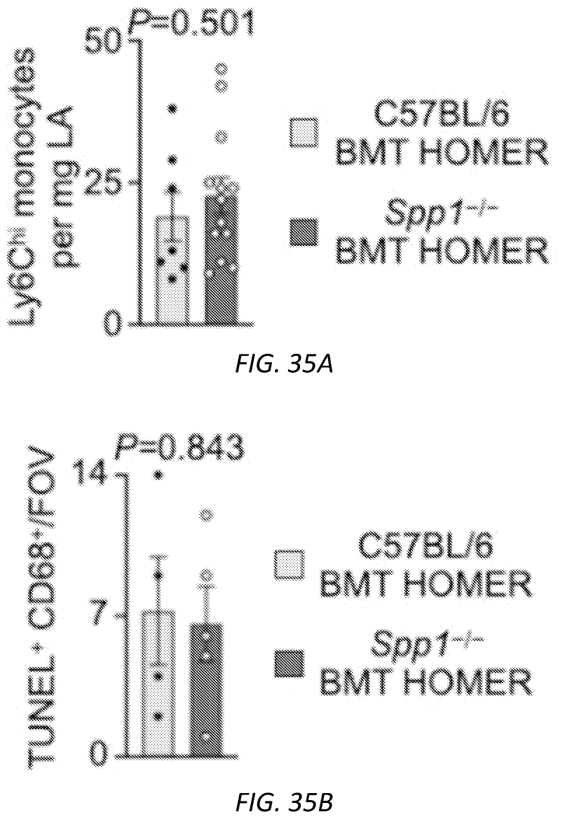

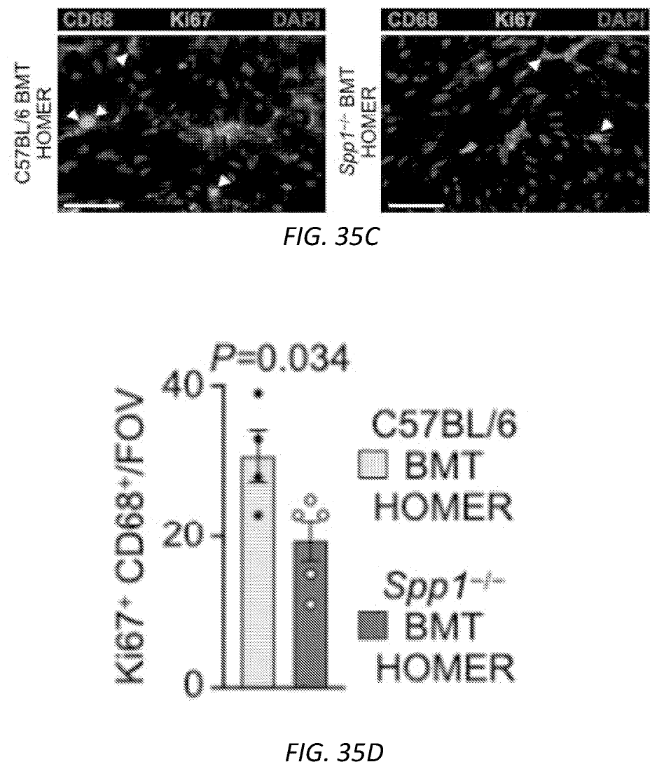

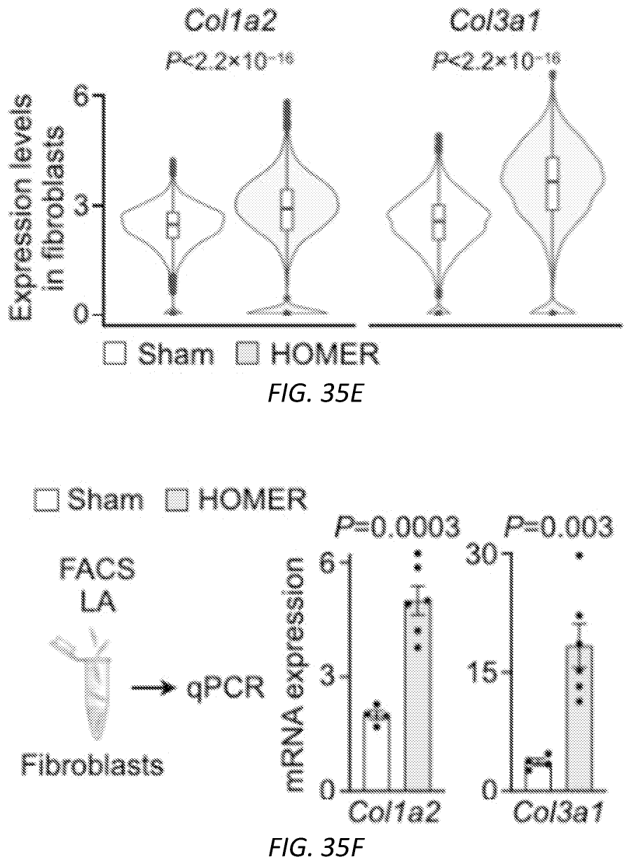

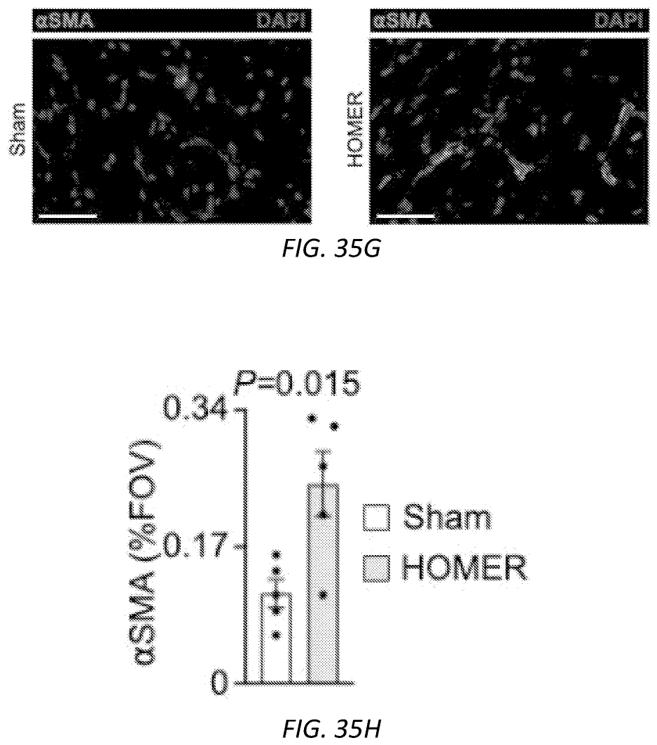

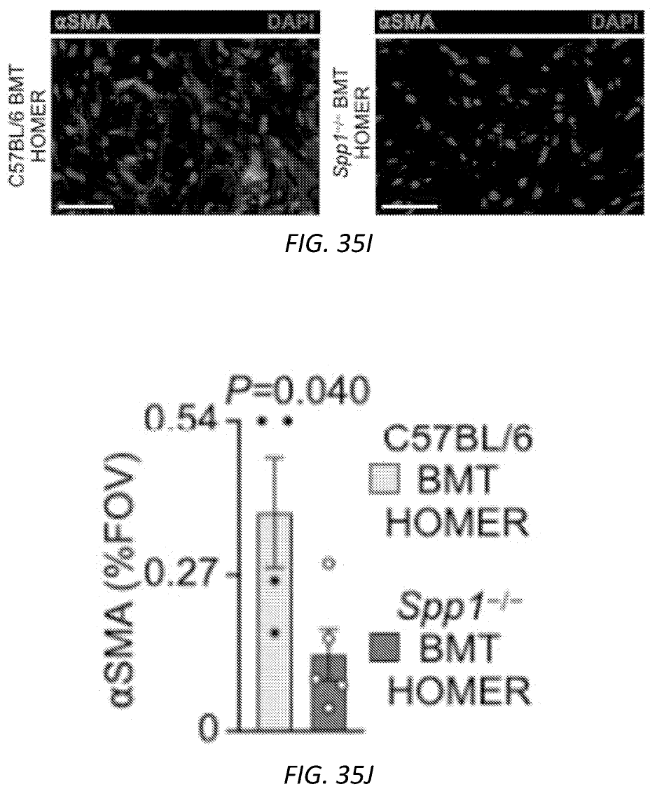

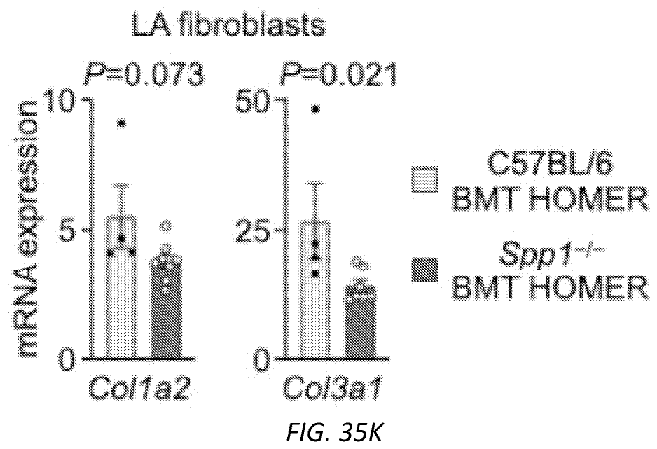

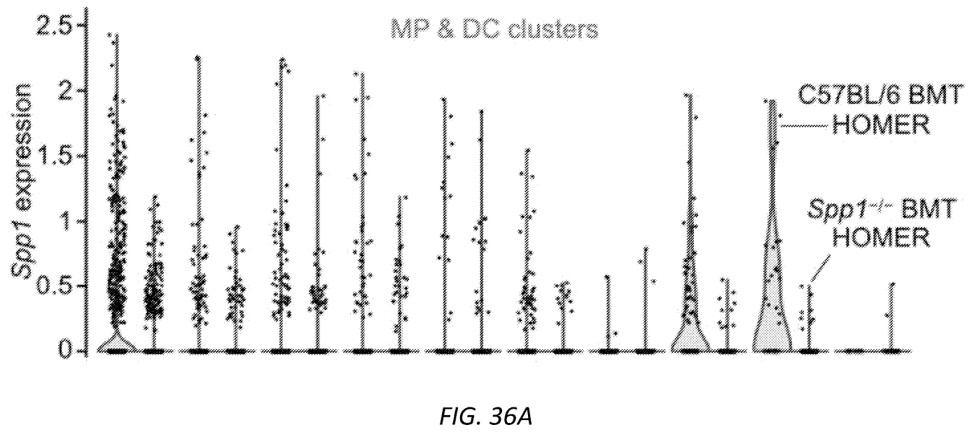

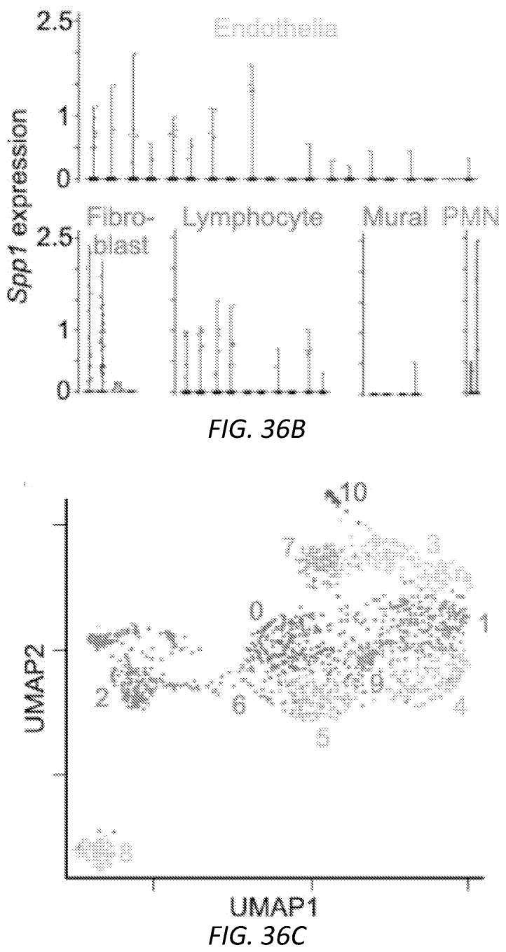

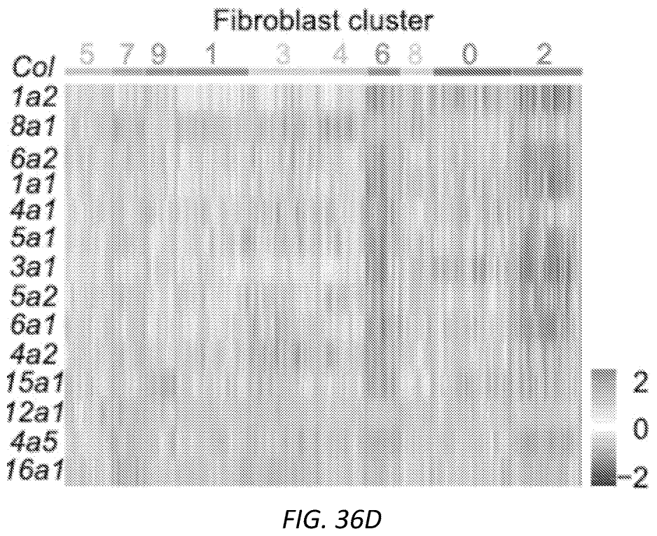

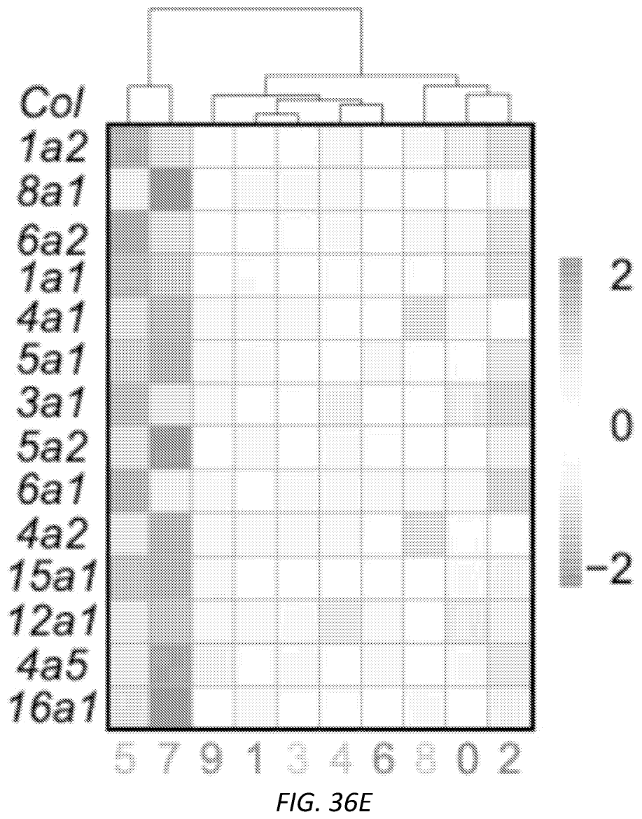

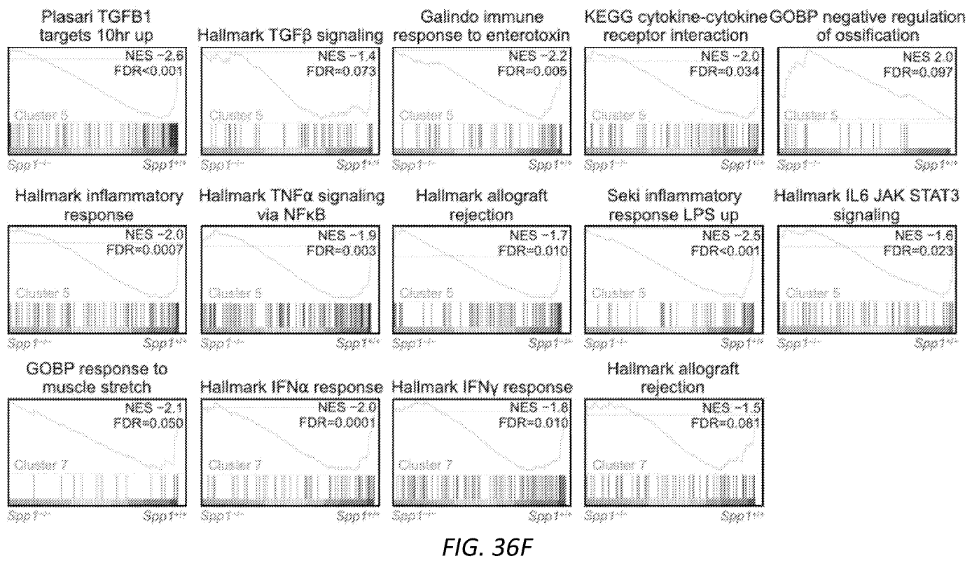

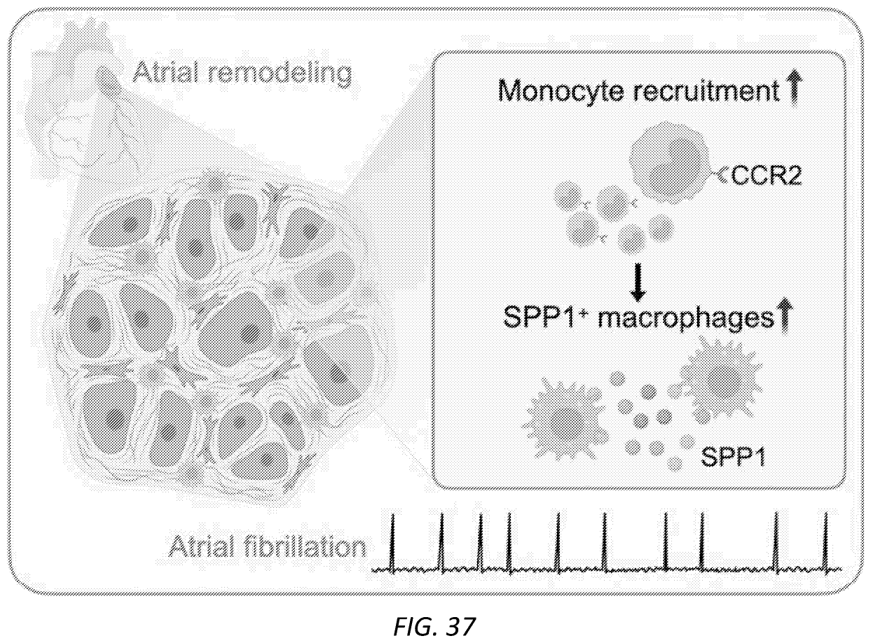

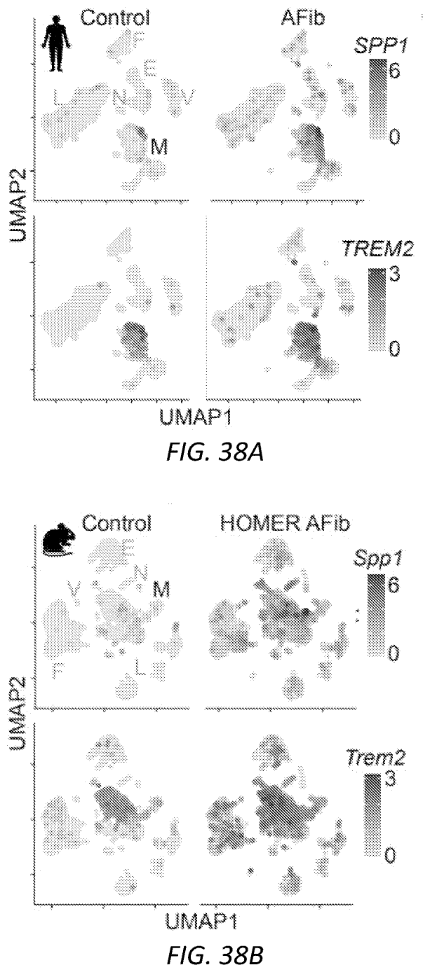

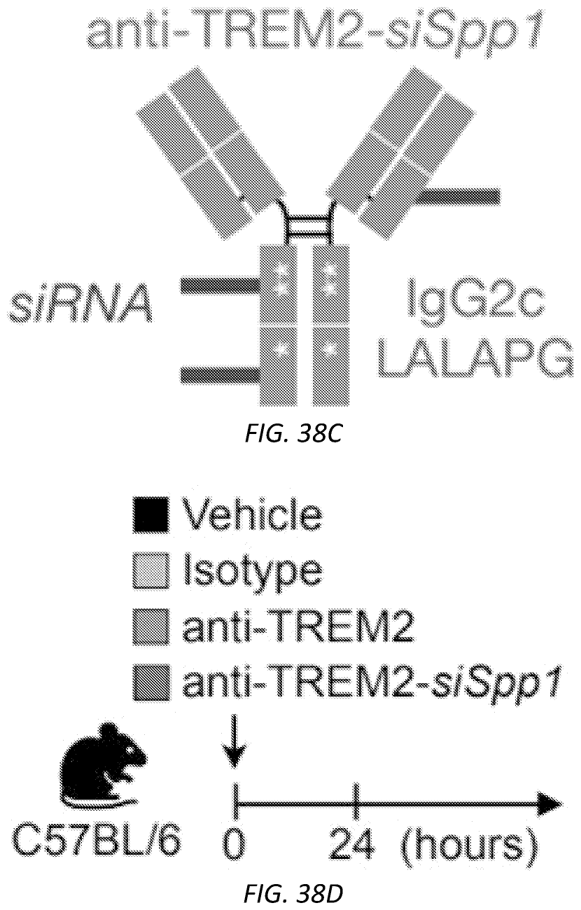

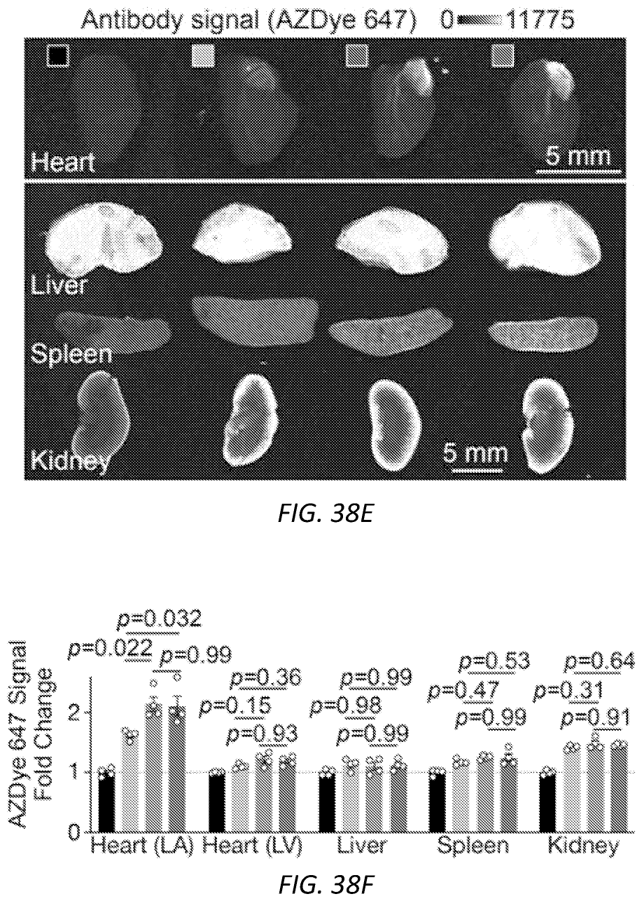

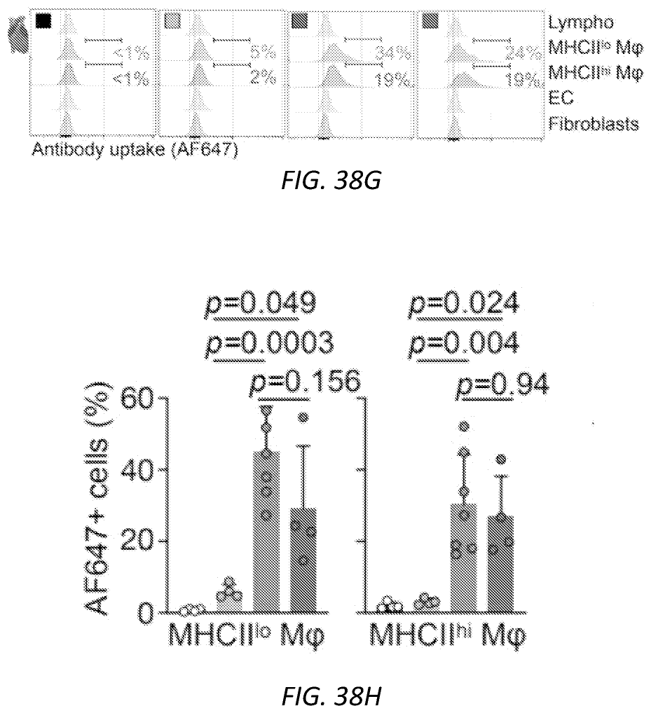

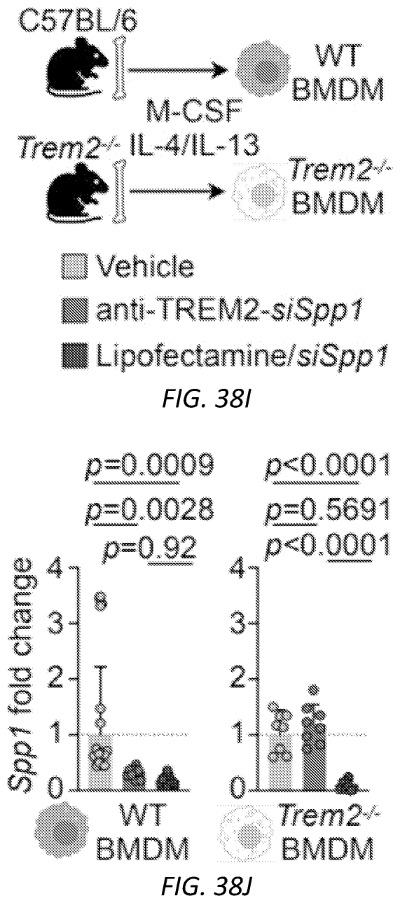

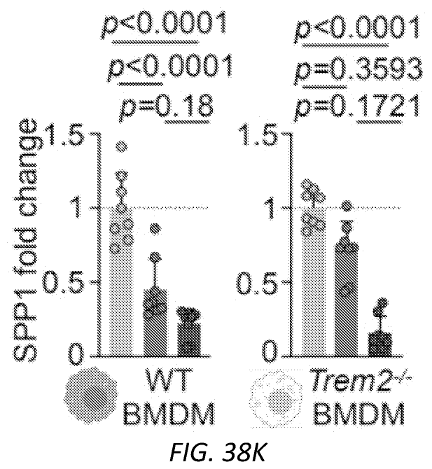

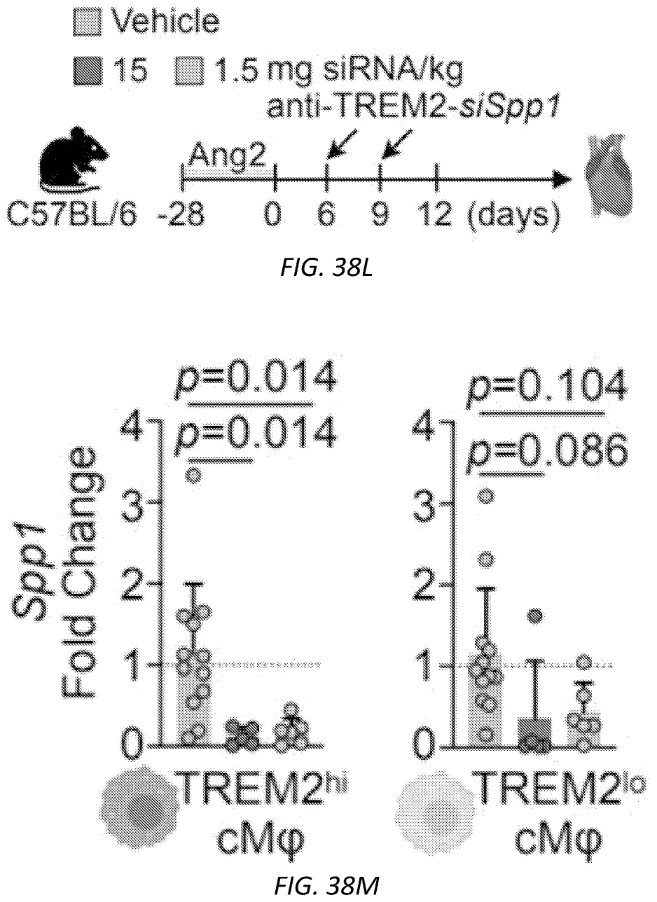

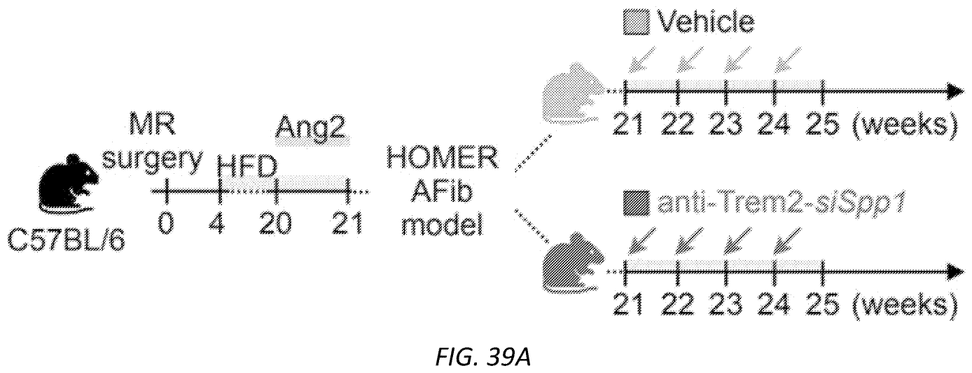

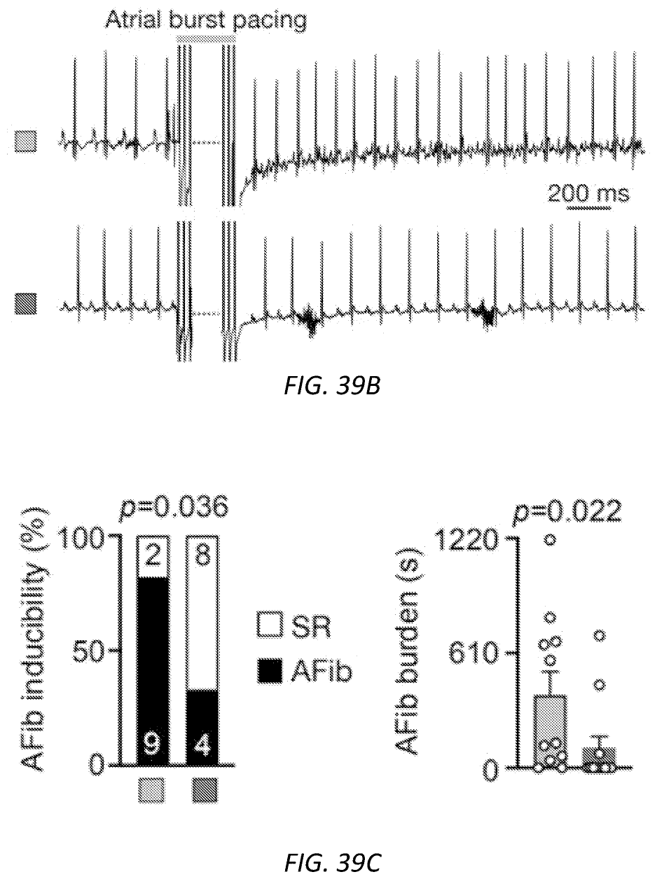

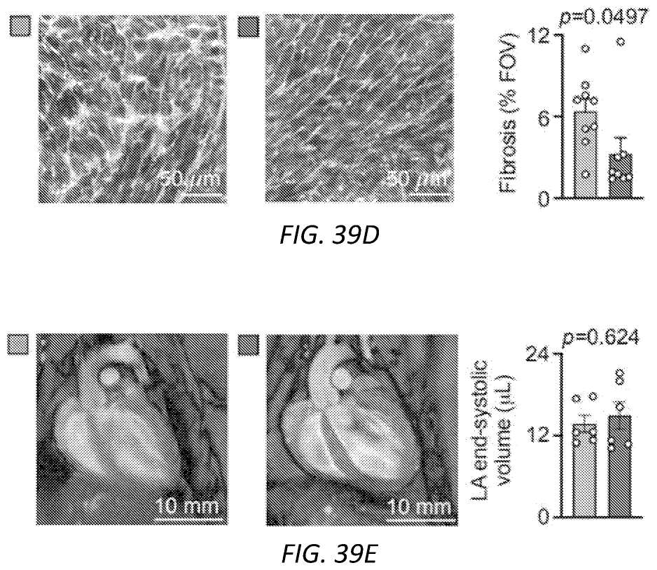

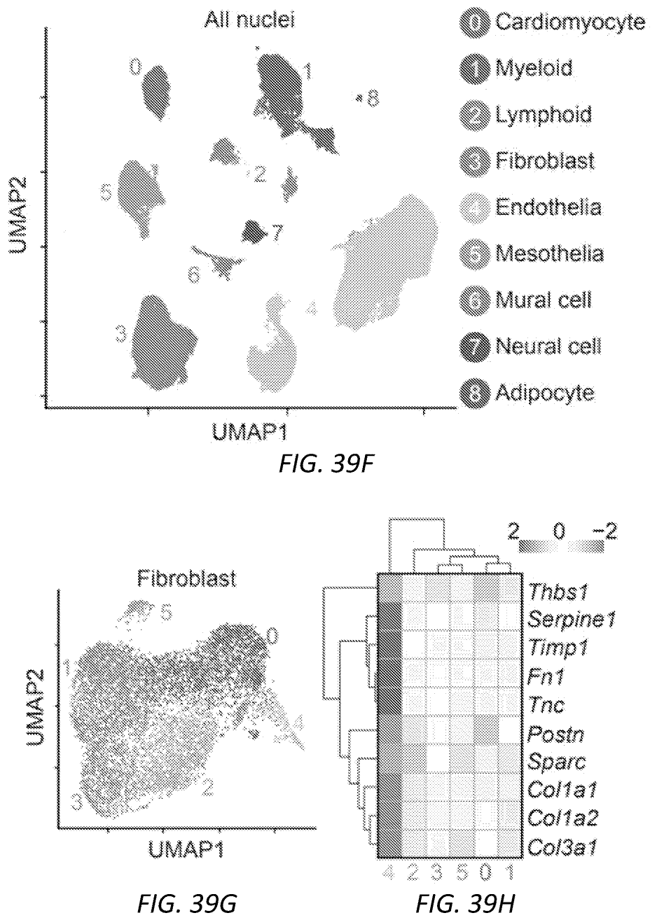

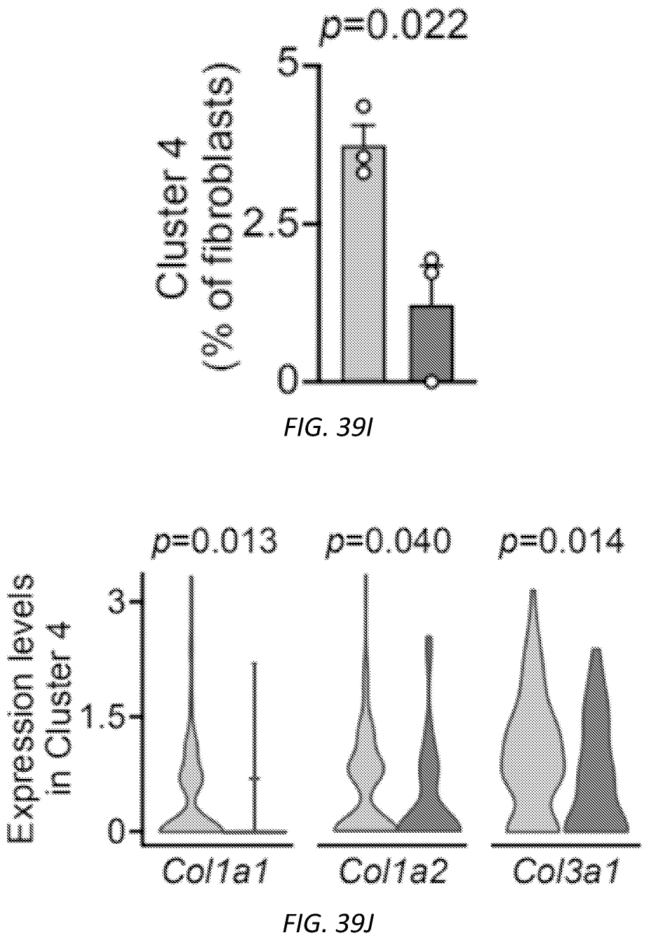

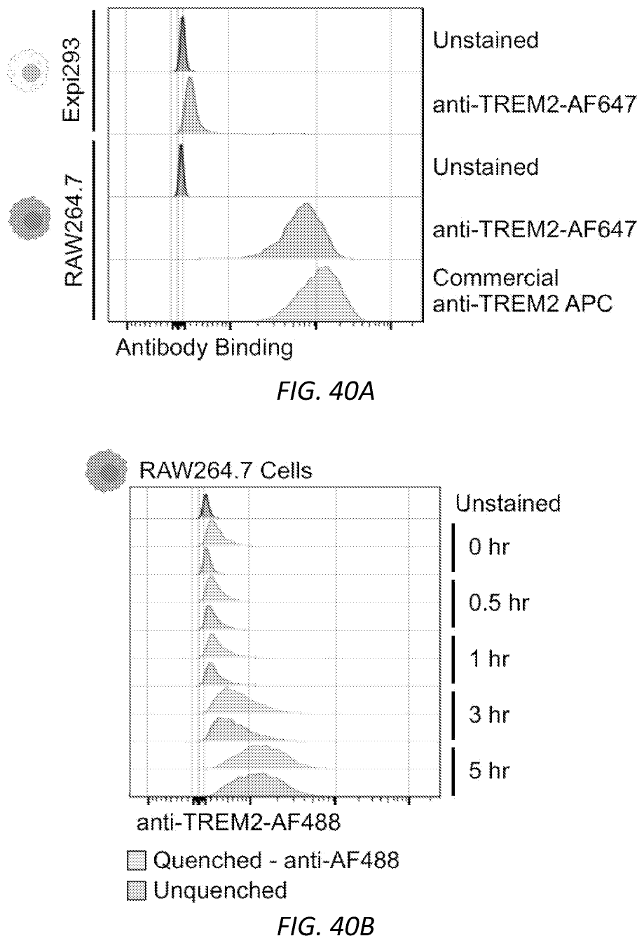

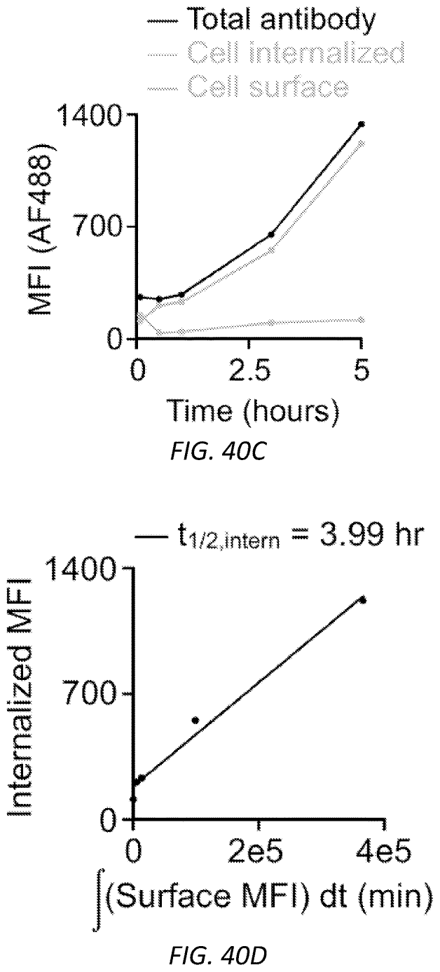

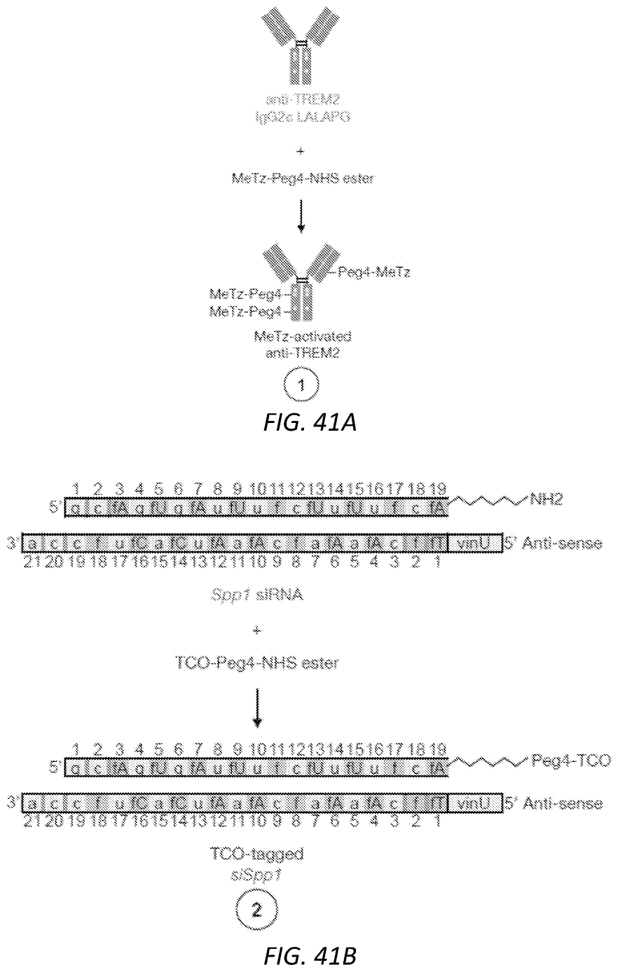

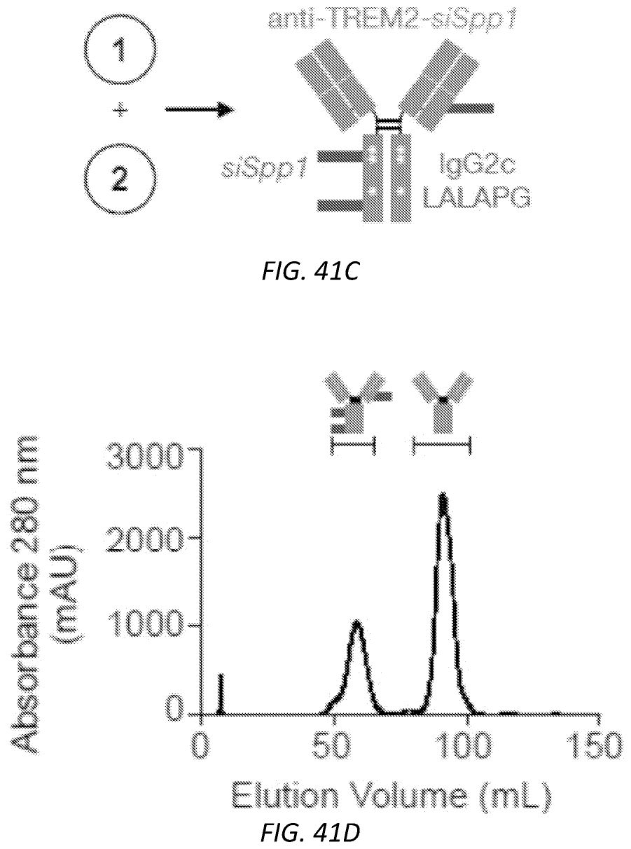

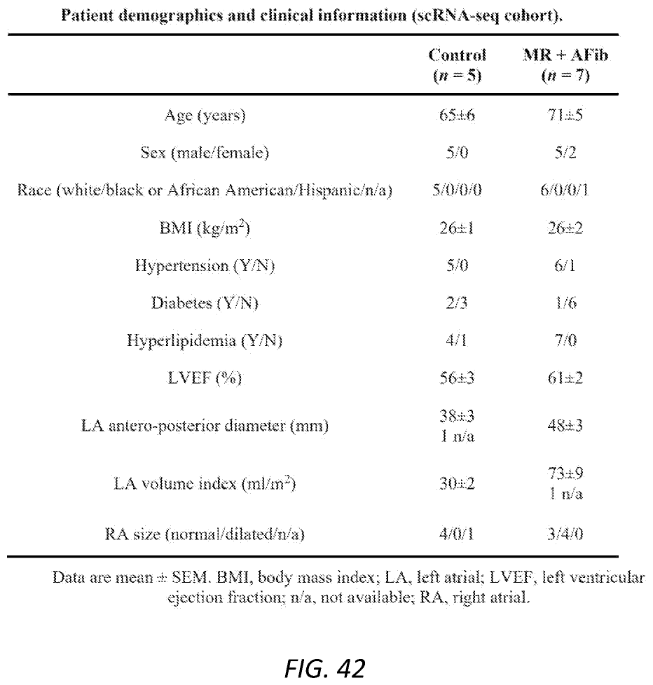

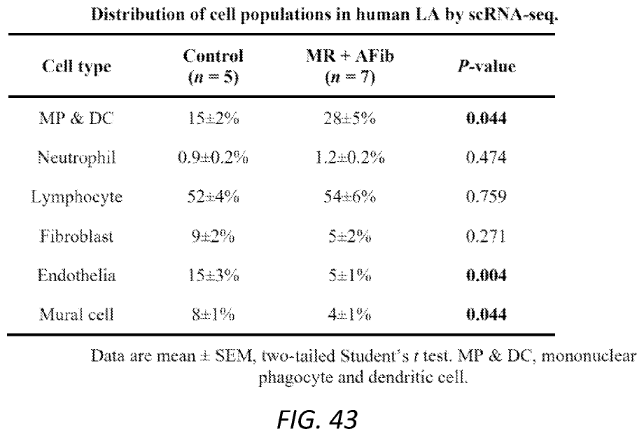

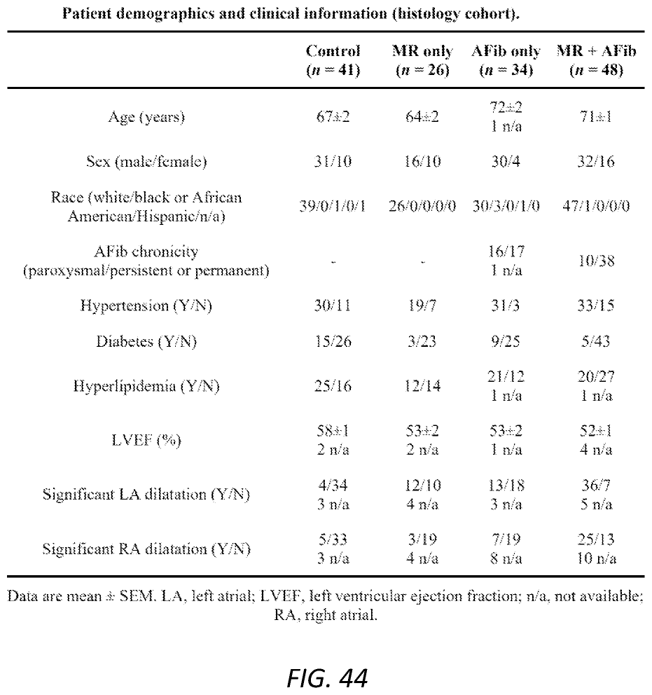

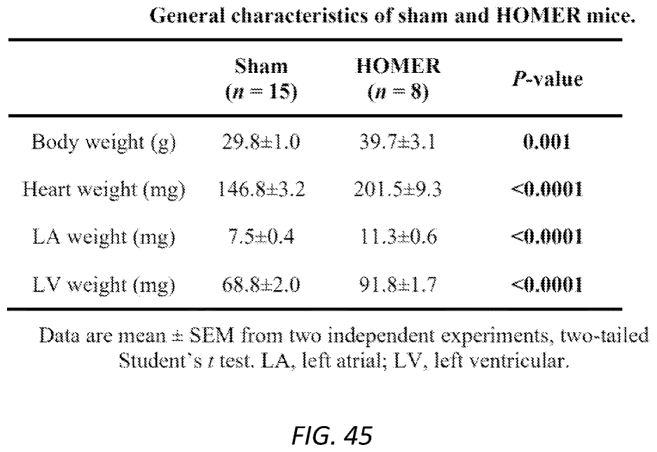

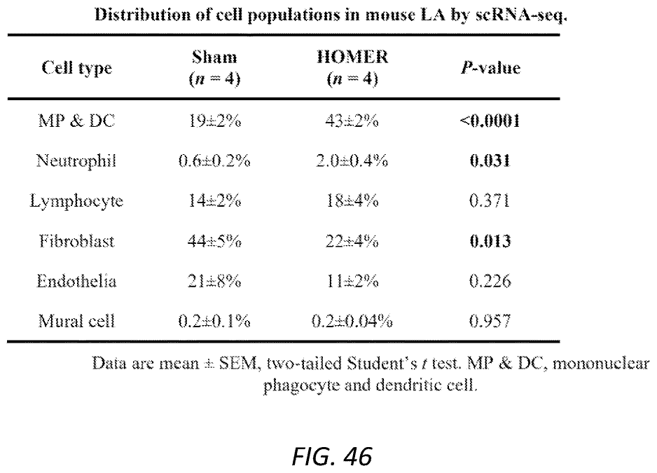

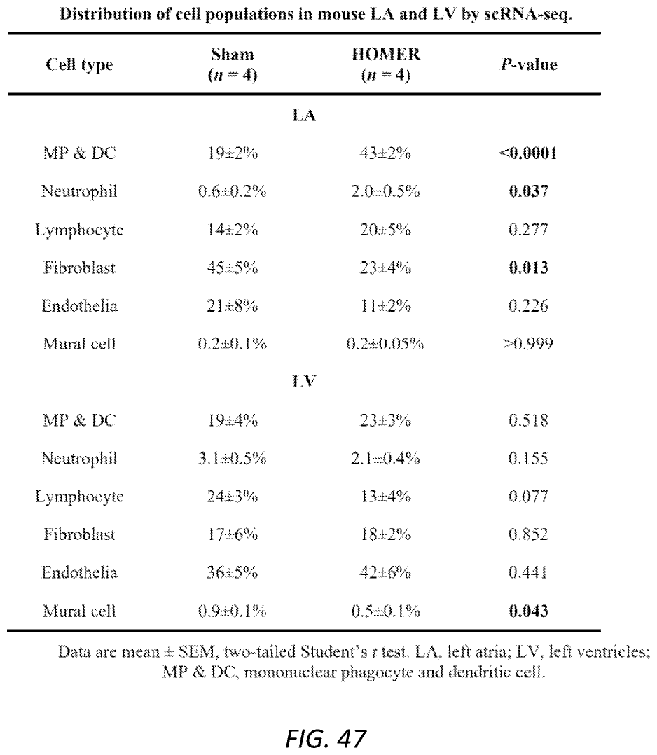

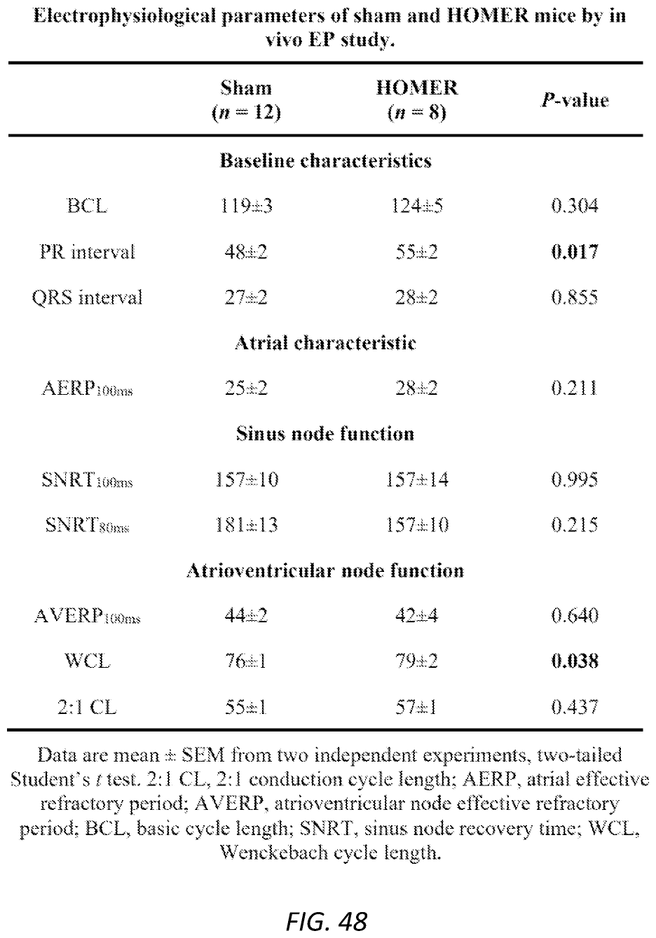

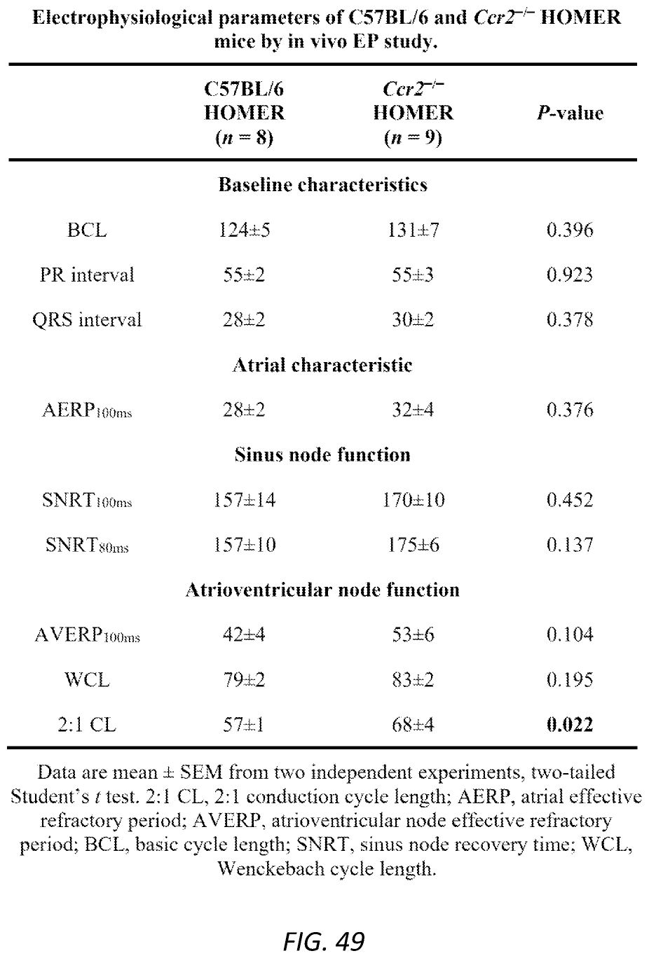

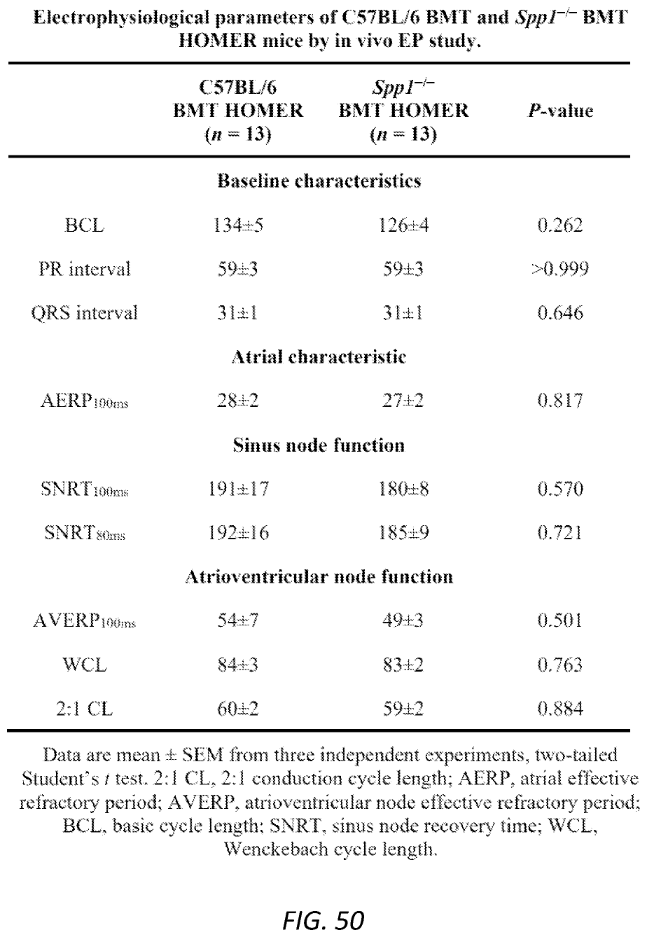

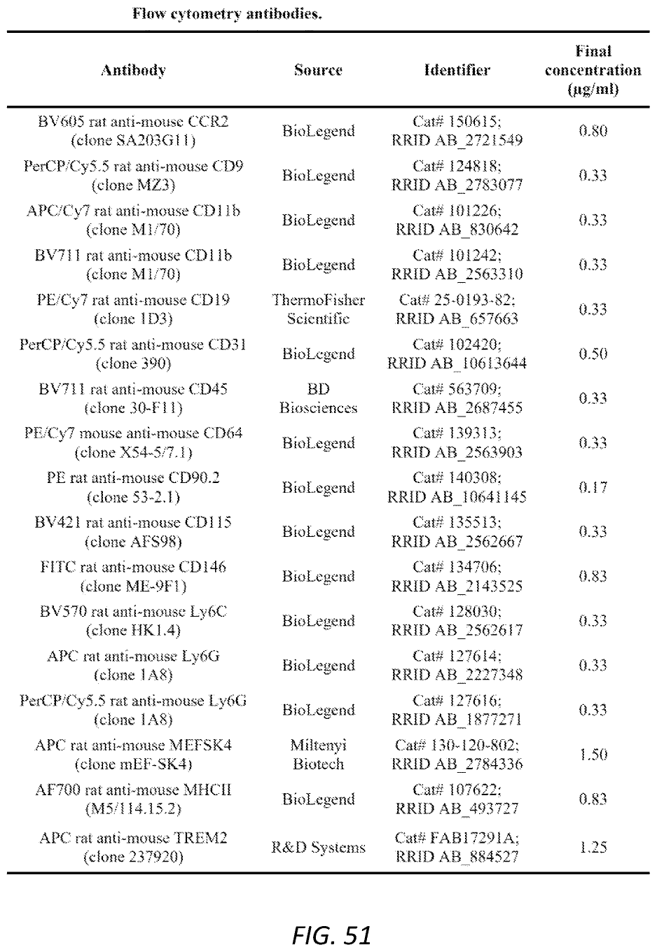

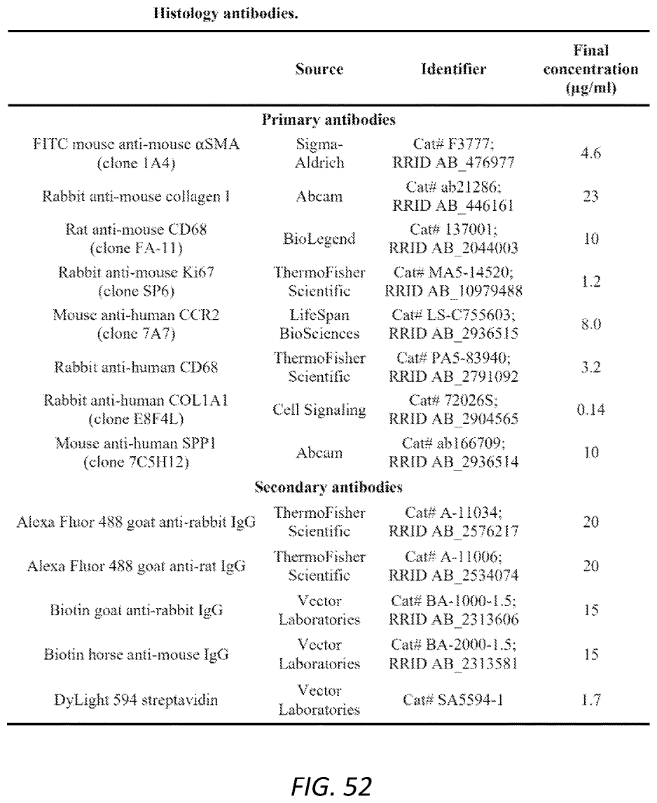

We next assayed contributions of individual HOMER components. Ccr2 was expressed highest in HOMER atria but also increased in hypertensive mice (FIG. 27A). Atrial expression of Csf1, chemokines, adhesion molecules, inflammatory markers, and matrix increased the most in HOMER mice (FIGS. 27B to 27F). Spp1 expression increased in HOMER mice but not after exposure to single components (FIG. 27G). Thus, HOMER components have additive (e.g., Ccr2 expression) and some synergistic effects (e.g., Spp1 expression). Two out of three HOMER components elicited intermediate atrial remodeling and Spp1 expression (FIG. 28). Although combining all HOMER components reflected the risk factors present in our patient cohort most faithfully, further studies of single conditions (e.g., metabolic consequences of high-fat diet on conducting cells) are warranted.

We then contrasted left atrial gene expression changes to the left ventricular myocardium. Unlike the atria, we observed no significant expansion of ventricular leukocyte frequencies in HOMER mice (FIGS. 29A to 29C, and FIG. 47). The decline of fibroblast frequencies that occurred in HOMER atria was absent in the ventricles (FIGS. 29A to 29C, and FIG. 47). Compared to ventricular cells, more genes changed in atrial myeloid cells and fibroblasts. For instance, there were 1982 atrial but only 19 ventricular differentially expressed genes (with FDR<0.05) in MP/DCs (with nine differentially regulated genes shared between the atria and ventricles) (FIGS. 29D and 29E). This pattern was inversed for endothelial cells, which had more differentially expressed genes in the ventricles than in the atria (FIGS. 29D and 29E, and). A principal component analysis revealed that in controls, atrial MP/DCs were located at a large distance from ventricular MP/DCs. The distance between atrial and ventricular MP/DCs was less pronounced in HOMER mice (FIG. 29F). We hypothesize that HOMER-induced activation of inflammatory programs equalizes some of the differences normally imprinted by the atrial versus the ventricular environment. That the HOMER model affects left ventricular leukocytes to a lesser degree than in the left atria corresponds with the more severe atrial than ventricular dilation observed by MRI (FIG. 2C and FIG. 21A). The ventricular data document that hypertension, high-fat diet, and mitral valve disease affect not only atria but the entire heart, a notion reflected by the clinical association of AFib with heart failure (31).

The generation of the HOMER mouse and its scRNA-seq validation against AFib patients provided us with a tool to assess whether immune cell expansion merely associates with—or alternatively contributes to—AFib. We employed the HOMER mouse for two follow-up studies that tested causal implications for key observations in AFib patients: (i) expansion of inflammatory atrial macrophages and (ii) increased atrial SPP1 expression by those macrophages.

### CCR2-Dependent Macrophage Recruitment Promotes AFib

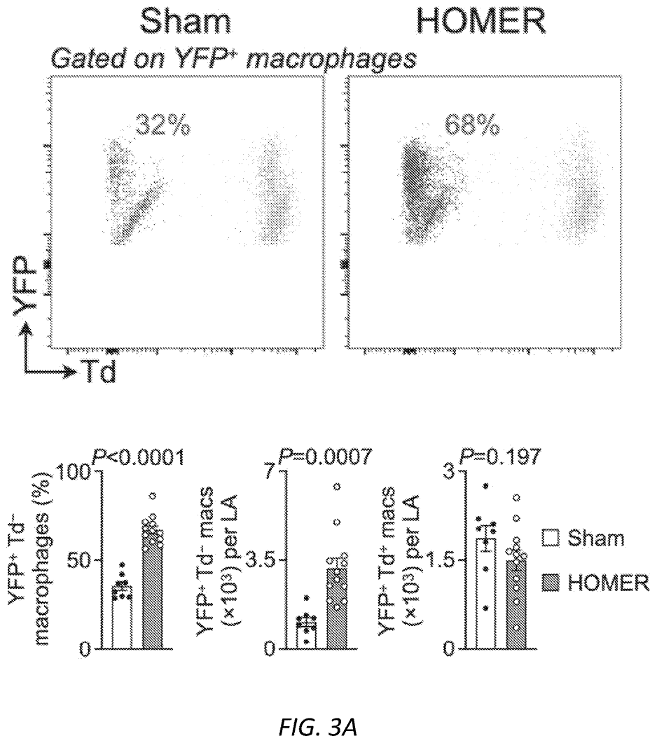

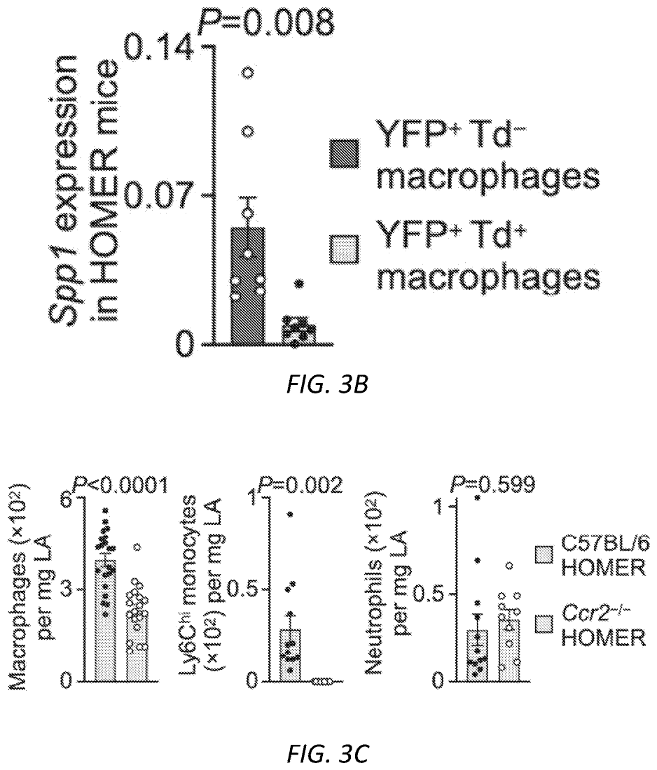

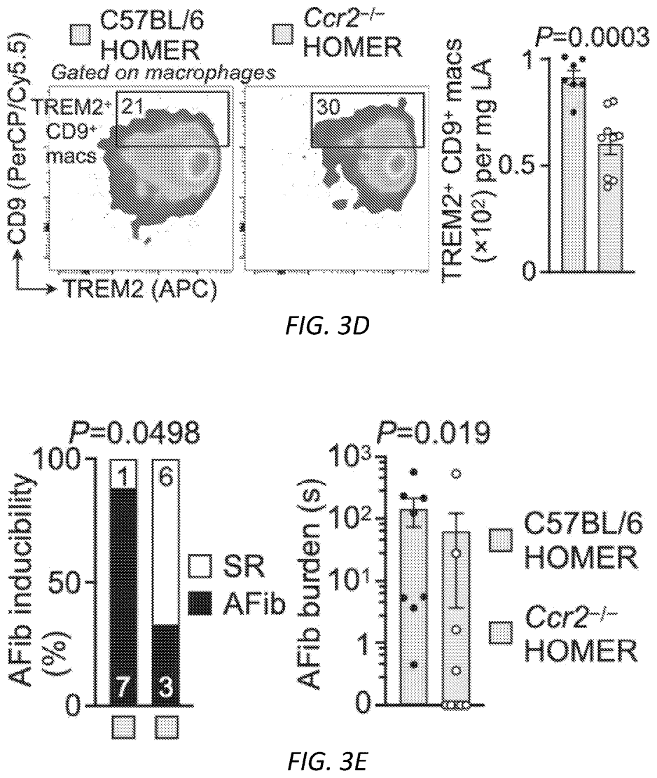

Macrophage ontogeny often dictates function (32). For instance, steady-state resident macrophages in the heart and brain are distinctly non-inflammatory (33), whereas recruited infarct or stroke macrophages are inflammatory and damage tissues (34). We consequently sought to investigate macrophage origins in normal and diseased atria. Since atrial macrophages expand in diseased atria of HOMER mice (FIGS. 2F and 2H, and FIG. 46) and enrich for the chemokine receptor CCR2 (FIG. 24D), which recruited macrophages express (34), we combined the HOMER procedure with genetic fate mapping in Cx3cr1^{CreER/+}Ai9^{fl/+} mice (FIG. 30A). Though atrial macrophages were more likely to be derived from circulating monocytes than ventricular macrophages, most atrial macrophages were not sourced from monocytes in steady state (FIG. 30B). In HOMER mice, recruitment expanded so that 67% of atrial macrophages derived from monocytes, a significant increase from the steady state (36%) (FIG. 3A). In line with increased atrial expression of Csf1 (FIG. 27B) and upregulation of the “Leukocyte proliferation” gene set by MP/DCs (FIG. 30C), macrophage proliferation increased in HOMER atria (FIGS. 30D and 30E). Recruited macrophages in atria of HOMER mice were the primary source of Spp1, whereas locally sourced macrophages did not express this gene at high levels (FIG. 3B).

We employed the HOMER procedure in Ccr2^{-/-} mice to test the relevance of expanded macrophage recruitment for AFib. Blocking monocyte migration in Ccr2^{-/-} HOMER mice decreased blood monocyte, atrial Ly6C^{hi} monocyte, and macrophage expansion when compared to C57BL/6 HOMER mice, whereas other cell densities and blood pressure were unaffected (FIG. 3C and FIGS. 31A to 31D). TREM2⁺CD9⁺ macrophages were also lower in Ccr2^{-/-} HOMER mice (FIG. 3D). Cardiac MRI revealed smaller left atrial volumes in Ccr2^{-/-} HOMER mice (FIG. 31E). Ccr2^{-/-} HOMER mice exhibited lower AFib inducibility and AFib burden than C57BL/6 HOMER mice (FIG. 3E). Thus, macrophage recruitment expands during atrial remodeling, recruited macrophages highly express Spp1 and inhibiting macrophage recruitment reduces AFib.

To test whether CCR2 inhibition could alleviate established disease, we treated mice with the CCR2 antagonist CCL2-Fc (35) for 4 weeks after the last HOMER treatment component (FIG. 32A). CCR2 inhibition reduced fibrosis in atria of HOMER mice (FIGS. 32B and 32C). AFib inducibility and burden were lower in the treatment group, although this effect was not statistically significant (FIG. 32D).

### Macrophage-Derived SPP1 is a Pleiotropic AFib Catalyst

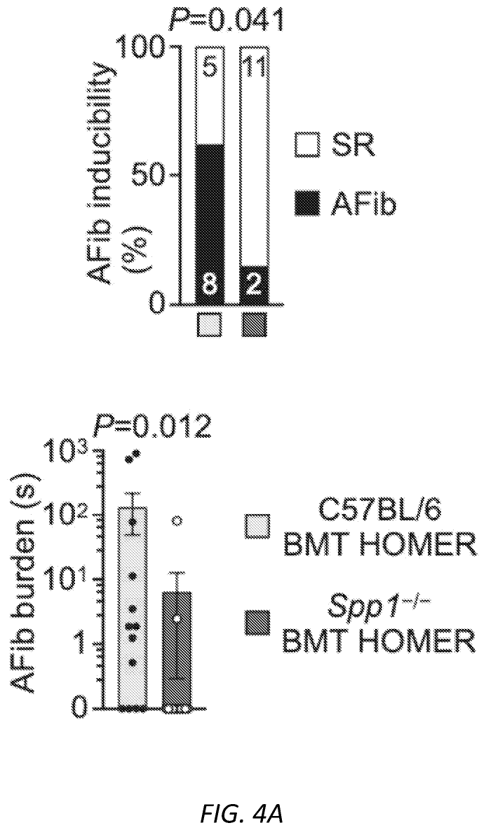

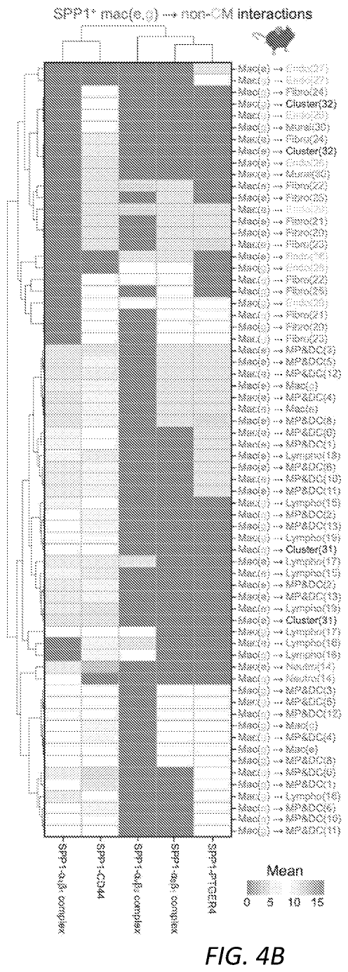

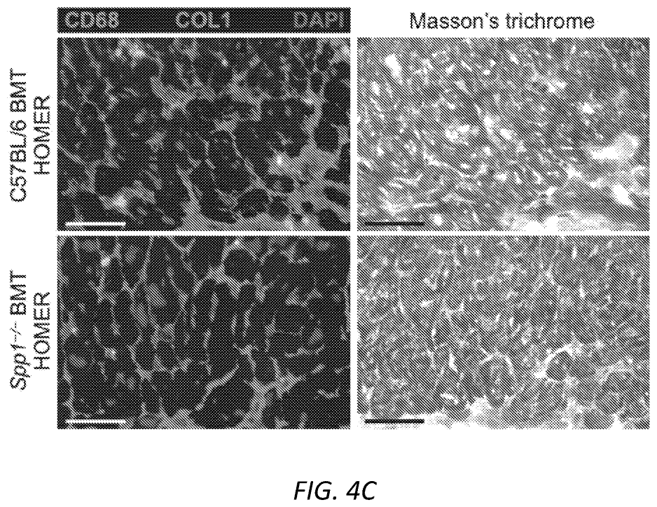

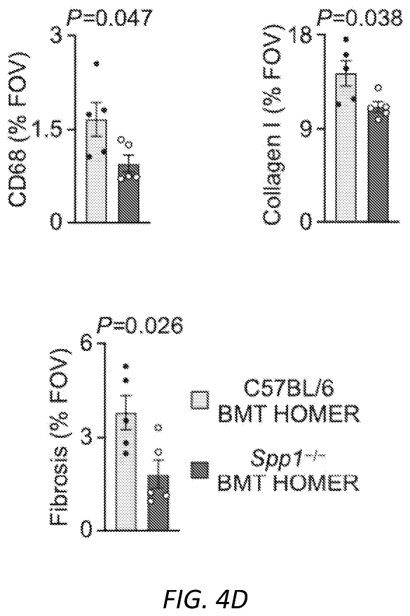

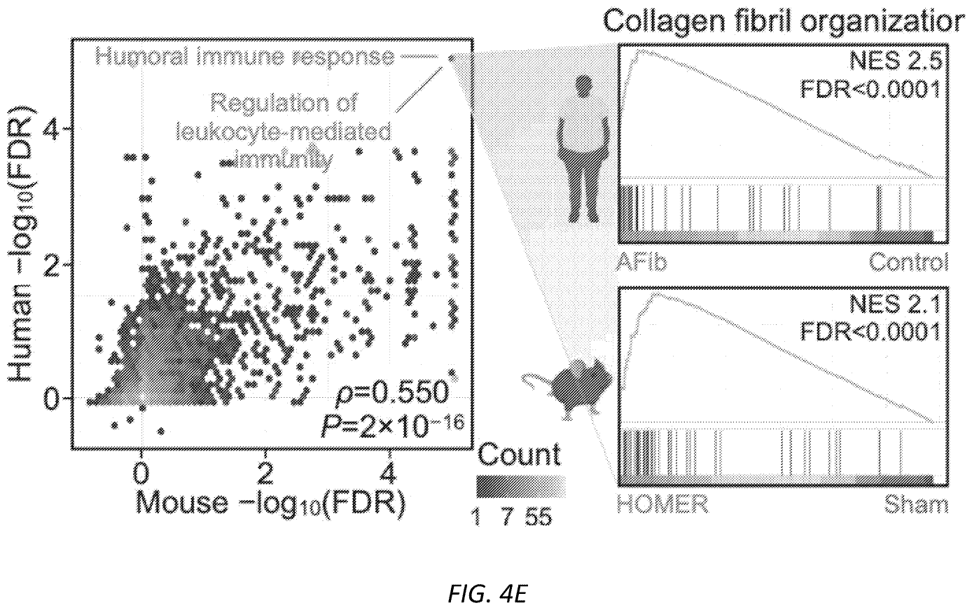

Given that Spp1 is among the most upregulated macrophage genes in AFib, we examined the causal role of this matricellular protein. We transplanted bone marrow from Spp1^{-/-} mice into wild-type recipients (Spp1^{-/-} BMT HOMER) to delete Spp1 in recruited macrophages (FIGS. 33A and 33B). Wild-type recipients that received wild-type bone marrow and then underwent HOMER (C57BL/6 BMT HOMER) served as controls. Blood pressure and leukocyte counts were similar in both cohorts (FIGS. 33C to 33E). Left atrial and ventricular volumes were preserved and left ventricular hypertrophy was lessened in knockout mice (FIG. 33F). Deleting Spp1 in bone marrow-derived cells reduced AFib inducibility and AFib burden (FIG. 4A).

To outline mechanisms by which osteopontin exacerbates AFib, we investigated the crosstalk between Spp1-expressing macrophages and other atrial cells using CellPhoneDB (36). In HOMER mice and AFib patients, paracrine and autocrine interactions were found between macrophage-derived SPP1 and cognate SPP1 receptors expressed by immune and stromal cells (FIG. 4B and FIG. 34). In HOMER mice, CellPhoneDB analysis implied SPP1⁺ macrophage interactions with client cells via three integrins (α₄β₁, αᵥβ₃, and α₉β₁), CD44, and prostaglandin E₂ receptor 4 (EP₄/PTGER4). These interactions, which included signaling to various macrophage and fibroblast clusters, suggested that SPP1 acts as a pleiotropic amplifier for atrial inflammation and fibrosis, which in turn contribute to an AFib substrate (37).

In support of this idea, histology of atria collected from Spp1^{-/-} BMT HOMER mice showed fewer CD68⁺ macrophages when compared to C57BL/6 BMT HOMER mice (FIGS. 4C and 4D), indicating that SPP1 may influence atrial macrophage supply. Atrial monocyte numbers were unchanged in Spp1^{-/-} BMT HOMER mice (FIG. 35A), making a role of SPP1 in monocyte recruitment unlikely. TUNEL/CD68 staining of atria indicated that macrophage death was also unchanged in the knockout cohort (FIG. 35B). However, macrophage proliferation, which is upregulated by HOMER conditions (FIGS. 30D and 30E), declined in Spp1^{-/-} BMT HOMER compared to C57BL/6 BMT HOMER mice (FIGS. 35C and 35D). Thus, SPP1 augments atrial inflammation via increased macrophage proliferation.

Matrix production by fibroblasts increased in HOMER mice (FIGS. 35E and 35F) and immunoreactive alpha smooth muscle actin (aSMA) staining of HOMER atria documented an expansion of activated fibroblasts which were reduced in Spp1^{-/-} BMT HOMER mice (FIGS. 35G to 35J). Atrial fibroblasts isolated from Spp1^{-/-} BMT HOMER mice expressed less collagen than those from C57BL/6 BMT HOMER (FIG. 35K). Furthermore, we detected reductions in collagen I deposition and Masson's trichrome staining in atria of Spp1^{-/-} BMT HOMER mice (FIGS. 4C and 4D), which points to reduced fibroblast matrix production if these cells do not sense SPP1. This interaction may be particularly relevant because integrins—which based on our CellPhoneDB analysis fibroblasts employ to sense SPP1—regulate fibroblast migration, proliferation, and activation, processes that precede increased tissue fibrosis (38, 39).

Comparing left atrial cells from Spp1^{-/-} BMT HOMER to C57BL/6 BMT HOMER mice by scRNA-seq (FIGS. 36A and 36B), we identified fibroblast clusters that expressed collagens at high levels (FIGS. 36C to E). For these clusters, we pursued GSEA to examine via which pathways SPP1 activates atrial fibroblasts (FIG. 36F). Fibroblast clusters from C57BL/6 BMT HOMER mice upregulated Tgfb1 gene sets. Inflammatory activation of fibroblasts by SPP1 was supported by the upregulation of inflammatory gene sets in fibroblasts from C57BL/6 BMT HOMER when compared to Spp1^{-/-} BMT HOMER mice (FIG. 36F). Thus, SPP1 participates in inflammatory fibroblast activation partially via TGF-β pathways. In atrial tissue from AFib patients, SPP1 colocalization with collagen 1A1 expanded (FIGS. 17C and 17D), lending further credence to SPP1 promotion of AFib via enhanced fibrosis.

To ascertain whether the transcriptional changes that occurred in atrial fibroblasts of HOMER mice are representative of alterations in humans, we compared human and mouse fibroblast transcriptomes. We first performed GSEA (again using all C5 ontology MSigDB gene sets) on differentially expressed genes from a pseudobulk analysis comparing all fibroblast clusters isolated from atria of AFib patients versus humans without AFib, as well as HOMER versus control mice. We then plotted the significance and direction of change for all gene sets for both species. Both GSEA data sets correlated strongly (Spearman's ρ=0.55, P<2×10⁻¹⁶), with gene sets related to immune response and collagen organization enriched in diseased atria (FIG. 4E). This pathway-level interspecies correlation further bolstered the physiological relevance of the HOMER model, while also underscoring inflammation and fibrosis as central processes in AFib.

### Example 2. SiRNA Knockdown of Spp1

TABLE 2
Abbreviations of nucleotide monomers

| Abbreviation | Nucleotide |
|---|---|
| Am | 2′-O-methyladenosine-3′-phosphate |
| Ams | 2′-O-methyladenosine-3′-phosphorothioate |
| Af | 2′-fluoroadenosine-3′-phosphate |
| Afs | 2′-fluoroladenosine-3′-phosphorothioate |
| Gm | 2′-O-methylguanosine-3′-phosphate |
| Gms | 2′-O-methylguanosine-3′-phosphorothioate |
| Gf | 2′-fluoroguanosine-3′-phosphate |
| Gfs | 2′-fluoroguanosine-3′-phosphorothioate |
| Cm | 2′-O-methylcytidine-3′-phosphate |
| Cms | 2′-O-methylcytidine-3′-phosphorothioate |
| Cf | 2′-fluorocytidine-3′-phosphate |
| Cfs | 2′-fluorocytidine-3′-phosphorothioate |
| Um | 2′-O-methyluridine-3′-phosphate |
| Ums | 2′-O-methyluridine-3′-phosphorothioate |
| Uf | 2′-O-fluorouridine-3′-phosphate |
| Ufs | 2′-O-fluorouridine-3′-phosphorothioate |

Spp1-silencing siRNAs were evaluated in vitro and in vivo. The sequences are shown in the following Table 2. Modifications include N-RNA, dN-DNA, n-2′-OMe-RNA, s-phosphorothioate.

TABLE 3
siRNAs with Modifications
siRNAs with modifications

| | 23 mer target | | | | | | | | |
|---|---|---|---|---|---|---|---|---|---|
| | site | SEQ | | | | SEQ | | | SEQ |
| Targeting | sequence | ID | duplex | SS | sense strand | ID | | antisense strand | ID |
| site* | (5′-3′) | NO | ID | ID | sequence (5′-3′) | NO | AS ID | sequence (5′-3′) | NO |
| 87 | AGGACUAACUACG | 1 | XD- | X120 | gaCuaaCUAcgAccauG | 25 | X120129 | dTUCAUGGUCGUAgUUAGUCc | 49 |
| | ACCAUGAGAU | | 43678 | 128 | asa | | | su | |
| 110 | UGGCAGUGAUUUG | 2 | XD- | X120 | gcAgugAUUugCuuuuG | 26 | X120131 | dTGCAAAAGCAAAuCACUGCC | 50 |
| | CUUUUGCCUG | | 43679 | 130 | csa | | | sa | |
| 111 | GGCAGUGAUUUGC | 3 | XD- | X120 | caGugaUUUgcUuuugC | 27 | X120133 | dTGGCAAAAGCAAaUCACUGc | 51 |
| | UUUUGCCUGU | | 43680 | 132 | csa | | | SC | |
| 311 | AAGAAACUCUUCC | 4 | XD- | X120 | gaAacuCUUccAagcaA | 28 | X120135 | dTAUUGCUUGGAAgAGUUUCu | 52 |
| | AAGCAAUUCC | | 43681 | 134 | usa | | | su | |
| 418 | UCGGAUGAAUCUG | 5 | XD- | X120 | ggAugaAUCugAcgaaU | 29 | X120137 | dTGAUUCGUCAGAuUCAUCCg | 53 |
| | ACGAAUCUCA | | 43682 | 136 | csa | | | sa | |
| 440 | ACCAUUCGGAUGA | 6 | XD- | X120 | caUucgGAUgaGucugA | 30 | X120139 | dTAUCAGACUCAUcCGAAUGg | 54 |
| | GUCUGAUGAG | | 43683 | 138 | usa | | | su | |
| 487 | GACACUUUCACUC | 7 | XD- | X120 | caCuuuCACucCaaucG | 31 | X120141 | dTACGAUUGGAGUgAAAGUGu | 55 |
| | CAAUCGUCCC | | 43684 | 140 | usa | | | SC | |
| 546 | GGCUUAUGGACUG | 8 | XD- | X120 | cuUaugGACugAggucA | 32 | X120143 | dTUUGACCUCAGUCCAUAAGC | 56 |
| | AGGUCAAAGU | | 43685 | 142 | asa | | | SC | |
| 558 | GAGGUCAAAGUCU | 9 | XD- | X120 | ggUcaaAGUcuAggagU | 33 | X120145 | dTAACUCCUAGACuUUGACCu | 57 |
| | AGGAGUUUCC | | 43686 | 144 | usa | | | SC | |
| 706 | CAGGACAACAACG | 10 | XD- | X120 | ggAcaaCAAcgGaaagG | 34 | X120147 | dTCCCUUUCCGUUgUUGUCCu | 58 |
| | GAAAGGGCAG | | 43687 | 146 | gsa | | | sg | |
| 813 | GUCGGAUGUGAUC | 11 | XD- | X120 | cgGaugUGAucGauagU | 35 | X120149 | dTGACUAUCGAUCaCAUCCGa | 59 |
| | GAUAGUCAAG | | 43688 | 148 | csa | | | SC | |
| 814 | UCGGAUGUGAUCG | 12 | XD- | X120 | ggAuguGAUcgAuaguC | 36 | X120151 | dTUGACUAUCGAUCACAUCCg | 60 |
| | AUAGUCAAGC | | 43689 | 150 | asa | | | sa | |
| 820 | GUGAUCGAUAGUC | 13 | XD- | X120 | gaUcgaUAGucAagcaA | 37 | X120153 | dTCUUGCUUGACUaUCGAUCa | 61 |
| | AAGCAAGUUC | | 43690 | 152 | gsa | | | SC | |
| 916 | AAGGAAGAUGAUA | 14 | XD- | X120 | ggAagaUGAuaGguauC | 38 | X120155 | dTAGAUACCUAUCaUCUUCCu | 62 |
| | GGUAUCUGAA | | 43691 | 154 | usa | | | su | |
| 927 | UAGGUAUCUGAAA | 15 | XD- | X120 | ggUaucUGAaaUuccgA | 39 | X120157 | dTUUCGGAAUUUCaGAUACCu | 63 |
| | UUCCGAAUUU | | 43692 | 156 | asa | | | sa | |
| 943 | CGAAUUUCUCAUG | 16 | XD- | X120 | aaUuucUCAugAauuaG | 40 | X120159 | dTUCUAAUUCAUGaGAAAUUc | 64 |
| | AAUUAGAGAG | | 43693 | 158 | asa | | | sg | |
| 1050 | AGGGUUAAGCAGG | 17 | XD- | X120 | ggUuaaGCAggAauacU | 41 | X120161 | dTUAGUAUUCCUGCUUAACCc | 65 |
| | AAUACUAACU | | 43694 | 160 | asa | | | su | |
| 1059 | CAGGAAUACUAAC | 18 | XD- | X120 | ggAauaCUAacUgcucA | 42 | X120163 | dTAUGAGCAGUUAgUAUUCCu | 66 |
| | UGCUCAUUUC | | 43695 | 162 | usa | | | sg | |
| 1314 | AACUGUAACAGUC | 19 | XD- | X120 | cuGuaaCAGucUauauC | 43 | X120165 | dTUGAUAUAGACUgUUACAGu | 67 |
| | UAUAUCACUA | | 43696 | 164 | asa | | | su | |
| 1316 | CUGUAACAGUCUA | 20 | XD- | X120 | guAacaGUCuaUaucaC | 44 | X120167 | dTAGUGAUAUAGACUGUUACa | 68 |
| | UAUCACUAUA | | 43697 | 166 | usa | | | sg | |
| 1320 | AACAGUCUAUAUC | 21 | XD- | X120 | caGucuAUAucAcuauA | 45 | X120169 | dTGUAUAGUGAUAuAGACUGu | 69 |
| | ACUAUACCCU | | 43698 | 168 | csa | | | su | |
| 1330 | AUCACUAUACCCU | 22 | XD- | X120 | caCuauACCcuUuuagU | 46 | X120171 | dTAACUAAAAGGGuAUAGUGa | 70 |
| | UUUAGUUUUA | | 43699 | 170 | usa | | | su | |
| 1331 | UCACUAUACCCUU | 23 | XD- | X120 | acUauaCCCuuUuaguU | 47 | X120173 | dTAAACUAAAAGGgUAUAGUg | 71 |
| | UUAGUUUUAU | | 43700 | 172 | usa | | | sa | |
| 1389 | UUGGUGUGAAUAA | 24 | XD- | X120 | ggUgugAAUaaAucuuG | 48 | X120175 | dTACAAGAUUUAUuCACACCa | 72 |
| | AUCUUGUAAC | | 43701 | 174 | usa | | | sa | |

*5′ position of 19 mer target site in NM_009263.3

TABLE 4
siRNA Sequences and Cross-reactivity

| | | | | | | siRNA strands 19/21 mer | | | |
|---|---|---|---|---|---|---|---|---|---|
| Cross- | siRNA strands 19/19 mer, unmodified | | | | | (SS pos 19 = “A”, AS pos 1 = “U”), unmodified | | | |
| reacti- | | sense strand | SEQ | | SEQ | | SEQ | | SEQ |
| vity** | | sequence | ID | antisense strand | ID | sense strand | ID | antisense strand | ID |
| HUMAN | name | (5′-3′) | NO | sequence (5′-3′) | NO | sequence (5′-3′) | NO | sequence (5′-3′) | NO |
| 0 | 87 | GACUAACUACGAC | 73 | CUCAUGGUCGUAGUUA | 97 | GACUAACUACGACCAUGAA | 121 | UUCAUGGUCGUAGUU | 145 |
| | | CAUGAG | | GUC | | | | AGUCCU | |
| 1 | 110 | GCAGUGAUUUGCU | 74 | GGCAAAAGCAAAUCAC | 98 | GCAGUGAUUUGCUUUUGCA | 122 | UGCAAAAGCAAAUCA | 146 |
| | | UUUGCC | | UGC | | | | CUGCCA | |
| 1 | 111 | CAGUGAUUUGCUU | 75 | AGGCAAAAGCAAAUCA | 99 | CAGUGAUUUGCUUUUGCCA | 123 | UGGCAAAAGCAAAUC | 147 |
| | | UUGCCU | | CUG | | | | ACUGCC | |
| 0 | 311 | GAAACUCUUCCAA | 76 | AAUUGCUUGGAAGAGU | 100 | GAAACUCUUCCAAGCAAUA | 124 | UAUUGCUUGGAAGAG | 148 |
| | | GCAAUU | | UUC | | | | UUUCUU | |
| 0 | 418 | GGAUGAAUCUGAC | 77 | AGAUUCGUCAGAUUCA | 101 | GGAUGAAUCUGACGAAUCA | 125 | UGAUUCGUCAGAUUC | 149 |
| | | GAAUCU | | UCC | | | | AUCCGA | |
| 0 | 440 | CAUUCGGAUGAGU | 78 | CAUCAGACUCAUCCGA | 102 | CAUUCGGAUGAGUCUGAUA | 126 | UAUCAGACUCAUCCG | 150 |
| | | CUGAUG | | AUG | | | | AAUGGU | |
| 0 | 487 | CACUUUCACUCCA | 79 | GACGAUUGGAGUGAAA | 103 | CACUUUCACUCCAAUCGUA | 127 | UACGAUUGGAGUGAA | 151 |
| | | AUCGUC | | GUG | | | | AGUGUC | |
| 1 | 546 | CUUAUGGACUGAG | 80 | UUUGACCUCAGUCCAU | 104 | CUUAUGGACUGAGGUCAAA | 128 | UUUGACCUCAGUCCA | 152 |
| | | GUCAAA | | AAG | | | | UAAGCC | |
| 0 | 558 | GGUCAAAGUCUAG | 81 | AAACUCCUAGACUUUG | 105 | GGUCAAAGUCUAGGAGUUA | 129 | UAACUCCUAGACUUU | 153 |
| | | GAGUUU | | ACC | | | | GACCUC | |
| 0 | 706 | GGACAACAACGGA | 82 | GCCCUUUCCGUUGUUG | 106 | GGACAACAACGGAAAGGGA | 130 | UCCCUUUCCGUUGUU | 154 |
| | | AAGGGC | | UCC | | | | GUCCUG | |
| 0 | 813 | CGGAUGUGAUCGA | 83 | UGACUAUCGAUCACAU | 107 | CGGAUGUGAUCGAUAGUCA | 131 | UGACUAUCGAUCACA | 155 |
| | | UAGUCA | | CCG | | | | UCCGAC | |
| 0 | 814 | GGAUGUGAUCGAU | 84 | UUGACUAUCGAUCACA | 108 | GGAUGUGAUCGAUAGUCAA | 132 | UUGACUAUCGAUCAC | 156 |
| | | AGUCAA | | UCC | | | | AUCCGA | |
| 0 | 820 | GAUCGAUAGUCAA | 85 | ACUUGCUUGACUAUCG | 109 | GAUCGAUAGUCAAGCAAGA | 133 | UCUUGCUUGACUAUC | 157 |
| | | GCAAGU | | AUC | | | | GAUCAC | |
| 0 | 916 | GGAAGAUGAUAGG | 86 | CAGAUACCUAUCAUCU | 110 | GGAAGAUGAUAGGUAUCUA | 134 | UAGAUACCUAUCAUC | 158 |
| | | UAUCUG | | UCC | | | | UUCCUU | |
| 0 | 927 | GGUAUCUGAAAUU | 87 | AUUCGGAAUUUCAGAU | 111 | GGUAUCUGAAAUUCCGAAA | 135 | UUUCGGAAUUUCAGA | 159 |
| | | CCGAAU | | ACC | | | | UACCUA | |
| 1 | 943 | AAUUUCUCAUGAA | 88 | CUCUAAUUCAUGAGAA | 112 | AAUUUCUCAUGAAUUAGAA | 136 | UUCUAAUUCAUGAGA | 160 |
| | | UUAGAG | | AUU | | | | AAUUCG | |
| 0 | 1050 | GGUUAAGCAGGAA | 89 | UUAGUAUUCCUGCUUA | 113 | GGUUAAGCAGGAAUACUAA | 137 | UUAGUAUUCCUGCUU | 161 |
| | | UACUAA | | ACC | | | | AACCCU | |
| 0 | 1059 | GGAAUACUAACUG | 90 | AAUGAGCAGUUAGUAU | 114 | GGAAUACUAACUGCUCAUA | 138 | UAUGAGCAGUUAGUA | 162 |
| | | CUCAUU | | UCC | | | | UUCCUG | |
| 0 | 1314 | CUGUAACAGUCUA | 91 | GUGAUAUAGACUGUUA | 115 | CUGUAACAGUCUAUAUCAA | 139 | UUGAUAUAGACUGUU | 163 |
| | | UAUCAC | | CAG | | | | ACAGUU | |
| 0 | 1316 | GUAACAGUCUAUA | 92 | UAGUGAUAUAGACUGU | 116 | GUAACAGUCUAUAUCACUA | 140 | UAGUGAUAUAGACUG | 164 |
| | | UCACUA | | UAC | | | | UUACAG | |
| 0 | 1320 | CAGUCUAUAUCAC | 93 | GGUAUAGUGAUAUAGA | 117 | CAGUCUAUAUCACUAUACA | 141 | UGUAUAGUGAUAUAG | 165 |
| | | UAUACC | | CUG | | | | ACUGUU | |
| 0 | 1330 | CACUAUACCCUUU | 94 | AAACUAAAAGGGUAUA | 118 | CACUAUACCCUUUUAGUUA | 142 | UAACUAAAAGGGUAU | 166 |
| | | UAGUUU | | GUG | | | | AGUGAU | |
| 0 | 1331 | ACUAUACCCUUUU | 95 | AAAACUAAAAGGGUAU | 119 | ACUAUACCCUUUUAGUUUA | 143 | UAAACUAAAAGGGUA | 167 |
| | | AGUUUU | | AGU | | | | UAGUGA | |
| 0 | 1389 | GGUGUGAAUAAAU | 96 | UACAAGAUUUAUUCAC | 120 | GGUGUGAAUAAAUCUUGUA | 144 | UACAAGAUUUAUUCA | 168 |
| | | CUUGUA | | ACC | | | | CACCAA | |

**17 mer x-reactivity (of relevant transcripts)

Our transcriptomics data in patients with atrial fibrillation and HOMER mice indicated that SPP1 was highly upregulated in atrial macrophages that also express the surface receptor TREM2. Based on those data, we genetically deleted SPP1 in bone marrow macrophages, which reduced atrial fibrillation as described above.

We then performed the siRNA screen described above which yielded a number of top-performing siRNA including those in the tables above, especially siRNA 110 and 111.

### REFERENCES FOR EXAMPLES 1 AND 2

1. M. Litvinukova et al., Cells of the adult human heart. Nature 588, 466-472 (2020).
2. J. A. Nicolás-Ávila, A. Hidalgo, I. Ballesteros, Specialized functions of resident macrophages in brain and heart. J Leukoc Biol 104, 743-756 (2018).
3 E. Forte, M. B. Furtado, N. Rosenthal, The interstitium in cardiac repair: role of the immune-stromal cell interplay. Nat Rev Cardiol 15, 601-616 (2018).
4 E. J. Benjamin et al., Heart Disease and Stroke Statistics-2019 Update: A Report From the American Heart Association. Circulation 139, e56-e528 (2019).
5. T. Yamashita et al., Recruitment of immune cells across atrial endocardium in human atrial fibrillation. Circ J 74, 262-270 (2010).
6. N. Smorodinova et al., Analysis of immune cell populations in atrial myocardium of patients with atrial fibrillation or sinus rhythm. PLOS One 12, e0172691 (2017).
7. C. Yao et al., Enhanced Cardiomyocyte NLRP3 Inflammasome Signaling Promotes Atrial Fibrillation. Circulation 138, 2227-2242 (2018).
8. Y. K. Iwasaki, K. Nishida, T. Kato, S. Nattel, Atrial fibrillation pathophysiology: implications for management. Circulation 124, 2264-2274 (2011).
9. V. Rudolph et al., Myeloperoxidase acts as a profibrotic mediator of atrial fibrillation. Nat Med 16, 470-474 (2010).
10. M. D. Robinson, D. J. McCarthy, G. K. Smyth, edgeR: a Bioconductor package for differential expression analysis of digital gene expression data. Bioinformatics 26, 139-140 (2010).
11. A. Subramanian et al., Gene set enrichment analysis: a knowledge-based approach for interpreting genome-wide expression profiles. Proc Natl Acad Sci USA 102, 15545-15550 (2005).
12. P. Langfelder, S. Horvath, WGCNA: an R package for weighted correlation network analysis. BMC Bioinformatics 9, 559 (2008).
13. N. G. Frangogiannis, Matricellular proteins in cardiac adaptation and disease. Physiol Rev 92, 635-688 (2012).
14. I. F. Charo, R. M. Ransohoff, The many roles of chemokines and chemokine receptors in inflammation. N Engl J Med 354, 610-621 (2006).
15. M. Hulsmans et al., Cardiac macrophages promote diastolic dysfunction. J Exp Med 215, 423-440 (2018).
16. N. V. Serbina, E. G. Pamer, Monocyte emigration from bone marrow during bacterial infection requires signals mediated by chemokine receptor CCR2. Nat Immunol 7, 311-317 (2006).
17. X. Tang et al., SIRT2 Acts as a Cardioprotective Deacetylase in Pathological Cardiac Hypertrophy. Circulation 136, 2051-2067 (2017).
18. X. Liao et al., Distinct roles of resident and nonresident macrophages in nonischemic cardiomyopathy. Proc Natl Acad Sci USA 115, E4661-E4669 (2018).
19. Q. Zhang et al., Tet2 is required to resolve inflammation by recruiting Hdac2 to specifically repress IL-6. Nature 525, 389-393 (2015).
20. D. M. Hardbower et al., Ornithine decarboxylase regulates M1 macrophage activation and mucosal inflammation via histone modifications. Proc Natl Acad Sci USA 114, E751-E760 (2017).
21. E. K. Koltsova, C. C. Hedrick, K. Ley, Myeloid cells in atherosclerosis: a delicate balance of anti-inflammatory and proinflammatory mechanisms. Curr Opin Lipidol 24, 371-380 (2013).
22. P. Ramachandran et al., Resolving the fibrotic niche of human liver cirrhosis at single-cell level. Nature 575, 512-518 (2019).
23. R. R. Huxley et al., Absolute and attributable risks of atrial fibrillation in relation to optimal and borderline risk factors: the Atherosclerosis Risk in Communities (ARIC) study. Circulation 123, 1501-1508 (2011).
24. T. S. Tsang et al., Left atrial volume: important risk marker of incident atrial fibrillation in 1655 older men and women. Mayo Clin Proc 76, 467-475 (2001).
25. A. Xintarakou, S. Tzeis, S. Psarras, D. Asvestas, P. Vardas, Atrial fibrosis as a dominant factor for the development of atrial fibrillation: facts and gaps. Europace 22, 342-351 (2020).
26. J. D. Schilling, H. M. Machkovech, A. H. Kim, R. Schwendener, J. E. Schaffer, Macrophages modulate cardiac function in lipotoxic cardiomyopathy. Am J Physiol Heart Circ Physiol 303, H1366-73 (2012).
27. L. Wang et al., CXCL1-CXCR2 axis mediates angiotensin II-induced cardiac hypertrophy and remodelling through regulation of monocyte infiltration. Eur Heart J 39, 1818-1831 (2018).
28. S. Epelman et al., Embryonic and adult-derived resident cardiac macrophages are maintained through distinct mechanisms at steady state and during inflammation. Immunity 40, 91-104 (2014).
29. G. Bajpai et al., The human heart contains distinct macrophage subsets with divergent origins and functions. Nat Med 24, 1234-1245 (2018).
30. L. Ziegler-Heitbrock et al., Nomenclature of monocytes and dendritic cells in blood. Blood 116, e74-80 (2010).
31. R. Gopinathannair et al., Managing Atrial Fibrillation in Patients With Heart Failure and Reduced Ejection Fraction: A Scientific Statement From the American Heart Association. Circ Arrhythm Electrophysiol 14, HAE0000000000000078 (2021).
32. C. Blériot, S. Chakarov, F. Ginhoux, Determinants of Resident Tissue Macrophage Identity and Function. Immunity 52, 957-970 (2020).
33. F. F. Hoyer et al., Tissue-Specific Macrophage Responses to Remote Injury Impact the Outcome of Subsequent Local Immune Challenge. Immunity 51, 899-914.e7 (2019).
34. S. Epelman, K. J. Lavine, G. J. Randolph, Origin and functions of tissue macrophages. Immunity 41, 21-35 (2014).
35. C. D. Paavola et al., Monomeric monocyte chemoattractant protein-1 (MCP-1) binds and activates the MCP-1 receptor CCR2B. J Biol Chem 273, 33157-33165 (1998).
36. M. Efremova, M. Vento-Tormo, S. A. Teichmann, R. Vento-Tormo, CellPhoneDB: inferring cell-cell communication from combined expression of multi-subunit ligand-receptor complexes. Nat Protoc 15, 1484-1506 (2020).
37. M. Harada, S. Nattel, Implications of Inflammation and Fibrosis in Atrial Fibrillation Pathophysiology. Card Electrophysiol Clin 13, 25-35 (2021).
38. K. Graf et al., Angiotensin II and alpha (v) beta (3) integrin expression in rat neonatal cardiac fibroblasts. Hypertension 35, 978-984 (2000).
39. V. Sarrazy et al., Integrins αvβ5 and αvβ3 promote latent TGF-β1 activation by human cardiac fibroblast contraction. Cardiovasc Res 102, 407-417 (2014).
40. J. Molvin et al., Exploration of pathophysiological pathways for incident atrial fibrillation using a multiplex proteomic chip. Open Heart 7, e001190 (2020).
41. L. Liaw et al., Altered wound healing in mice lacking a functional osteopontin gene (spp1). J Clin Invest 101, 1468-1478 (1998).
42. S. R. Künzel et al., Diminished PLK2 Induces Cardiac Fibrosis and Promotes Atrial Fibrillation. Circ Res 129, 804-820 (2021).
43. A. R. Collins et al., Osteopontin modulates angiotensin II-induced fibrosis in the intact murine heart. J Am Coll Cardiol 43, 1698-1705 (2004).
44. Y. Matsui et al., Role of osteopontin in cardiac fibrosis and remodeling in angiotensin II-induced cardiac hypertrophy. Hypertension 43, 1195-1201 (2004).
45. B. Lopez et al., Osteopontin-mediated myocardial fibrosis in heart failure: a role for lysyl oxidase. Cardiovasc Res 99, 111-120 (2013).
46. J. A. Nicolas-Avila et al., A Network of Macrophages Supports Mitochondrial Homeostasis in the Heart. Cell 183, 94-109.e23 (2020).
47. M. Hulsmans et al., Macrophages Facilitate Electrical Conduction in the Heart. Cell 169, 510-522.e20 (2017).
48. M. Ashburner et al., Gene ontology: tool for the unification of biology. The Gene Ontology Consortium. Nat Genet 25, 25-29 (2000).
49. Gene Ontology Consortium, The Gene Ontology resource: enriching a Gold mine. Nucleic Acids Res 49, D325-D334 (2021).
50. A. Liberzon et al., The Molecular Signatures Database (MSigDB) hallmark gene set collection. Cell Syst 1, 417-425 (2015).
51. S. Kou et al., Echocardiographic reference ranges for normal cardiac chamber size: results from the NORRE study. Eur Heart J Cardiovasc Imaging 15, 680-690 (2014).
52. Echocardiographic Normal Ranges Meta-Analysis of the Left Heart Collaboration, Ethnic-Specific Normative Reference Values for Echocardiographic LA and LV Size, LV Mass, and Systolic Function: The EchoNORMAL Study. JACC Cardiovasc Imaging 8, 656-665 (2015).
53. A. R. Pinto et al., Revisiting Cardiac Cellular Composition. Circ Res 118, 400-409 (2016).
54. D. A. Skelly et al., Single-Cell Transcriptional Profiling Reveals Cellular Diversity and Intercommunication in the Mouse Heart. Cell Rep 22, 600-610 (2018).
55. T. Stuart et al., Comprehensive Integration of Single-Cell Data. Cell 177, 1888-1902.e21 (2019).
56. A. Butler, P. Hoffman, P. Smibert, E. Papalexi, R. Satija, Integrating single-cell transcriptomic data across different conditions, technologies, and species. Nat Biotechnol 36, 411-420 (2018).
57. C. Hafemeister, R. Satija, Normalization and variance stabilization of single-cell RNA-seq data using regularized negative binomial regression. Genome Biol 20, 296 (2019).
58. A. T. L. Lun, J. C. Marioni, Overcoming confounding plate effects in differential expression analyses of single-cell RNA-seq data. Biostatistics 18, 451-464 (2017).
59. Y. Chen, A. T. Lun, G. K. Smyth, From reads to genes to pathways: differential expression analysis of RNA-Seq experiments using Rsubread and the edgeR quasi-likelihood pipeline. F1000Res 5, 1438 (2016).
60. D. J. McCarthy, Y. Chen, G. K. Smyth, Differential expression analysis of multifactor RNA-Seq experiments with respect to biological variation. Nucleic Acids Res 40, 4288-4297 (2012).
61. V. K. Mootha et al., PGC-1alpha-responsive genes involved in oxidative phosphorylation are coordinately downregulated in human diabetes. Nat Genet 34, 267-273 (2003).
62. A. Liberzon et al., Molecular signatures database (MSigDB) 3.0. Bioinformatics 27, 1739-1740 (2011).
63. B. Zhang, S. Horvath, A general framework for weighted gene co-expression network analysis. Stat Appl Genet Mol Biol 4, Article17 (2005).
64. E. I. Boyle et al., GO::TermFinder—open source software for accessing Gene Ontology information and finding significantly enriched Gene Ontology terms associated with a list of genes. Bioinformatics 20, 3710-3715 (2004).
65. T. Wu et al., clusterProfiler 4.0: A universal enrichment tool for interpreting omics data. Innovation (NY) 2, 100141 (2021).
66. R. Vento-Tormo et al., Single-cell reconstruction of the early maternal-fetal interface in humans. Nature 563, 347-353 (2018).
67. C. S. McAlpine et al., Astrocytic interleukin-3 programs microglia and limits Alzheimer's disease. Nature 595, 701-706 (2021).

### Example 3. Engineered Antibody-siRNA Conjugates (ARCs) for Silencing Spp1

Here, we engineered an ARC to direct a therapeutic siRNA against Spp1 to a pathogenic macrophage subset. We found that ARC therapy suppresses AFib by reducing atrial fibrosis in HOMER mice.

### Materials and Methods

### Mice

C57BL/6 (stock 000664), and C57BL/6J-Trem2^{em2Adiuj}/J (Trem2^{-/-}; stock 027197) mice were purchased from The Jackson Laboratory. All experiments were performed with 8-to-12-week-old male animals and using age-matched control groups. Where appropriate, mice were randomly assigned to groups. Housing facilities followed a 12-hour dark/light cycle, room temperature was 18-22° C. and maintained humidity was between 40% and 60%. Mice were fed with respective diets ad libitum. All animal experiments were approved by the Institutional Animal Care and Use Committee at the Massachusetts General Hospital (2014N000078) in accordance with federal, state and local guidelines.

### HOMER Model of Inducible AFib

The HOMER mouse model, described previously to induce atrial pathology by combining hypertension, obesity, and mitral valve regurgitation⁴, was used to assess the therapeutic efficacy of Spp1 silencing on AFib inducibility. In brief, mice underwent surgery to generate mitral valve regurgitation. Four weeks after surgery, high-fat diet (Research Diets, D12492) was initiated and continued for at least 17 weeks. Finally, immediately prior to treatment initiation, mice were exposed to continuous infusion of Ang2 (1 μg/kg/min) for one week via a subcutaneously implanted osmotic minipump (Alzet). These HOMER mice were treated with a total of four weekly intravenous injection of either vehicle (100 μL of PBS) or anti-TREM2-siSpp1 (1.5 mg siRNA/kg mouse weight in 100 μL of PBS). During the course of treatment, HOMER mice remained on a high-fat diet.

### Hypertension Model to Assess Spp1 Silencing

The expression of Spp1 is low in a healthy naive animal (FIG. 38B). Therefore, we induced hypertension, described previously to upregulate Spp1 expression in the heart³³ to assess the in vivo silencing of anti-TREM2-siSpp1 in a high-throughput manner. Ang2 (1 μg/kg/min) was continuously administered through osmotic minipumps (Alzet) for 28 days after which they were removed. After a 6-day recovery period, mice were randomized into treatment groups and intravenously injected with a total of 15 or 1.5 mg siRNA/kg body weight of anti-TREM2-siSpp1 or vehicle (PBS) divided into two consecutive doses, three days apart. Hearts were excised three days or six days after the last injection.

### Electrophysiological Study

Electrophysiological studies were performed as previously described by us⁴. Mice were anesthetized with isoflurane and temperature controlled using a homeothermic monitoring system (Harvard Apparatus). A Millar EPR-800 octapolar catheter was inserted into the right jugular vein and positioned in the right atrium and ventricle. AFib inducibility was tested via atrial burst pacing for 3 seconds with decremental cycle lengths starting at 50 ms and progressing to 10 ms in intervals of 5 ms. The protocol was repeated with a burst pacing duration of 6 ms. AFib was defined as irregular ventricular response with rapid atrial rhythm. Mice were considered inducible if they had at least 1 episode of AFib longer than 1 second.

### Organ and Cellular Biodistribution

For organ biodistribution assessments, male C57BL/6 mice at 8 weeks old were injected intravenously with 100 μL of PBS vehicle or 1 nmol of AZDye647-labeled isotype antibody, anti-TREM2, or anti-TREM2-siSpp1 in an equivalent volume. Exactly 24 hours later, 20 mL of ice-cold PBS was used to perfuse the mouse via the left ventricle. The heart, spleen, kidney, and liver piece were excised and collectively imaged on an Azure Sapphire FL Biomolecular Imager.

### Cell Lines

Expi293 cells were obtained from Thermo Fisher Scientific and maintained in Expi293 Expression medium. RAW264.7 cells were obtained from the American Type Culture Collection and maintained in Dulbecco's modified Eagle medium (DMEM) enriched with 10% (v/v) heat-inactivated FBS. All cells tested negative for mycoplasma and were maintained at 37° C. and 5% CO₂.

### Bone Marrow-Derived Macrophage Differentiation and Cell Assays

Femurs from C57BL/6 or Trem2^{-/-} mice were flushed and filtered to isolate bone marrow cells. These cells were cultured in DMEM enriched with 10% (v/v) heat-inactivated FBS supplemented with 30 ng/ml of M-CSF. After 5 days of culture, bone-marrow derived macrophages (BMDM) were polarized by adding 10 ng/ml of IL-4 and IL-13 to the medium for 24 hours. BMDM were treated with either vehicle OptiMEM (Thermo Fisher Scientific), anti-TREM2-siSpp1, or siSpp1 complexed with lipofectamine RNAiMAX transfection reagent (Thermo Fisher Scientific) for 48 hours.

siRNA Identification, Validation, Development

With assistance from Axolabs, we employed a bioinformatic approach to identify a screening set of 24 siRNA candidates directed against mouse Spp1 mRNA (secreted phosphoprotein 1, NCBI Gene ID 20750). This approach assumes a canonical 19-mer dsRNA structure whereby positions 2-18 (5′-3′) of the sense and antisense strand were used for specificity calculations. Positions 2-7 (5′-3′) were considered the seed region and used to exclude candidates with identical seed regions to known miRNAs. Positions 1-19 (5′-3′) of the antisense strand were used for human cross-reactivity analysis; perfect matches or single nucleotide mismatches were considered cross-reactive hits.

Candidate siRNAs were synthesized and purified with RNA, DNA, 2′-O-methyl residues and phosphorothioate modifications at defined positions. Each single strand was HPLC purified to >85% purity and its mass confirmed by LC/MS. Candidate siRNA's specific silencing was validated in murine Hepa1-6 cells using lipofectamine as a transfection reagent and after 24 hours of exposure. Spp1 and Gapdh mRNA were measured by branched DNA assay. siRNA against F-luc, Ahsa1 and FVII were used as negative controls.

For the siRNA candidate selected for antibody conjugation, the siRNA sense and antisense strands were generated using the standard pattern of alternating 2′-O-methyl and 2′-fluoro nucleotides and phosphorothioate bonds at the extremities of the strands. For the anti-sense strand, a 5′-vinylphosphonate 2′-O-Methyl uridine was included for additional tissue accumulation and stability³⁴. For the sense strand, an aminohexyl linker was appended to the 3′ end to facilitate the addition of a pegylated methyl tetrazine group for downstream chemical conjugation.

### Antibody Production

Plasmids encoding the variable heavy and light chain of a human/mouse cross-reactive antibody directed against TREM2 clone 52 were derived from a patent application (WO 2016/023019). The variable heavy chain was fused to mouse IgG2c fragment crystallizable region (Fc) bearing LALAPG mutations rendering the Fc region inert. The plasmids were transfected into Expi293 cells following manufacturer's recommendations (Thermo Fisher Scientific). The antibodies produced by these cells were isolated by protein A Sepharose affinity chromatography. Antibody was then further purified by size exclusion chromatography using a HiLoad 16/600 Superdex 200 μg column on an ÄKTA FPLC system (GE Healthcare) that had been pretreated for 4 hours with 1 M NaOH to remove endotoxin and subsequently equilibrated in sterile PBS (Corning). After purification, all proteins were buffer exchanged into sterile PBS, 0.2 μm sterile-filtered (Pall Corporation), and confirmed to contain minimal endotoxin (<0.1 EU/mL) using a chromogenic LAL assay (Lonza).

### Antibody-siRNA Conjugation and Validation

To conjugate the siRNA to the antibody, we employed our validated linkage strategy. Lysine residues on the antibody were coupled with pegylated methyl tetrazine (MeTz) and covalently linked to a trans-cyclooctene (TCO) handle on the 3′ end of the sense strand of the hybridized siRNA at a ratio of 1:10. ARCs were subsequently purified by size exclusion chromatography, removing uncoupled siRNA and antibody. The RNA content was quantified by UV or ribogreen assay and the protein content by BCA assay (both Thermo Fisher Scientific) according to the manufacturer's protocol.

### Fluorophore Labeling

Antibodies or ARC were labeled with Alexa Fluor 647 (AF647)N-hydroxysuccinimide ester, Alexa Fluor 488 (AF488)N-hydroxysuccinimide ester (both Thermo Fisher Scientific), or AZDye 647 Hydrazide (Vector Labs). Excess dye was removed using a PD10 desalting column (GE Healthcare), and the degree of labeling for each protein was calculated. Anti-TREM2 labeled with AF488 were used to confirm TREM2 binding and measure net internalization rate. Antibodies and ARC injected for cellular biodistribution assays possessed equimolar AF647, 1 nmol. Antibodies and ARC injected for organ biodistribution assays possessed equimolar AZDye647, 1 nmol.

### Anti-TREM2 Binding Confirmation

Expi293 cells or RAW264.7 cells in suspension were incubated with 100 nM of anti-TREM2-AF647 in FACS buffer (PBS containing 0.5% w/v of bovine serum albumin and 2 mM of EDTA) on ice for 30 minutes. After treatment, cells were washed and antibody binding was measured by flow cytometry on an LSRII (BD Biosciences) and analyzed with FlowJo software.

### Antibody Net Internalization Assay

RAW264.7 cells were treated with 100 nM anti-TREM2-AF488 for time points between 0-9 hours. Based on the dissociation and association rates, this concentration range ensured a rapid equilibration rate, with the resulting equilibrium favoring saturated surface receptors. After treatment, cells were washed once with PBS and then detached from the plate using 0.25% Trypsin/EDTA. Cells were pelleted at 1000×g for 5 minutes and then resuspended in FACS buffer.

To determine what fraction of the total signal from anti-TREM2-AF488 derived from surface-bound antibody rather than internalized antibody, we used an anti-AF488 polyclonal antibody (Thermo Fisher Scientific) which exhibits >90% quenching efficiency. Half of the pelleted sample was diverted for staining with 20 μg/mL of anti-AF488, followed by incubation of both samples on ice for 10 minutes. The mean fluorescence intensity (MFI) of AF488 signal for all samples (quenched versus unquenched; different timepoints; and replicates) was measured via flow cytometry on an LSRII and analyzed with FlowJo software.

To calculate the internalized fraction, we assumed that the difference in MFI between the unquenched and quenched fixed cells arises from cell surface-bound antibody. The internalized fraction was then determined by subtracting the cell surface-bound antibody AF488 MFI from total unquenched cell AF488 MFI. Besides the initial time point, the surface TREM2 receptors were considered fully saturated. Assuming internalization is a first-order rate process, we fitted a linear relationship between the internalized fraction and the surface integral to determine the net internalization rate.

For cellular biodistribution assessments, 8-week-old male C57BL/6 mice were injected intravenously with 100 μL of PBS vehicle or 1 nmol of AF647-labeled isotype antibody, anti-TREM2, or anti-TREM2-siSpp1 in an equivalent volume. Exactly 24 hours later, 20 mL of ice-cold PBS was used to perfuse the mouse via the left ventricle. The heart was processed for flow cytometry as described below; however, TREM2-APC antibody was excluded from the staining mixture.

Flow Cytometry Cell Preparation from Heart Tissue

For isolation of heart tissue, 20 mL of ice-cold PBS was used to perfuse the mouse via the left ventricle. Hearts were excised and dissociated into small (1 mm³) pieces using scissors, and enzymatically digested with 450 U/mL of collagenase I, 125 U/mL of collagenase XI, 60 U/mL of DNase I, and 60 U/mL of hyaluronidase (all Sigma-Aldrich) for 60 minutes at 37° C. under agitation. Tissue pieces were mechanically dissociated through a 40-μm nylon mesh filter, washed with ice-cold FACS buffer and then centrifuged (340×g for 7 minutes) to obtain single-cell suspensions ready for staining.

### Flow Cytometry Staining, Acquisition, and Analysis

To detect cellular subsets in the heart, cell suspensions were stained at 4° C. in FACS buffer. Heart single cell suspensions were first stained with Zombie Violet viability dye (BioLegend) and blocked with CD16/CD32 antibody (clone 93, 1:100, eBioscience). Cells were then stained with CD45-BV711 (clone 30-F11, 1; 600, BioLegend), CD3-BV785 (clone 17A2, 1:100, BioLegend), CD19-BV785 (clone 6D5, 1:100, BioLegend), Ly6G-FITC (clone 1A8, 1:600, BioLegend), CD31-PerCP/Cy5.5 (clone 390, 1:400, BioLegend), MEFSK4-PE (clone mEF-SK4, 1:100, Miltenyi Biotec), CD64-PE/Cy7 (clone X54-5/7.1, 1:250, BioLegend), MHCII-AF700 (clone M5/114.15.2, 1:600, BioLegend), CD11b-APC/Cy7 (clone M1/70, 1:600, BioLegend), and TREM2-APC (clone 237920, 1:20, R&D Biosystems). Data were acquired on an Attune NxT (Thermo Fisher Scientific) and analyzed with FlowJo software.

### Flow Cytometry Gating

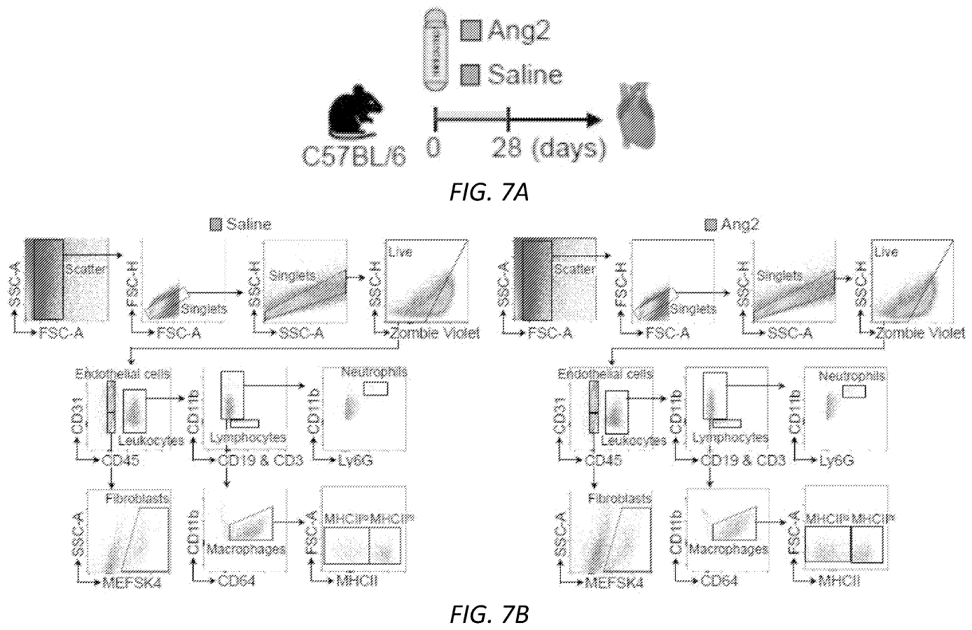

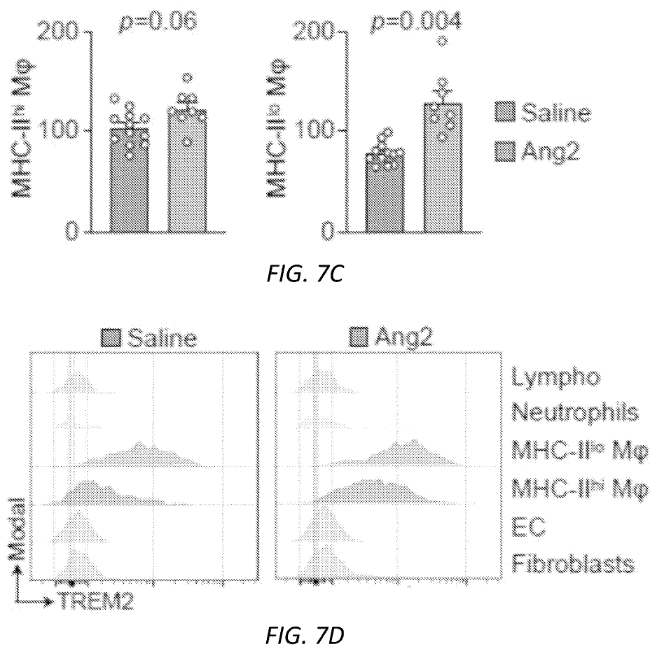

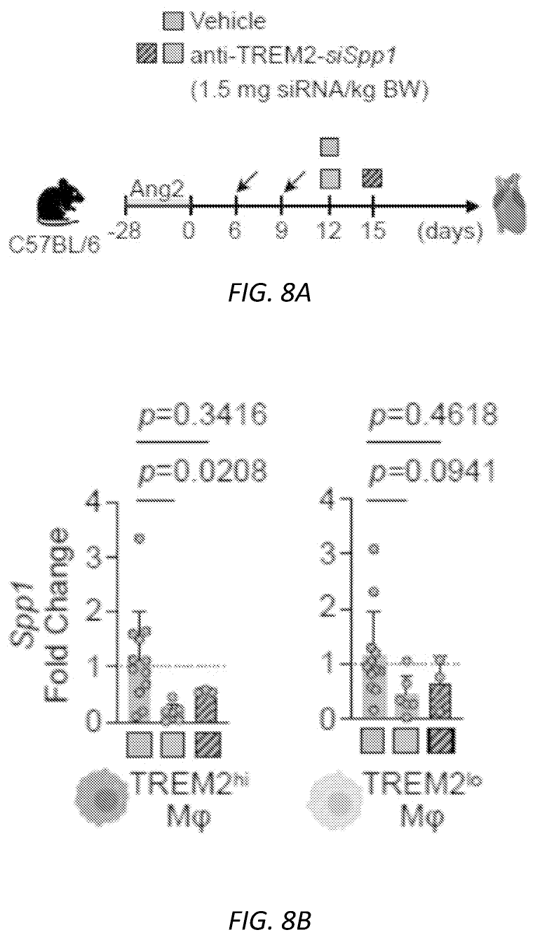

Heart cell subsets were defined based on marker expression as follows: endothelial cells as CD45⁻CD31⁺, fibroblasts as CD45 CD31⁻ MEFSK4⁺, neutrophils as CD45⁺CD11b⁺Ly6G⁺, lymphocytes as CD45⁺CD11b⁻ CD3⁺ and CD45⁺CD11b⁻ CD19⁺, and cardiac macrophages as CD45⁺CD11b⁺CD64⁺. Cardiac macrophages were further parsed by MHCII expression. The gating strategy employed is shown in FIG. 7B.

### FACS Isolation of Cardiac Macrophages

Cardiac cell suspensions were stained as mentioned above. Macrophages were sorted according to their MHCII expression on a FACSAria II cell sorter (BD Biosciences) directly into 350 μL RLT Plus lysis buffer (Qiagen).

Real-Time qPCR

Total RNA was extracted from BMDM using the AllPrep RNA/Protein kit or from FACS-purified macrophages using the RNeasy Plus Micro kit (both Qiagen) according to the manufacturer's protocol. RNA was reverse transcribed using the High-Capacity RNA-to-cDNA kit (Thermo Fisher Scientific) according to the manufacturer's instructions. TaqMan Fast Universal PCR Master Mix (Thermo Fisher Scientific) and TaqMan gene expression assays for Spp1 (Mm00436767_m1, FAM-MGB) and Gapdh (Mm99999915_g1, VIC-MGB, both Thermo Fisher Scientific) were used to quantify Spp1 mRNA. The relative changes were normalized to Gapdh mRNA using the 2^{-ΔΔCt} method.

### Osteopontin ELISA

Total protein extracted from BMDM using the AllPrep RNA/Protein kit (Qiagen) was analyzed with the Osteopontin ELISA kit (Abcam) according to the manufacturer's protocol.

### Histology

To detect left atrial fibrosis, hearts were embedded in OCT compound and frozen 7-μm sections were stained with Masson's trichrome. Briefly, sections were fixed with 10% formalin for 30 minutes at room temperature and incubated in Bouin's fixative solution (Electron Microscopy Sciences) overnight at room temperature, and the nuclei were stained with a mixture of Weigert's iron hematoxylin solution A and B (Electron Microscopy Sciences). The sections were then stained with Biebrich scarlet-acid fuchsin (StatLab), followed by phosphomolybdic acid/phosphotungstic acid solution (Sigma-Aldrich) and aniline blue stain (StatLab). All slides were scanned by a digital slide scanner NanoZoomer 2.0RS (Hamamatsu). To analyze fibrosis from stained tissue sections, ten fields of views (FOV) from the left atrium per animal were randomly selected by a person blinded to cohort assignment and exported at 40× magnification using NanoZoomer NDP.view2 software (Hamamatsu). For each FOV, the percentage of positive signal was measured using ImageJ v2.9.0 (NIH) software.

### Cardiac MRI

MRI was performed with a 4.7-Tesla horizontal bore Pharmascan system (Bruker) equipped with a custom-built mouse cardiac coil (Rapid Biomedical) to obtain cine images of the left atrial and ventricular short axes and a cine loop of the horizontal long-axis. ECG and respiratory gating (SA Instruments), a gradient echo FLASHsequence and a dedicated mouse cardiac volume coil in bird cage design were used. Imaging parameters were as follows: echo time (TE), 2.7 to 5 ms; 16 frames per RR interval (TR 7.0-15 ms depending on heart rate); in-plane resolution, 150×150 μm; slice thickness, 1 mm; NEX 4; flip angle, 30 degrees. Left atrial size was measured using Horos software.

snRNA-seq

For snRNA-seq experiments, left atrial tissues from vehicle- and anti-Trem2-siSpp 1-treated HOMER mice were microdissected using a dissection microscope and subsequently snap-frozen in liquid nitrogen. Nuclei were then isolated using the 10× Chromium Nuclei Isolation kit according to the manufacturer's protocol. In detail, frozen samples were homogenized using a pestle and lysis buffer and passed through a column. Next, debris was removed via centrifugation in debris removal buffer. The nuclei were then washed, resuspended and visualized under a microscope to determine nuclei concentration and quality. The isolated nuclei were immediately loaded on a 10× Genomics Chromium Single Cell G chip as outlined by the 10× Genomics Chromium Single Cell 3′ Reagent kit v3.1 user guide. As nuclei have a lower RNA content than whole cells, the cDNA amplification cycles were increased by 1-2 cycles, as recommended by 10× Genomics. Libraries were then prepared according to the manufacturer's protocol with modifications for PCR cycles based on the cDNA concentration. Constructed libraries were validated and quantified with the Agilent High Sensitivity DNA chip and by real-time qPCR and subsequently sequenced on an Illumina NovaSeq 6000 system as recommended by 10× Genomics.

scRNA-Seq and snRNA-Seq Analysis

We analyzed deposited scRNA-seq data (GSE224959) as previously described by us⁴. For snRNA-seq data, the sequencing reads were aligned to the mouse (mm 10) reference genome and processed with 10× Genomics Cell Ranger v7.1.0, which generated a gene-cell barcode count matrix for each sample. These count matrices were modified by removal of ambient RNA contamination using CellBender v0.3.0³⁵. Quality-control metrics, including the number of unique molecular identifiers (nUMI) detected, the number of unique genes (nGene) detected, and the percentage of reads mapped to the mitochondrial genes (percent.mt), were used to filter out low-quality nuclei. Specifically, for each sample, nuclei were filtered out if their nUMI or nGene were lower than their respective sample median by more than three times the median absolute deviation (MAD), or if their percent.mt were higher than the sample median by more than three MADs. Further hard cutoffs of nUMI >150, nGene >150, and percent.mt<5 are imposed across the samples. Doublets were flagged with scDblFinder³⁶. Seurat v4.3.0³⁷⁻³⁹ was used to carry out further analyses, including count normalization with sctransform v2^{40,41} followed by an integration workflow to identify shared cell states present across all samples under consideration, as well as nuclei clustering and cluster marker identification. Clusters with more than 40% of nuclei flagged as doublets were removed, and the same Seurat analyses were repeated with the remaining nuclei.

### Statistics and Reproducibility

Statistical analyses were performed using GraphPad Prism 9. Normality of data was assessed using Kolmogorov Smirnov test. The specific statistical methods and number of replicates are indicated in the figure legends. P-values of 0.05 or less were considered significant. Animal group sizes were mindfully and empirically chosen. Animals were randomly assigned to treatment groups. Data were analyzed in a blinded fashion where appropriate.

### Results

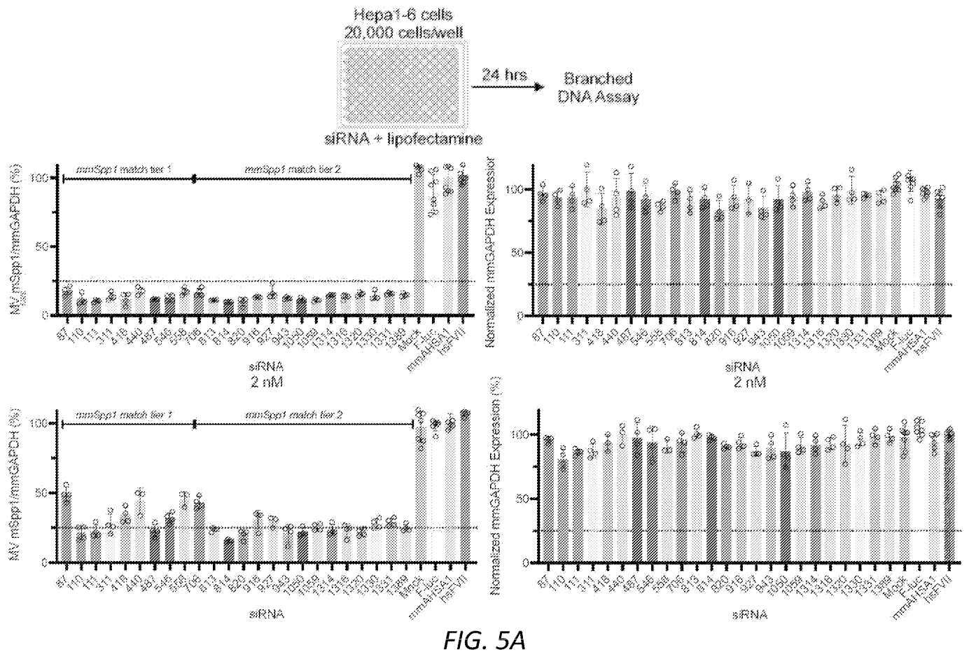

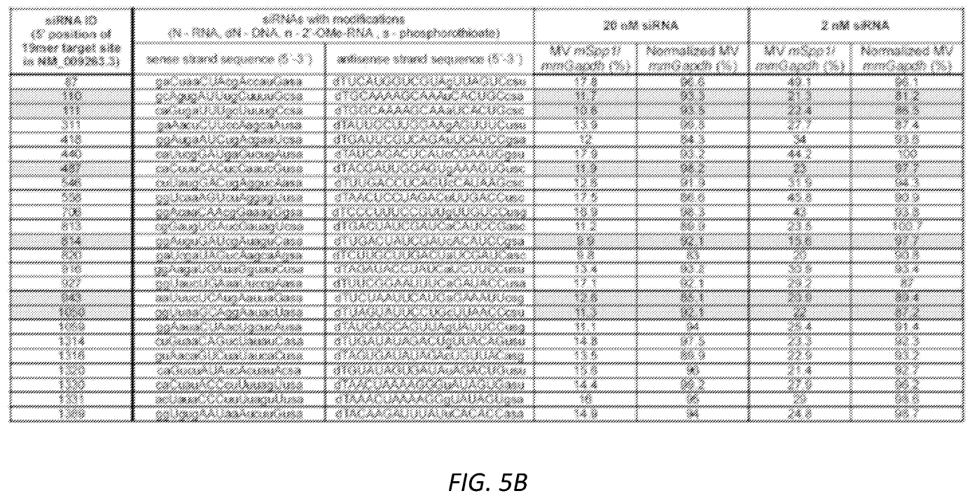

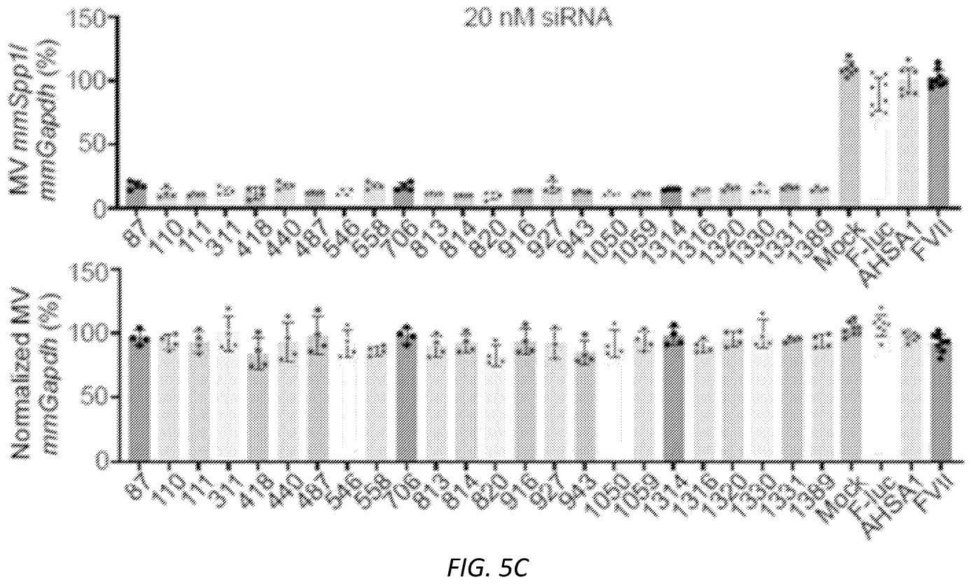

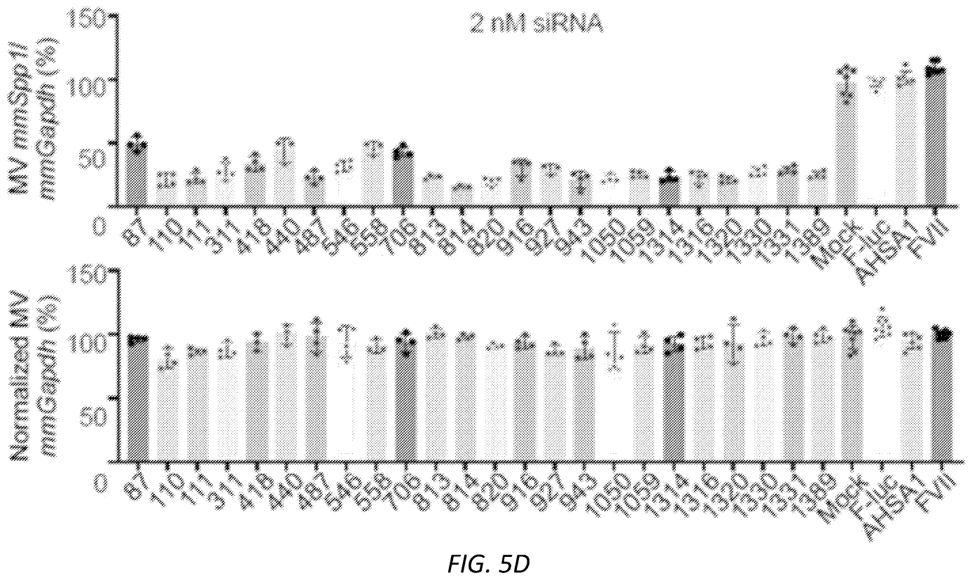

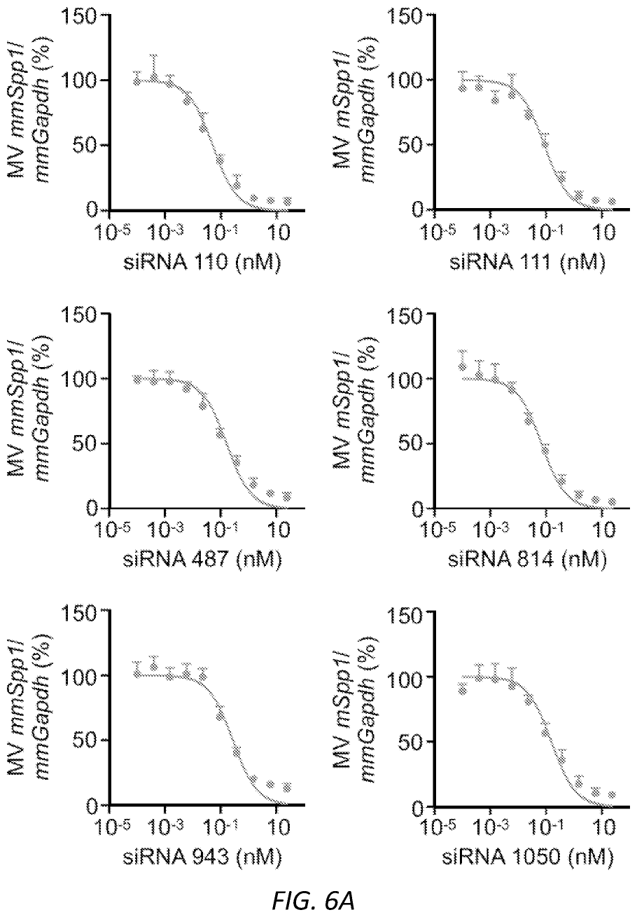

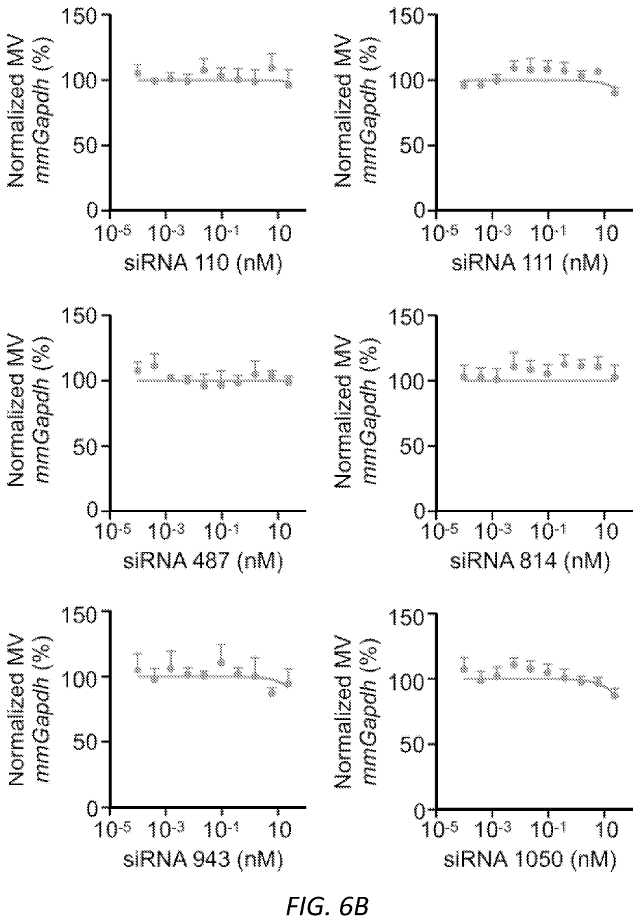

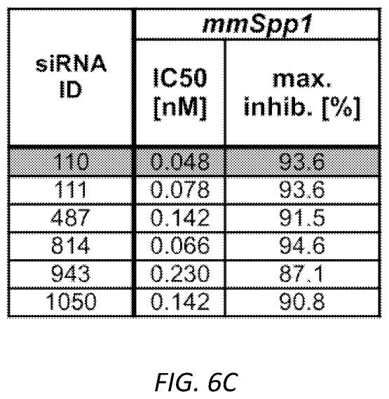

To achieve gene silencing, we first generated a panel of 24 predicted siRNA candidates targeting Spp1 (FIGS. 5A-5D). Based on silencing potency in murine Hepa1-6 cells and posited human cross-reactivity, we selected siRNA clone 110 (siSpp1) to carry forward (FIGS. 6A-6C). Our prior work showed human SPP1 and mouse Spp1 are expressed by a specific subset of macrophages that upregulate triggering receptor expressed on myeloid cells 2 (TREM2) (FIGS. 38A-38B). TREM2 is a myeloid receptor expressed on macrophages that enables phagocytosis of various ligands including phospholipids, myelin, and apoptotic cells. Therefore, we selected TREM2 as an antibody target to mediate siSpp1 delivery. We recombinantly expressed a TREM2 full-length antibody (AL002c clone 52), with previously demonstrated human and mouse TREM2 binding¹², with an inert mouse IgG2c Fc region (FIG. 40A). Cellular uptake is a prerequisite for an effective siRNA delivery vehicle, therefore we confirmed rapid internalization of anti-TREM2 by murine RAW264.7 macrophages, with a half-life of 3.99 hours (FIGS. 40B-40D). Then, anti-TREM2 and siSpp1 were successfully conjugated using click chemistry (FIGS. 38C and 41A-41C). Size exclusion chromatography was used to remove unbound siRNA and antibody and to isolate the monomeric ARC, anti-TREM2-siSpp1 (FIG. 41D). Mass spectrometry analysis revealed a drug-antibody-ratio of 2:1.

Next, we assessed the organ and cellular biodistribution of the ARC. Both fluorescently labeled anti-TREM2 and anti-TREM2-siSpp1 were preferentially retained in the left atria over isotype control antibody 24 hours after intravenous injection (FIGS. 38D-38F). No differences in drug uptake in other organs were observed (FIGS. 38E and 38F). Flow cytometry on the heart revealed internalization of anti-TREM2 and anti-TREM2-siSpp1 by macrophages, preferentially by MHCII^{lo} macrophages (FIGS. 38G and 38H). The heart contains distinct macrophage subsets with divergent origins, functions, and prevalence in inflammation¹³. In AFib, recruited monocyte-derived macrophages accumulate in the heart⁴. We found that MHCII^{lo} macrophages, which expand in cardiac injury, expressed higher levels of the TREM2 receptor (FIGS. 7A-7D), explaining why higher internalized anti-TREM2 and ARC signals were detected in this macrophage subset. Negligible internalized antibody and ARC signal could be detected in other immune cells, fibroblasts, or endothelial cells in the heart (FIG. 38G), highlighting the cell specificity of antibody-driven uptake.

Next, we evaluated the silencing capacity of anti-TREM2-siSpp1. ARC exposure to wild-type C57BL/6 bone marrow-derived macrophages (BMDM) reduced Spp1 mRNA by 70% and intracellular osteopontin protein by 50% (FIGS. 38I-38K).

The silencing achieved by antibody-mediated siSpp1 delivery was indistinguishable from that of the gold-standard transfection reagent lipofectamine (FIGS. 381-38K). Unlike lipofectamine/Spp1, however, anti-TREM2-siSpp1 did not silence Spp1 or osteopontin in BMDM from a Trem2^{-/-}-mouse strain, indicating that anti-TREM2-siSpp1's silencing relies on binding to its target TREM2 receptor (FIGS. 38I-38K). Target-mediated silencing was also confirmed in vivo using hypertensive C57BL/6 mice injected with anti-TREM2-siSpp1 or vehicle control (FIGS. 38L and 38M). Only TREM2 hi, but not TREM21 cardiac macrophages demonstrated a knockdown in Spp1 expression by 90% at 15 mg siRNA/kg body weight dose and still 82% at a ten-fold lower dose (FIG. 38M). This effect was transient; Spp1 expression rose to 50% of baseline in TREM2^{hi} cardiac macrophages 6 days after the last dose, indicating that weekly dosing of 1.5 mg siRNA/kg body weight will be required for sustained therapeutic silencing (FIGS. 43A-43B). The kinetics of siRNA silencing depends on many factors including the siRNA's potency, cellular uptake, and target mRNA or cell turnover¹⁴. Durable silencing of up to 6 months has been observed using GalNAc-siRNA conjugates in a clinical setting¹⁵. Therefore, exploring ways to prolong the silencing effect of future ARC iterations will be worthwhile.

Finally, we tested the feasibility of Spp1 silencing to treat AFib. HOMER mice with established atrial disease received weekly intravenous injections of anti-TREM2-siSpp1 or the vehicle saline (FIG. 39A). After one month of treatment, these mice were subjected to an invasive electrophysiological study to examine AFib susceptibility (FIG. 39B). Compared to vehicle, ARC treatment reduced AFib inducibility to 33% (from 82%) and total AFib episode burden by four-fold (FIG. 39C). ARC treatment significantly reduced atrial fibrosis (FIG. 39D). Interestingly, we did not observe a change in atria size from treatment (FIG. 39E). Collectively, these results suggest that silencing Spp1 in TREM2⁺ macrophages is a tractable immunotherapy for AFib.

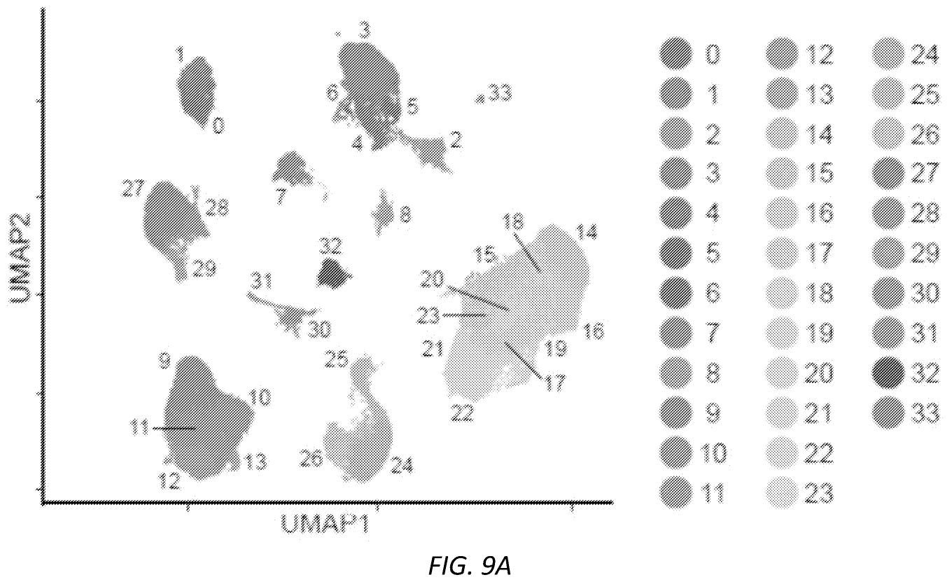

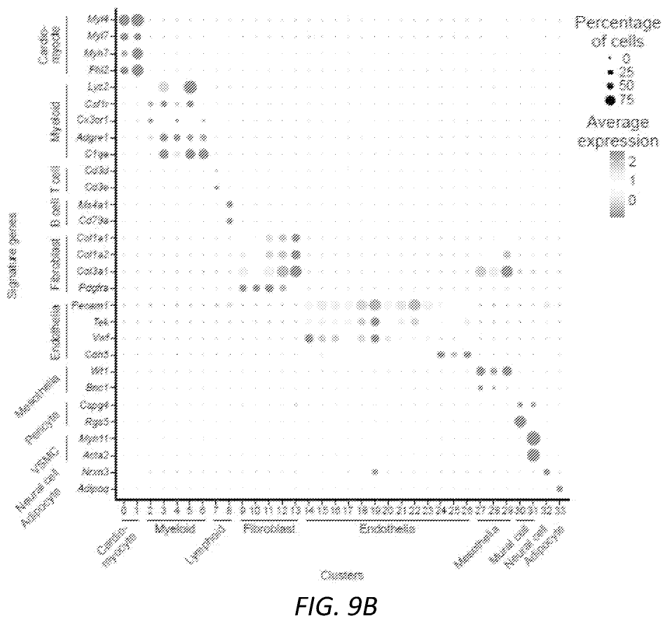

To interrogate the molecular changes driving treatment benefit, we collected left atria from HOMER mice after anti-TREM2-siSpp1 (n=3) or vehicle (n=3) treatment and performed single nuclei RNA sequencing (snRNA-seq) using the 10× Genomics Chromium platform. A total of 48,647 single nuclei were partitioned into clusters (FIG. 39F). We identified 8 noncardiomyocyte cell types: myeloid, lymphoid, fibroblasts, endothelial, mesothelia, mural cells, neural cells, and adipocytes (FIG. 39F and FIGS. 9A-9B). Given the reduction of atrial fibrosis after ARC treatment, we closely examined the fibroblasts (FIG. 39G). All fibroblast subclusters express matrix-related genes to varying degrees, but we observed that Col1a1, Col1a2, Col3a1, and others were most expressed in cluster 4 (FIG. 39H). Cluster 4 fibroblasts were reduced in the ARC-treated group compared to the vehicle arm (FIG. 39I). Furthermore, the expression of several collagen genes in cluster 4 cells was significantly lowered by ARC treatment (FIG. 39J). Although cluster 4 comprises <5% of total fibroblasts, their small proportion is consistent with the notion that a niche of cells can regulate global tissue changes during disease^{16,17}. Reduction of both matrix-producing cells (FIG. 39I) and genes (FIG. 39J) aligns with less fibrosis (FIG. 39D) and suppression of AFib (FIGS. 39B and 39C) achieved by ARC therapy.

To our knowledge, our study provides the first demonstration of an antibody leveraged for siRNA delivery to a specific subset of heart macrophages. While we and others have previously used lipid nanoparticles to deliver siRNA to cardiovascular macrophages^{18,19}, they were not directed to a subset of macrophages, and their uptake by other cell types and tissues remained an issue. ARCs, as evidenced by anti-TREM2-siSpp1, are suited to overcome biodistribution challenges and hone siRNA uptake and gene silencing to specific cell subsets within a tissue. Efficient silencing of Spp1 in TREM2⁺ macrophages may provide a means to treat other fibrosis-driven pathologies such as metabolic-associated fatty liver disease²⁰, idiopathic pulmonary fibrosis²¹, systemic sclerosis²², and chronic kidney disease²³.

While no immunotherapies have been approved for treating AFib, modest effects have been observed in small trials using colchicine^{24,25}, an anti-inflammatory small molecule, and canakinumab²⁶, an IL-1β neutralizing antibody. Unfortunately, these agents also disrupt essential immune functions in healthy tissues, leading to an increased risk of life-threatening infections which limits their chronic clinical use²⁷⁻²⁹ A daunting challenge in the development of safe and effective immunotherapies is ensuring their effects are isolated and concentrated in the diseased cells and tissue^{30,31}. By conferring cell specificity to gene silencing, ARCs emerge as a promising immunotherapy modality. Specifically, our study using anti-TREM2-siSpp1 alongside other recent efforts³² showcases the feasibility of targeted immunotherapy to treat AFib.

Although silencing Spp1 in TREM2⁺ cardiac macrophages suppressed AFib in a majority of HOMER mice, a subset of treated mice still experienced short episodes of AFib. It is possible other molecular pathways driving AFib may remain active. Our study opens the possibility of engineering new ARCs targeting other genes and cells to treat the multifaceted etiology of AFib.

### REFERENCES FOR EXAMPLE 3

1. Kornej, J., Börschel, C. S., Benjamin, E. J. & Schnabel, R. B. Epidemiology of Atrial Fibrillation in the 21st Century: Novel Methods and New Insights. Circ Res 127, 4-20 (2020).
2. Michaud, G. F. & Stevenson, W. G. Atrial Fibrillation. N Engl J Med 384, 353 361 (2021).
3. Dobrev, D., Heijman, J., Hiram, R., Li, N. & Nattel, S. Inflammatory signalling in atrial cardiomyocytes: a novel unifying principle in atrial fibrillation pathophysiology. Nat Rev Cardiol 20, 145-167 (2023).
4. Hulsmans, M. et al. Recruited macrophages elicit atrial fibrillation. Science 381, 231-239 (2023).
5. Lund, S. A., Giachelli, C. M. & Scatena, M. The role of osteopontin in inflammatory processes. J Cell Commun Signal 3, 311-322 (2009).
6. Harada, M. & Nattel, S. Implications of Inflammation and Fibrosis in Atrial Fibrillation Pathophysiology. Card Electrophysiol Clin 13, 25-35 (2021).
7. Ahn, I., Kang, C. S. & Han, J. Where should siRNAs go: applicable organs for siRNA drugs. Exp Mol Med 55, 1283-1292 (2023).
8. Kulkarni, J. A. et al. The current landscape of nucleic acid therapeutics. Nat Nanotechnol 16, 630-643 (2021).
9. Dong, Y., Siegwart, D. J. & Anderson, D. G. Strategies, design, and chemistry in siRNA delivery systems. Adv Drug Deliv Rev 144, 133-147 (2019).
10. Dugal-Tessier, J., Thirumalairajan, S. & Jain, N. Antibody-Oligonucleotide Conjugates: A Twist to Antibody-Drug Conjugates. J Clin Med 10, 838 (2021).
11. Cuellar, T. L. et al. Systematic evaluation of antibody-mediated siRNA delivery using an industrial platform of THIOMAB-siRNA conjugates. Nucleic Acids Res 43, 1189-1203 (2015).
12. Wang, S. et al. Anti-human TREM2 induces microglia proliferation and reduces pathology in an Alzheimer's disease model. J Exp Med 217, e20200785 (2020).
13. Epelman, S., Lavine, K. J. & Randolph, G. J. Origin and functions of tissue macrophages. Immunity 41, 21-35 (2014).
14. Ayyar, V. S. & Song, D. Mechanistic Pharmacokinetics and Pharmacodynamics of GalNAc-siRNA: Translational Model Involving Competitive Receptor-Mediated Disposition and RISC-Dependent Gene Silencing Applied to Givosiran. J Pharm Sci 113, 176-190 (2024).
15. Ray, K. K. et al. Effect of inclisiran on lipids in primary prevention: the ORION-11 trial. Eur Heart J 43, 5047-5057 (2022).
16. Ramachandran, P. et al. Resolving the fibrotic niche of human liver cirrhosis at single-cell level. Nature 575, 512-518 (2019).
17. Kanemaru, K. et al. Spatially resolved multiomics of human cardiac niches. Nature 619, 801-810 (2023).
18. Leuschner, F. et al. Therapeutic siRNA silencing in inflammatory monocytes in mice. Nat Biotechnol 29, 1005-1010 (2011).
19. Huang, X. et al. Synthesis of siRNA nanoparticles to silence plaque-destabilizing gene in atherosclerotic lesional macrophages. Nat Protoc 17, 748-780 (2022).
20. Remmerie, A. et al. Osteopontin Expression Identifies a Subset of Recruited Macrophages Distinct from Kupffer Cells in the Fatty Liver. Immunity 53, 641-657.e14 (2020).
21. Morse, C. et al. Proliferating SPP1/MERTK-expressing macrophages in idiopathic pulmonary fibrosis. Eur Respir J 54, 1802441 (2019).
22. Gao, X. et al. Osteopontin Links Myeloid Activation and Disease Progression in Systemic Sclerosis. Cell Rep Med 1, 100140 (2020).
23. Steinbrenner, I. et al. Association of osteopontin with kidney function and kidney failure in chronic kidney disease patients: the GCKD study. Nephrol Dial Transplant 38, 1430-1438 (2023).
24. Deftereos, S. et al. Colchicine for prevention of early atrial fibrillation recurrence after pulmonary vein isolation: a randomized controlled study. J Am Coll Cardiol 60, 1790-1796 (2012).
25. Deftereos, S. et al. Colchicine for prevention of atrial fibrillation recurrence after pulmonary vein isolation: mid-term efficacy and effect on quality of life. Heart Rhythm 11, 620-628 (2014).
26. Krisai, P. et al. Canakinumab After Electrical Cardioversion in Patients With Persistent Atrial Fibrillation: A Pilot Randomized Trial. Circ Arrhythm Electrophysiol 13, e008197 (2020).
27. Ridker, P. M. et al. Antiinflammatory Therapy with Canakinumab for Atherosclerotic Disease. N Engl J Med 377, 1119-1131 (2017).
28. Deftereos, S. G. et al. Colchicine in Cardiovascular Disease: In-Depth Review. Circulation 145, 61-78 (2022).
29. Ahmed, A. S. et al. Prophylactic Colchicine After Radiofrequency Ablation of Atrial Fibrillation: The PAPERS Study. JACC Clin Electrophysiol 9, 1060-1066 (2023).
30. Rurik, J. G., Aghajanian, H. & Epstein, J. A. Immune Cells and Immunotherapy for Cardiac Injury and Repair. Circ Res 128, 1766-1779 (2021).
31. Momin, N. Balancing safety and efficacy: tuning the biodistribution and pharmacokinetics of cytokine immunotherapies. Curr Opin Biotechnol 84, 102994 (2023).
32. Hiram, R. et al. An inflammation resolution-promoting intervention prevents atrial fibrillation due to left-ventricular dysfunction. Cardiovasc Res cvad175 (2023).
33. Collins, A. R. et al. Osteopontin modulates angiotensin II-induced fibrosis in the intact murine heart. J Am Coll Cardiol 43, 1698-1705 (2004).
34. Haraszti, R. A. et al. 5′-Vinylphosphonate improves tissue accumulation and efficacy of conjugated siRNAs in vivo. Nucleic Acids Res 45, 7581-7592 (2017).
35. Fleming, S. J. et al. Unsupervised removal of systematic background noise from droplet-based single-cell experiments using CellBender. Nat Methods 20, 1323-1335 (2023).
36. Germain, P. L., Lun, A., Garcia Meixide, C., Macnair, W. & Robinson, M. D. Doublet identification in single-cell sequencing data using scDblFinder. F1000Res 10, 979 (2021).
37. Butler, A., Hoffman, P., Smibert, P., Papalexi, E. & Satija, R. Integrating single-cell transcriptomic data across different conditions, technologies, and species. Nat Biotechnol 36, 411-420 (2018).
38. Stuart, T. et al. Comprehensive Integration of Single-Cell Data. Cell 177, 1888-1902.e21 (2019).
39. Hao, Y. et al. Dictionary learning for integrative, multimodal and scalable single-cell analysis. Nat Biotechnol 42, 293-304 (2024).
40. Hafemeister, C. & Satija, R. Normalization and variance stabilization of single-cell RNA-seq data using regularized negative binomial regression. Genome Biol 20, 296 (2019).
41. Choudhary, S. & Satija, R. Comparison and evaluation of statistical error models for scRNA-seq. Genome Biol 23, 27 (2022).
42. Frangogiannis, N. G. Cardiac fibrosis. Cardiovasc Res 117, 1450-1488 (2021).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a double-stranded RNA (dsRNA) agent targeting human secreted phosphoprotein 1 (SPP1), wherein the dsRNA agent comprises a sense strand and an antisense strand forming a double stranded region, wherein the antisense strand comprises a nucleotide sequence comprising at least 15 contiguous nucleotides, with 0, 1, 2, or 3 mismatches, from one of the antisense sequences of SEQ ID NOs: 49-72, 97-120, or 145-168 and wherein the sense strand comprises a nucleotide sequence comprising at least 15 contiguous nucleotides, with 0, 1, 2, or 3 mismatches, from a sense sequence of SEQ ID NOs: 25-48, 73-96 or 121-144 that corresponds to the antisense sequence.

2. (canceled)

3. (canceled)

4. (canceled)

5. The composition of claim 1, wherein the dsRNA agent comprises at least one modified nucleotide.

6. The composition of claim 5, wherein no more than five of the sense strand nucleotides and not more than five of the nucleotides of the antisense strand are unmodified nucleotides, or wherein all of the nucleotides of the sense strand and all of the nucleotides of the antisense strand comprise a modification.

7. (canceled)

8. The composition of claim 5, wherein at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide; and combinations thereof.

9. The composition of claim 5, wherein the sense strand comprises a nucleotide sequence selected from any one of SEQ ID NOs: 25-48, and the antisense strand comprises a nucleotide sequence selected from any one of SEQ ID NOS: 49-72 that corresponds to the antisense sequence.

10. (canceled)

11. The composition of claim 1, wherein the sense strand comprises a nucleotide sequence of SEQ ID NO: 74 and antisense strand comprises a nucleotide sequence of SEQ ID NO: 98.

12. The composition of claim 1, wherein the sense strand comprises a nucleotide sequence of SEQ ID NO: 122 and antisense strand comprises a nucleotide sequence of SEQ ID NO: 146.

13. The composition of claim 1, wherein the sense strand comprises a nucleotide sequence of SEQ ID NO: 26 and the antisense strand comprises a nucleotide sequence of SEQ ID NO: 50.

14. The composition of claim 1, wherein the sense strand comprises a nucleotide sequence of SEQ ID NO: 75 and the antisense strand comprises a nucleotide sequence of SEQ ID NO: 99.

15. The composition of claim 1, wherein the sense strand comprises a nucleotide sequence of SEQ ID NO: 123 and the antisense strand comprises a nucleotide sequence of SEQ ID NO: 147.

16. The composition of claim 1, wherein the sense strand comprises a nucleotide sequence of SEQ ID NO: 27 and the antisense strand comprises a nucleotide sequence of SEQ ID NO: 51.

17. The composition of claim 1, wherein at least one strand comprises a 3′ overhang of at least 2 nucleotides.

18. The composition of claim 1, wherein the double stranded region is 15-30 nucleotide pairs in length, or 17-23 nucleotide pairs in length.

19. (canceled)

20. The composition of claim 1, wherein each strand is about 19-30 nucleotides in length.

21. A pharmaceutical composition comprising the composition of claim 1 and a pharmaceutically acceptable carrier.

22. The pharmaceutical composition of claim 21, wherein the dsRNA agent is conjugated with an antibody that binds to TREM2, optionally via a linker.

23. (canceled)

24. A method for treating or reducing the risk of progression of development or recurrence of an atrial disease, comprising administering the composition of claim 1.

25. The method of claim 24, wherein the atrial disease is an osteopontin-associated disease.

26. The method of claim 24, wherein the atrial disease is atrial fibrillation (AFib).

27. The method of claim 24, wherein the composition or pharmaceutical composition is administered via intravenous injection.

28. The method of claim 24, wherein the subject is a human subject.

29. (canceled)

30. (canceled)

31. (canceled)

32. (canceled)

33. (canceled)


SEQUENCE LISTING (168 sequences)

SEQ ID NO: 1 - RNA, 23 nt, Homo sapiens
  source 1..23: mol_type = genomic RNA
aggactaactacgaccatgagat

SEQ ID NO: 2 - RNA, 23 nt, Homo sapiens
  source 1..23: mol_type = genomic RNA
tggcagtgatttgcttttgcctg

SEQ ID NO: 3 - RNA, 23 nt, Homo sapiens
  source 1..23: mol_type = genomic RNA
ggcagtgatttgcttttgcctgt

SEQ ID NO: 4 - RNA, 23 nt, Homo sapiens
  source 1..23: mol_type = genomic RNA
aagaaactcttccaagcaattcc

SEQ ID NO: 5 - RNA, 23 nt, Homo sapiens
  source 1..23: mol_type = genomic RNA
tcggatgaatctgacgaatctca

SEQ ID NO: 6 - RNA, 23 nt, Homo sapiens
  source 1..23: mol_type = genomic RNA
accattcggatgagtctgatgag

SEQ ID NO: 7 - RNA, 23 nt, Homo sapiens
  source 1..23: mol_type = genomic RNA
gacactttcactccaatcgtccc

SEQ ID NO: 8 - RNA, 23 nt, Homo sapiens
  source 1..23: mol_type = genomic RNA
ggcttatggactgaggtcaaagt

SEQ ID NO: 9 - RNA, 23 nt, Homo sapiens
  source 1..23: mol_type = genomic RNA
gaggtcaaagtctaggagtttcc

SEQ ID NO: 10 - RNA, 23 nt, Homo sapiens
  source 1..23: mol_type = genomic RNA
caggacaacaacggaaagggcag

SEQ ID NO: 11 - RNA, 23 nt, Homo sapiens
  source 1..23: mol_type = genomic RNA
gtcggatgtgatcgatagtcaag

SEQ ID NO: 12 - RNA, 23 nt, Homo sapiens
  source 1..23: mol_type = genomic RNA
tcggatgtgatcgatagtcaagc

SEQ ID NO: 13 - RNA, 23 nt, Homo sapiens
  source 1..23: mol_type = genomic RNA
gtgatcgatagtcaagcaagttc

SEQ ID NO: 14 - RNA, 23 nt, Homo sapiens
  source 1..23: mol_type = genomic RNA
aaggaagatgataggtatctgaa

SEQ ID NO: 15 - RNA, 23 nt, Homo sapiens
  source 1..23: mol_type = genomic RNA
taggtatctgaaattccgaattt

SEQ ID NO: 16 - RNA, 23 nt, Homo sapiens
  source 1..23: mol_type = genomic RNA
cgaatttctcatgaattagagag

SEQ ID NO: 17 - RNA, 23 nt, Homo sapiens
  source 1..23: mol_type = genomic RNA
agggttaagcaggaatactaact

SEQ ID NO: 18 - RNA, 23 nt, Homo sapiens
  source 1..23: mol_type = genomic RNA
caggaatactaactgctcatttc

SEQ ID NO: 19 - RNA, 23 nt, Homo sapiens
  source 1..23: mol_type = genomic RNA
aactgtaacagtctatatcacta

SEQ ID NO: 20 - RNA, 23 nt, Homo sapiens
  source 1..23: mol_type = genomic RNA
ctgtaacagtctatatcactata

SEQ ID NO: 21 - RNA, 23 nt, Homo sapiens
  source 1..23: mol_type = genomic RNA
aacagtctatatcactataccct

SEQ ID NO: 22 - RNA, 23 nt, Homo sapiens
  source 1..23: mol_type = genomic RNA
atcactatacccttttagtttta

SEQ ID NO: 23 - RNA, 23 nt, Homo sapiens
  source 1..23: mol_type = genomic RNA
tcactatacccttttagttttat

SEQ ID NO: 24 - RNA, 23 nt, Homo sapiens
  source 1..23: mol_type = genomic RNA
ttggtgtgaataaatcttgtaac

SEQ ID NO: 25 - RNA, 19 nt, synthetic construct
  source 1..19: mol_type = other RNA
gactaactacgaccatgaa

SEQ ID NO: 26 - RNA, 19 nt, synthetic construct
  source 1..19: mol_type = other RNA
gcagtgatttgcttttgca

SEQ ID NO: 27 - RNA, 19 nt, synthetic construct
  source 1..19: mol_type = other RNA
cagtgatttgcttttgcca

SEQ ID NO: 28 - RNA, 19 nt, synthetic construct
  source 1..19: mol_type = other RNA
gaaactcttccaagcaata

SEQ ID NO: 29 - RNA, 19 nt, synthetic construct
  source 1..19: mol_type = other RNA
ggatgaatctgacgaatca

SEQ ID NO: 30 - RNA, 19 nt, synthetic construct
  source 1..19: mol_type = other RNA
cattcggatgagtctgata

SEQ ID NO: 31 - RNA, 19 nt, synthetic construct
  source 1..19: mol_type = other RNA
cactttcactccaatcgta

SEQ ID NO: 32 - RNA, 19 nt, synthetic construct
  source 1..19: mol_type = other RNA
cttatggactgaggtcaaa

SEQ ID NO: 33 - RNA, 19 nt, synthetic construct
  source 1..19: mol_type = other RNA
ggtcaaagtctaggagtta

SEQ ID NO: 34 - RNA, 19 nt, synthetic construct
  source 1..19: mol_type = other RNA
ggacaacaacggaaaggga

SEQ ID NO: 35 - RNA, 19 nt, synthetic construct
  source 1..19: mol_type = other RNA
cggatgtgatcgatagtca

SEQ ID NO: 36 - RNA, 19 nt, synthetic construct
  source 1..19: mol_type = other RNA
ggatgtgatcgatagtcaa

SEQ ID NO: 37 - RNA, 19 nt, synthetic construct
  source 1..19: mol_type = other RNA
gatcgatagtcaagcaaga

SEQ ID NO: 38 - RNA, 19 nt, synthetic construct
  source 1..19: mol_type = other RNA
ggaagatgataggtatcta

SEQ ID NO: 39 - RNA, 19 nt, synthetic construct
  source 1..19: mol_type = other RNA
ggtatctgaaattccgaaa

SEQ ID NO: 40 - RNA, 19 nt, synthetic construct
  source 1..19: mol_type = other RNA
aatttctcatgaattagaa

SEQ ID NO: 41 - RNA, 19 nt, synthetic construct
  source 1..19: mol_type = other RNA
ggttaagcaggaatactaa

SEQ ID NO: 42 - RNA, 19 nt, synthetic construct
  source 1..19: mol_type = other RNA
ggaatactaactgctcata

SEQ ID NO: 43 - RNA, 19 nt, synthetic construct
  source 1..19: mol_type = other RNA
ctgtaacagtctatatcaa

SEQ ID NO: 44 - RNA, 19 nt, synthetic construct
  source 1..19: mol_type = other RNA
gtaacagtctatatcacta

SEQ ID NO: 45 - RNA, 19 nt, synthetic construct
  source 1..19: mol_type = other RNA
cagtctatatcactataca

SEQ ID NO: 46 - RNA, 19 nt, synthetic construct
  source 1..19: mol_type = other RNA
cactatacccttttagtta

SEQ ID NO: 47 - RNA, 19 nt, synthetic construct
  source 1..19: mol_type = other RNA
actatacccttttagttta

SEQ ID NO: 48 - RNA, 19 nt, synthetic construct
  source 1..19: mol_type = other RNA
ggtgtgaataaatcttgta

SEQ ID NO: 49 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
ttcatggtcgtagttagtcct

SEQ ID NO: 50 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
tgcaaaagcaaatcactgcca

SEQ ID NO: 51 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
tggcaaaagcaaatcactgcc

SEQ ID NO: 52 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
tattgcttggaagagtttctt

SEQ ID NO: 53 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
tgattcgtcagattcatccga

SEQ ID NO: 54 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
tatcagactcatccgaatggt

SEQ ID NO: 55 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
tacgattggagtgaaagtgtc

SEQ ID NO: 56 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
tttgacctcagtccataagcc

SEQ ID NO: 57 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
taactcctagactttgacctc

SEQ ID NO: 58 - RNA, 21 nt, synthetic construct
  source 1..21: mol_type = other RNA
  modified_base 1: mod_base = OTHER; note = thymine
  modified_base 21: mod_base = OTHER; note = phosphorothioate guanine
tccctttccgttgttgtcctg

SEQ ID NO: 59 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
tgactatcgatcacatccgac

SEQ ID NO: 60 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
ttgactatcgatcacatccga

SEQ ID NO: 61 - RNA, 21 nt, synthetic construct
  source 1..21: mol_type = other RNA
  modified_base 1: mod_base = OTHER; note = thymine
  modified_base 21: mod_base = OTHER; note = phosphorothioate cytosine
tcttgcttgactatcgatcac

SEQ ID NO: 62 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
tagatacctatcatcttcctt

SEQ ID NO: 63 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
tttcggaatttcagataccta

SEQ ID NO: 64 - RNA, 21 nt, synthetic construct
  source 1..21: mol_type = other RNA
  modified_base 1: mod_base = OTHER; note = thymine
  modified_base 21: mod_base = OTHER; note = phosphorothioate guanine
ttctaattcatgagaaattcg

SEQ ID NO: 65 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
ttagtattcctgcttaaccct

SEQ ID NO: 66 - RNA, 21 nt, synthetic construct
  source 1..21: mol_type = other RNA
  modified_base 1: mod_base = OTHER; note = thymine
  modified_base 21: mod_base = OTHER; note = phosphorothioate guanine
tatgagcagttagtattcctg

SEQ ID NO: 67 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
ttgatatagactgttacagtt

SEQ ID NO: 68 - RNA, 21 nt, synthetic construct
  source 1..21: mol_type = other RNA
  modified_base 1: mod_base = OTHER; note = thymine
  modified_base 21: mod_base = OTHER; note = phosphorothioate guanine
tagtgatatagactgttacag

SEQ ID NO: 69 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
tgtatagtgatatagactgtt

SEQ ID NO: 70 - RNA, 21 nt, synthetic construct
  source 1..21: mol_type = other RNA
  modified_base 1: mod_base = OTHER; note = thymine
  modified_base 21: mod_base = OTHER; note = phosphorothioate uracil
taactaaaagggtatagtgat

SEQ ID NO: 71 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
taaactaaaagggtatagtga

SEQ ID NO: 72 - RNA, 21 nt, synthetic construct
  source 1..21: mol_type = other RNA
  modified_base 1: mod_base = OTHER; note = thymine
  modified_base 21: mod_base = OTHER; note = phosphorothioate adenosine
tacaagatttattcacaccaa

SEQ ID NO: 73 - RNA, 19 nt, synthetic construct
  source 1..19: mol_type = other RNA
gactaactacgaccatgag

SEQ ID NO: 74 - RNA, 19 nt, synthetic construct
  source 1..19: mol_type = other RNA
gcagtgatttgcttttgcc

SEQ ID NO: 75 - RNA, 19 nt, synthetic construct
  source 1..19: mol_type = other RNA
cagtgatttgcttttgcct

SEQ ID NO: 76 - RNA, 19 nt, synthetic construct
  source 1..19: mol_type = other RNA
gaaactcttccaagcaatt

SEQ ID NO: 77 - RNA, 19 nt, synthetic construct
  source 1..19: mol_type = other RNA
ggatgaatctgacgaatct

SEQ ID NO: 78 - RNA, 19 nt, synthetic construct
  source 1..19: mol_type = other RNA
cattcggatgagtctgatg

SEQ ID NO: 79 - RNA, 19 nt, synthetic construct
  source 1..19: mol_type = other RNA
cactttcactccaatcgtc

SEQ ID NO: 80 - RNA, 19 nt, synthetic construct
  source 1..19: mol_type = other RNA
cttatggactgaggtcaaa

SEQ ID NO: 81 - RNA, 19 nt, synthetic construct
  source 1..19: mol_type = other RNA
ggtcaaagtctaggagttt

SEQ ID NO: 82 - RNA, 19 nt, synthetic construct
  source 1..19: mol_type = other RNA
ggacaacaacggaaagggc

SEQ ID NO: 83 - RNA, 19 nt, synthetic construct
  source 1..19: mol_type = other RNA
cggatgtgatcgatagtca

SEQ ID NO: 84 - RNA, 19 nt, synthetic construct
  source 1..19: mol_type = other RNA
ggatgtgatcgatagtcaa

SEQ ID NO: 85 - RNA, 19 nt, synthetic construct
  source 1..19: mol_type = other RNA
gatcgatagtcaagcaagt

SEQ ID NO: 86 - RNA, 19 nt, synthetic construct
  source 1..19: mol_type = other RNA
ggaagatgataggtatctg

SEQ ID NO: 87 - RNA, 19 nt, synthetic construct
  source 1..19: mol_type = other RNA
ggtatctgaaattccgaat

SEQ ID NO: 88 - RNA, 19 nt, synthetic construct
  source 1..19: mol_type = other RNA
aatttctcatgaattagag

SEQ ID NO: 89 - RNA, 19 nt, synthetic construct
  source 1..19: mol_type = other RNA
ggttaagcaggaatactaa

SEQ ID NO: 90 - RNA, 19 nt, synthetic construct
  source 1..19: mol_type = other RNA
ggaatactaactgctcatt

SEQ ID NO: 91 - RNA, 19 nt, synthetic construct
  source 1..19: mol_type = other RNA
ctgtaacagtctatatcac

SEQ ID NO: 92 - RNA, 19 nt, synthetic construct
  source 1..19: mol_type = other RNA
gtaacagtctatatcacta

SEQ ID NO: 93 - RNA, 19 nt, synthetic construct
  source 1..19: mol_type = other RNA
cagtctatatcactatacc

SEQ ID NO: 94 - RNA, 19 nt, synthetic construct
  source 1..19: mol_type = other RNA
cactatacccttttagttt

SEQ ID NO: 95 - RNA, 19 nt, synthetic construct
  source 1..19: mol_type = other RNA
actatacccttttagtttt

SEQ ID NO: 96 - RNA, 19 nt, synthetic construct
  source 1..19: mol_type = other RNA
ggtgtgaataaatcttgta

SEQ ID NO: 97 - RNA, 19 nt, synthetic construct
  source 1..19: mol_type = other RNA
ctcatggtcgtagttagtc

SEQ ID NO: 98 - RNA, 19 nt, synthetic construct
  source 1..19: mol_type = other RNA
ggcaaaagcaaatcactgc

SEQ ID NO: 99 - RNA, 19 nt, synthetic construct
  source 1..19: mol_type = other RNA
aggcaaaagcaaatcactg

SEQ ID NO: 100 - RNA, 19 nt, synthetic construct
  source 1..19: mol_type = other RNA
aattgcttggaagagtttc

SEQ ID NO: 101 - RNA, 19 nt, synthetic construct
  source 1..19: mol_type = other RNA
agattcgtcagattcatcc

SEQ ID NO: 102 - RNA, 19 nt, synthetic construct
  source 1..19: mol_type = other RNA
catcagactcatccgaatg

SEQ ID NO: 103 - RNA, 19 nt, synthetic construct
  source 1..19: mol_type = other RNA
gacgattggagtgaaagtg

SEQ ID NO: 104 - RNA, 19 nt, synthetic construct
  source 1..19: mol_type = other RNA
tttgacctcagtccataag

SEQ ID NO: 105 - RNA, 19 nt, synthetic construct
  source 1..19: mol_type = other RNA
aaactcctagactttgacc

SEQ ID NO: 106 - RNA, 19 nt, synthetic construct
  source 1..19: mol_type = other RNA
gccctttccgttgttgtcc

SEQ ID NO: 107 - RNA, 19 nt, synthetic construct
  source 1..19: mol_type = other RNA
tgactatcgatcacatccg

SEQ ID NO: 108 - RNA, 19 nt, synthetic construct
  source 1..19: mol_type = other RNA
ttgactatcgatcacatcc

SEQ ID NO: 109 - RNA, 19 nt, synthetic construct
  source 1..19: mol_type = other RNA
acttgcttgactatcgatc

SEQ ID NO: 110 - RNA, 19 nt, synthetic construct
  source 1..19: mol_type = other RNA
cagatacctatcatcttcc

SEQ ID NO: 111 - RNA, 19 nt, synthetic construct
  source 1..19: mol_type = other RNA
attcggaatttcagatacc

SEQ ID NO: 112 - RNA, 19 nt, synthetic construct
  source 1..19: mol_type = other RNA
ctctaattcatgagaaatt

SEQ ID NO: 113 - RNA, 19 nt, synthetic construct
  source 1..19: mol_type = other RNA
ttagtattcctgcttaacc

SEQ ID NO: 114 - RNA, 19 nt, synthetic construct
  source 1..19: mol_type = other RNA
aatgagcagttagtattcc

SEQ ID NO: 115 - RNA, 19 nt, synthetic construct
  source 1..19: mol_type = other RNA
gtgatatagactgttacag

SEQ ID NO: 116 - RNA, 19 nt, synthetic construct
  source 1..19: mol_type = other RNA
tagtgatatagactgttac

SEQ ID NO: 117 - RNA, 19 nt, synthetic construct
  source 1..19: mol_type = other RNA
ggtatagtgatatagactg

SEQ ID NO: 118 - RNA, 19 nt, synthetic construct
  source 1..19: mol_type = other RNA
aaactaaaagggtatagtg

SEQ ID NO: 119 - RNA, 19 nt, synthetic construct
  source 1..19: mol_type = other RNA
aaaactaaaagggtatagt

SEQ ID NO: 120 - RNA, 19 nt, synthetic construct
  source 1..19: mol_type = other RNA
tacaagatttattcacacc

SEQ ID NO: 121 - RNA, 19 nt, synthetic construct
  source 1..19: mol_type = other RNA
gactaactacgaccatgaa

SEQ ID NO: 122 - RNA, 19 nt, synthetic construct
  source 1..19: mol_type = other RNA
gcagtgatttgcttttgca

SEQ ID NO: 123 - RNA, 19 nt, synthetic construct
  source 1..19: mol_type = other RNA
cagtgatttgcttttgcca

SEQ ID NO: 124 - RNA, 19 nt, synthetic construct
  source 1..19: mol_type = other RNA
gaaactcttccaagcaata

SEQ ID NO: 125 - RNA, 19 nt, synthetic construct
  source 1..19: mol_type = other RNA
ggatgaatctgacgaatca

SEQ ID NO: 126 - RNA, 19 nt, synthetic construct
  source 1..19: mol_type = other RNA
cattcggatgagtctgata

SEQ ID NO: 127 - RNA, 19 nt, synthetic construct
  source 1..19: mol_type = other RNA
cactttcactccaatcgta

SEQ ID NO: 128 - RNA, 19 nt, synthetic construct
  source 1..19: mol_type = other RNA
cttatggactgaggtcaaa

SEQ ID NO: 129 - RNA, 19 nt, synthetic construct
  source 1..19: mol_type = other RNA
ggtcaaagtctaggagtta

SEQ ID NO: 130 - RNA, 19 nt, synthetic construct
  source 1..19: mol_type = other RNA
ggacaacaacggaaaggga

SEQ ID NO: 131 - RNA, 19 nt, synthetic construct
  source 1..19: mol_type = other RNA
cggatgtgatcgatagtca

SEQ ID NO: 132 - RNA, 19 nt, synthetic construct
  source 1..19: mol_type = other RNA
ggatgtgatcgatagtcaa

SEQ ID NO: 133 - RNA, 19 nt, synthetic construct
  source 1..19: mol_type = other RNA
gatcgatagtcaagcaaga

SEQ ID NO: 134 - RNA, 19 nt, synthetic construct
  source 1..19: mol_type = other RNA
ggaagatgataggtatcta

SEQ ID NO: 135 - RNA, 19 nt, synthetic construct
  source 1..19: mol_type = other RNA
ggtatctgaaattccgaaa

SEQ ID NO: 136 - RNA, 19 nt, synthetic construct
  source 1..19: mol_type = other RNA
aatttctcatgaattagaa

SEQ ID NO: 137 - RNA, 19 nt, synthetic construct
  source 1..19: mol_type = other RNA
ggttaagcaggaatactaa

SEQ ID NO: 138 - RNA, 19 nt, synthetic construct
  source 1..19: mol_type = other RNA
ggaatactaactgctcata

SEQ ID NO: 139 - RNA, 19 nt, synthetic construct
  source 1..19: mol_type = other RNA
ctgtaacagtctatatcaa

SEQ ID NO: 140 - RNA, 19 nt, synthetic construct
  source 1..19: mol_type = other RNA
gtaacagtctatatcacta

SEQ ID NO: 141 - RNA, 19 nt, synthetic construct
  source 1..19: mol_type = other RNA
cagtctatatcactataca

SEQ ID NO: 142 - RNA, 19 nt, synthetic construct
  source 1..19: mol_type = other RNA
cactatacccttttagtta

SEQ ID NO: 143 - RNA, 19 nt, synthetic construct
  source 1..19: mol_type = other RNA
actatacccttttagttta

SEQ ID NO: 144 - RNA, 19 nt, synthetic construct
  source 1..19: mol_type = other RNA
ggtgtgaataaatcttgta

SEQ ID NO: 145 - RNA, 21 nt, synthetic construct
  source 1..21: mol_type = other RNA
ttcatggtcgtagttagtcct

SEQ ID NO: 146 - RNA, 21 nt, synthetic construct
  source 1..21: mol_type = other RNA
tgcaaaagcaaatcactgcca

SEQ ID NO: 147 - RNA, 21 nt, synthetic construct
  source 1..21: mol_type = other RNA
tggcaaaagcaaatcactgcc

SEQ ID NO: 148 - RNA, 21 nt, synthetic construct
  source 1..21: mol_type = other RNA
tattgcttggaagagtttctt

SEQ ID NO: 149 - RNA, 21 nt, synthetic construct
  source 1..21: mol_type = other RNA
tgattcgtcagattcatccga

SEQ ID NO: 150 - RNA, 21 nt, synthetic construct
  source 1..21: mol_type = other RNA
tatcagactcatccgaatggt

SEQ ID NO: 151 - RNA, 21 nt, synthetic construct
  source 1..21: mol_type = other RNA
tacgattggagtgaaagtgtc

SEQ ID NO: 152 - RNA, 21 nt, synthetic construct
  source 1..21: mol_type = other RNA
tttgacctcagtccataagcc

SEQ ID NO: 153 - RNA, 21 nt, synthetic construct
  source 1..21: mol_type = other RNA
taactcctagactttgacctc

SEQ ID NO: 154 - RNA, 21 nt, synthetic construct
  source 1..21: mol_type = other RNA
tccctttccgttgttgtcctg

SEQ ID NO: 155 - RNA, 21 nt, synthetic construct
  source 1..21: mol_type = other RNA
tgactatcgatcacatccgac

SEQ ID NO: 156 - RNA, 21 nt, synthetic construct
  source 1..21: mol_type = other RNA
ttgactatcgatcacatccga

SEQ ID NO: 157 - RNA, 21 nt, synthetic construct
  source 1..21: mol_type = other RNA
tcttgcttgactatcgatcac

SEQ ID NO: 158 - RNA, 21 nt, synthetic construct
  source 1..21: mol_type = other RNA
tagatacctatcatcttcctt

SEQ ID NO: 159 - RNA, 21 nt, synthetic construct
  source 1..21: mol_type = other RNA
tttcggaatttcagataccta

SEQ ID NO: 160 - RNA, 21 nt, synthetic construct
  source 1..21: mol_type = other RNA
ttctaattcatgagaaattcg

SEQ ID NO: 161 - RNA, 21 nt, synthetic construct
  source 1..21: mol_type = other RNA
ttagtattcctgcttaaccct

SEQ ID NO: 162 - RNA, 21 nt, synthetic construct
  source 1..21: mol_type = other RNA
tatgagcagttagtattcctg

SEQ ID NO: 163 - RNA, 21 nt, synthetic construct
  source 1..21: mol_type = other RNA
ttgatatagactgttacagtt

SEQ ID NO: 164 - RNA, 21 nt, synthetic construct
  source 1..21: mol_type = other RNA
tagtgatatagactgttacag

SEQ ID NO: 165 - RNA, 21 nt, synthetic construct
  source 1..21: mol_type = other RNA
tgtatagtgatatagactgtt

SEQ ID NO: 166 - RNA, 21 nt, synthetic construct
  source 1..21: mol_type = other RNA
taactaaaagggtatagtgat

SEQ ID NO: 167 - RNA, 21 nt, synthetic construct
  source 1..21: mol_type = other RNA
taaactaaaagggtatagtga

SEQ ID NO: 168 - RNA, 21 nt, synthetic construct
  source 1..21: mol_type = other RNA
tacaagatttattcacaccaa